(19) United States
(12) Patent Application Publication
(10) Pub. No.: US 2010/0069647 A1
(43) Pub. Date: Mar. 18, 2010

(54) Carbazole Derivative, Light-Emitting Element Material, Light-Emitting Element, and Light-Emitting Device

(75) Inventors: Hiroki Suzuki, Atsugi (JP); Sachiko Kawakami, Atsugi (JP); Nobuharu Ohsawa, Zama (JP); Tsunenori Suzuki, Yokohama (JP); Satoshi Seo, Kawasaki (JP)

Correspondence Address: COOK ALEX LTD, SUITE 2850, 200 WEST ADAMS STREET, CHICAGO, IL, 60606, US

(73) Assignee: Semiconductor Energy Laboratory Co., Ltd.

(21) Appl. No.: 12/498,763

(22) Filed: Jul. 7, 2009

(30) Foreign Application Priority Data
Jul. 8, 2008 (JP) 2008-177836
Jul. 11, 2008 (JP) 2008-181752
Sep. 19, 2008 (JP) 2008-240355
Sep. 19, 2008 (JP) 2008-240529
Dec. 25, 2008 (JP) 2008-331007

(51) Int. Cl. C07D 209/86

(52) U.S. Cl. 548/445

(57) ABSTRACT

An object is to provide a carbazole derivative which has a wide band gap and with which excellent blue color purity is obtained. In addition, another object is to provide highly reliable light-emitting elements, light-emitting devices, lighting devices, and electronic devices in which the carbazole derivative is used. Carbazole derivatives represented by the general formulas (1), (P1), and (M1) are provided. Further, light-emitting elements, light-emitting devices, and electronic devices which are formed using the carbazole derivative represented any of the general formulas (1), (P1), and (M1) are provided.

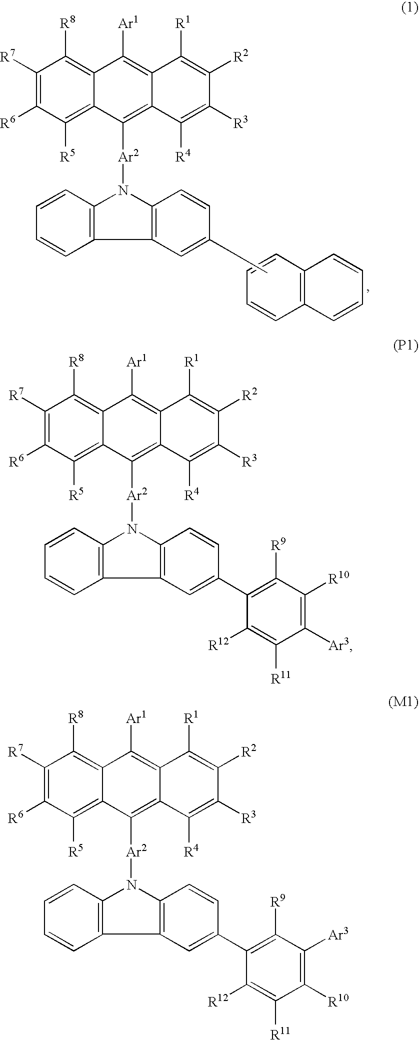

### TECHNICAL FIELD

The present invention relates to carbazole derivatives. In addition, the present invention relates to light-emitting element materials, light-emitting elements, and electronic devices in which the carbazole derivative is used.

### BACKGROUND ART

A light-emitting element in which a light-emitting material is used has features of thinness and lightweight, fast response, direct-current low-voltage drive, and the like, and is expected to be applied to next-generation flat panel displays. It is said that a light-emitting device in which light-emitting elements are arranged in a matrix has an advantage in wide viewing angle and excellent visibility over conventional liquid crystal display devices.

A light-emitting element is said to have the following light-emission mechanism: when voltage is applied to a light-emitting layer interposed between a pair of electrodes, electrons injected from a cathode and holes injected from an anode are recombined at an emission center of the light-emitting layer to form molecular excitons, and the molecular excitons release energy to emit light when returning to a ground state. As excited states, a singlet excited state and a triplet excited state are known, and it is believed that light emission is possible through either of the excited states.

The emission wavelength of a light-emitting element is determined by energy difference between a ground state and an excited state of light-emitting molecules included in the light-emitting element, that is, a band gap of the light-emitting molecules. Therefore, various emission colors can be obtained by contravening structures of light-emitting molecules. By using light-emitting elements capable of emitting red light, blue light, and green light, which are the three primary colors of light, a full-color light-emitting device can be manufactured.

However, there is a problem in a full-color light-emitting device that a light-emitting element with excellent color purity can not always be manufactured easily. This is because it is difficult to realize a light-emitting element with high reliability and excellent color purity, although light-emitting elements for red, blue, and green with excellent color purity are needed for manufacturing a light-emitting device having excellent color reproducibility. As a result of recent development of materials, high reliability and excellent color purity of light-emitting elements for red and green have been achieved. However, in particular, sufficient reliability and color purity of a light-emitting element for blue has not been realized, and many researches are still in progress (for example, see Patent Document 1).

### [Patent Document]

### [Patent Document 1] Japanese Published Patent Application No. 2003-31371

### DISCLOSURE OF INVENTION

The present invention has been made in view of the above problems. An object of the present invention is to provide a carbazole derivative which has a wide band gap and with which excellent blue color purity is obtained. In addition, another object is to provide highly reliable light-emitting elements, light-emitting devices, and electronic devices in which the carbazole derivative is used.

An aspect of the present invention is a carbazole derivative represented by the general formula (1).

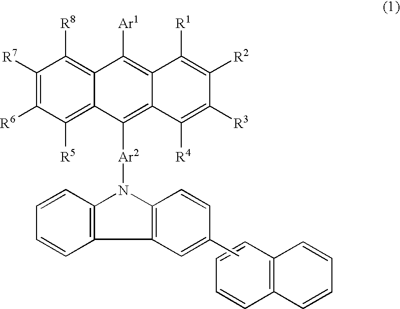

In the formula, Ar¹ represents an aryl group having 6 to 13 carbon atoms, Ar² represents an arylene group having 6 to 13 carbon atoms, and R¹ to R⁸ independently represent hydrogen or an alkyl group having 1 to 4 carbon atoms. Ar¹ and Ar² may independently have a substituent or substituents: when Ar¹ and Ar² independently have two or more substituents, the substituents may be bonded to each other to form a ring structure, and when one carbon atom of any of Ar¹ and Ar² has two substituents, the substituents may be bonded to each other to form a spiro ring structure.

An aspect of the present invention is a carbazole derivative represented by the general formula (2).

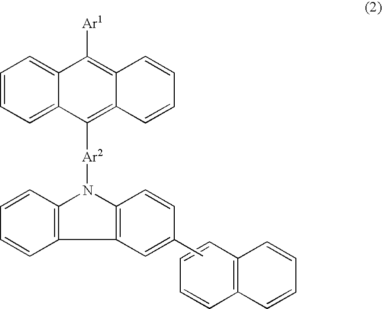

In the formula, Ar¹ represents an aryl group having 6 to 13 carbon atoms and Ar² represents an arylene group having 6 to 13 carbon atoms. Ar¹ and Ar² may independently have a substituent or substituents: when Ar¹ and Ar² independently have two or more substituents, the substituents may be bonded to each other to form a ring structure, and when one carbon atom of any of Ar¹ and Ar² has two substituents, the substituents may be bonded to each other to form a spiro ring structure.

An aspect of the present invention is a carbazole derivative represented by the general formula (3).

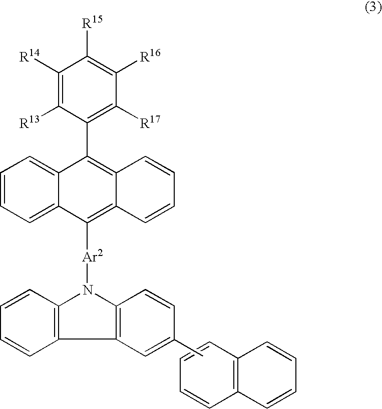

In the formula, Ar² represents an arylene group having 6 to 13 carbon atoms and R¹³ to R¹⁷ independently represent hydrogen, an aryl group having 6 to 10 carbon atoms, an alkyl group having 1 to 4 carbon atoms, or a haloalkyl group having 1 carbon atom. Ar² and R¹³ to R¹⁷ may independently have a substituent or substituents: when Ar² and R¹³ to R¹⁷ independently have two or more substituents, the substituents may be bonded to each other to form a ring structure, and when one carbon atom of any of Ar² and R¹³ to R¹⁷ has two substituents, the substituents may be bonded to each other to form a spiro ring structure.

An aspect of the present invention is a carbazole derivative represented by the general formula (4).

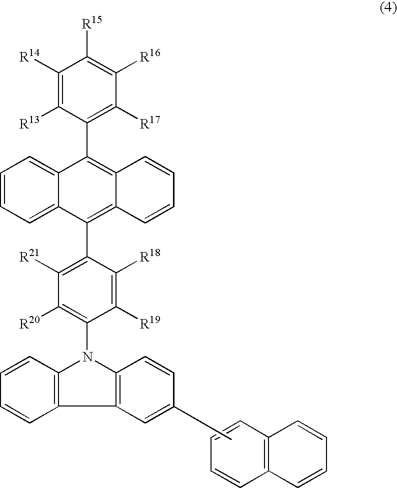

In the formula, R¹³ to R¹⁷ independently represent hydrogen, an aryl group having 6 to 10 carbon atoms, an alkyl group having 1 to 4 carbon atoms, or a haloalkyl group having 1 carbon atom; and R¹⁸ to R²¹ independently represent hydrogen or an alkyl group having 1 to 4 carbon atoms. R¹³ to R¹⁷ may independently have a substituent or substituents: when R¹³ to R¹⁷ independently have two or more substituents, the substituents may be bonded to each other to form a ring structure, and when one carbon atom of any of R¹³ to R¹⁷ has two substituents, the substituents may be bonded to each other to form a spiro ring structure.

Another aspect of the present invention is a carbazole derivative represented by the structural formula (101).

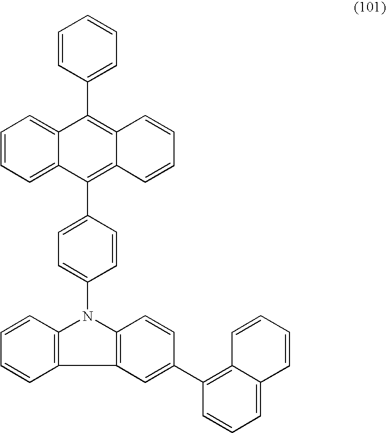

Another aspect of the present invention is a carbazole derivative represented by the structural formula (201).

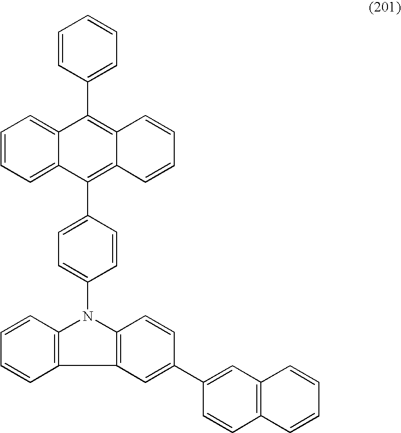

An aspect of the present invention is a carbazole derivative represented by the general formula (P1).

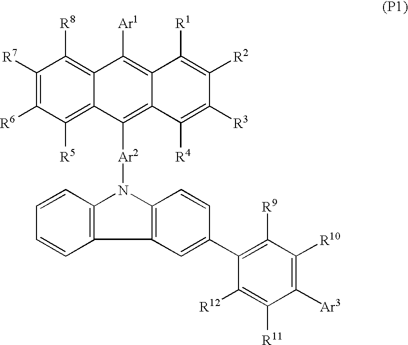

In the formula, R¹ to R¹² independently represent hydrogen or an alkyl group having 1 to 4 carbon atoms, Ar¹ and Ar³ independently represent an aryl group having 6 to 13 carbon atoms, and Ar² represents an arylene group having 6 to 13 carbon atoms. The aryl group having 6 to 13 carbon atoms and the arylene group having 6 to 13 carbon atoms may independently have a substituent or substituents: when the aryl group having 6 to 13 carbon atoms and the arylene group having 6 to 13 carbon atoms independently have two or more substituents, the substituents may be bonded to each other to form a ring structure, and when one carbon atom of any of the aryl group having 6 to 13 carbon atoms and the arylene group having 6 to 13 carbon atoms has two substituents, the substituents may be bonded to each other to form a spiro ring structure. In addition, a substituent of Ar³ may be bonded to R¹⁰ or R¹¹ to form a ring structure, which structure may be a spiro ring structure.

An aspect of the present invention is a carbazole derivative represented by the general formula (P2).

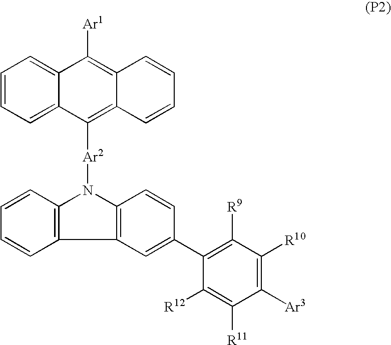

In the formula, R⁹ to R¹² independently represent hydrogen or an alkyl group having 1 to 4 carbon atoms, Ar¹ and Ar³ independently represent an aryl group having 6 to 13 carbon atoms, and Ar² represents an arylene group having 6 to 13 carbon atoms. The aryl group having 6 to 13 carbon atoms and the arylene group having 6 to 13 carbon atoms may independently have a substituent or substituents: when the aryl group having 6 to 13 carbon atoms and the arylene group having 6 to 13 carbon atoms independently have two or more substituents, the substituents may be bonded to each other to form a ring structure, and when one carbon atom of any of the aryl group having 6 to 13 carbon atoms and the arylene group having 6 to 13 carbon atoms has two substituents, the substituents may be bonded to each other to form a spiro ring structure. In addition, a substituent of Ar³ may be bonded to R¹⁰ or R¹¹ to form a ring structure which may be a spiro ring structure.

An aspect of the present invention is a carbazole derivative represented by the general formula (P3).

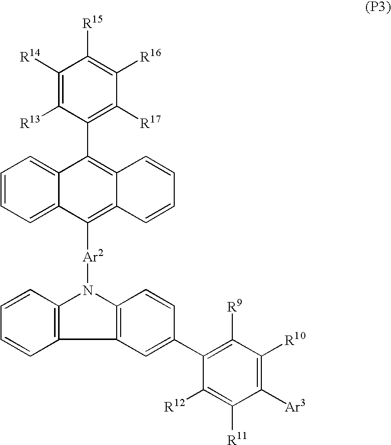

In the formula, R⁹ to R¹² independently represent hydrogen or an alkyl group having 6 to 10 carbon atoms; R¹³ to R¹⁷ independently represent hydrogen, an alkyl group having 1 to 4 carbon atoms, or an aryl group having 6 to 10 carbon atoms; Ar² represents an arylene group having 6 to 13 carbon atoms; and Ar³ represents an aryl group having 6 to 13 carbon atoms. The aryl group having 6 to 10 carbon atoms, the arylene group having 6 to 13 carbon atoms, and the aryl group having 6 to 13 carbon atoms may independently have a substituent or substituents: when the aryl group having 6 to 10 carbon atoms, the arylene group having 6 to 13 carbon atoms, and the aryl group having 6 to 13 carbon atoms independently have two or more substituents, the substituents may be bonded to each other to form a ring structure, and when one carbon atom of any of the aryl group having 6 to 10 carbon atoms, the arylene group having 6 to 13 carbon atoms, and the aryl group having 6 to 13 carbon atoms has two substituents, the substituents may be bonded to each other to form a spiro ring structure. In addition, a substituent of Ar³ may be bonded to R¹⁰ or R¹¹ to form a ring structure which may be a spiro ring structure.

An aspect of the present invention is a carbazole derivative represented by the general formula (P4).

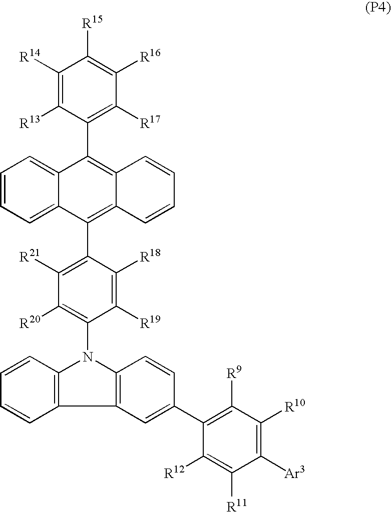

In the formula, R⁹ to R¹² and R¹⁸ to R²¹ independently represent hydrogen or an alkyl group having 1 to 4 carbon atoms; R¹³ to R¹⁷ independently represent hydrogen, an alkyl group having 1 to 4 carbon atoms, or an aryl group having 6 to 10 carbon atoms; and Ar³ represents an aryl group having 6 to 13 carbon atoms. The aryl group having 6 to 10 carbon atoms and the aryl group having 6 to 13 carbon atoms may independently have a substituent or substituents: when the aryl group having 6 to 10 carbon atoms and the aryl group having 6 to 13 carbon atoms independently have two or more substituents, the substituents may be bonded to each other to form a ring structure, and when one carbon atom of any of the aryl group having 6 to 10 carbon atoms and the aryl group having 6 to 13 carbon atoms has two substituents, the substituents may be bonded to each other to form a spiro ring structure. In addition, a substituent of Ar³ may be bonded to R¹⁰ or R¹¹ to form a ring structure which may be a spiro ring structure.

Another aspect of the present invention is a carbazole derivative represented by the structural formula (31).

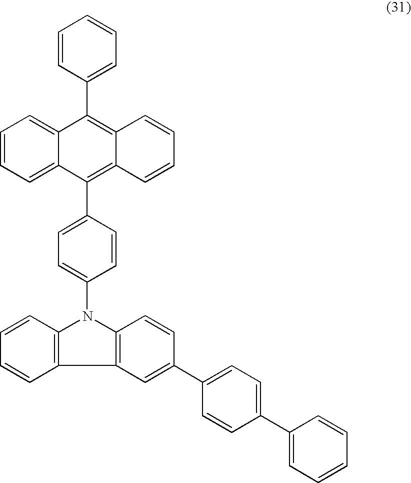

Another aspect of the present invention is a carbazole derivative represented by the structural formula (63).

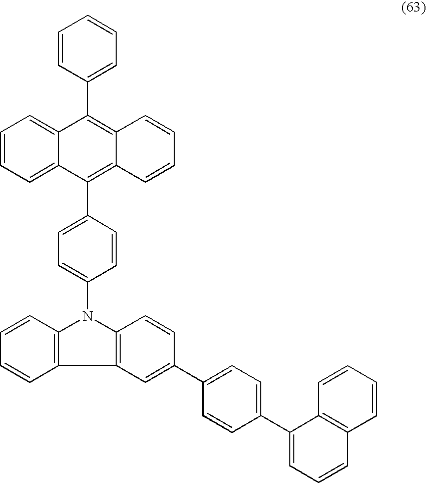

Another aspect of the present invention is a carbazole derivative represented by the structural formula (76).

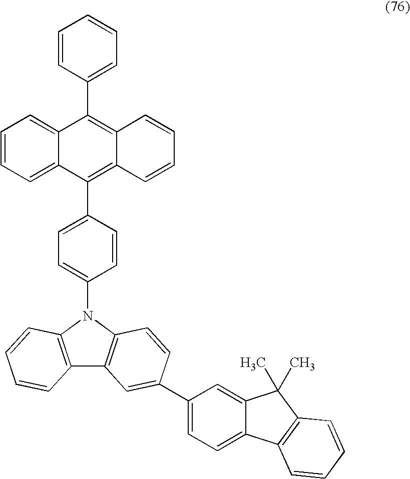

An aspect of the present invention is a carbazole derivative represented by the general formula (M1).

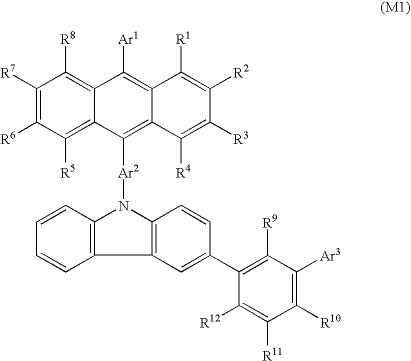

In the formula, R¹ to R¹² independently represent hydrogen or an alkyl group having 1 to 4 carbon atoms, Ar¹ and Ar³ independently represent an aryl group having 6 to 13 carbon atoms, and Ar² represents an arylene group having 6 to 13 carbon atoms. The aryl group having 6 to 13 carbon atoms and the arylene group having 6 to 13 carbon atoms may independently have a substituent or substituents: when the aryl group having 6 to 13 carbon atoms and the arylene group having 6 to 13 carbon atoms independently have two or more substituents, the substituents may be bonded to each other to form a ring structure, and when one carbon atom of any of the aryl group having 6 to 13 carbon atoms and the arylene group having 6 to 13 carbon atoms has two substituents, the substituents may be bonded to each other to form a spiro ring structure. In addition, a substituent of Ar³ may be bonded to R⁹ or R¹⁰ to form a ring structure which may be a spiro ring structure.

Another aspect of the present invention is a carbazole derivative represented by the general formula (M2).

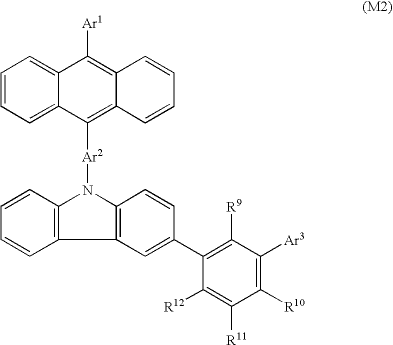

In the formula, R⁹ to R¹² independently represent hydrogen or an alkyl group having 1 to 4 carbon atoms, Ar¹ and Ar³ independently represent an aryl group having 6 to 13 carbon atoms, and Ar² represents an arylene group having 6 to 13 carbon atoms. The aryl group having 6 to 13 carbon atoms and the arylene group having 6 to 13 carbon atoms may independently have a substituent or substituents: when the aryl group having 6 to 13 carbon atoms and the arylene group having 6 to 13 carbon atoms independently have two or more substituents, the substituents may be bonded to each other to form a ring structure, and when one carbon atom of any of the aryl group having 6 to 13 carbon atoms and the arylene group having 6 to 13 carbon atoms has two substituents, the substituents may be bonded to each other to form a spiro ring structure. In addition, a substituent of Ar³ may be bonded to R⁹ or R¹⁰ to form a ring structure which may be a spiro ring structure.

An aspect of the present invention is a carbazole derivative represented by the general formula (M3).

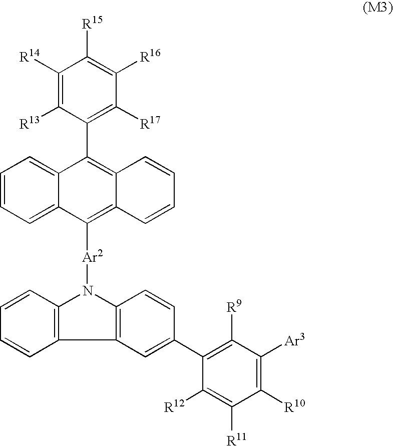

In the formula, R⁹ to R¹² independently represent hydrogen or an alkyl group having 1 to 4 carbon atoms; R¹³ to R¹⁷ independently represent hydrogen, an alkyl group having 1 to 4 carbon atoms, or an aryl group having 6 to 10 carbon atoms; Ar² represents an arylene group having 6 to 13 carbon atoms; and Ar³ represents an aryl group having 6 to 13 carbon atoms. The aryl group having 6 to 10 carbon atoms, the arylene group having 6 to 13 carbon atoms, and the aryl group having 6 to 13 carbon atoms may independently have a substituent or substituents: when the aryl group having 6 to 10 carbon atoms, the arylene group having 6 to 13 carbon atoms, and the aryl group having 6 to 13 carbon atoms independently have two or more substituents, the substituents may be bonded to each other to form a ring structure, and when one carbon atom of any of the aryl group having 6 to 10 carbon atoms, the arylene group having 6 to 13 carbon atoms, and the aryl group having 6 to 13 carbon atoms has two substituents, the substituents may be bonded to each other to form a spiro ring structure. In addition, a substituent of Ar³ may be bonded to R⁹ or R¹⁰ to form a ring structure which may be a spiro ring structure.

An aspect of the present invention is a carbazole derivative represented by the general formula (M4).

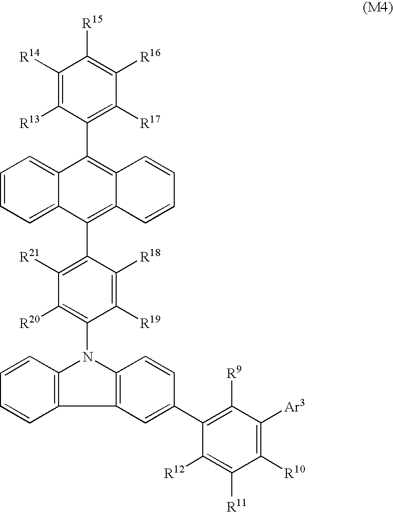

In the formula, R⁹ to R¹² and R¹⁸ to R²¹ independently represent hydrogen or an alkyl group having 1 to 4 carbon atoms; R¹³ to R¹⁷ independently represent hydrogen, an alkyl group having 1 to 4 carbon atoms, or an aryl group having 6 to 10 carbon atoms; and Ar³ represents an aryl group having 6 to 13 carbon atoms. The aryl group having 6 to 10 carbon atoms and the aryl group having 6 to 13 carbon atoms may independently have a substituent or substituents: when the aryl group having 6 to 10 carbon atoms and the aryl group having 6 to 13 carbon atoms independently have two or more substituents, the substituents may be bonded to each other to form a ring structure, and when one carbon atom of any of the aryl group having 6 to 10 carbon atoms and the aryl group having 6 to 13 carbon atoms has two substituents, the substituents may be bonded to each other to form a spiro ring structure. In addition, a substituent of Ar³ may be bonded to R⁹ or R¹⁰ to form a ring structure which may be a spiro ring structure.

Another aspect of the present invention is a carbazole derivative represented by the structural formula (331).

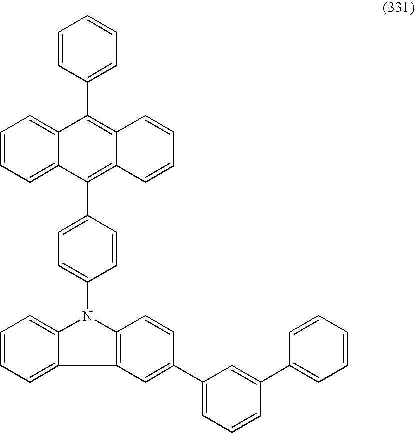

Further, another aspect of the present invention is a light-emitting element material including any of the above carbazole derivatives.

Further, another aspect of the present invention is a light-emitting element in which any of the above carbazole derivatives is used; specifically, a light-emitting element in which any of the above carbazole derivatives is included between a pair of electrode.

In addition, another aspect of the present invention is a light-emitting element which includes a light-emitting layer containing any of the above carbazole derivatives between a pair of electrodes.

In addition, the light-emitting device of the present invention includes a light-emitting element and a controller for controlling light emission of the light-emitting element. The light-emitting element includes a layer containing a light-emitting substance between a pair of electrodes. The layer containing a light-emitting substance contains any of the above carbazole derivatives. Note that a light-emitting device in this specification refers to an image display device, a light-emitting device, or a light source (e.g., a lighting device). In addition, the light-emitting device also includes the following modules in its category: a module in which a panel is connected to a connector such as a flexible printed circuit (FPC), a tape automated bonding (TAB) tape, or a tape carrier package (TCP); a module in which a printed wiring board is provided on the tip of a TAB tape or a TCP; and a module in which an integrated circuit (IC) is directly mounted onto a light-emitting element by chip on glass (COG) method.

The present invention also covers an electronic device which includes a light-emitting element of the present invention in its display portion. Accordingly, the electronic device of the present invention includes a display portion which is provided with the above light-emitting element and a controller for controlling light emission of the light-emitting element.

### EFFECT OF THE INVENTION

A carbazole derivative according to one mode of the present invention has a large band gap, and therefore light emission with a relatively short wavelength can be obtained with the carbazole derivative. Accordingly, blue-light emission with good color purity can be obtained with the carbazole derivative. In addition, the carbazole derivative according to one mode of the present invention has high electrochemical stability.

Further, by adding, to a layer formed by the carbazole derivative according to one mode of the present invention, a light-emitting material (hereinafter, referred to as a dopant) having a smaller band gap than the carbazole derivative, light emission from the dopant can be obtained. Here, since the carbazole derivative according to one mode of the present invention has a large band gap, if a dopant which emits light with a relatively short wavelength is used, light emission not from the carbazole derivative but from the dopant can be sufficiently obtained. In specific, by using a light-emitting material having an emission peak at around 450 nm to 470 nm which exhibits blue-light emission with excellent color purity as a dopant, a light-emitting element which can exhibit blue-light emission with good color purity can be obtained.

Further, by manufacturing a light-emitting element in which the carbazole derivative according to one mode of the present invention is added to a layer formed from a material (hereinafter, referred to as a host) having a larger band gap than the carbazole derivative, light emission from the carbazole derivative according to one mode of the present invention can be obtained. In other words, the carbazole derivative according to one mode of the present invention also functions as a dopant. Since the carbazole derivative according to one mode of the present invention has a large band gap and light emission with a relatively short wavelength can be obtained, a light-emitting element which can exhibit blue-light emission with good color purity can be manufactured by using the carbazole derivative.

The carbazole derivative according to one mode of the present invention has a wide band gap and is a bipolar material having a high electron- and hole-injecting and transporting properties. Therefore, by using the carbazole derivative according to one mode of the present invention for a light-emitting element, a highly reliable light-emitting element with good carrier balance can be obtained.

Further, a light-emitting element according to one mode of the present invention which includes any of the above carbazole derivatives can exhibit blue-light emission with excellent color purity. In addition, the light-emitting element according to one mode of the present invention which includes any of the above carbazole derivatives has high reliability.

Further, a light-emitting device according to one mode of the present invention which includes the above light-emitting element has high color reproducibility and display quality. The light-emitting device according to one mode of the present invention which includes the above light-emitting element has high reliability.

Further, an electronic device according to one mode of the present invention which includes the above light-emitting element has high color reproducibility and display quality. In addition, the electronic device according to one mode of the present invention which includes the above light-emitting element has high reliability.

### BRIEF DESCRIPTION OF DRAWING

In the accompanying drawings:

FIGS. 1A to 1C each illustrate a light-emitting element;

FIG. 2 illustrates a light-emitting element;

FIG. 3 illustrates a light-emitting element;

FIGS. 4A and 4B illustrate a light-emitting device;

FIGS. 5A and 5B illustrate a light-emitting device;

FIGS. 6A to 6F each illustrate an electronic device;

FIG. 7 illustrates an electronic device;

FIGS. 8A and 8B each illustrate a lighting device;

FIG. 9 illustrates lighting devices;

FIGS. 10A and 10B are the ¹H-NMR charts of CzPAαN;

FIG. 11 illustrates an absorption spectrum of CzPAαN included in a toluene solution;

FIG. 12 illustrates an absorption spectrum of a thin film of CzPAαN;

FIG. 13 illustrates an emission spectrum of CzPAαN included in the toluene solution;

FIG. 14 illustrates an emission spectrum of the thin film of CzPAαN;

FIG. 15 illustrates CV measurement results of CzPAαN;

FIG. 16 illustrates CV measurement results of CzPAαN;

FIG. 17 illustrates luminance-current efficiency characteristics of a light-emitting element 1-1 and a light-emitting element 1-3;

FIG. 18 illustrates emission spectra of the light-emitting element 1-1 and the light-emitting element 1-3;

FIG. 19 illustrates current density-luminance characteristics of the light-emitting element 1-1 and the light-emitting element 1-3;

FIG. 20 illustrates voltage-luminance characteristics of the light-emitting element 1-1 and the light-emitting element 1-3;

FIG. 21 illustrates luminance-current efficiency characteristics of a light-emitting element 1-2;

FIG. 22 illustrates an emission spectrum of the light-emitting element 1-2;

FIG. 23 illustrates current density-luminance characteristics of the light-emitting element 1-2;

FIG. 24 illustrates voltage-luminance characteristics of the light-emitting element 1-2;

FIG. 25 illustrates results of reliability tests of the light-emitting element 1-1 and the light-emitting element 1-3;

FIGS. 26A and 26B illustrate light-emitting elements of Examples;

FIGS. 27A and 27B illustrate light-emitting elements;

FIGS. 28A and 28B are the ¹H-NMR charts of CzPAβN;

FIG. 29 illustrates an absorption spectrum of CzPAβN included in a toluene solution;

FIG. 30 illustrates an absorption spectrum of a thin film of CzPAβN;

FIG. 31 illustrates an emission spectrum of CzPAβN included in the toluene solution;

FIG. 32 illustrates an emission spectrum of the thin film of CzPAβN;

FIG. 33 illustrates CV measurement results of CzPAβN;

FIG. 34 illustrates CV measurement results of CzPAβN;

FIGS. 35A and 35B are the ¹H-NMR charts of CzPApB;

FIG. 36 illustrates an absorption spectrum of CzPApB included in a toluene solution;

FIG. 37 illustrates an absorption spectrum of a thin film of CzPApB;

FIG. 38 illustrates an emission spectrum of CzPApB included in the toluene solution;

FIG. 39 illustrates an emission spectrum of the thin film of CzPApB;

FIG. 40 illustrates current density-luminance characteristics of a light-emitting element 2-1 and a comparative light-emitting element 2-1;

FIG. 41 illustrates voltage-luminance characteristics of the light-emitting element 2-1 and the comparative light-emitting element 2-1;

FIG. 42 illustrates luminance-current efficiency characteristics of the light-emitting element 2-1 and the comparative light-emitting element 2-1;

FIG. 43 illustrates emission spectra of the light-emitting element 2-1 and the comparative light-emitting element 2-1;

FIG. 44 illustrates results of reliability tests of the light-emitting element 2-1 and the comparative light-emitting element 2-1;

FIGS. 45A and 45B are the ¹H-NMR charts of CzPAoB;

FIG. 46 illustrates an absorption spectrum of CzPAoB included in a toluene solution;

FIG. 47 illustrates an absorption spectrum of a thin film of CzPAoB;

FIG. 48 illustrates an emission spectrum of CzPAoB included in the toluene solution;

FIG. 49 illustrates an emission spectrum of the thin film of CzPAoB;

FIG. 50 illustrates CV measurement results of CzPAoB;

FIG. 51 illustrates CV measurement results of CzPAoB;

FIGS. 52A and 52B are the ¹H-NMR charts of CzPAαNP;

FIG. 53 illustrates an absorption spectrum of CzPAαNP included in a toluene solution;

FIG. 54 illustrates an absorption spectrum of a thin film of CzPAαNP;

FIG. 55 illustrates an emission spectrum of CzPAαNP included in the toluene solution;

FIG. 56 illustrates an emission spectrum of the thin film of CzPAαNP;

FIG. 57 illustrates CV measurement results of CzPAαNP;

FIG. 58 illustrates CV measurement results of CzPAαNP;

FIGS. 59A and 59B are the ¹H-NMR charts of CzPAFL;

FIG. 60 illustrates an absorption spectrum of CzPAFL included in a toluene solution;

FIG. 61 illustrates an absorption spectrum of a thin film of CzPAFL;

FIG. 62 illustrates an emission spectrum of CzPAFL included in the toluene solution;

FIG. 63 illustrates an emission spectrum of the thin film of CzPAFL;

FIG. 64 illustrates CV measurement results of CzPAFL;

FIG. 65 illustrates CV measurement results of CzPAFL;

FIG. 66 illustrates current density-luminance characteristics of a light-emitting element 2-2 and a light-emitting element 2-3;

FIG. 67 illustrates voltage-luminance characteristics of the light-emitting element 2-2 and the light-emitting element 2-3;

FIG. 68 illustrates luminance-current efficiency characteristics of the light-emitting element 2-2 and the light-emitting element 2-3;

FIG. 69 illustrates emission spectra of the light-emitting element 2-2 and the light-emitting element 2-3;

FIG. 70 illustrates results of reliability tests of the light-emitting element 2-2 and the light-emitting element 2-3;

FIGS. 71A and 71B are the ¹H-NMR charts of CzPAmB;

FIG. 72 illustrates an absorption spectrum of CzPAmB included in a toluene solution;

FIG. 73 illustrates an absorption spectrum of a thin film of CzPAmB;

FIG. 74 illustrates an emission spectrum of CzPAmB included in the toluene solution;

FIG. 75 illustrates an emission spectrum of the thin film of CzPAmB;

FIG. 76 illustrates CV measurement results of CzPAmB;

FIG. 77 illustrates CV measurement results of CzPAmB;

FIG. 78 illustrates current density-luminance characteristics of a light-emitting element 3-1 and a comparative light-emitting element 3-1;

FIG. 79 illustrates voltage-luminance characteristics of the light-emitting element 3-1 and the comparative light-emitting element 3-1;

FIG. 80 illustrates luminance-current efficiency characteristics of the light-emitting element 3-1 and the comparative light-emitting element 3-1;

FIG. 81 illustrates emission spectra of the light-emitting element 3-1 and the comparative light-emitting element 3-1;

FIG. 82 illustrates results of reliability tests of the light-emitting element 3-1 and the comparative light-emitting element 3-1;

FIG. 83 illustrates current density-luminance characteristics of a light-emitting element 3-2 and a comparative light-emitting element 3-2;

FIG. 84 illustrates voltage-luminance characteristics of the light-emitting element 3-2 and the comparative light-emitting element 3-2;

FIG. 85 illustrates luminance-current efficiency characteristics of the light-emitting element 3-2 and the comparative light-emitting element 3-2;

FIG. 86 illustrates emission spectra of the light-emitting element 3-2 and the comparative light-emitting element 3-2;

FIG. 87 illustrates results of reliability tests of the light-emitting element 3-2 and the comparative light-emitting element 3-2;

FIG. 88 illustrates current density-luminance characteristics of a light-emitting element 3-3 and a comparative light-emitting element 3-3;

FIG. 89 illustrates voltage-luminance characteristics of the light-emitting element 3-3 and the comparative light-emitting element 3-3;

FIG. 90 illustrates luminance-current efficiency characteristics of the light-emitting element 3-3 and the comparative light-emitting element 3-3;

FIG. 91 illustrates emission spectra of the light-emitting element 3-3 and the comparative light-emitting element 3-3; and

FIG. 92 illustrates results of reliability tests of the light-emitting element 3-3 and the comparative light-emitting element 3-3.

### BEST MODE FOR CARRYING OUT THE INVENTION

Hereinafter, embodiments and examples of the present invention will be described in detail with reference to the drawings. Note that the present invention is not limited to the following description and it will be readily appreciated by those skilled in the art that modes and details can be modified in various ways without departing from the spirit and the scope of the present invention. Accordingly, the present invention should not be construed as being limited to the description of the embodiments and the examples given below.

### Embodiment 1

In this embodiment, one mode of a carbazole derivative of the present invention will be described.

One mode of a carbazole derivative according to this embodiment is represented by the general formula (1).

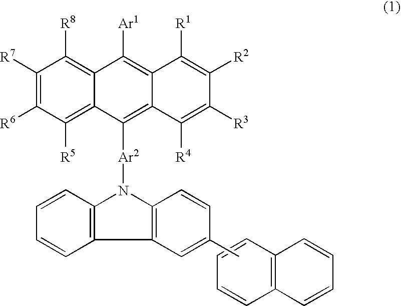

In the formula, Ar¹ represents an aryl group having 6 to 13 carbon atoms, Ar² represents an arylene group having 6 to 13 carbon atoms, and R¹ to R⁸ independently represent hydrogen or an alkyl group having 1 to 4 carbon atoms. Ar¹ and Ar² may independently have a substituent or substituents: when Ar¹ and Ar² independently have two or more substituents, the substituents may be bonded to each other to form a ring structure, and when one carbon atom of any of Ar¹ and Ar² has two substituents, the substituents may be bonded to each other to form a spiro ring structure.

One mode of a carbazole derivative according to this embodiment is represented by the general formula (2).

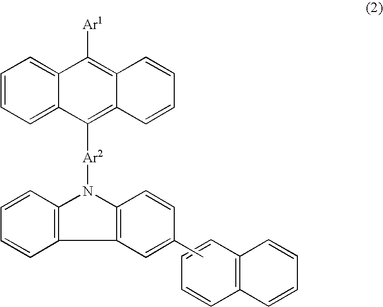

In the formula, Ar¹ represents an aryl group having 6 to 13 carbon atoms and Ar² represents an arylene group having 6 to 13 carbon atoms. Ar¹ and Ar² may independently have a substituent or substituents: when Ar¹ and Ar² independently have two or more substituents, the substituents may be bonded to each other to form a ring structure, and when one carbon atom of Ar¹ and Ar² has two substituents, the substituents may be bonded to each other to form a spiro ring structure.

One mode of a carbazole derivative according to this embodiment is represented by the general formula (3).

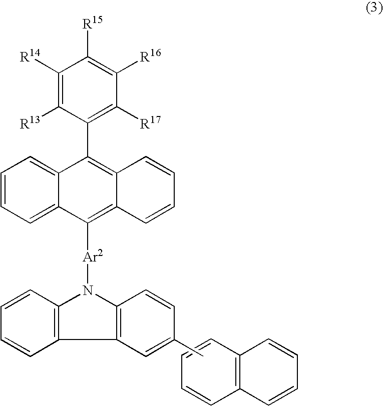

In the formula, Ar² represents an arylene group having 6 to 13 carbon atoms, and R¹³ to R¹⁷ independently represent hydrogen, an aryl group having 6 to 10 carbon atoms, an alkyl group having 1 to 4 carbon atoms, or haloalkyl group having 1 carbon atom. Ar² and R¹³ to R¹⁷ may independently have a substituent or substituents: when Ar² and R¹³ to R¹⁷ independently have two or more substituents, the substituents may be bonded to each other to form a ring structure, and when one carbon atom of any of Ar² and R¹³ to R¹⁷ has two substituents, the substituents may be bonded to each other to form a spiro ring structure.

One mode of a carbazole derivative according to this embodiment is represented by the general formula (4).

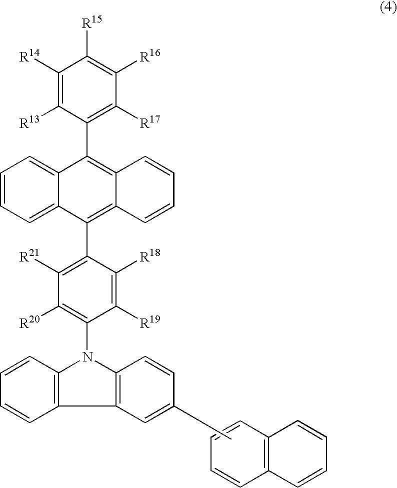

In the formula, R¹³ to R¹⁷ independently represent hydrogen, an aryl group having 6 to 10 carbon atoms, an alkyl group having 1 to 4 carbon atoms, or a haloalkyl group having 1 carbon atom; and R¹⁸ to R²¹ independently represent hydrogen or an alkyl group having 1 to 4 carbon atoms. R¹³ to R¹⁷ may independently have a substituent or substituents: when R¹³ to R¹⁷ independently have two or more substituents, the substituents may be bonded to each other to form a ring structure, and when one carbon atom of any of R¹³ to R¹⁷ has two substituents, the substituents may be bonded to each other to form a spiro ring structure.

Note that the number of carbon atoms of the aryl group and the arylene group in this specification refers to the number of carbon atoms forming a ring structure of the main skeleton and does not include the number of carbon atoms of a substituent bonded to the main skeleton. As substituents which are bonded to an aryl group or an arylene group, an alkyl group having 1 to 4 carbon atoms, an aryl group having 6 to 13 carbon atoms, and a haloalkyl group having 1 carbon atom can be given. In specific, a methyl group, an ethyl group, a propyl group, a butyl group, a phenyl group, a naphthyl group, a fluorenyl group, and a trifluoromethyl group can be given. Further, an aryl group or an arylene group may have either single or plural substituents. When an aryl group or an arylene group has two substituents, the substituents may be bonded to each other to form a ring structure. For example, when an aryl group is a fluorenyl group, carbon at a 9-position of the fluorene skeleton may have two phenyl groups, and the two phenyl groups may be bonded to each other to form a spiro ring structure.

In the general formulas (1) to (4), an aryl group having 6 to 13 carbon atoms or an arylene group may have a substituent or substituents. When the aryl group having 6 to 13 carbon atoms or the arylene group has plural substituents, the substituents may be bonded to each other to form a ring structure. In addition, when one carbon atom has two substituents, the substituents may be bonded to each other to form a spiro ring structure. For example, as a group represented by Ar¹, a substituent represented by the structural formula (11-1) to the structural formula (11-16) can be specifically given.

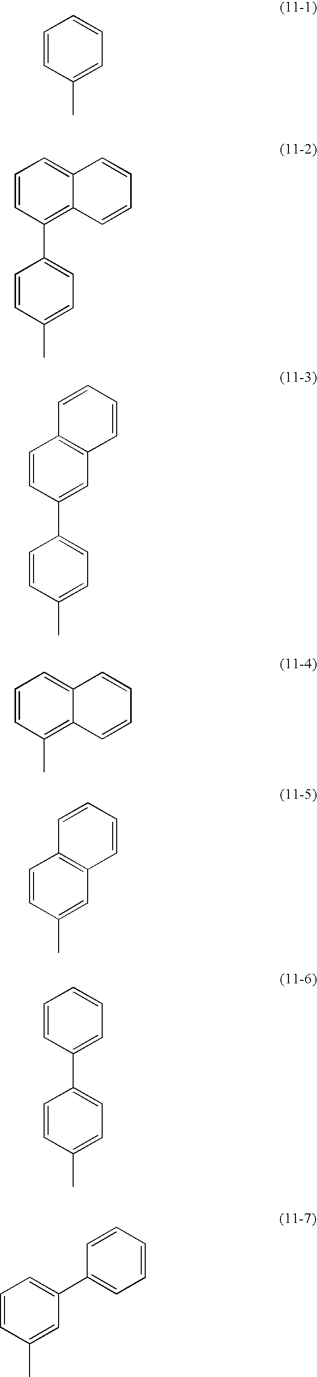

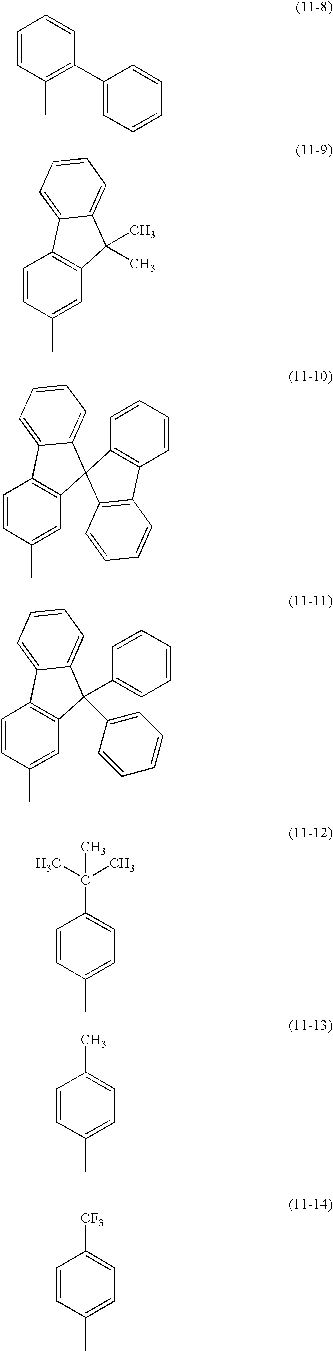

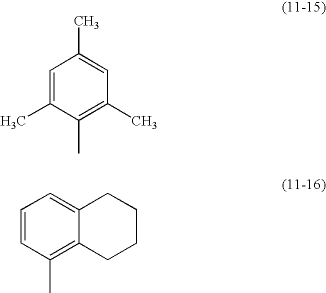

For example, as a group represented by Ar², a substituent represented by the structural formula (12-1) to the structural formula (12-11) can be specifically given.

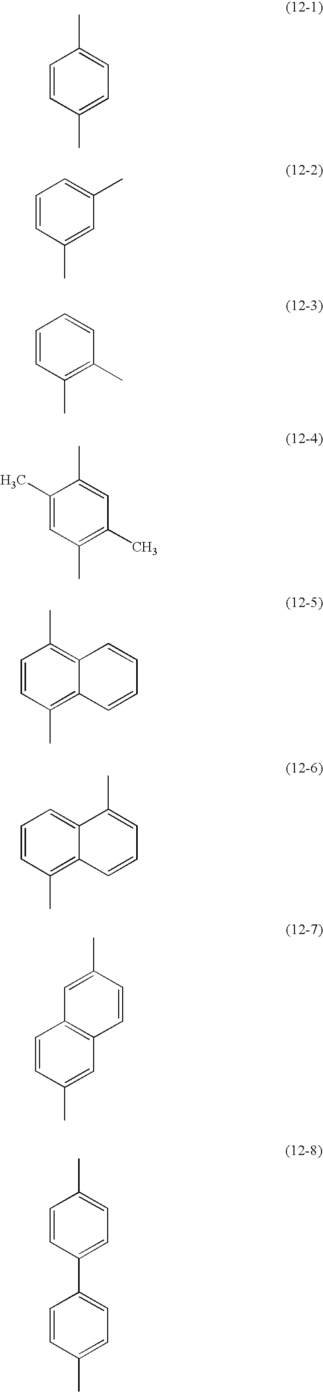

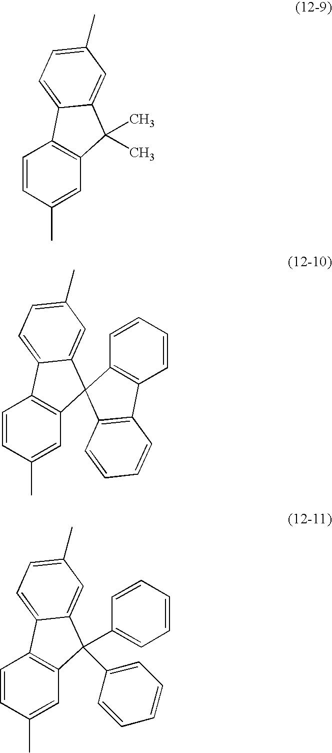

For example, as a group represented by R¹³ to R²¹, a substituent represented by the structural formula (13-1) to the structural formula (13-10) can be specifically given.

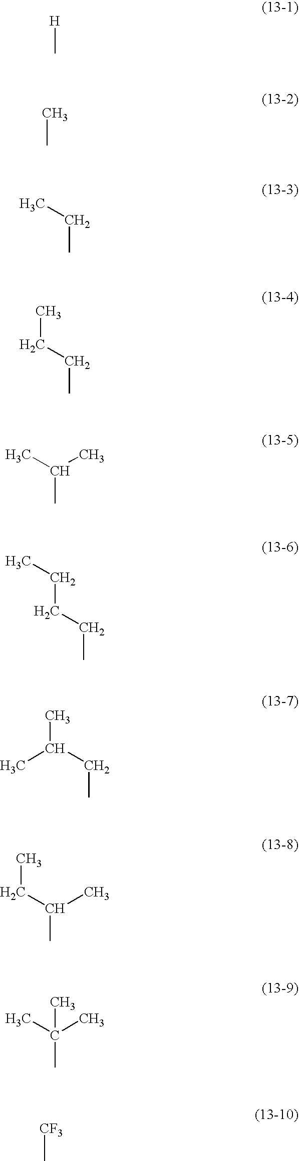

Further, in the carbazole derivatives represented by the general formulas (1) to (4), Ar¹ and Ar² preferably are a phenyl group and a phenylene group, respectively, for their ease of synthesis and purification.

As specific examples of the carbazole derivatives represented by the general formulas (1) to (4), carbazole derivative represented by the structural formula (101) to the structural formula (125) and the structural formula (201) to the structural formula (231) can be given. However, the present invention is not limited thereto.

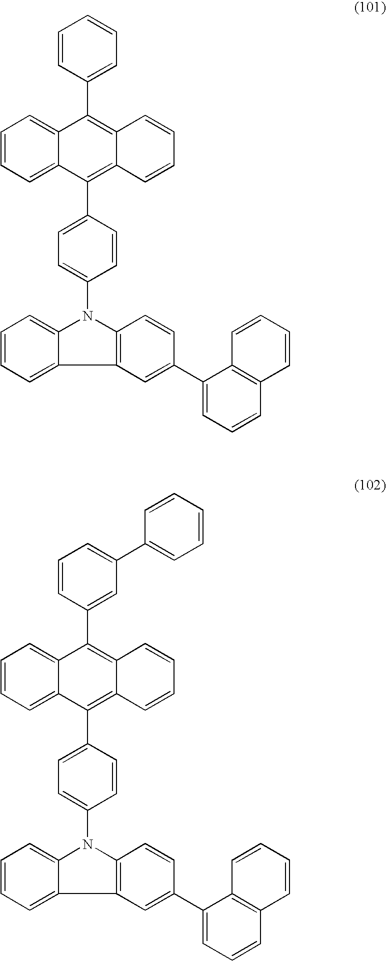

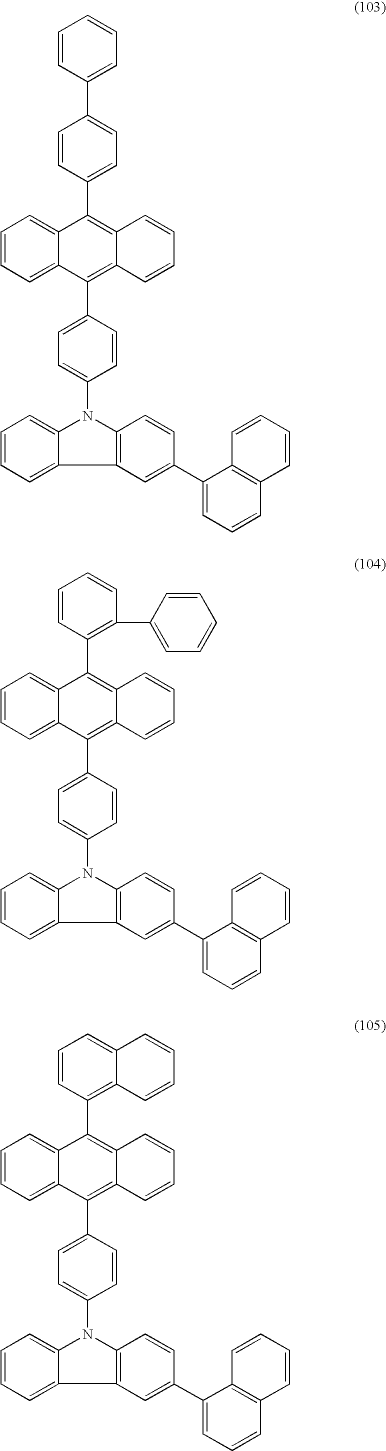

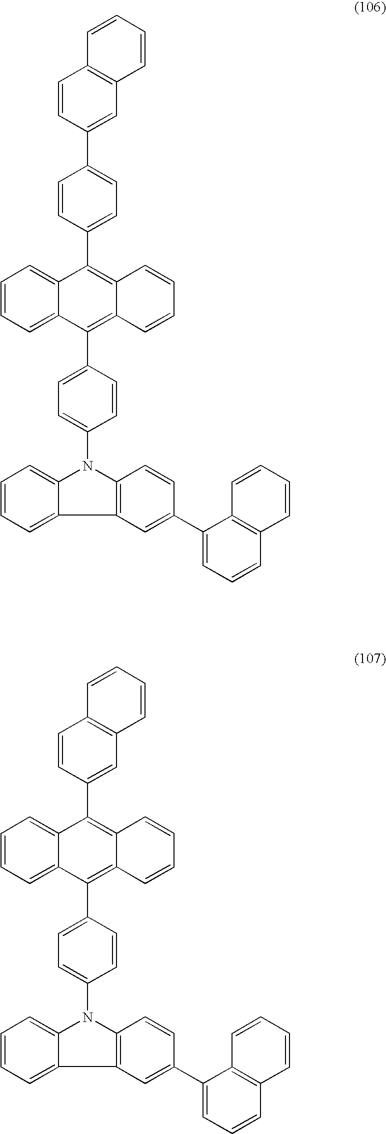

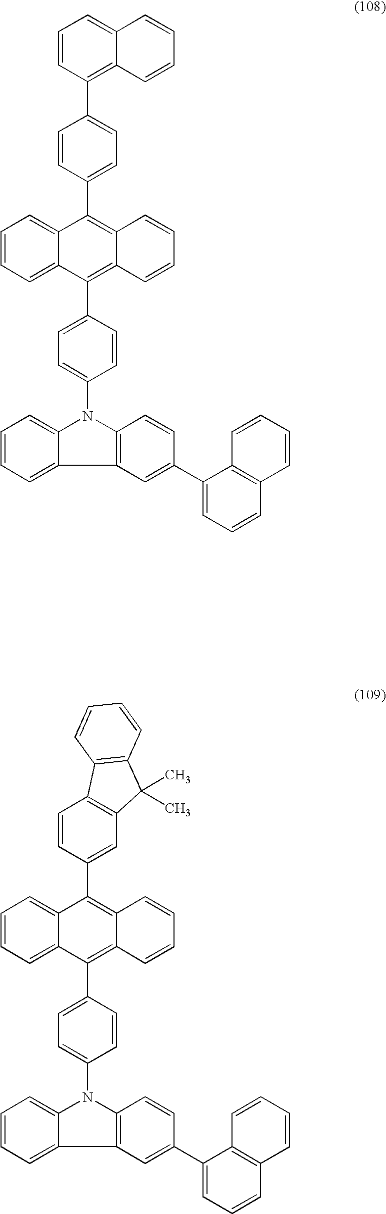

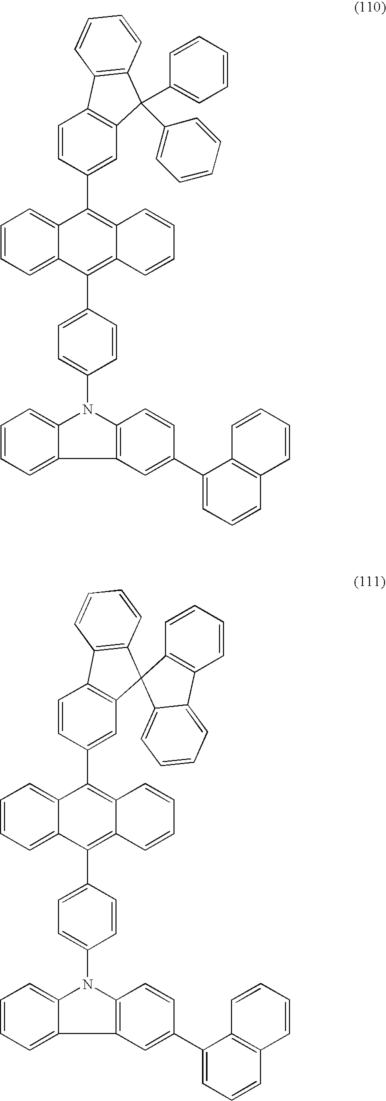

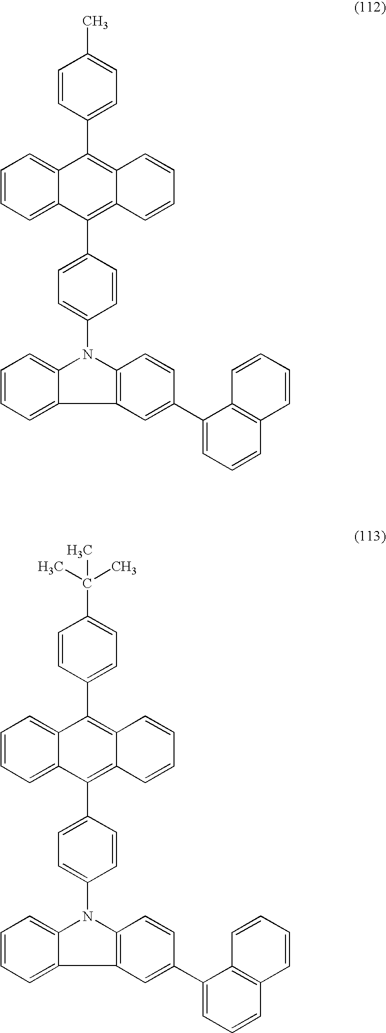

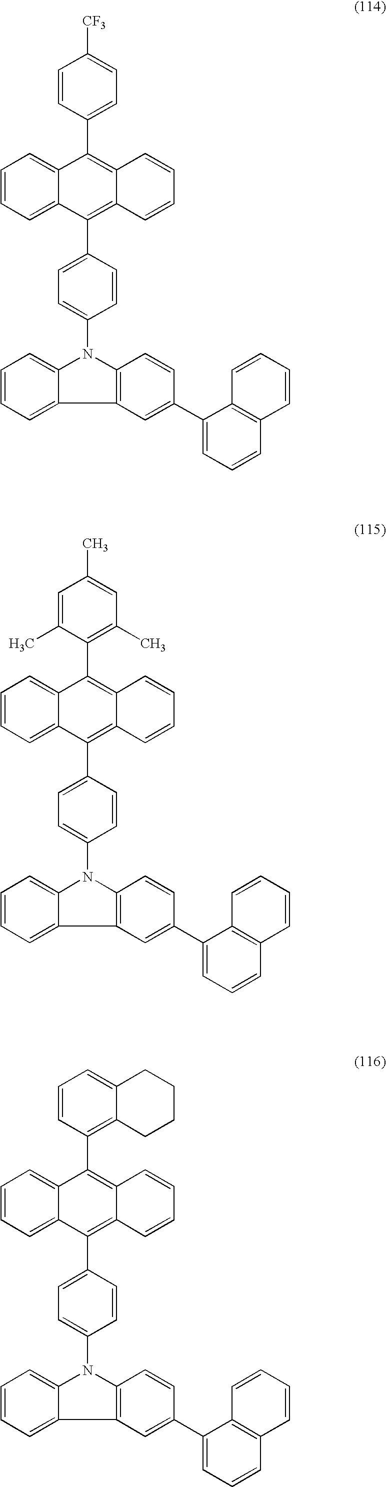

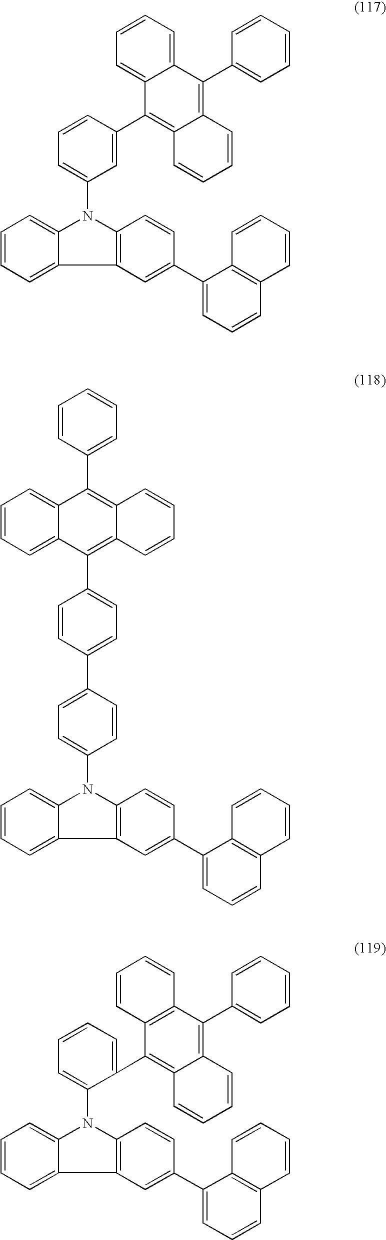

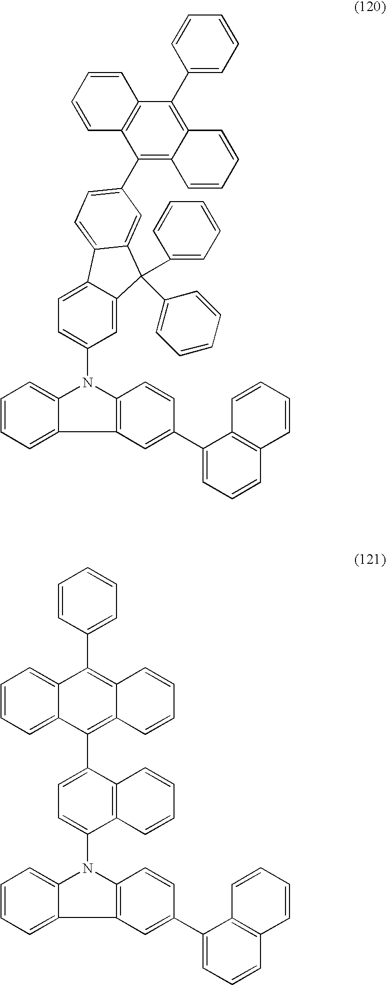

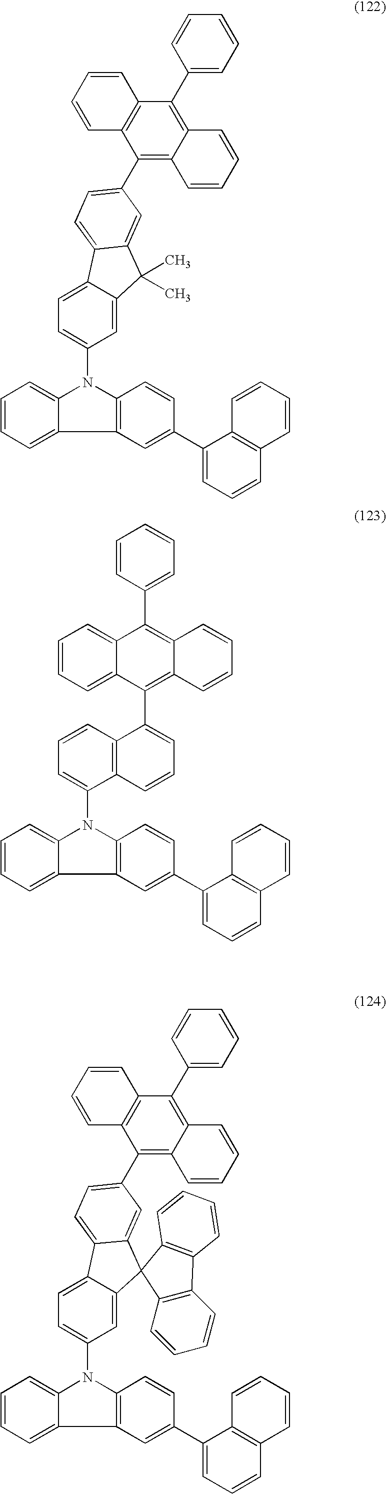

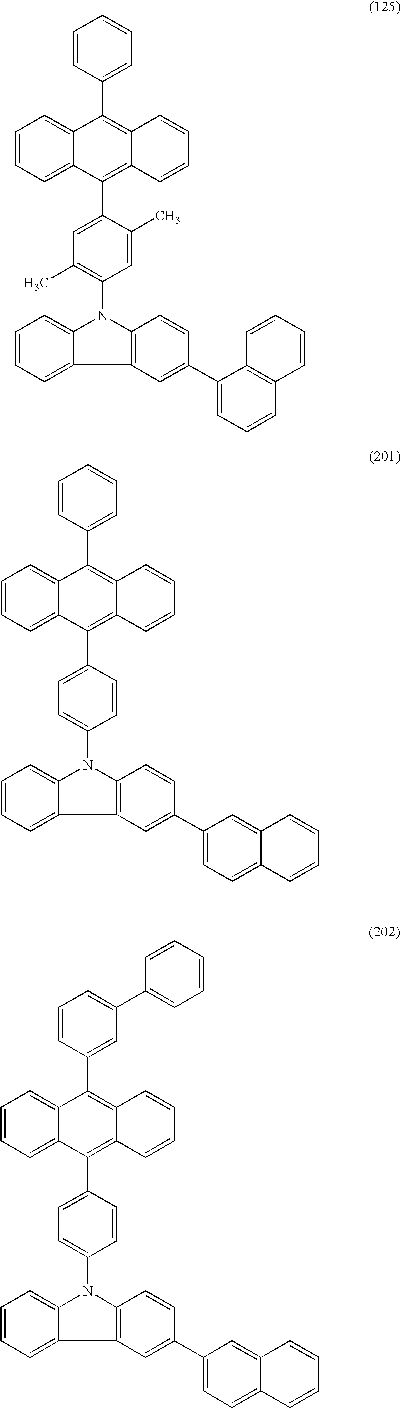

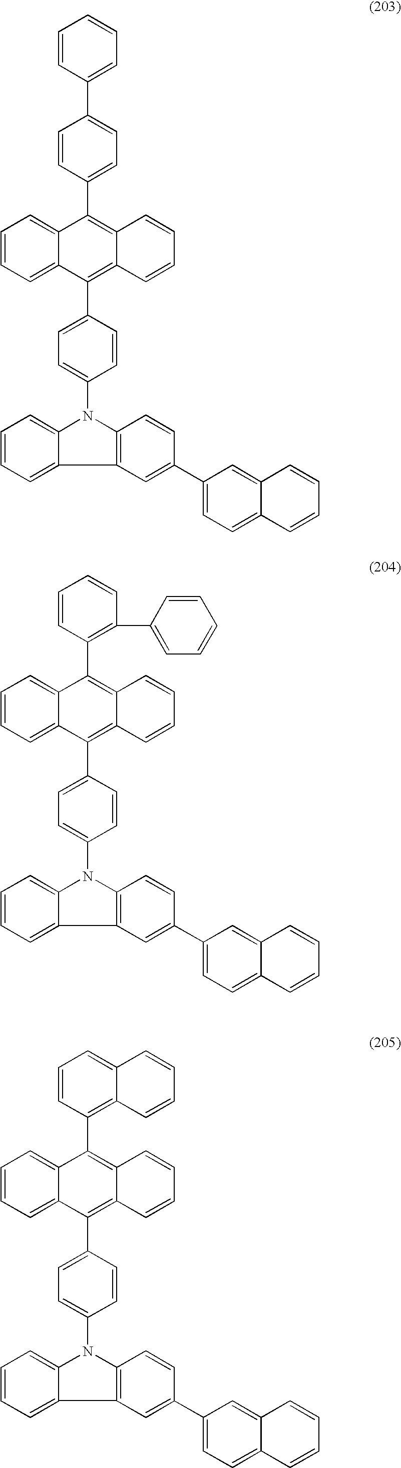

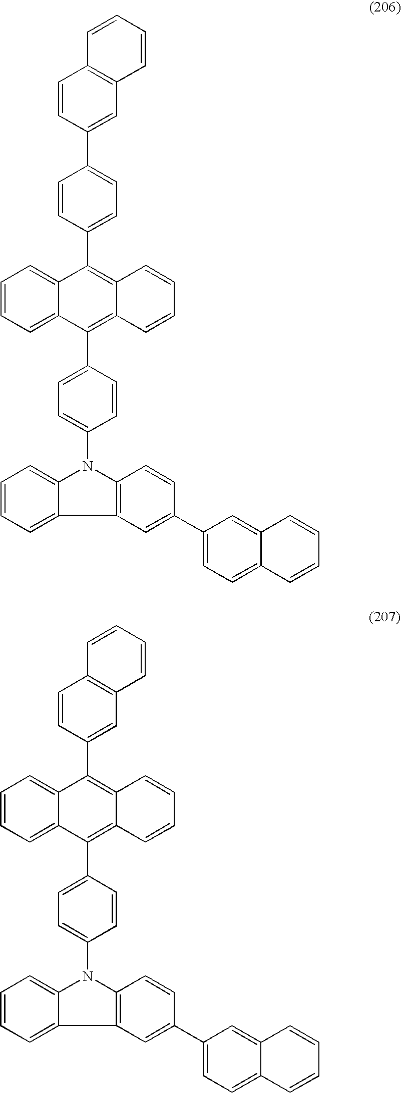

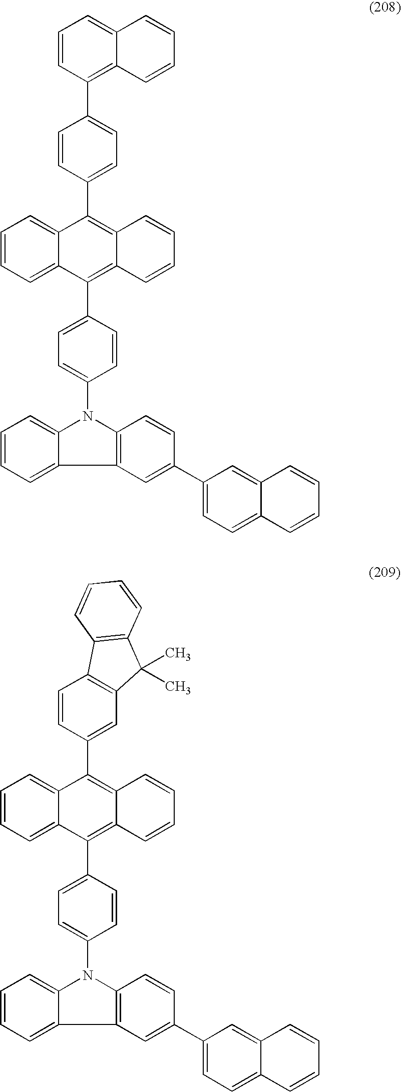

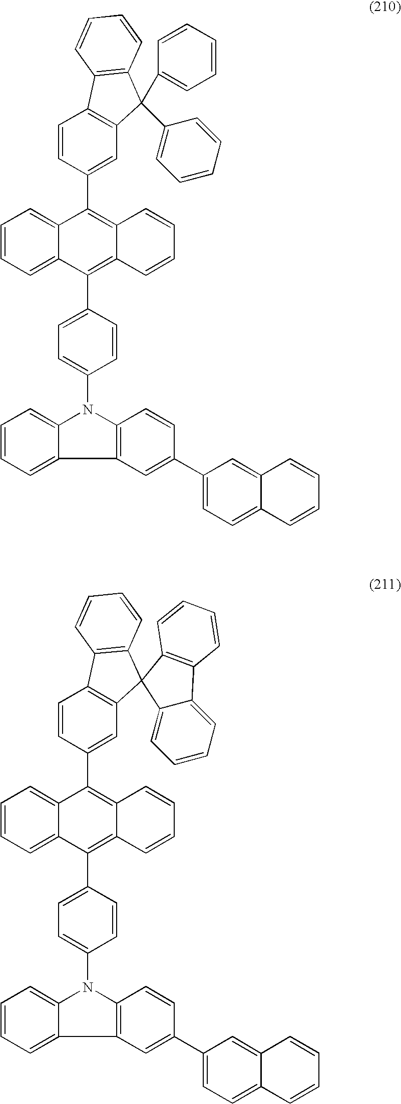

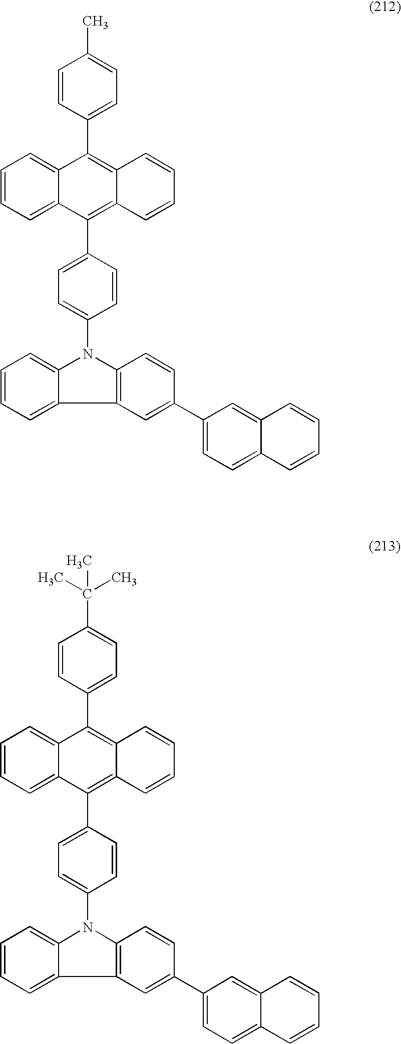

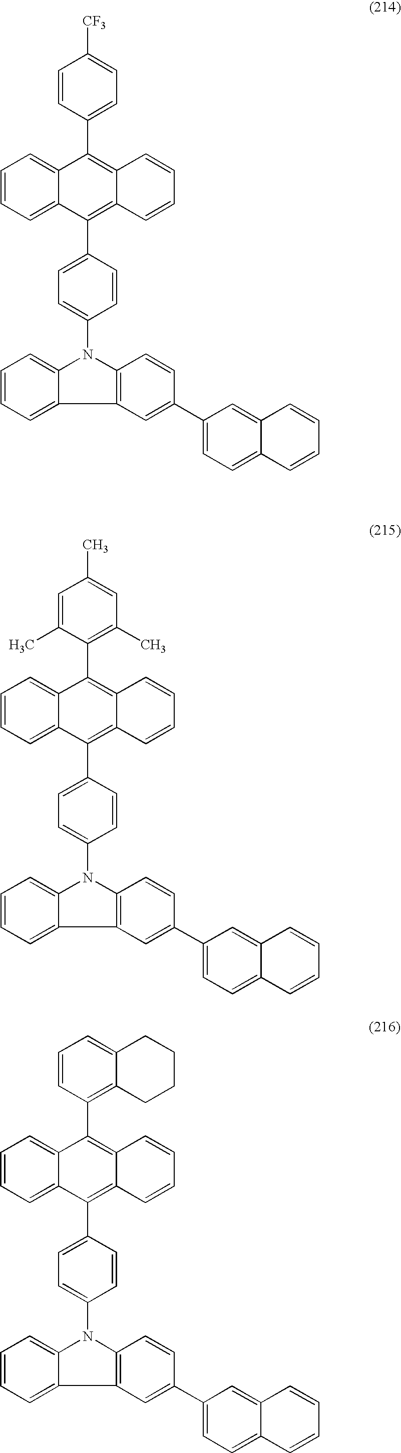

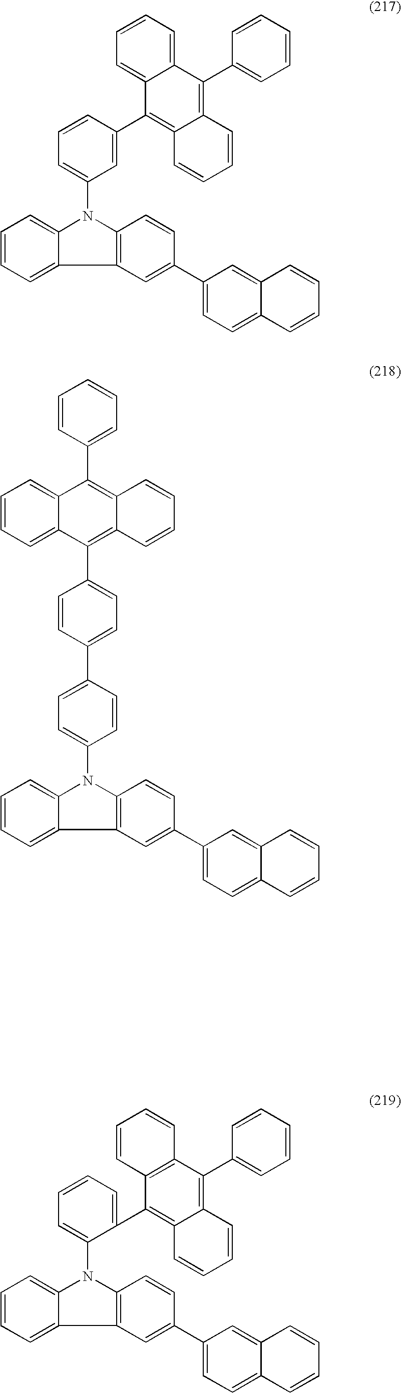

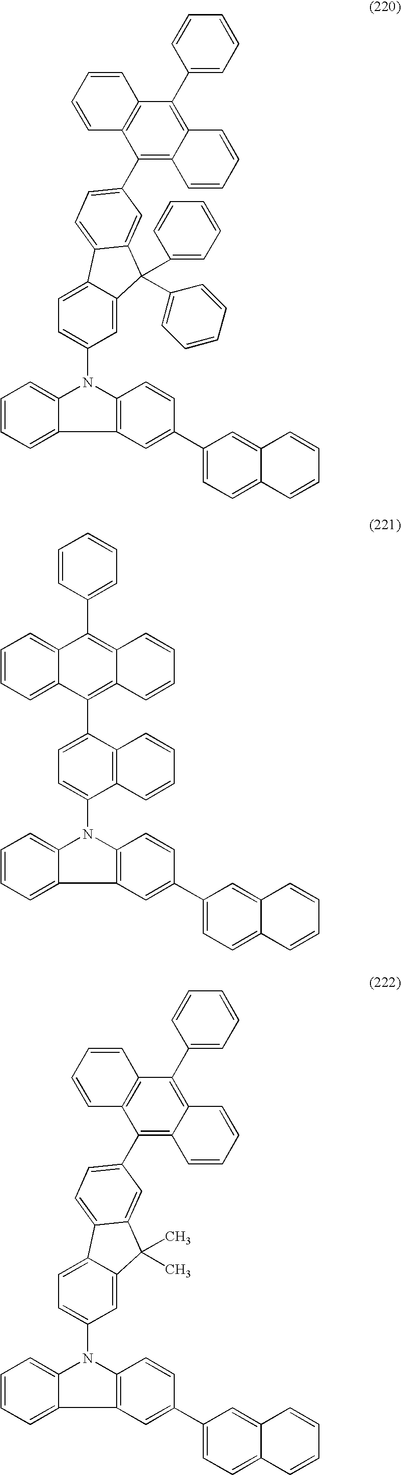

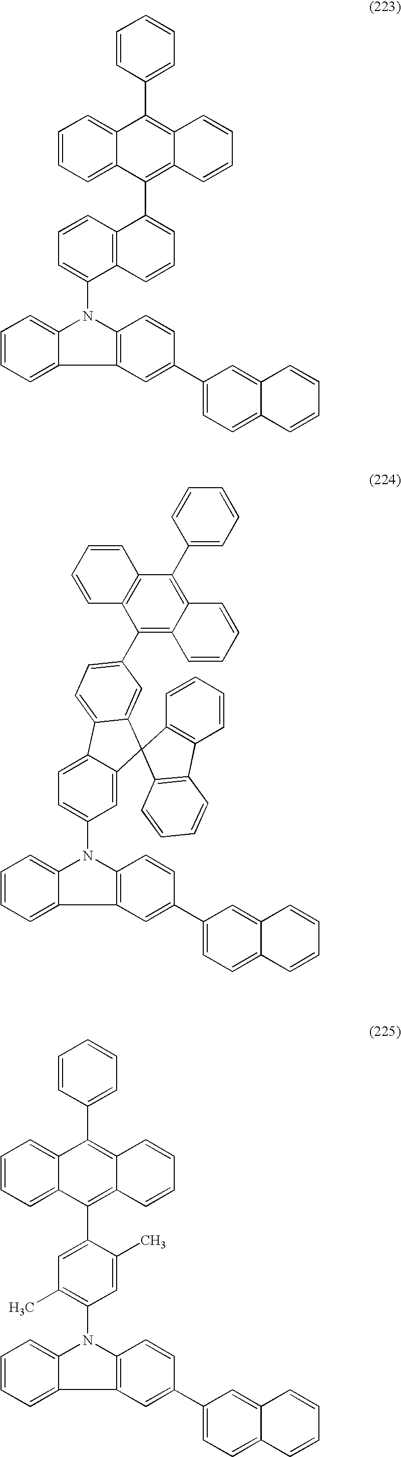

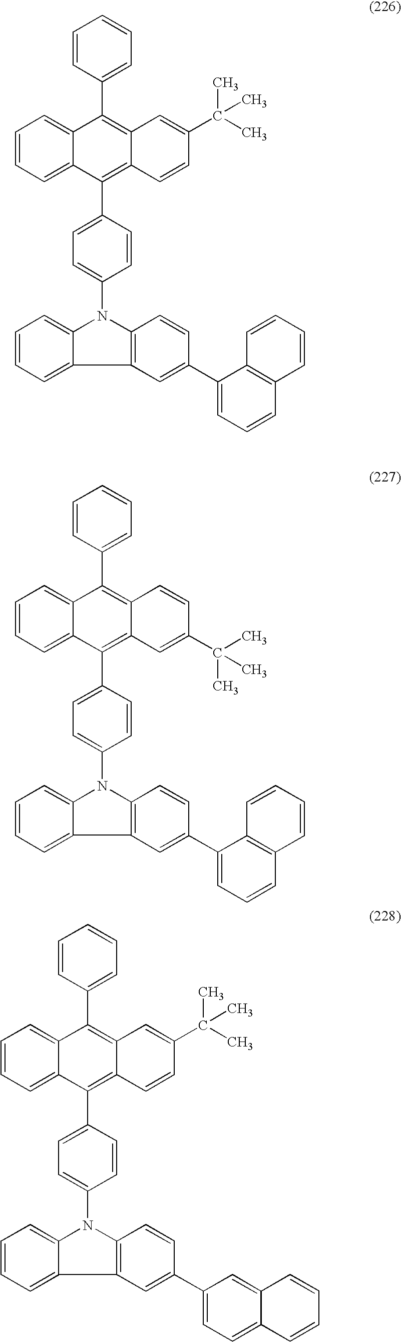

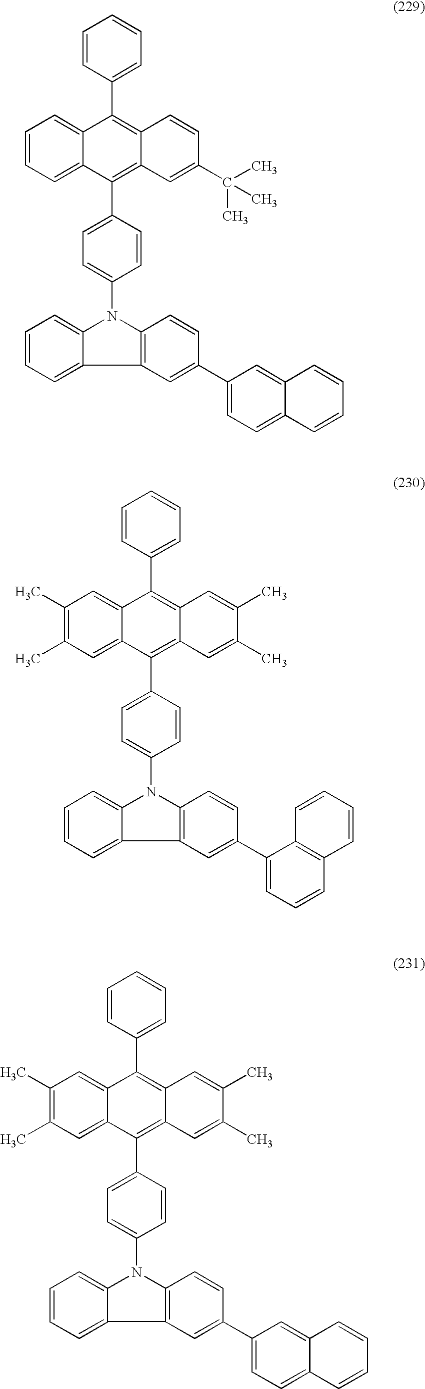

Various reactions can be applied to a synthesis method of the carbazole derivative according to this embodiment. For example, the carbazole derivative can be synthesized by synthesis reactions represented by the synthetic schemes (Z-1) to (Z-5) shown below.

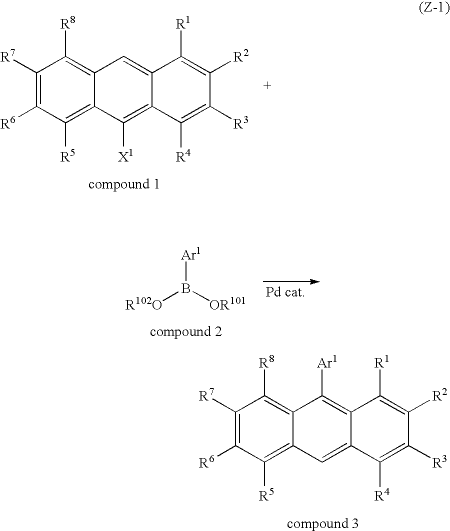

As shown by the synthetic scheme (Z-1), a 9-arylanthracene derivative (compound 3) can be obtained by Suzuki-Miyaura coupling of an anthracene derivative (compound 1) and an arylboronic acid or arylorganoboron compound (compound 2) in the presence of a palladium catalyst.

In the synthetic scheme (Z-1), X¹ represents a halogen or a triflate group and the halogen is preferably iodine, bromine, or chlorine; R¹ to R⁸ independently represent hydrogen or an alkyl group having 1 to 4 carbon atoms; Ar¹ represents an aryl group having 6 to 13 carbon atoms which may have a substituent or substituents which may be bonded to each other to form a ring structure which may be a spiro ring structure; and R¹⁰¹ and R¹⁰² independently represent hydrogen or an alkyl group having 1 to 6 carbon atoms and R¹⁰¹ and R¹⁰² may be bonded to each other to form a ring structure.

Examples of a palladium catalyst which can be used in the synthetic scheme (Z-1) include, but are not limited to, palladium(II) acetate, tetrakis(triphenylphosphine)palladium(0).

Examples of a ligand of the palladium catalyst which can be used in the synthetic scheme (Z-1) include, but are not limited to, tri(ortho-tolyl)phosphine, triphenylphosphine, and tricyclohexylphosphine.

Examples of a base which can be used in the synthetic scheme (Z-1) include, but are not limited to, an organic base such as sodium tert-butoxide and an inorganic base such as potassium carbonate.

Examples of a solvent which can be used in the synthetic scheme (Z-1) include, but are not limited to, a mixed solvent of toluene and water; a mixed solvent of toluene, alcohol such as ethanol, and water; a mixed solvent of xylene and water; a mixed solvent of xylene, alcohol such as ethanol, and water; a mixed solvent of benzene and water; a mixed solvent of benzene, alcohol such as ethanol, and water; and a mixed solvent of ether such as ethylene glycol dimethyl ether and water. Further, a mixed solvent of toluene and water or a mixed solvent of toluene, ethanol, and water is more preferable.

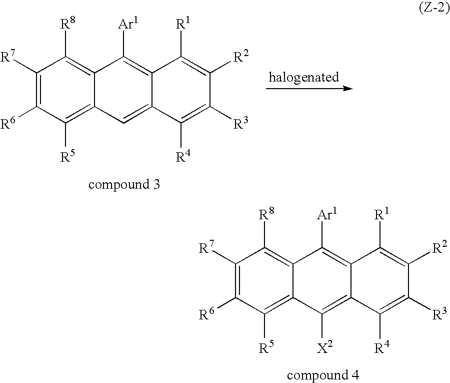

As shown by the synthetic scheme (Z-2), a halogenated arylanthracene derivative (compound 4) can be obtained by halogenating the 9-arylanthracene derivative (compound 3).

In the synthetic scheme (Z-2), X² represents a halogen and the halogen is preferably iodine or bromine; R¹ to R⁸ independently represent hydrogen or an alkyl group having 1 to 4 carbon atoms; Ar¹ represents an aryl group having 6 to 13 carbon atoms which may have a substituent or substituents which may be bonded to each other to form a ring structure which may be a spiro ring structure.

In the case of brominating the 9-arylanthracene derivative (compound 3) in the synthetic scheme (Z-2), examples of a brominating agent which can be used include, but are not limited to, bromine and N-bromosuccinimide. Examples of a solvent which can be used in the case of brominating the 9-arylanthracene derivative (compound 3) using bromine include, but are not limited to, a halogen-based solvent such as chloroform or carbon tetrachloride. Examples of a solvent which can be used in the case of brominating the 9-arylanthracene derivative (compound 3) using N-bromosuccinimide include, but are not limited to, ethyl acetate, tetrahydrofuran, dimethylformamide, acetic acid, water, and toluene.

In the case of iodinating the 9-arylanthracene derivative (compound 3) in the synthetic scheme (Z-2), examples of an iodinating agent which can be used include, but are not limited to, N-iodosuccinimide, 1,3-diiodo-5,5-dimethylimidazolidine-2,4-dione (DIH), 2,4,6,8-tetraiodo-2,4,6,8-tetraazabicyclo[3,3,0]octane-3,7-dion, and 2-iodo-2,4,6,8-tetraazabicyclo[3,3,0]octane-3,7-dion. Further, examples of a solvent which can be used in the case of iodinating the 9-arylanthracene derivative (compound 3) using any of those iodinating agents include, acetic acid (glacial acetic acid); water; aromatic hydrocarbons such as benzene, toluene, and xylene; ethers such as 1,2-dimethoxyethane, diethyl ether, methyl-t-butyl ether, tetrahydrofuran, and dioxane; saturated hydrocarbons such as pentane, hexane, heptane, octane, and cyclohexane; halogenated carbons such as dichloromethane, chloroform, carbon tetrachloride, 1,2-dichloroethane, and 1,1,1-trichloroethane; nitriles such as acetonitrile and benzonitrile; and esters such as ethyl acetate, methyl acetate, and butyl acetate. Those solvents can be used alone or in combination. When water is used, it is preferably mixed with an organic solvent. In addition, in this reaction, an acid such as sulfuric acid or acetic acid is preferably used as well and an acid which can be used is not limited thereto.

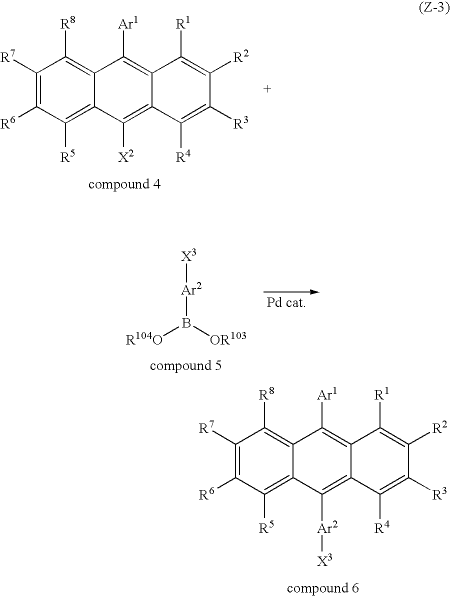

As shown by the synthetic scheme (Z-3), a halogenated diarylanthracene derivative (compound 6) can be obtained by Suzuki-Miyaura coupling of the halogenated arylanthracene derivative (compound 4) and an arylorganoboron compound such as a halogenated arylboronic acid (compound 5) in the presence of a palladium catalyst.

In the synthetic scheme (Z-3), X² represents a halogen and the halogen is preferably iodine or bromine; X³ represents a halogen or a triflate group and the halogen is preferably iodine, bromine, or chlorine; R¹ to R⁸ independently represent hydrogen or an alkyl group having 1 to 4 carbon atoms; Ar¹ represents an aryl group having 6 to 13 carbon atoms which may have a substituent or substituents which may be bonded to each other to form a ring structure which may be a spiro ring structure; Ar² represents an arylene group having 6 to 13 carbon atoms which may have a substituent or substituents which may be bonded to each other to form a ring structure which may be a spiro ring structure; and R¹⁰³ and R¹⁰⁴ independently represent hydrogen or an alkyl group having 1 to 6 carbon atoms and R¹⁰³ and R¹⁰⁴ may be bonded to each other to form a ring structure.

Examples of a palladium catalyst which can be used in the synthetic scheme (Z-3) include, but are not limited to, palladium(II) acetate, tetrakis(triphenylphosphine)palladium(0).

Examples of a ligand of the palladium catalyst which can be used in the synthetic scheme (Z-3) include, but are not limited to, tri(ortho-tolyl)phosphine, triphenylphosphine, and tricyclohexylphosphine.

Examples of a base which can be used in the synthetic scheme (Z-3) include, but are not limited to, an organic base such as sodium tert-butoxide and an inorganic base such as potassium carbonate.

Examples of a solvent which can be used in the synthetic scheme (Z-3) include, but are not limited to, a mixed solvent of toluene and water; a mixed solvent of toluene, alcohol such as ethanol, and water; a mixed solvent of xylene and water; a mixed solvent of xylene, alcohol such as ethanol, and water; a mixed solvent of benzene and water; a mixed solvent of benzene, alcohol such as ethanol, and water; and a mixed solvent of ether such as ethylene glycol dimethyl ether and water. Further, a mixed solvent of toluene and water or a mixed solvent of toluene, ethanol, and water is more preferable.

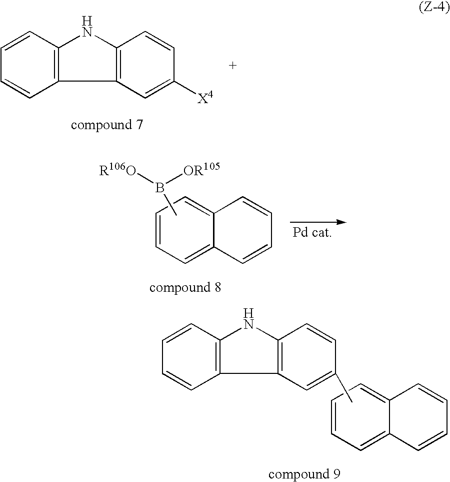

As shown by the synthetic scheme (Z-4), a carbazole derivative (compound 9) can be obtained by Suzuki-Miyaura coupling of a carbazole derivative (compound 7) and phenyl boronic acid such as a phenyl organoboron compound (compound 8) in the presence of a palladium catalyst.

In the synthetic scheme (Z-4), X⁴ represents a halogen or a triflate group and the halogen is preferably iodine, bromine, or chlorine; and R¹⁰⁵ and R¹⁰⁶ independently represent hydrogen or an alkyl group having 1 to 6 carbon atoms and R¹⁰⁵ and R¹⁰⁶ may be bonded to each other to form a ring structure.

Examples of a palladium catalyst which can be used in the synthetic scheme (Z-4) include, but are not limited to, palladium(II) acetate, tetrakis(triphenylphosphine)palladium(0).

Examples of a ligand of the palladium catalyst which can be used in the synthetic scheme (Z-4) include, but are not limited to, tri(ortho-tolyl)phosphine, triphenylphosphine, and tricyclohexylphosphine.

Examples of a base which can be used in the synthetic scheme (Z-4) include, but are not limited to, an organic base such as sodium tert-butoxide and an inorganic base such as potassium carbonate.

Examples of a solvent which can be used in the synthetic scheme (Z-4) include, but are not limited to, a mixed solvent of toluene and water; a mixed solvent of toluene, alcohol such as ethanol, and water; a mixed solvent of xylene and water; a mixed solvent of xylene, alcohol such as ethanol, and water; a mixed solvent of benzene and water; a mixed solvent of benzene, alcohol such as ethanol, and water; and a mixed solvent of ether such as ethylene glycol dimethyl ether and water. Further, a mixed solvent of toluene and water or a mixed solvent of toluene, ethanol, and water is more preferable.

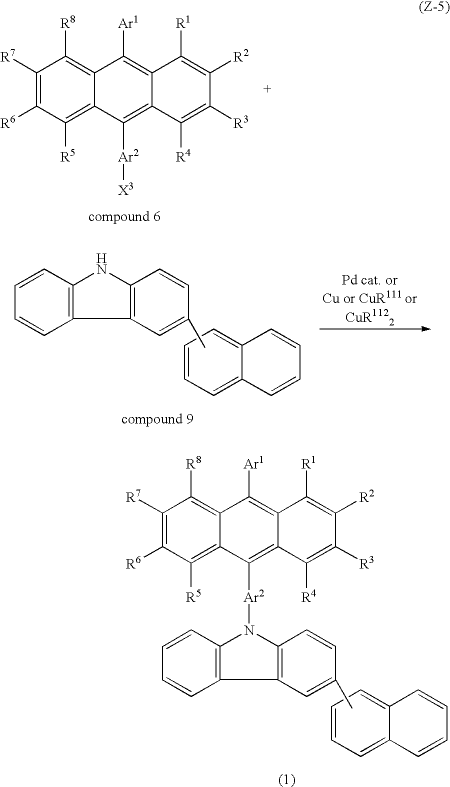

As shown by the synthetic scheme (Z-5), from the halogenated anthracene derivative (compound 6) and the carbazole derivative (compound 9), the object which is represented by the general formula (1) can be obtained by a coupling reaction of Buchwald-Hartwig reaction in the presence of a palladium catalyst or Ullmann reaction in the presence of copper or a copper compound.

In the synthetic scheme (Z-5), X³ represents a halogen or a triflate group and the halogen is preferably iodine, bromine, or chlorine; Ar¹ represents an aryl group having 6 to 13 carbon atoms which may have a substituent or substituents which may be bonded to each other to form a ring structure which may be a spiro ring structure; Ar² represents an arylene group having 6 to 13 carbon atoms which may have a substituent or substituents which may be bonded to each other to form a ring structure which may be a spiro ring structure; and R¹ to R⁸ independently represent hydrogen or an alkyl group having 1 to 4 carbon atoms.

The case in which Buchwald-Hartwig reaction is carried out in the synthetic scheme (Z-5) is described. Examples of a palladium catalyst which can be used include, but are not limited to, bis(dibenzylideneacetone)palladium(0) and palladium(II) acetate. Examples of a ligand of the palladium catalyst which can be used in the synthetic scheme (Z-5) include, but are not limited to, tri(tert-butyl)phosphine, tri(n-hexyl)phosphine, and tricyclohexylphosphine. Examples of a base which can be used in the synthetic scheme (Z-5) include, but are not limited to, an organic base such as sodium tert-butoxide and an inorganic base such as potassium carbonate. Examples of a solvent which can be used in the synthetic scheme (Z-5) include, but are not limited to, toluene, xylene, benzene, and tetrahydrofuran.

The case in which Ullmann reaction is carried out in the synthetic scheme (Z-5) is described. In the synthetic scheme (Z-5), R¹¹¹ and R¹¹² independently represent a halogen or an acetyl group and the halogen can be chlorine, bromine, or iodine. In addition, it is preferable to use copper(I) iodide where R¹¹¹ is iodine or copper(II) acetate where R¹¹² is an acetyl group, but the copper compound which is used for the reaction is not limited thereto. Further, copper can be used as an alternative to the copper compound. Examples of a base that can be used in the synthetic scheme (Z-5) include, but are not limited to, an inorganic base such as potassium carbonate. Examples of a solvent which can be used in the synthetic scheme (Z-5) include, but are not limited to, 1,3-dimethyl-3,4,5,6-tetrahydro-2(1H)pyrimidinone (DMPU), toluene, xylene, and benzene. In Ullmann reaction, DMPU or xylene which has a high boiling point is preferably used because the object can be obtained in a shorter time and at a higher yield when the reaction temperature is 100° C. or higher. DMPU is more preferably used because the reaction temperature is more preferably 150° C. or higher.

As described thus far, the carbazole derivative of this embodiment can be synthesized.

The carbazole derivative in this embodiment has a very large band gap, and therefore blue-light emission with good color purity can be exhibited. In addition, the carbazole derivative in this embodiment is a bipolar material having electron- and hole-transporting properties. In addition, the carbazole derivative in this embodiment has high electrochemical stability and thermal stability.

The carbazole derivative in this embodiment can be used alone as a light-emission center material and contained in a layer containing a light-emitting substance (a light-emitting layer). Further, the carbazole derivative in this embodiment can also be used as a host material in a light-emitting layer. Light emission from a dopant material that functions as a light-emitting substance can be obtained with a structure in which the dopant material is dispersed in the carbazole derivative in this embodiment. When the carbazole derivative is used as a host material in a light-emitting layer, blue-light emission with good color purity can be obtained.

Further, a layer in which the carbazole derivative in this embodiment is dispersed in a material (a host) which has a larger band gap than the carbazole derivative can be used as a layer containing a light-emitting substance. In that case, light emission from the carbazole derivative in this embodiment can be obtained. That is, the carbazole derivative of this embodiment can also function as a dopant material. At this time, since the carbazole derivative in this embodiment has an extremely large band gap and light with a short wavelength can be exhibited, a light-emitting element that can exhibit blue-light emission with good color purity can be manufactured.

The carbazole derivative in this embodiment can be used as a carrier-transporting material contained in a functional layer of a light-emitting element. For example, the carbazole derivative in this embodiment can be used in a carrier-transporting layer such as a hole-transporting layer, a hole-injecting layer, an electron-transporting layer, and an electron-injecting layer.

### Embodiment 2

In this embodiment, one mode of a carbazole derivative of the present invention will be described.

One mode of a carbazole derivative according to this embodiment is represented by the general formula (P1).

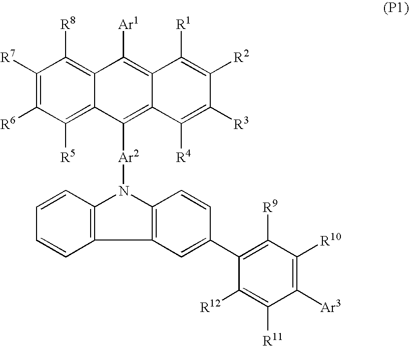

In the formula, R¹ to R¹² independently represent hydrogen or an alkyl group having 1 to 4 carbon atoms, Ar¹ and Ar³ independently represent an aryl group having 6 to 13 carbon atoms, and Ar² represents an arylene group having 6 to 13 carbon atoms. The aryl group having 6 to 13 carbon atoms and the arylene group having 6 to 13 carbon atoms may independently have a substituent or substituents: when the aryl group having 6 to 13 carbon atoms and the arylene group having 6 to 13 carbon atoms independently have two or more substituents, the substituents may be bonded to each other to form a ring structure, and when one carbon atom of any of the aryl group having 6 to 13 carbon atoms and the arylene group having 6 to 13 carbon atoms has two substituents, the substituents may be bonded to each other to form a spiro ring structure. In addition, a substituent of Ar³ may be bonded to R¹⁰ or R¹¹ to form a ring structure which may be a spiro ring structure.

In the general formula (P1), R¹ to R¹² independently represent hydrogen or an alkyl group having 1 to 4 carbon atoms. For example, substituents which are represented by the structural formula (21-1) to the structural formula (21-9) can be given.

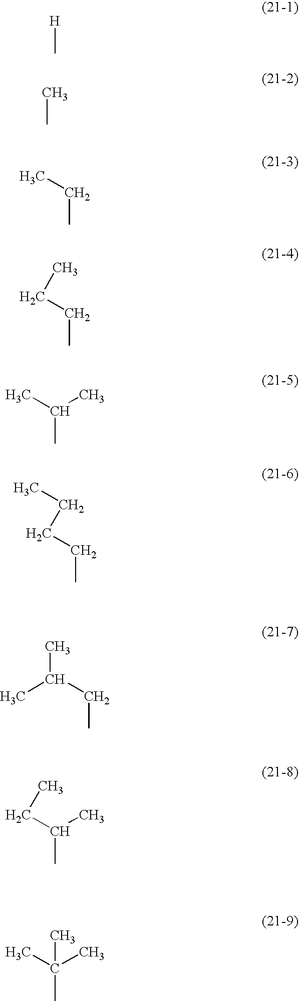

In the general formula (P1), Ar¹ and Ar³ independently represent an aryl group having 6 to 13 carbon atoms. The aryl group having 6 to 13 carbon atoms may have a substituent or substituents: when the aryl group having 6 to 13 carbon atoms has two or more substituents, the substituents may be bonded to each other to form a ring structure, and when one carbon atom has two substituents, the substituents may be bonded to each other to form a spiro ring structure. As Ar¹ and Ar³, for example, substituents which are represented by the structural formula (22-1) to the structural formula (22-16) can be given.

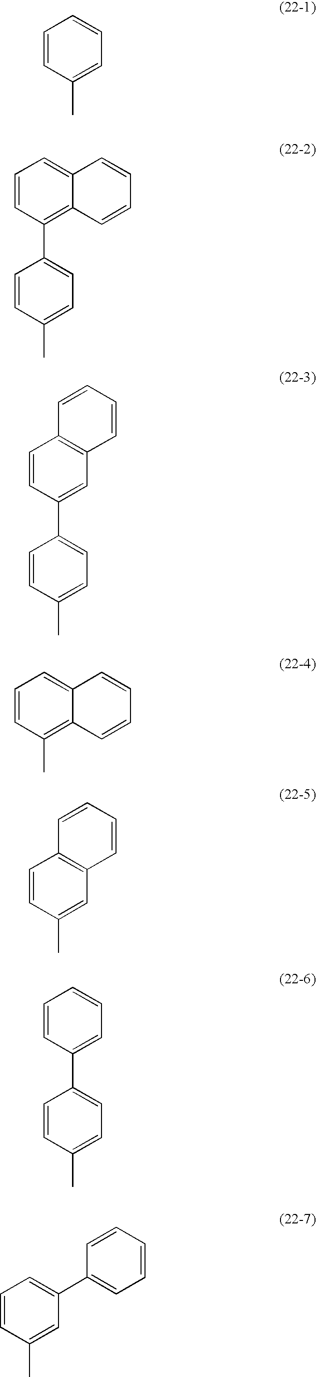

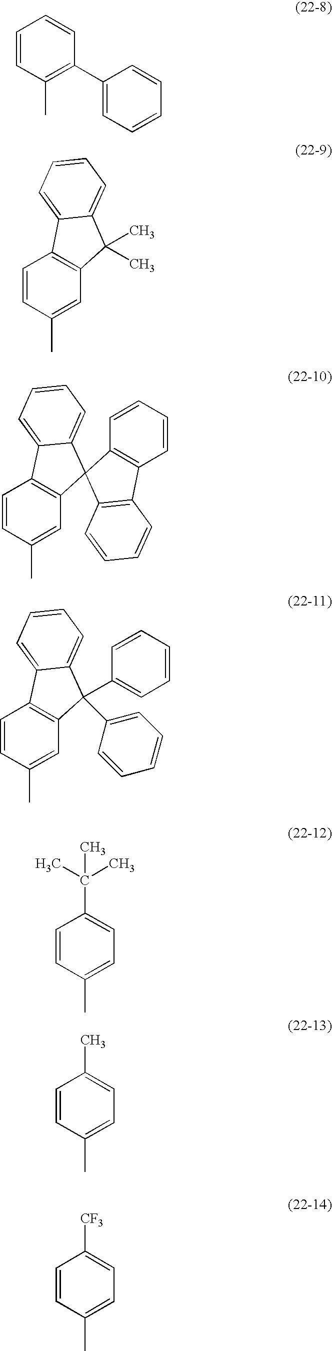

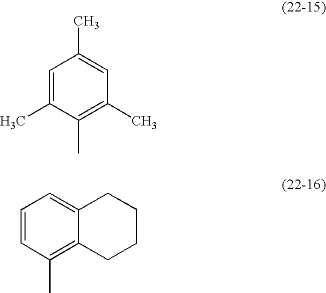

In the general formula (P1), a substituent of Ar³ may be bonded to R¹⁰ or R¹¹ to form a ring structure which may be a spiro ring structure. Examples in such a case are represented, together with a carbazole skeleton bonded to Ar², by the structural formula (23-1) to the structural formula (23-4).

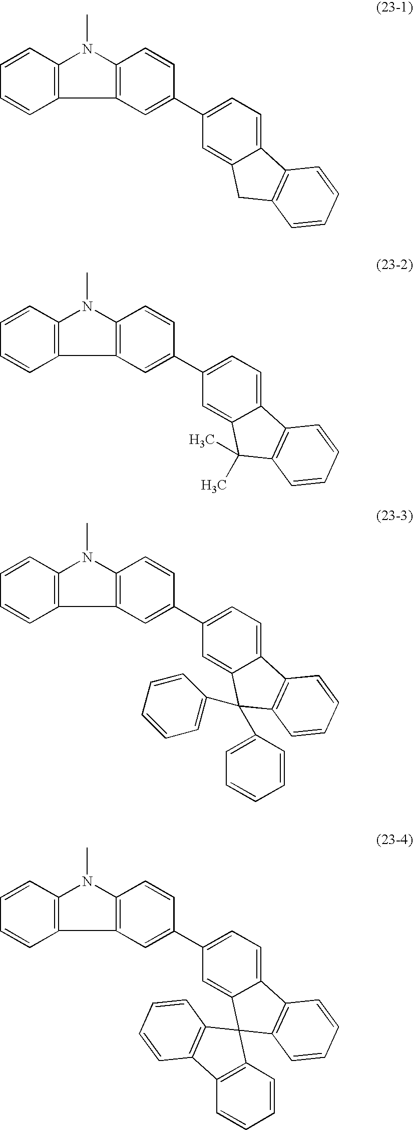

In the general formula (P1), Ar² represents an arylene group having 6 to 13 carbon atoms. The arylene group having 6 to 13 carbon atoms may have a substituent or substituents: when the arylene group having 6 to 13 carbon atoms has two or more substituents, the substituents may be bonded to each other to form a ring structure, and when one carbon atom has two substituents, the substituents may be bonded to each other to form a spiro ring structure. As Ar², substituents which are represented by the structural formula (24-1) to the structural formula (24-11) can be specifically given.

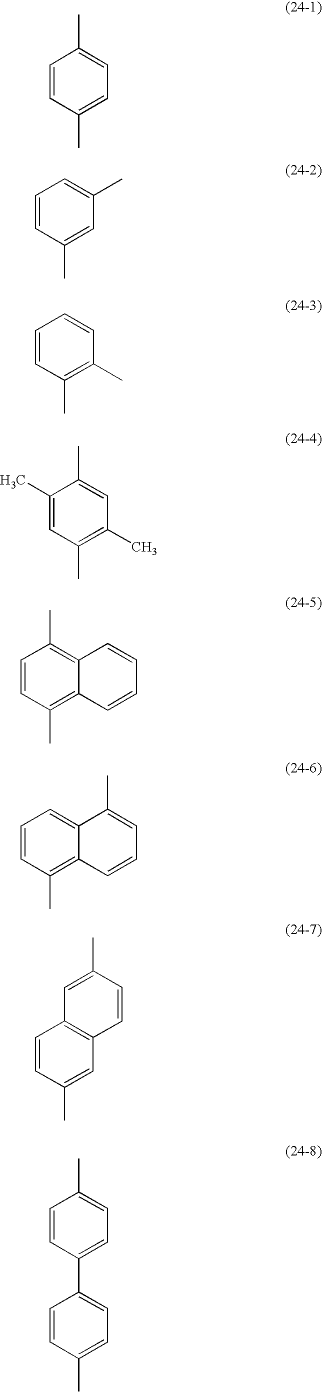

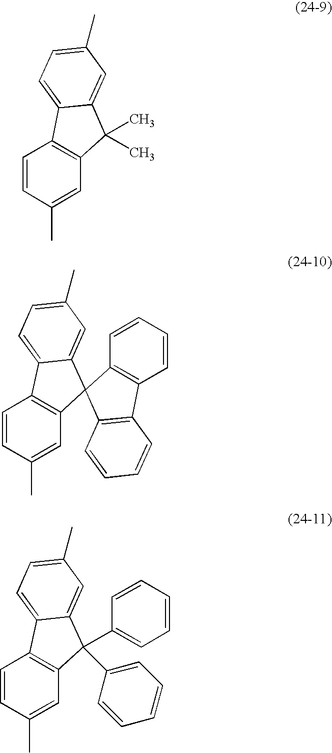

Among the carbazole derivatives represented by the general formula (P1), a carbazole derivative represented by the general formula (P2) is preferable.

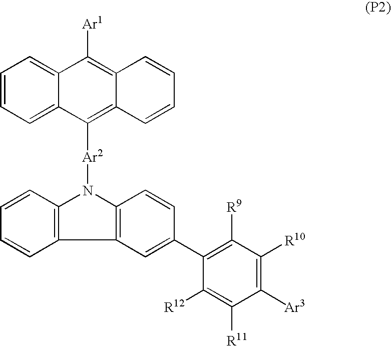

In the formula, R⁹ to R¹² independently represent hydrogen or an alkyl group having 1 to 4 carbon atoms, Ar¹ and Ar³ independently represent an aryl group having 6 to 13 carbon atoms, and Ar² represents an arylene group having 6 to 13 carbon atoms. The aryl group having 6 to 13 carbon atoms and the arylene group having 6 to 13 carbon atoms may independently have a substituent or substituents: when the aryl group having 6 to 13 carbon atoms and the arylene group having 6 to 13 carbon atoms independently have two or more substituents, the substituents may be bonded to each other to form a ring structure, and when one carbon atom of any of the aryl group having 6 to 13 carbon atoms and the arylene group having 6 to 13 carbon atoms has two substituents, the substituents may be bonded to each other to form a spiro ring structure. In addition, a substituent of Ar³ may be bonded to R¹⁰ or R¹¹ to form a ring structure which may be a spiro ring structure.

A carbazole derivative which is represented by the general formula (P3) is more preferable.

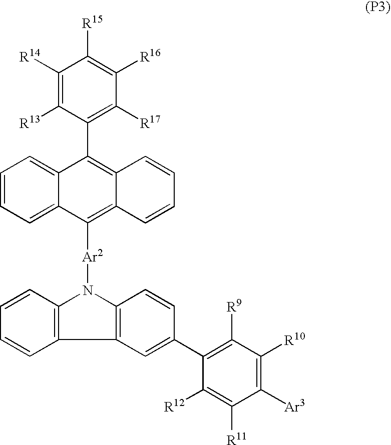

In the formula, R⁹ to R¹² independently represent hydrogen or an alkyl group having 6 to 10 carbon atoms; R¹³ to R¹⁷ independently represent hydrogen, an alkyl group having 1 to 4 carbon atoms, or an aryl group having 6 to 10 carbon atoms; Ar² represents an arylene group having 6 to 13 carbon atoms; and Ar³ represents an aryl group having 6 to 13 carbon atoms. The aryl group having 6 to 10 carbon atoms, the arylene group having 6 to 13 carbon atoms, and the aryl group having 6 to 13 carbon atoms may independently have a substituent or substituents: when the aryl group having 6 to 10 carbon atoms, the arylene group having 6 to 13 carbon atoms, and the aryl group having 6 to 13 carbon atoms independently have two or more substituents, the substituents may be bonded to each other to form a ring structure, and when one carbon atom of any of the aryl group having 6 to 10 carbon atoms, the arylene group having 6 to 13 carbon atoms, and the aryl group having 6 to 13 carbon atoms has two substituents, the substituents may be bonded to each other to form a spiro ring structure. In addition, a substituent of Ar³ may be bonded to R¹⁰ or R¹¹ to form a ring structure which may be a spiro ring structure.

A carbazole derivative which is represented by the general formula (P4) is more preferable.

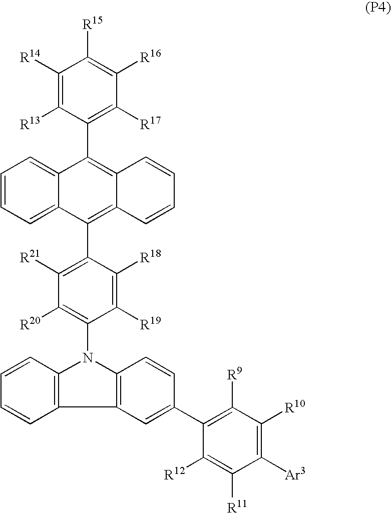

In the formula, R⁹ to R¹² and R¹⁸ to R²¹ independently represent hydrogen or an alkyl group having 1 to 4 carbon atoms; R¹³ to R¹⁷ independently represent hydrogen, an alkyl group having 1 to 4 carbon atoms, or an aryl group having 6 to 10 carbon atoms; and Ar³ represents an aryl group having 6 to 13 carbon atoms. The aryl group having 6 to 10 carbon atoms and the aryl group having 6 to 13 carbon atoms may independently have a substituent or substituents: when the aryl group having 6 to 10 carbon atoms and the aryl group having 6 to 13 independently have two or more substituents, the substituents may be bonded to each other to form a ring structure, and when one carbon atom of any of the aryl group having 6 to 10 carbon atoms and the aryl group having 6 to 13 carbon atoms has two substituents, the substituents may be bonded to each other to form a spiro ring structure. In addition, a substituent of Ar³ may be bonded to R¹⁰ or R¹¹ to form a ring structure which may be a spiro ring structure.

As specific examples of the carbazole derivatives of this embodiment, carbazole derivatives represented by the structural formula (31) to the structural formula (78) can be given. However, the present invention is not limited thereto.

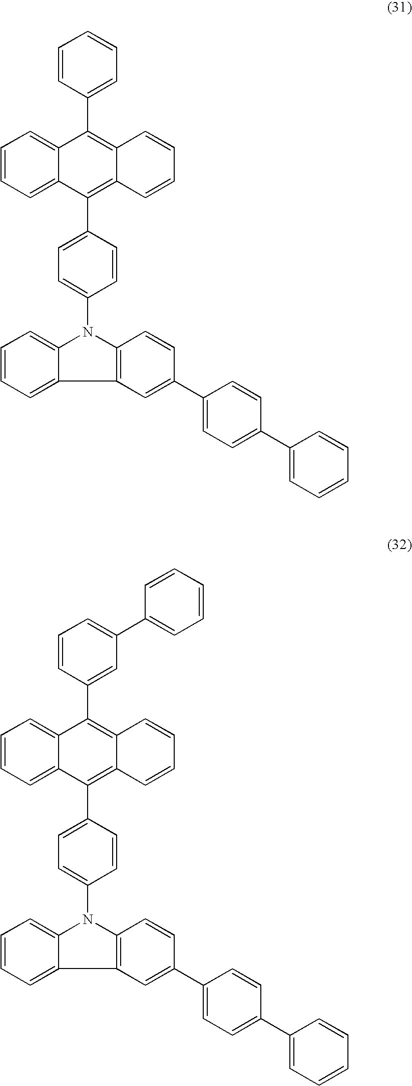

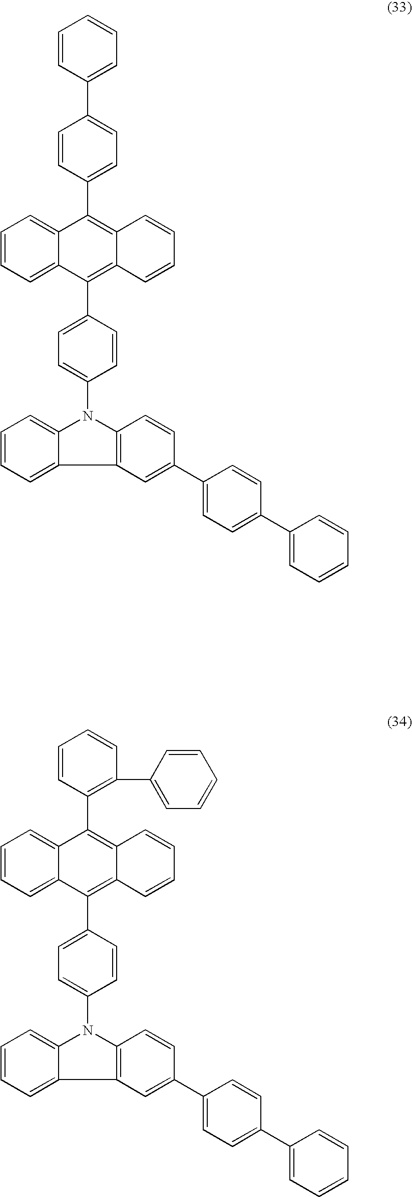

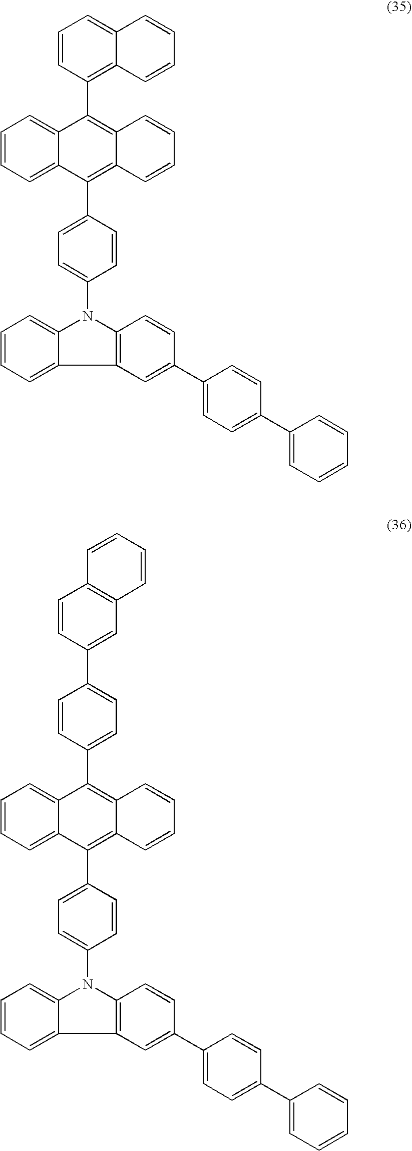

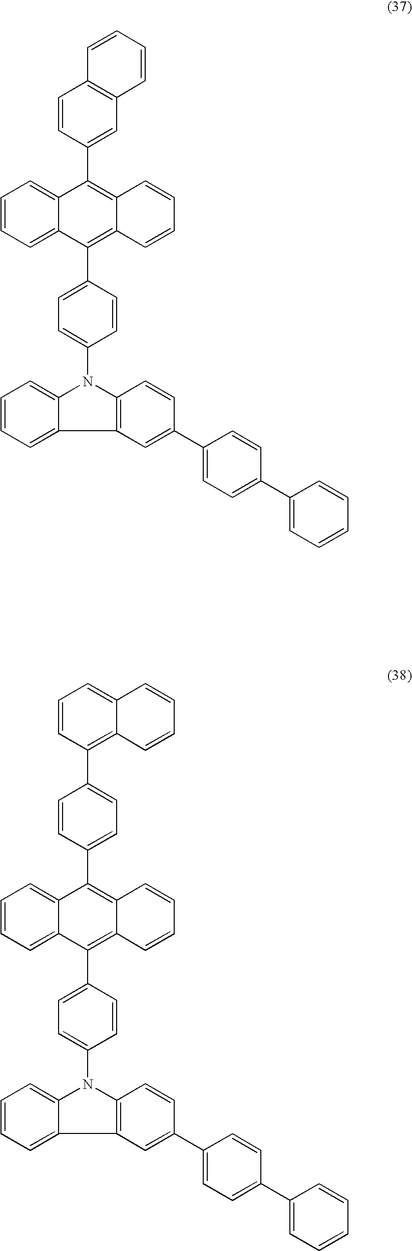

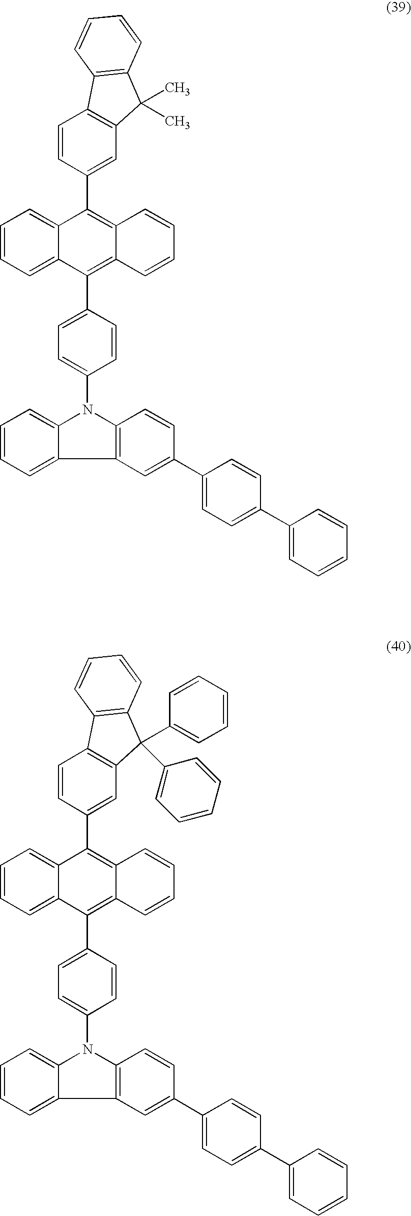

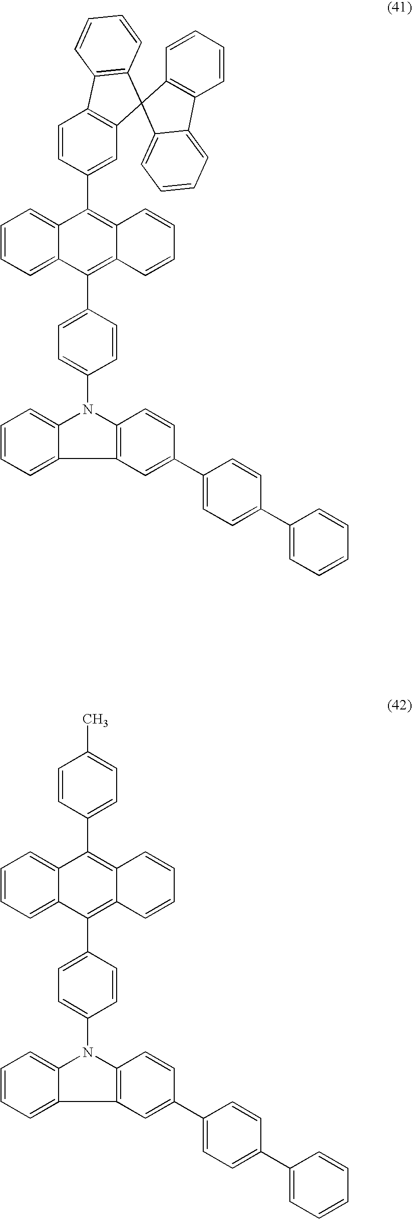

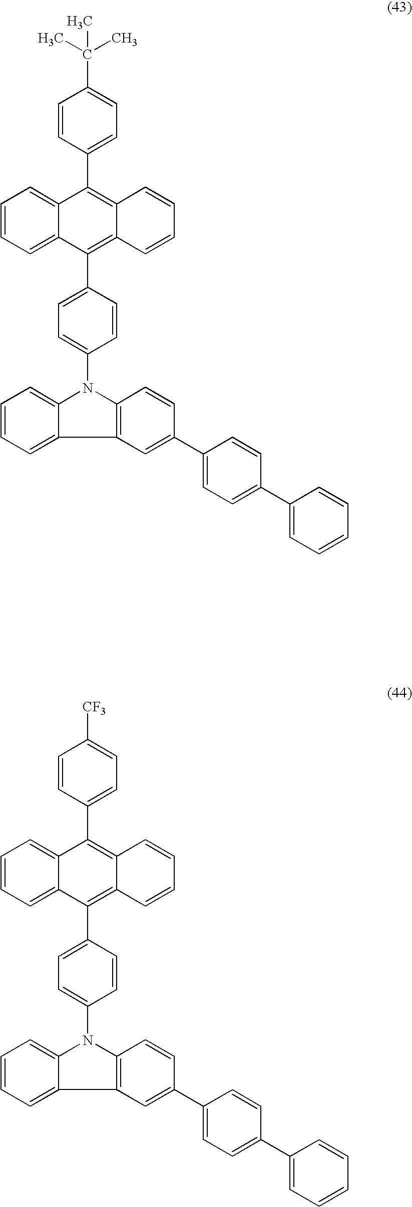

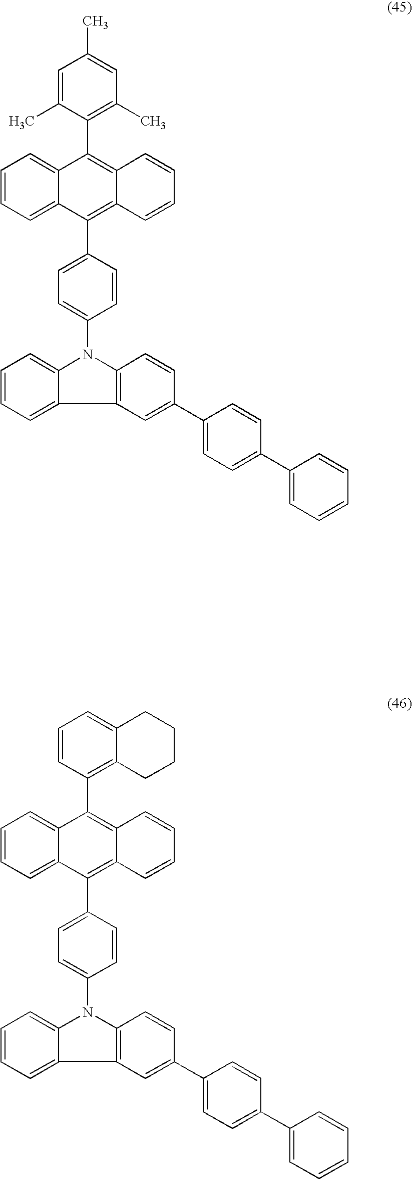

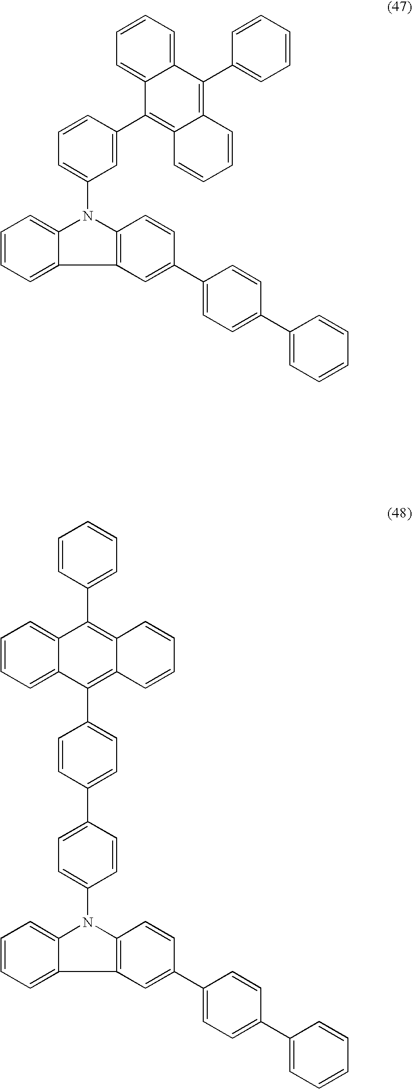

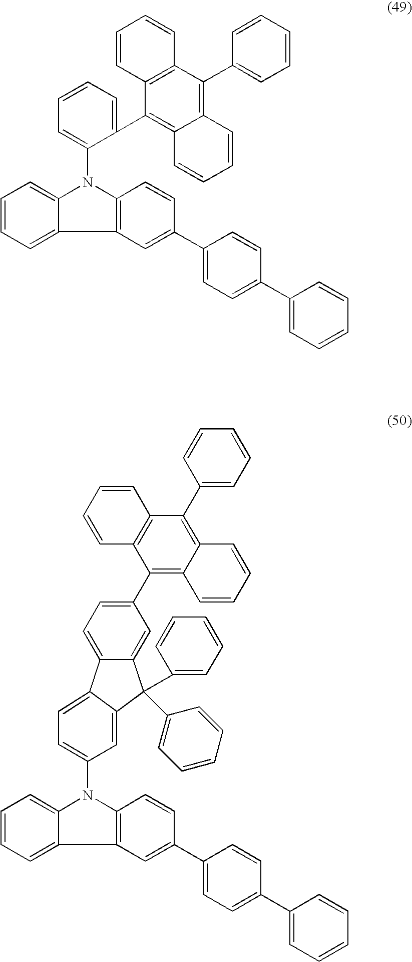

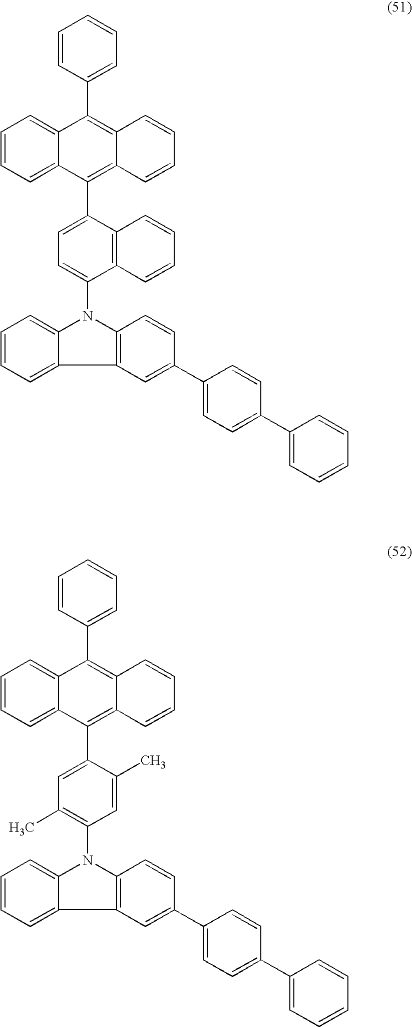

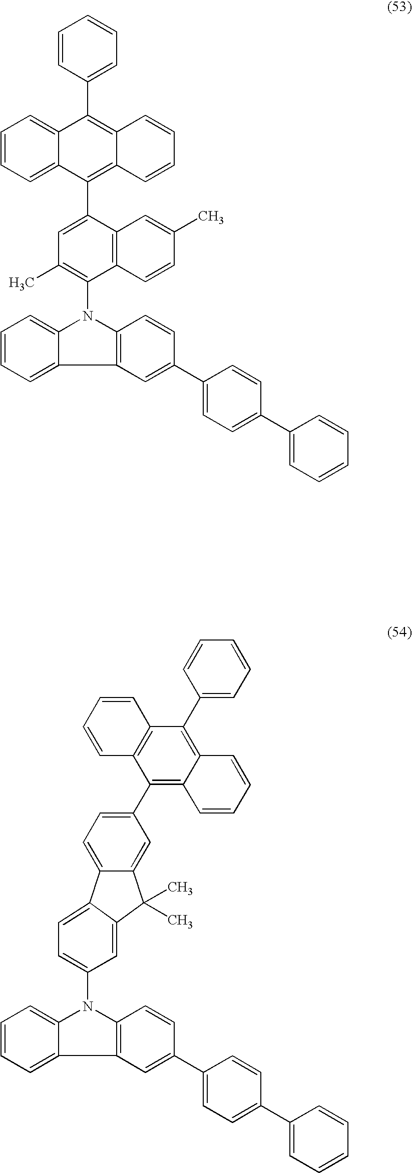

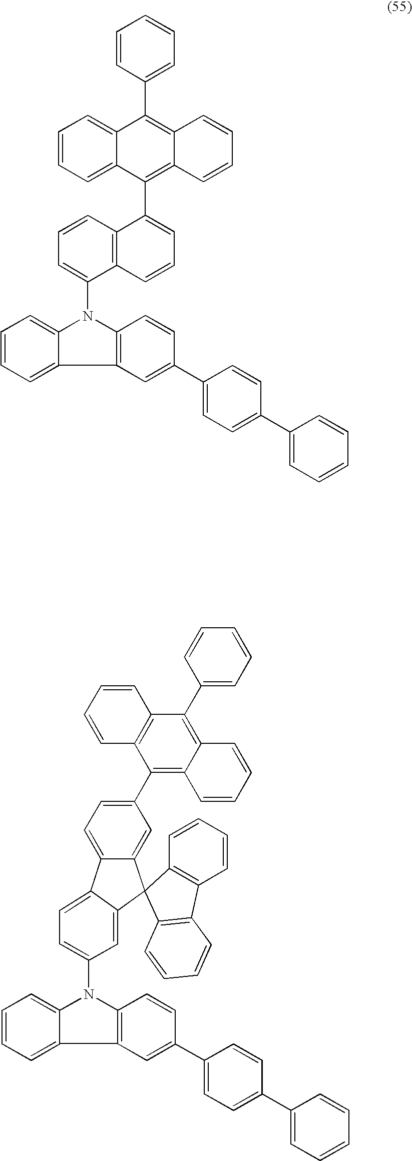

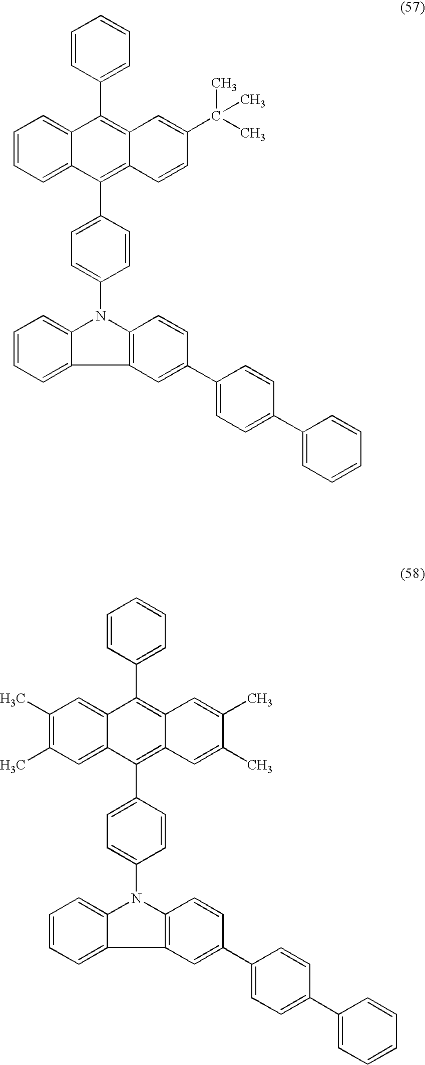

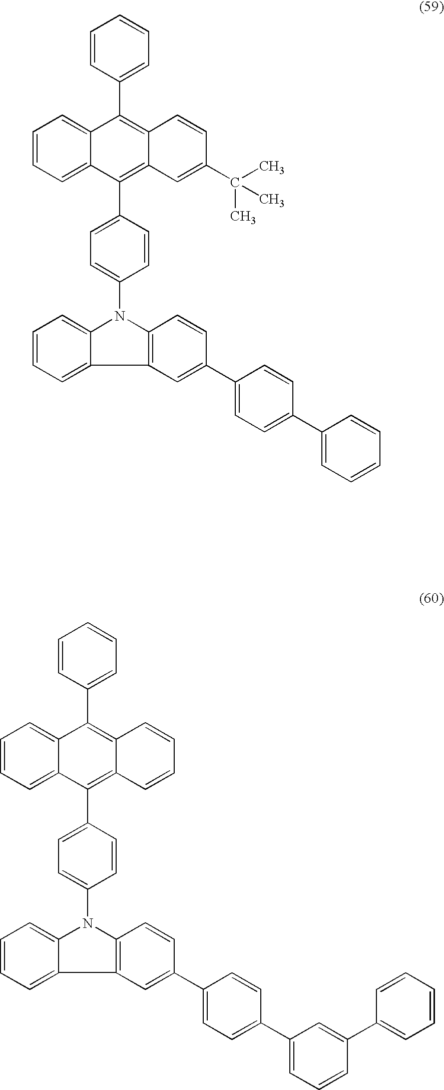

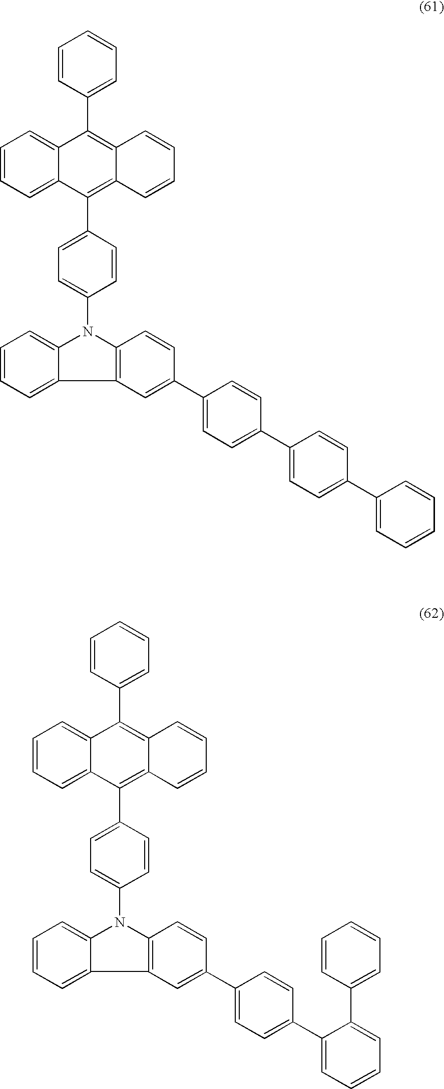

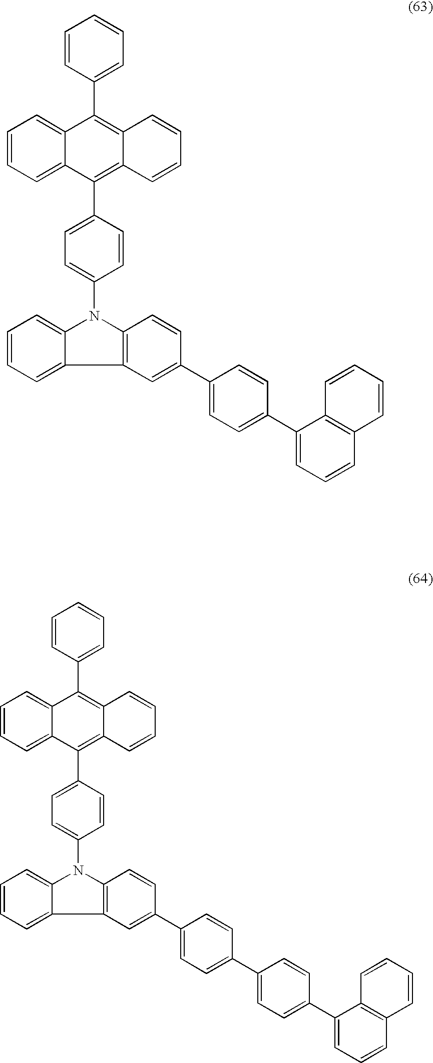

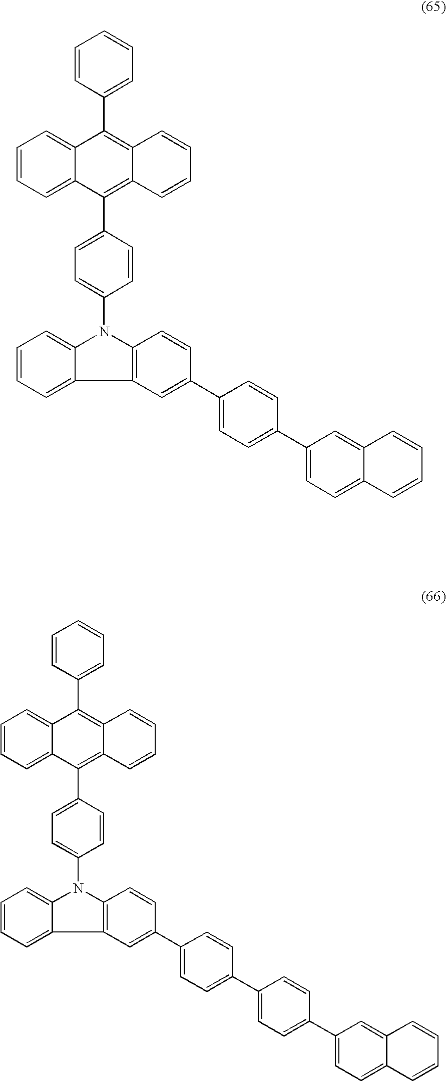

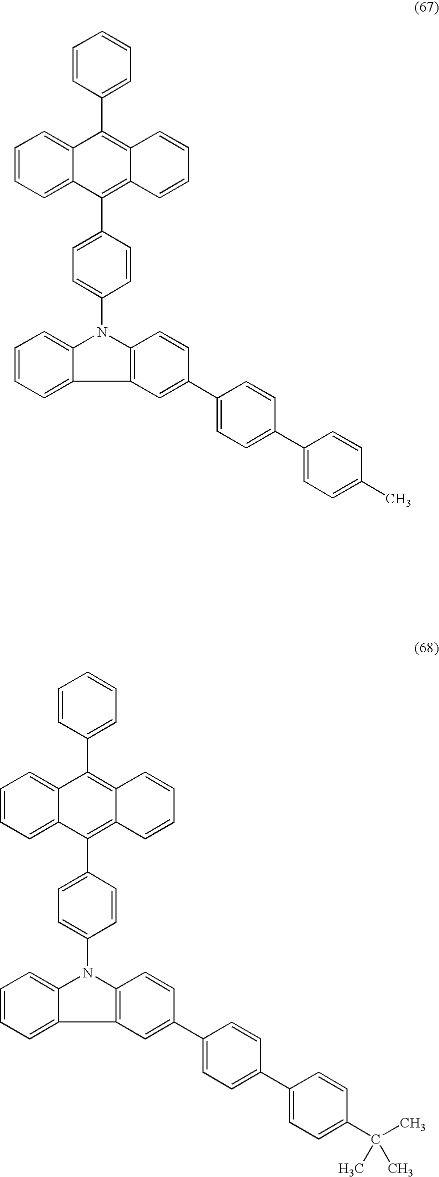

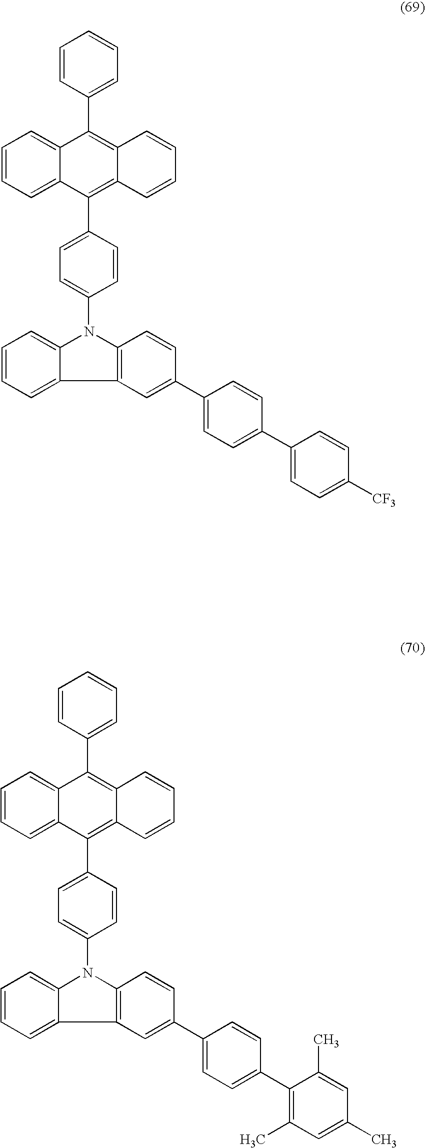

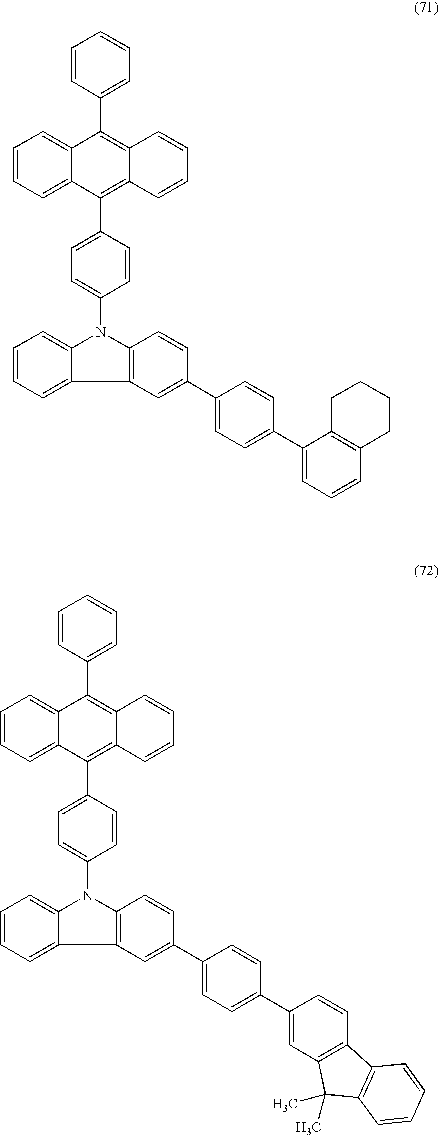

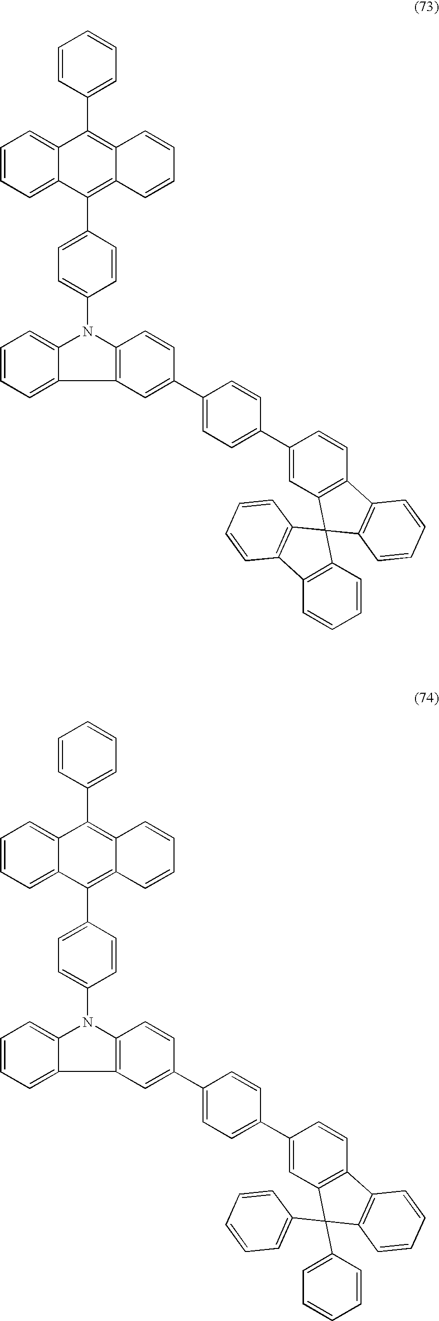

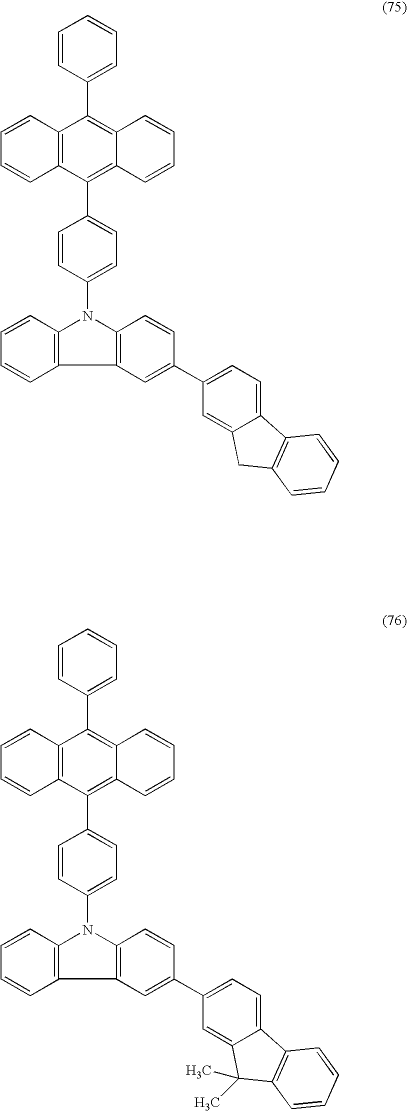

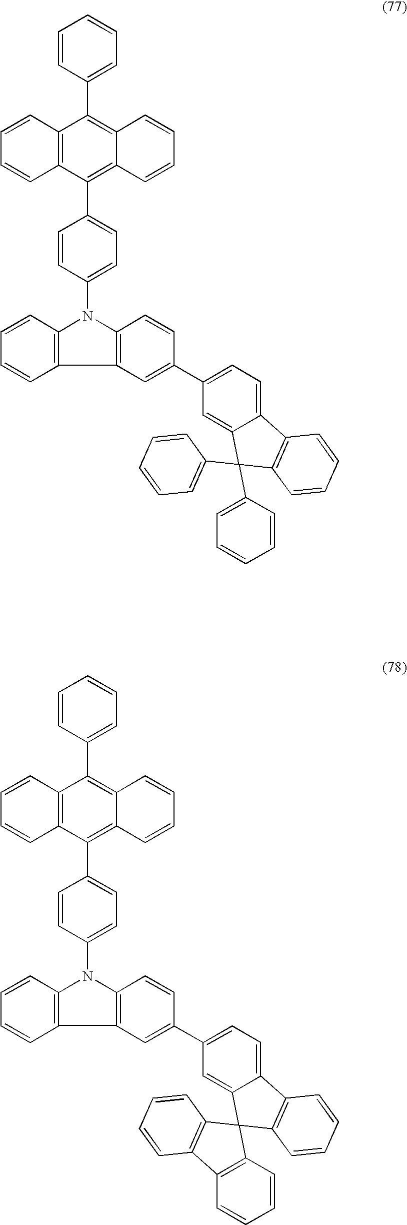

The carbazole derivative represented by the general formula (P1) can be synthesized by the synthesis methods represented by the synthetic schemes (H-1) to (H-3) and (I-1) and (J-1).

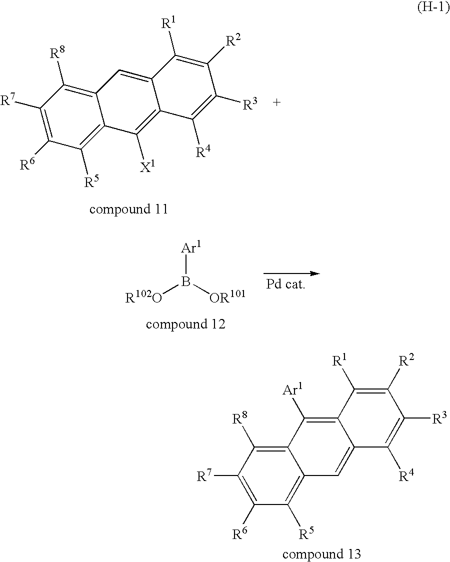

A 9-arylanthracene derivative (compound 13) can be obtained by Suzuki-Miyaura coupling of an anthracene derivative (compound 11) and an arylorganoboron compound such as an arylboronic acid (compound 12) in the presence of a palladium catalyst (the synthetic scheme (H-1)).

In the synthetic scheme (H-1), X¹ represents a halogen or a triflate group and the halogen is preferably iodine, bromine, or chlorine; R¹ to R⁸ independently represent hydrogen or an alkyl group having 1 to 4 carbon atoms; Ar¹ represents an aryl group having 6 to 13 carbon atoms which may have a substituent or substituents which may be bonded to each other to form a ring structure which may be a spiro ring structure; and R¹⁰¹ and R¹⁰² independently represent hydrogen or an alkyl group having 1 to 6 carbon atoms and R¹⁰¹ and R¹⁰² may be bonded to each other to form a ring structure.

Examples of a palladium catalyst which can be used in the synthetic scheme (H-1) include, but are not limited to, palladium(II) acetate, tetrakis(triphenylphosphine)palladium(0). Examples of a ligand of the palladium catalyst which can be used in the synthetic scheme (H-1) include, but are not limited to, tri(ortho-tolyl)phosphine, triphenylphosphine, and tricyclohexylphosphine.

Examples of a base which can be used in the synthetic scheme (H-1) include, but are not limited to, an organic base such as sodium tert-butoxide and an inorganic base such as potassium carbonate.

Examples of a solvent which can be used in the synthetic scheme (H-1) include, but are not limited to, a mixed solvent of toluene and water; a mixed solvent of toluene, alcohol such as ethanol, and water; a mixed solvent of xylene and water; a mixed solvent of xylene, alcohol such as ethanol, and water; a mixed solvent of benzene and water; a mixed solvent of benzene, alcohol such as ethanol, and water; and a mixed solvent of ether such as ethylene glycol dimethyl ether and water. Further, a mixed solvent of toluene and water or a mixed solvent of toluene, ethanol, and water is more preferable.

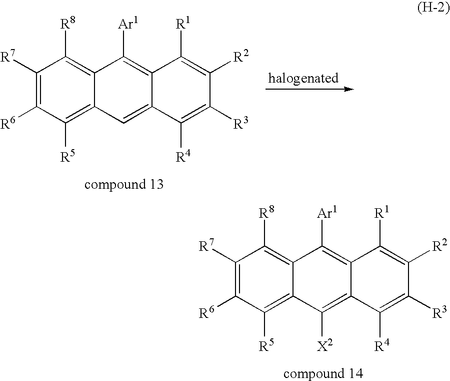

Then, a halogenated arylanthracene derivative (compound 14) can be obtained by halogenating the 9-arylanthracene derivative (compound 13) (the synthetic scheme (H-2)).

In the synthetic scheme (H-2), X² represents a halogen and the halogen is preferably iodine or bromine; R¹ to R⁸ independently represent hydrogen or an alkyl group having 1 to 4 carbon atoms; Ar¹ represents an aryl group having 6 to 13 carbon atoms which may have a substituent or substituents which may be bonded to each other to form a ring structure which may be a spiro ring structure.

In the case of brominating the 9-arylanthracene derivative (compound 13) in the synthetic scheme (H-2), examples of a brominating agent which can be used include, but are not limited to, bromine and N-bromosuccinimide. Examples of a solvent which can be used in the case of brominating the 9-arylanthracene derivative (compound 13) using bromine include, but are not limited to, a halogen-based solvent such as chloroform or carbon tetrachloride. Examples of a solvent which can be used in the case of brominating the 9-arylanthracene derivative (compound 13) using N-bromosuccinimide include, but are not limited to, ethyl acetate, tetrahydrofuran, dimethylformamide, acetic acid, water, and toluene.

In the case of iodinating the 9-arylanthracene derivative (compound 13) in the synthetic scheme (H-2), examples of an iodinating agent which can be used include, but are not limited to, N-iodosuccinimide, 1,3-diiodo-5,5-dimethylimidazolidine-2,4-dione (DIH), 2,4,6,8-tetraiodo-2,4,6,8-tetraazabicyclo[3,3,0]octane-3,7-dion, and 2-iodo-2,4,6,8-tetraazabicyclo[3,3,0]octane-3,7-dion. Further, examples of a solvent which can be used in the case of iodinating the 9-arylanthracene derivative (compound 13) using any of those iodinating agents include acetic acid (glacial acetic acid); water; aromatic hydrocarbons such as benzene, toluene, and xylene; ethers such as 1,2-dimethoxyethane, diethyl ether, methyl-t-butyl ether, tetrahydrofuran, and dioxane; saturated hydrocarbons such as pentane, hexane, heptane, octane, and cyclohexane; halogenated hydrocarbons such as dichloromethane, chloroform, carbon tetrachloride, 1,2-dichloroethane, and 1,1,1-trichloroethane; nitriles such as acetonitrile and benzonitrile; and esters such as ethyl acetate, methyl acetate, and butyl acetate. Those solvents can be used alone or in combination. When water is used, it is preferably mixed with an organic solvent. In addition, in this reaction, an acid such as sulfuric acid or acetic acid is preferably used as well and an acid which can be used is not limited thereto.

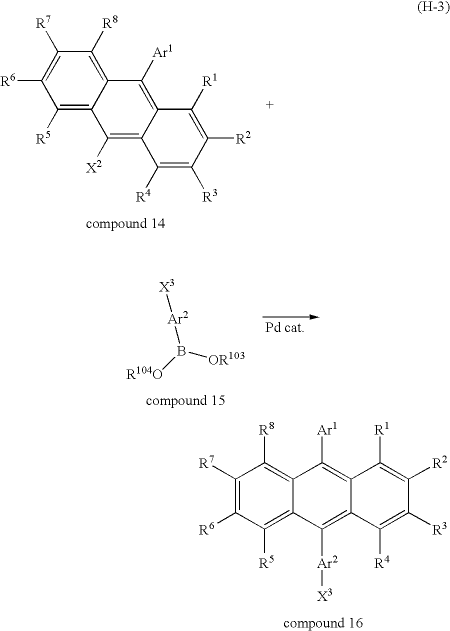

Then, a halogenated diarylanthracene derivative (compound 16) can be obtained by Suzuki-Miyaura coupling of the arylanthracene derivative (compound 14) and an arylorganoboron compound such as a halogenated arylboronic acid (compound 15) in the presence of a palladium catalyst (the synthetic scheme (H-3)).

In the synthetic scheme (H-3), X² represents a halogen and the halogen is preferably iodine or bromine; X³ represents a halogen or a triflate group and the halogen is preferably iodine, bromine, or chlorine; R¹ to R⁸ independently represent hydrogen or an alkyl group having 1 to 4 carbon atoms; Ar¹ represents an aryl group having 6 to 13 carbon atoms which may have a substituent or substituents which may be bonded to each other to form a ring structure which may be a spiro ring structure; Ar² represents an arylene group having 6 to 13 carbon atoms which may have a substituent or substituents which may be bonded to each other to form a ring structure which may be a spiro ring structure; and R¹⁰³ and R¹⁰⁴ independently represent hydrogen or an alkyl group having 1 to 6 carbon atoms and R¹⁰³ and R¹⁰⁴ may be bonded to each other to form a ring structure.

Examples of a palladium catalyst which can be used in the synthetic scheme (H-3) include, but are not limited to, palladium(II) acetate, tetrakis(triphenylphosphine)palladium(0). Examples of a ligand of the palladium catalyst which can be used in the synthetic scheme (H-3) include, but are not limited to, tri(ortho-tolyl)phosphine, triphenylphosphine, and tricyclohexylphosphine.

Examples of a base which can be used in the synthetic scheme (H-3) include, but are not limited to, an organic base such as sodium tert-butoxide and an inorganic base such as potassium carbonate.

Examples of a solvent which can be used in the synthetic scheme (H-3) i include, but are not limited to, a mixed solvent of toluene and water; a mixed solvent of toluene, alcohol such as ethanol, and water; a mixed solvent of xylene and water; a mixed solvent of xylene, alcohol such as ethanol, and water; a mixed solvent of benzene and water; a mixed solvent of benzene, alcohol such as ethanol, and water; and a mixed solvent of ether such as ethylene glycol dimethyl ether and water. Further, a mixed solvent of toluene and water or a mixed solvent of toluene, ethanol, and water is more preferable.

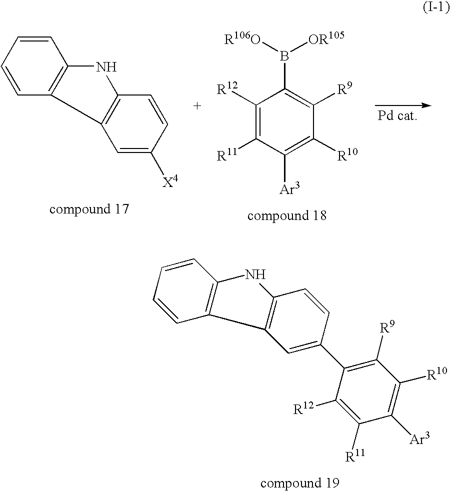

A carbazole derivative (compound 19) can be obtained by Suzuki-Miyaura coupling of a carbazole derivative (compound 17) and a phenyl organoboron compound such as a phenyl boronic acid (compound 18) in the presence of a palladium catalyst (the synthetic scheme (I-1)).

In the synthetic scheme (I-1), X⁴ represents a halogen or a triflate group and the halogen is preferably iodine, bromine, or chlorine; R⁹ to R¹² independently represent hydrogen or an alkyl group having 1 to 4 carbon atoms; Ar³ represents an aryl group having 6 to 13 carbon atoms which may have a substituent or substituents which may be bonded to each other to form a ring structure which may be a spiro ring structure, and a substituent of Ar³ may be bonded to R¹⁰ or R¹¹ to form a ring structure which may be a spiro ring structure; and R¹⁰⁵ and R¹⁰⁶ independently represent hydrogen or an alkyl group having 1 to 6 carbon atoms and R¹⁰⁵ and R¹⁰⁶ may be bonded to each other to form a ring structure.

Examples of a palladium catalyst which can be used in the synthetic scheme (I-1) include, but are not limited to, palladium(II) acetate, tetrakis(triphenylphosphine)palladium(0). Examples of a ligand of the palladium catalyst which can be used in the synthetic scheme (I-1) include, but are not limited to, tri(ortho-tolyl)phosphine, triphenylphosphine, and tricyclohexylphosphine.

Examples of a base which can be used in the synthetic scheme (I-1) include, but are not limited to, an organic base such as sodium tert-butoxide and an inorganic base such as potassium carbonate.

Examples of a solvent which can be used in the synthetic scheme (I-1) include, but are not limited to, a mixed solvent of toluene and water; a mixed solvent of toluene, alcohol such as ethanol, and water; a mixed solvent of xylene and water; a mixed solvent of xylene, alcohol such as ethanol, and water; a mixed solvent of benzene and water; a mixed solvent of benzene, alcohol such as ethanol, and water; and a mixed solvent of ether such as ethylene glycol dimethyl ether and water. Further, a mixed solvent of toluene and water or a mixed solvent of toluene, ethanol, and water is more preferable.

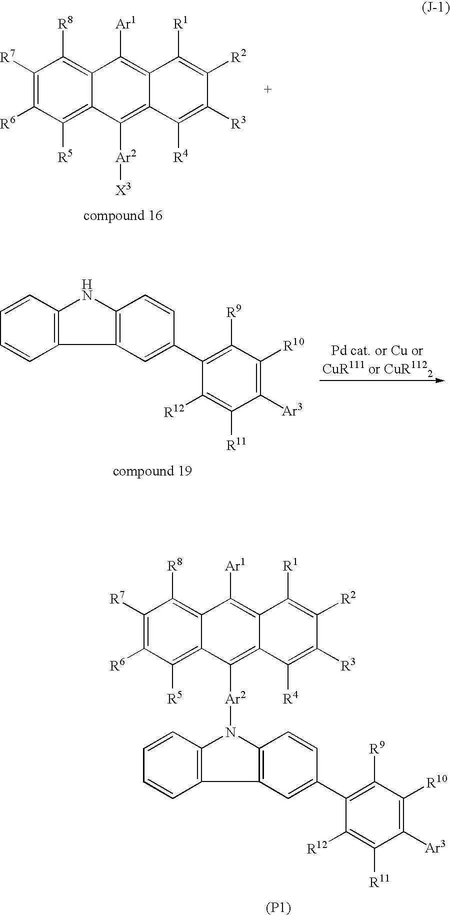

Then, from the halogenated anthracene derivative (compound 16) which is obtained through the synthetic schemes (H-1) to (H-3) and the carbazole derivative (compound 19) which is obtained through the synthetic scheme (I-1), the object which is represented by the general formula (P1) can be obtained by a coupling reaction of Buchwald-Hartwig reaction in the presence of a palladium catalyst or Ullmann reaction in the presence of copper or a copper compound (synthetic scheme (J-1)).

In the synthetic scheme (J-1), X³ represents a halogen or a triflate group and the halogen is preferably iodine, bromine, or chlorine; Ar¹ represents an aryl group having 6 to 13 carbon atoms which may have a substituent or substituents which may be bonded to each other to form a ring structure which may be a spiro ring structure; Ar² represents an arylene group having 6 to 13 carbon atoms which may have a substituent or substituents which may be bonded to each other to form a ring structure which may be a spiro ring structure; Ar³ represents an aryl group having 6 to 13 carbon atoms which may have a substituent or substituents which may be bonded to each other to form a ring structure which may be a spiro ring structure, and a substituent of Ar³ may be bonded to R¹⁰ or R¹¹ to form a ring structure which may be a spiro ring structure; R¹ to R⁸ independently represent hydrogen or an alkyl group having 1 to 4 carbon atoms; and R⁹ to R¹² independently represent hydrogen or an alkyl group having 1 to 4 carbon atoms.

The case in which Buchwald-Hartwig reaction is carried out in the synthetic scheme (J-1) is described. Examples of a palladium catalyst which can be used include, but are not limited to, bis(dibenzylideneacetone)palladium(0) and palladium(II) acetate. Examples of a ligand of the palladium catalyst which can be used in the synthetic scheme (J-1) include, but are not limited to, tri(tert-butyl)phosphine, tri(n-hexyl)phosphine, and tricyclohexylphosphine.

Examples of a base which can be used in the synthetic scheme (J-1) include, but are not limited to, an organic base such as sodium tert-butoxide and an inorganic base such as potassium carbonate.

Examples of a solvent which can be used in the synthetic scheme (J-1) include, but are not limited to, toluene, xylene, benzene, and tetrahydrofuran.

The case in which Ullmann reaction is carried out in the synthetic scheme (J-1) is described. In the synthetic scheme (J-1), R¹¹¹ and R¹¹² independently represent a halogen or an acetyl group and the halogen can be chlorine, bromine, or iodine. In addition, it is preferable to use copper(I) iodide where R¹¹¹ is iodine or copper(II) acetate where R¹¹² is an acetyl group, but the copper compound which is used for the reaction is not limited thereto. Further, copper can be used as an alternative to the copper compound.

Examples of a base that can be used in the synthetic scheme (J-1) include, but are not limited to, an inorganic base such as potassium carbonate.

Examples of a solvent which can be used in the synthetic scheme (J-1) include, but are not limited to, 1,3-dimethyl-3,4,5,6-tetrahydro-2(1H)pyrimidinone (DMPU), toluene, xylene, and benzene. In Ullmann reaction, DMPU or xylene which has a high boiling point is preferably used because the object can be obtained in a shorter time and at a higher yield when the reaction temperature is 100° C. or higher. DMPU is more preferably used because the reaction temperature is more preferably 150° C. or higher.

As described thus far, the carbazole derivative according to this embodiment can be synthesized.

The carbazole derivative according to this embodiment has a large band gap, and therefore light with a short wavelength can be exhibited. Accordingly, blue-light emission with good color purity can be exhibited. In addition, the carbazole derivative according to this embodiment is a bipolar material having electron- and hole-transporting properties. In addition, the carbazole derivative according to this embodiment has high electrochemical stability and thermal stability.

The carbazole derivative in this embodiment can be used alone for a layer containing a light-emitting substance. Further, the carbazole derivative in this embodiment can also be used as a host in a light-emitting layer. Light emission from a dopant that functions as a light-emitting substance can be obtained with a structure in which the dopant is dispersed in the carbazole derivative according to this embodiment. When the carbazole derivative according to this embodiment is used as a host in a light-emitting layer, blue-light emission with good color purity can be obtained.

Further, a light-emitting element can be manufactured in which the carbazole derivative according to this embodiment is added to a layer formed from a material (hereinafter, referred to as a host) which has a larger band gap than the carbazole derivative according to this embodiment. In that case, light emission from the carbazole derivative according to this embodiment can be obtained. That is, the carbazole derivative according to this embodiment can also function as a dopant. At this time, since the carbazole derivative according to this embodiment has a large band gap and light with a short wavelength can be exhibited, blue-light emission with good color purity can be exhibited. Accordingly, a highly reliable light-emitting element can be manufactured.

The carbazole derivative according to this embodiment can be used as a carrier-transporting material contained in a functional layer of a light-emitting element. For example, the carbazole derivative according to this embodiment can be used in a carrier-transporting layer such as a hole-transporting layer, a hole-injecting layer, an electron-transporting layer, and an electron-injecting layer.

### Embodiment 3

In this embodiment, one mode of a carbazole derivative of the present invention will be described.

A carbazole derivative of this embodiment is represented by the general formula (M1).

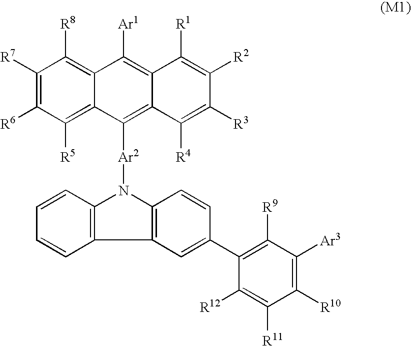

In the formula, R¹ to R¹² independently represent hydrogen or an alkyl group having 1 to 4 carbon atoms, Ar¹ and Ar³ independently represent an aryl group having 6 to 13 carbon atoms, and Ar² represents an arylene group having 6 to 13 carbon atoms. The aryl group having 6 to 13 carbon atoms and the arylene group having 6 to 13 carbon atoms may independently have a substituent or substituents: when the aryl group having 6 to 13 carbon atoms and the arylene group having 6 to 13 carbon atoms independently have two or more substituents, the substituents may be bonded to each other to form a ring structure, and when one carbon atom of any of the aryl group having 6 to 13 carbon atoms and the arylene group having 6 to 13 carbon atoms has two substituents, the substituents may be bonded to each other to form a spiro ring structure. In addition, a substituent of Ar³ may be bonded to R⁹ or R¹⁰ to form a ring structure which may be a spiro ring structure.

In the general formula (M1), R¹ to R¹² independently represent hydrogen or an alkyl group having 1 to 4 carbon atoms. For example, substituents which are represented by the structural formula (25-1) to the structural formula (25-9) can be given.

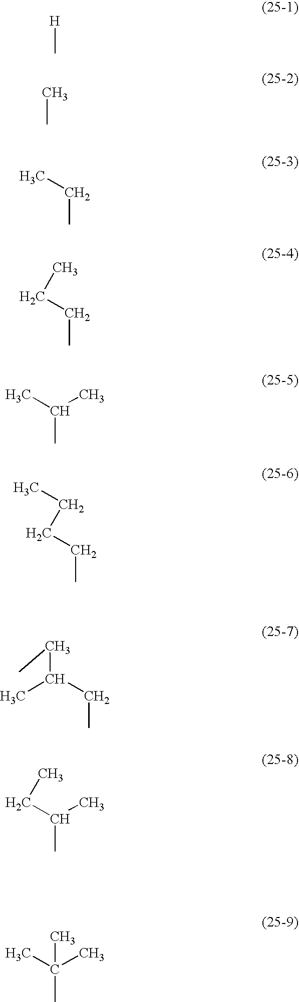

In the general formula (M1), Ar¹ and Ar³ independently represent an aryl group having 6 to 13 carbon atoms. The aryl group having 6 to 13 carbon atoms may have a substituent or substituents: when the aryl group having 6 to 13 carbon atoms has two or more substituents, the substituents may be bonded to each other to form a ring structure, and when one carbon atom has two substituents, the substituents may be bonded to each other to form a spiro ring structure. As Ar¹ and Ar³, for example, substituents which are represented by the structural formula (26-1) to the structural formula (26-20) can be given.

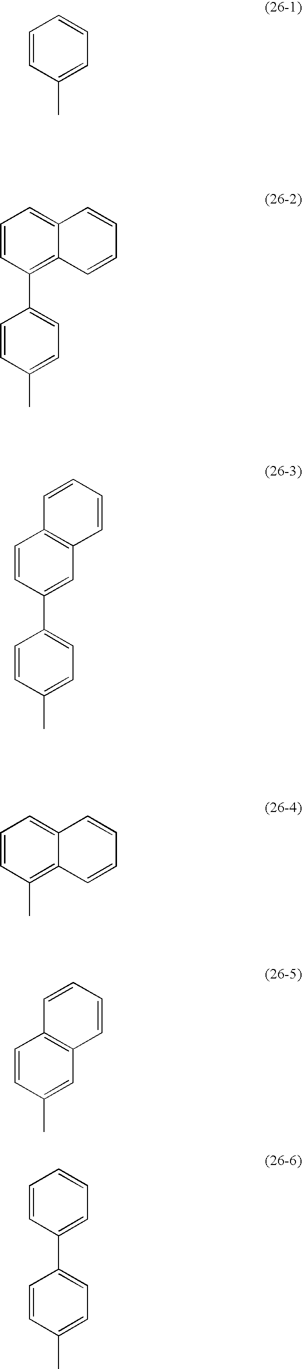

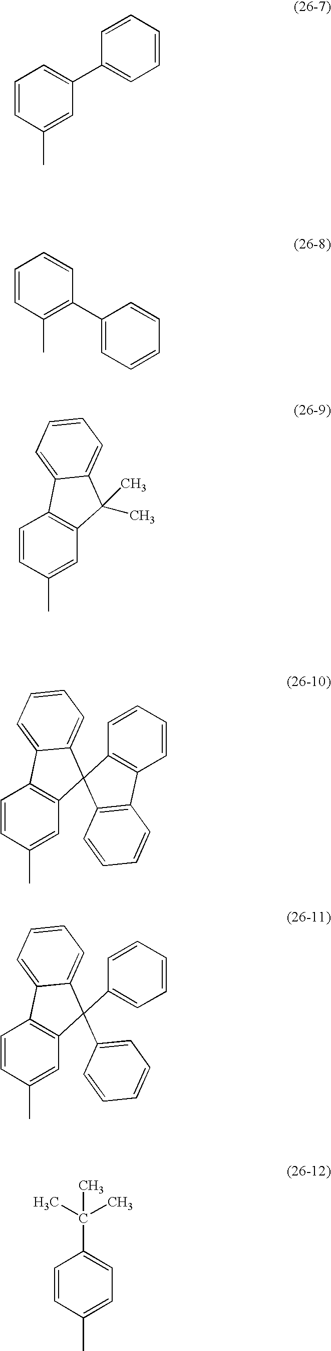

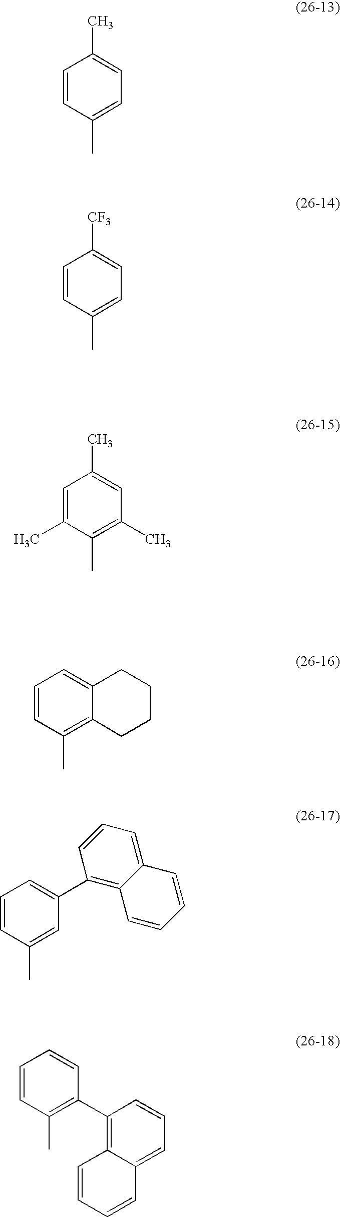

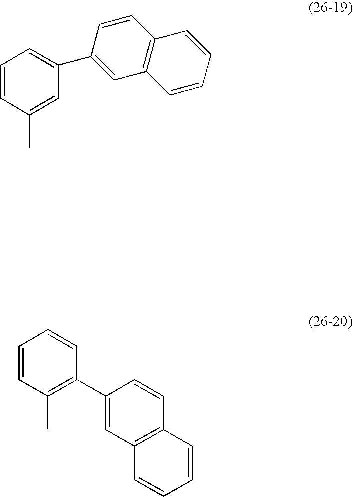

In the general formula (M1), a substituent of Ar³ may be bonded to R⁹ or R¹⁰ to form a ring structure which may be a spiro ring structure. Examples in such a case are represented, together with a carbazole skeleton bonded to Ar², by the structural formula (27-1) to the structural formula (27-8).

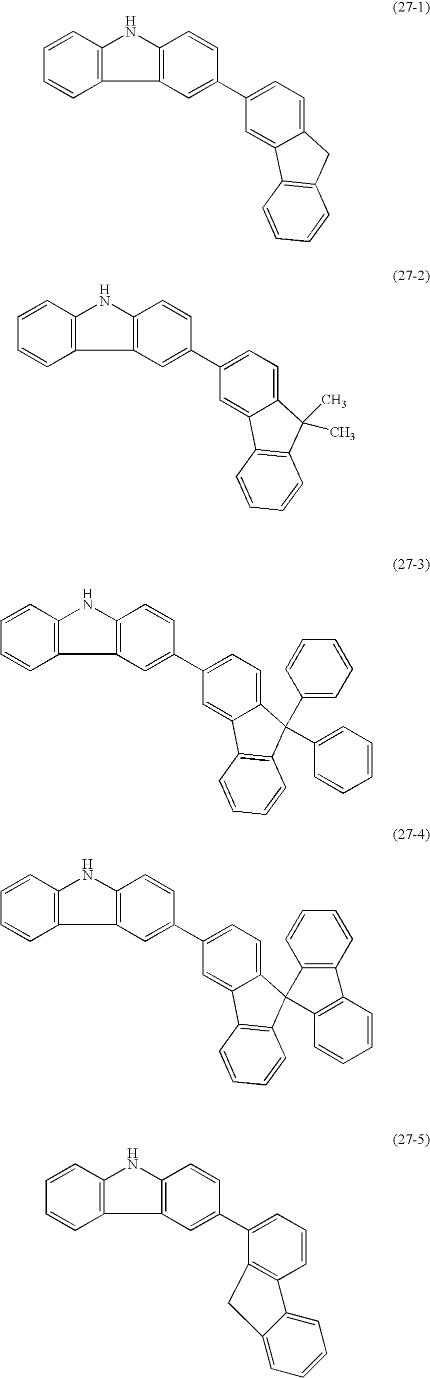

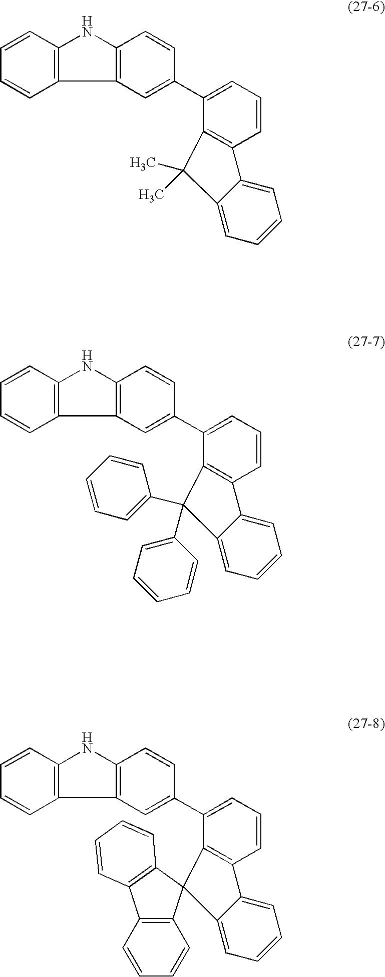

In the general formula (M1), Ar² represents an arylene group having 6 to 13 carbon atoms. The arylene group having 6 to 13 carbon atoms may have a substituent or substituents: when the arylene group having 6 to 13 carbon atoms has two or more substituents, the substituents may be bonded to each other to form a ring structure, and when one carbon atom has two substituents, the substituents may be bonded to each other to form a spiro ring structure. As Ar², substituents which are represented by the structural formula (28-1) to the structural formula (28-11) can be specifically given.

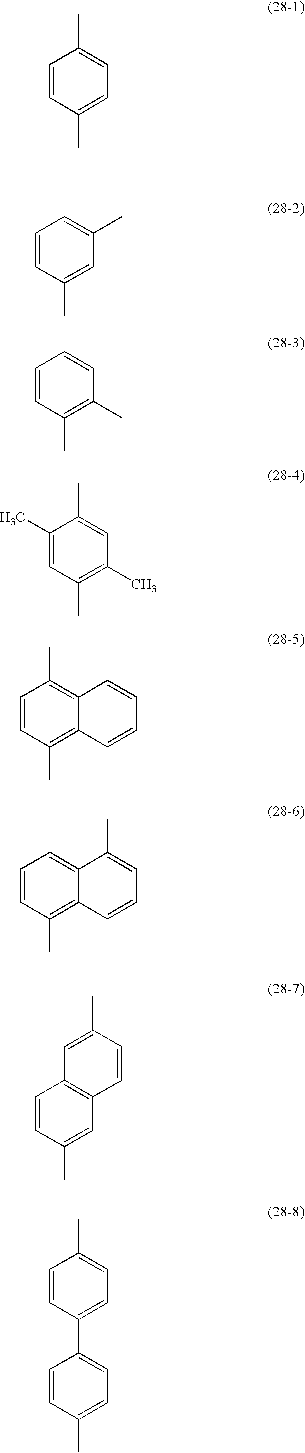

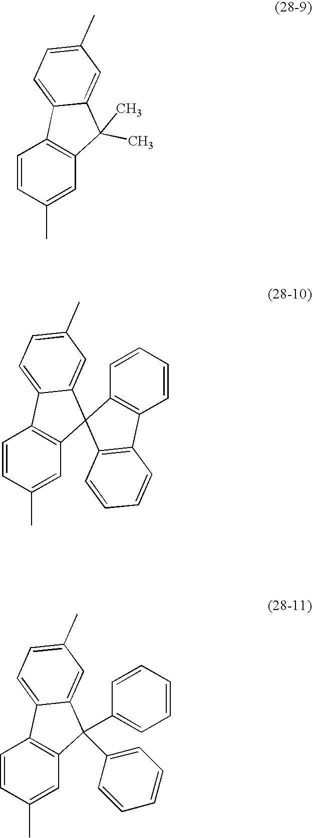

Among the carbazole derivatives represented by the general formula (M1), a carbazole derivative represented by the general formula (M2) is preferable.

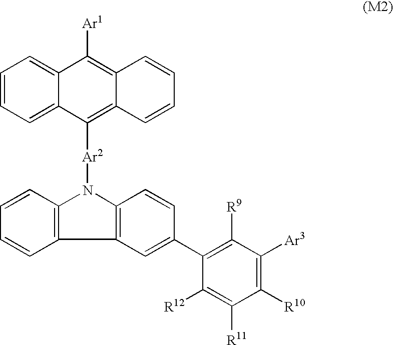

In the formula, R⁹ to R¹² independently represent hydrogen or an alkyl group having 1 to 4 carbon atoms, Ar¹ and Ar³ independently represent an aryl group having 6 to 13 carbon atoms, and Ar² represents an arylene group having 6 to 13 carbon atoms. The aryl group having 6 to 13 carbon atoms and the arylene group having 6 to 13 carbon atoms may independently have a substituent or substituents: when the aryl group having 6 to 13 carbon atoms and the arylene group having 6 to 13 carbon atoms independently have two or more substituents, the substituents may be bonded to each other to form a ring structure, and when one carbon atom of any of the aryl group having 6 to 13 carbon atoms and the arylene group having 6 to 13 carbon atoms has two substituents, the substituents may be bonded to each other to form a spiro ring structure. In addition, a substituent of Ar³ may be bonded to R⁹ or R¹⁰ to form a ring structure which may be a spiro ring structure.

A carbazole derivative which is represented by the general formula (M3) is more preferable.

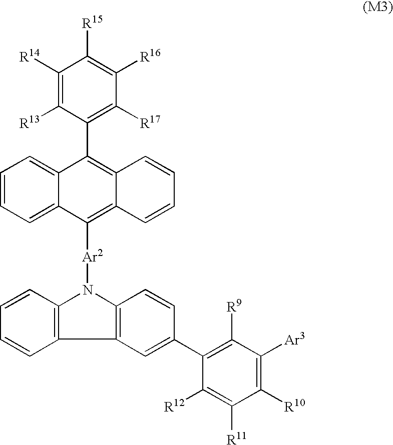

In the formula, R⁹ to R¹² independently represent hydrogen or an alkyl group having 1 to 4 carbon atoms; R¹³ to R¹⁷ independently represent hydrogen, an alkyl group having 1 to 4 carbon atoms, or an aryl group having 6 to 10 carbon atoms; Ar² represents an arylene group having 6 to 13 carbon atoms; and Ar³ represents an aryl group having 6 to 13 carbon atoms. The aryl group having 6 to 10 carbon atoms, the arylene group having 6 to 13 carbon atoms, and the aryl group having 6 to 13 carbon atoms may independently have a substituent or substituents: when the aryl group having 6 to 10 carbon atoms, the arylene group having 6 to 13 carbon atoms, and the aryl group having 6 to 13 carbon atoms independently have two or more substituents, the substituents may be bonded to each other to form a ring structure, and when one carbon atom of any of the aryl group having 6 to 10 carbon atoms, the arylene group having 6 to 13 carbon atoms, and the aryl group having 6 to 13 carbon atoms has two substituents, the substituents may be bonded to each other to form a spiro ring structure. In addition, a substituent of Ar³ may be bonded to R⁹ or R¹⁰ to form a ring structure which may be a spiro ring structure.

A carbazole derivative which is represented by the general formula (M4) is more preferable.

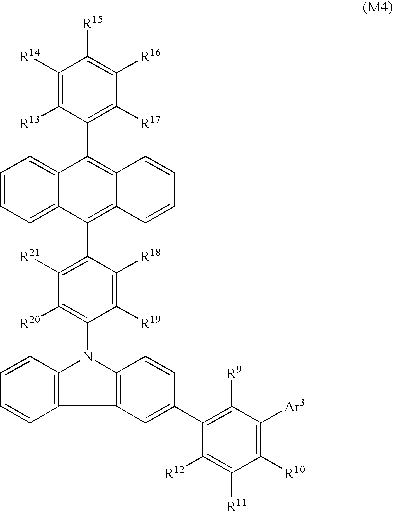

In the formula, R⁹ to R¹² and R¹⁸ to R²¹ independently represent hydrogen or an alkyl group having 1 to 4 carbon atoms; R¹³ to R¹⁷ independently represent hydrogen, an alkyl group having 1 to 4 carbon atoms, or an aryl group having 6 to 10 carbon atoms; and Ar³ represents an aryl group having 6 to 13 carbon atoms. The aryl group having 6 to 10 carbon atoms and the aryl group having 6 to 13 carbon atoms may independently have a substituent or substituents: when the aryl group having 6 to 10 carbon atoms and the aryl group having 6 to 13 independently have two or more substituents, the substituents may be bonded to each other to form a ring structure, and when one carbon atom of any of the aryl group having 6 to 10 carbon atoms and the aryl group having 6 to 13 carbon atoms has two substituents, the substituents may be bonded to each other to form a spiro ring structure. In addition, a substituent of Ar³ may be bonded to R⁹ or R¹⁰ to form a ring structure which may be a spiro ring structure.

As specific examples of the carbazole derivatives of this embodiment, carbazole derivatives represented by the structural formula (331) to the structural formula (377) can be given. However, this embodiment mode is not limited thereto.

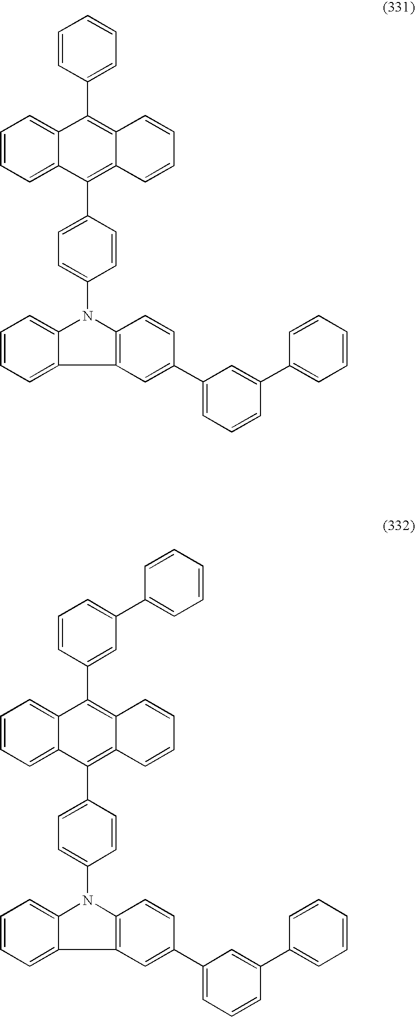

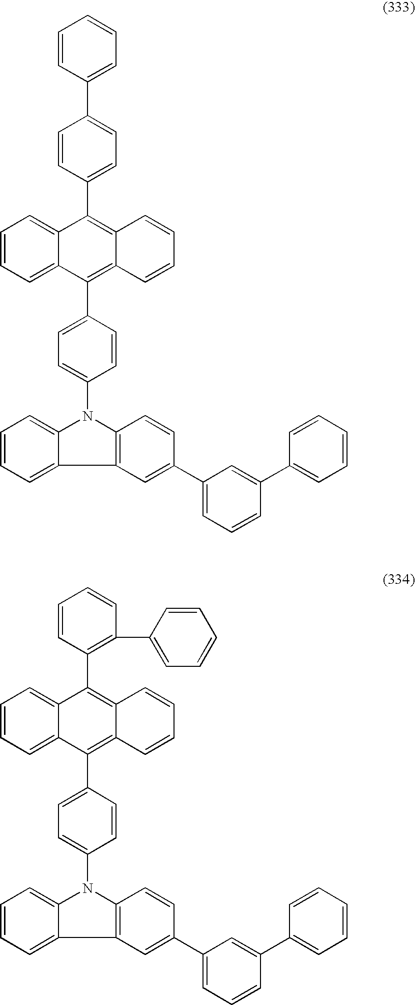

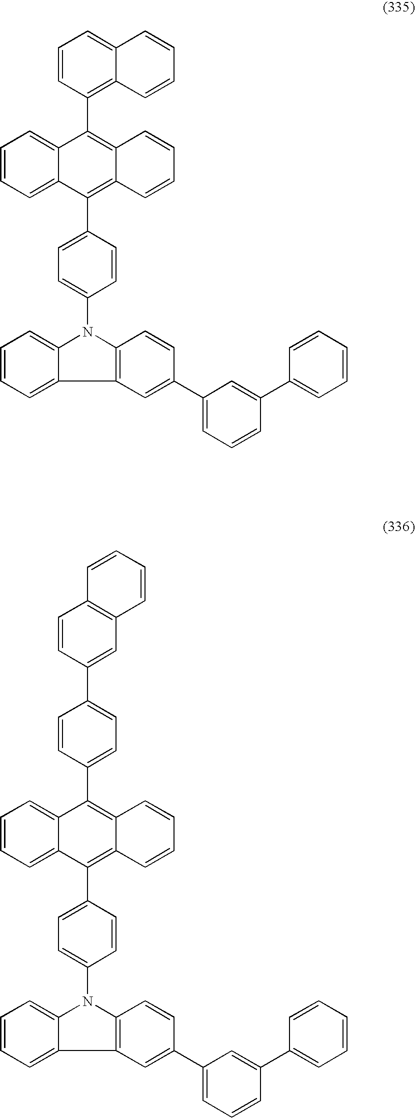

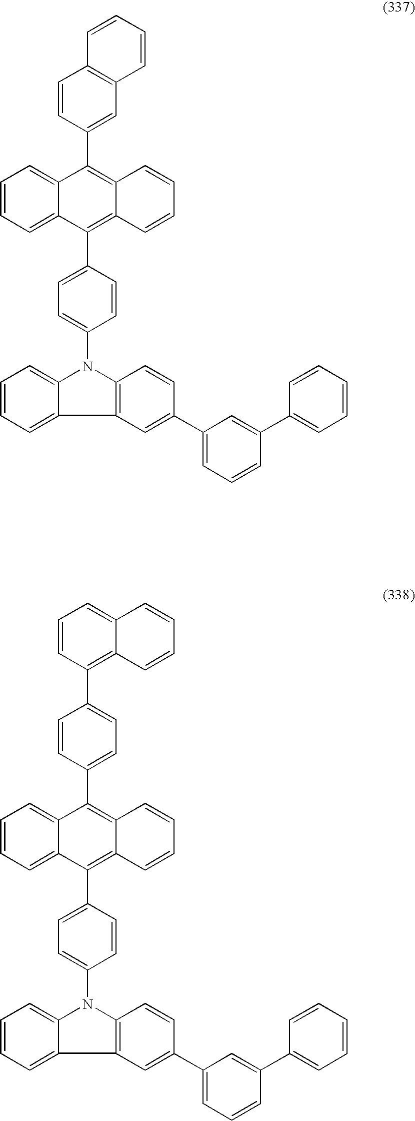

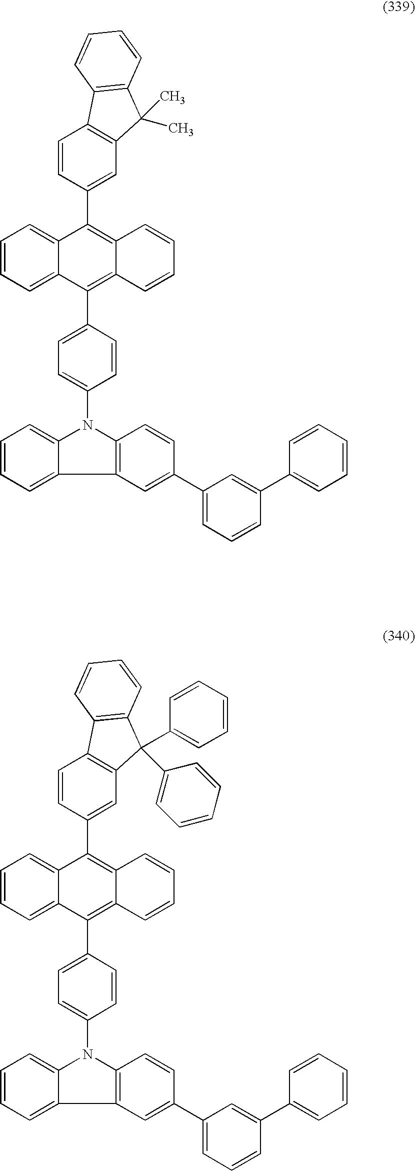

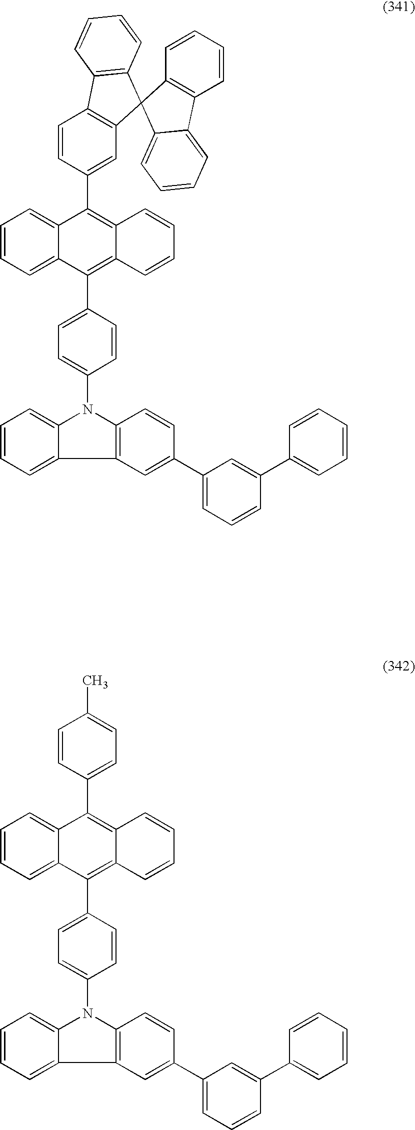

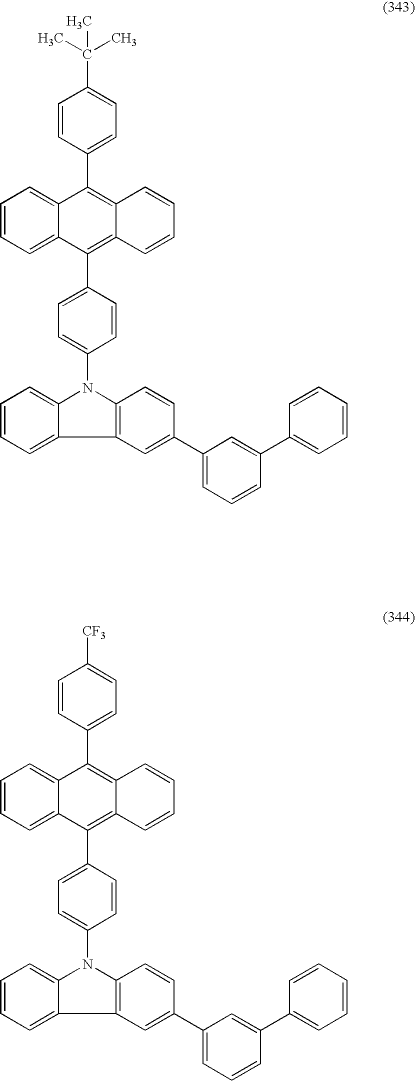

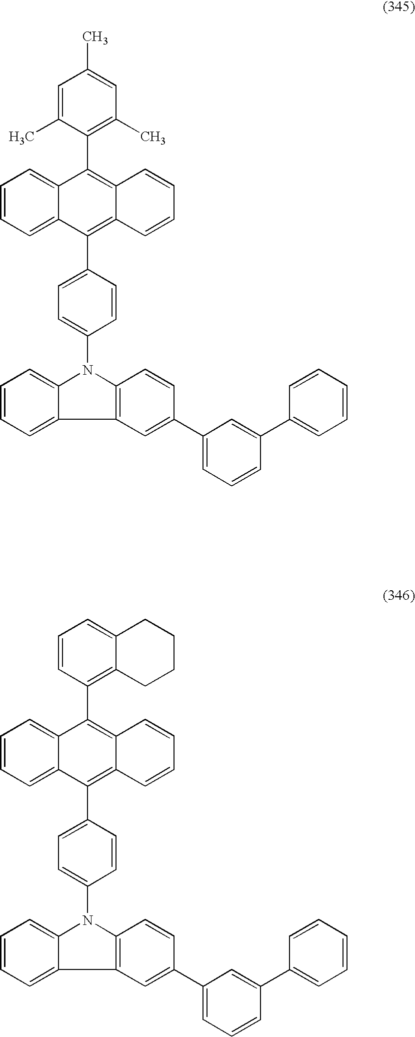

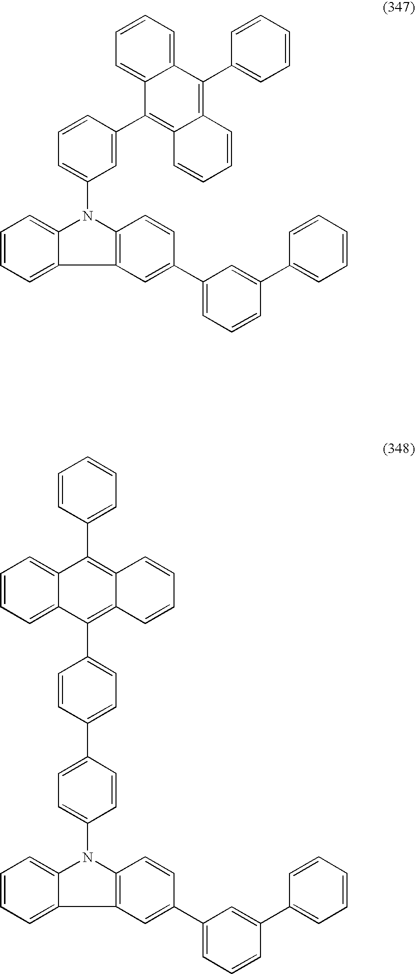

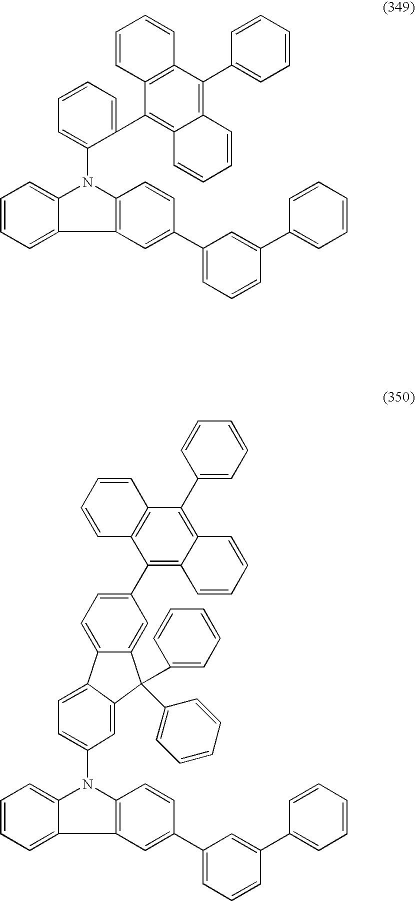

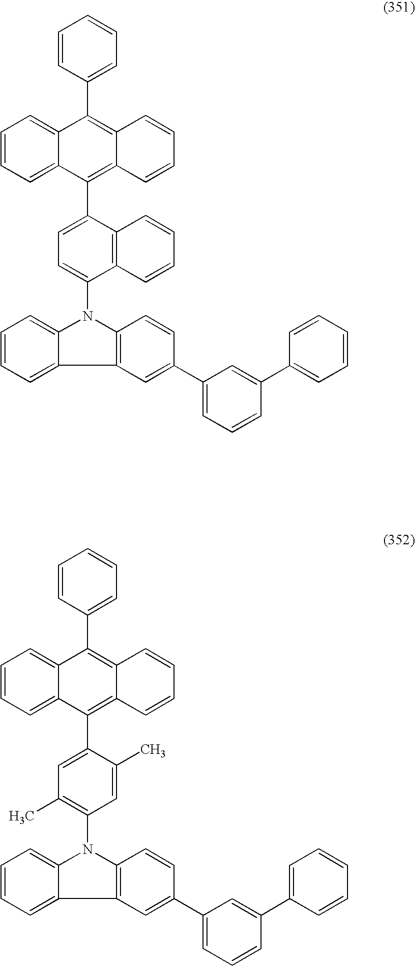

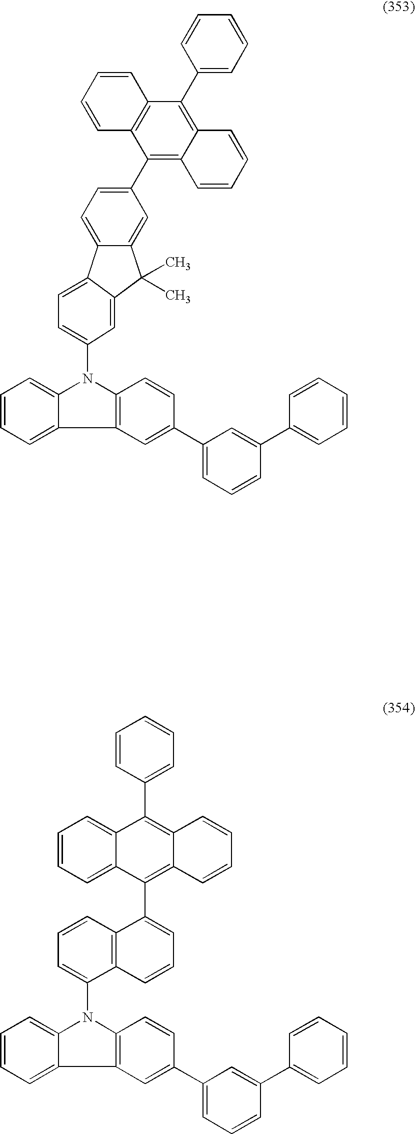

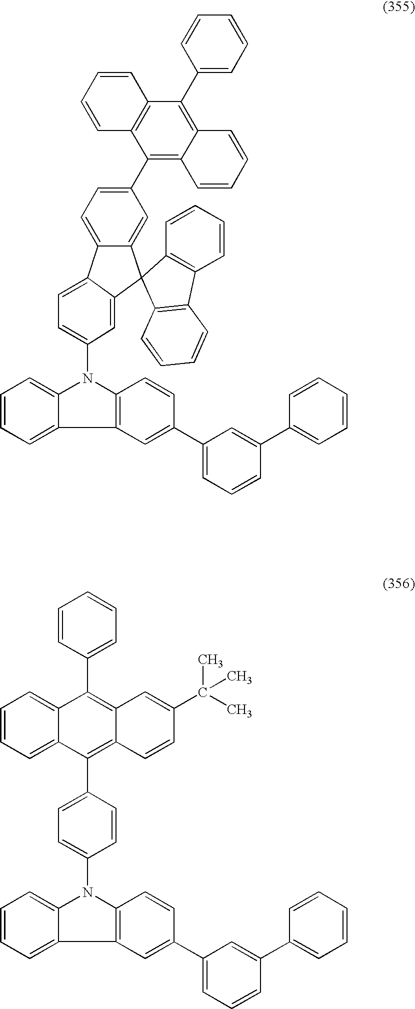

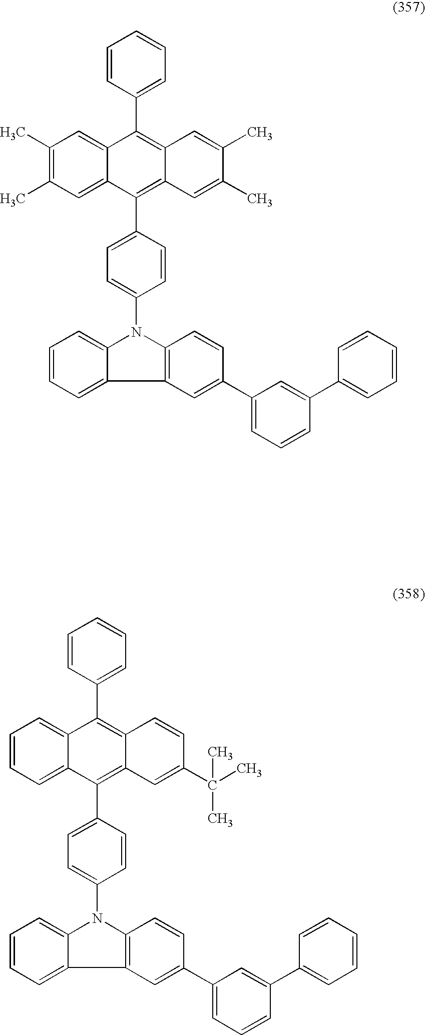

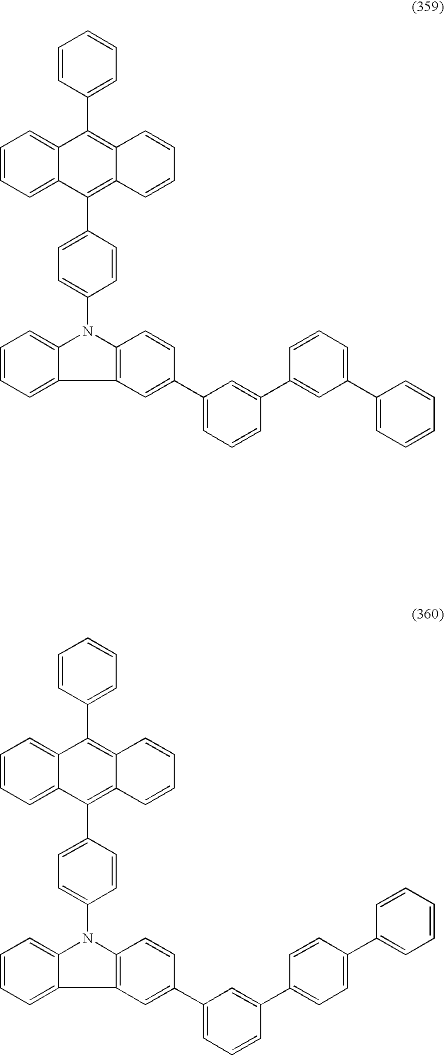

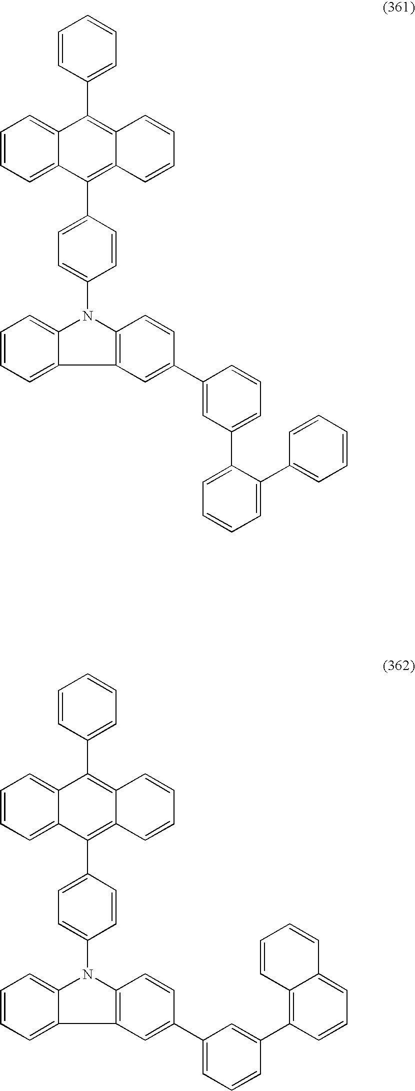

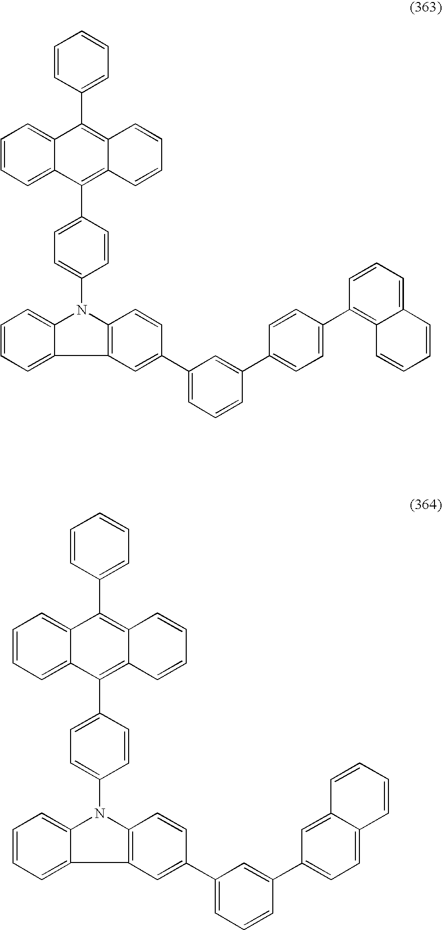

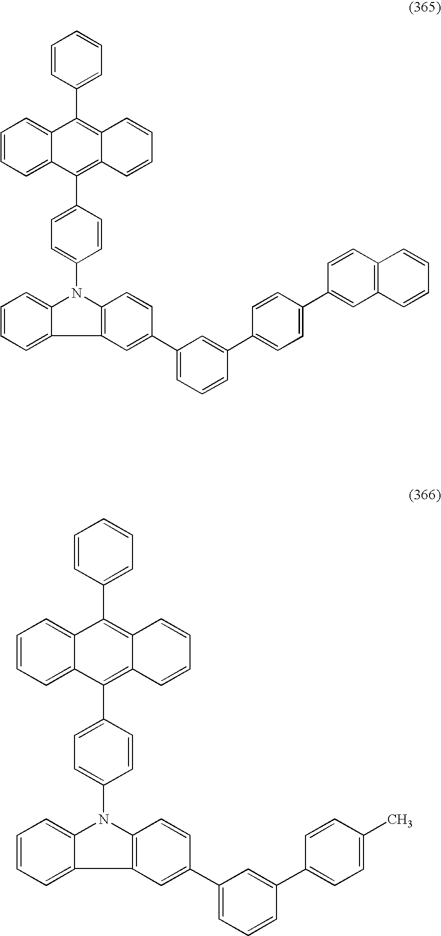

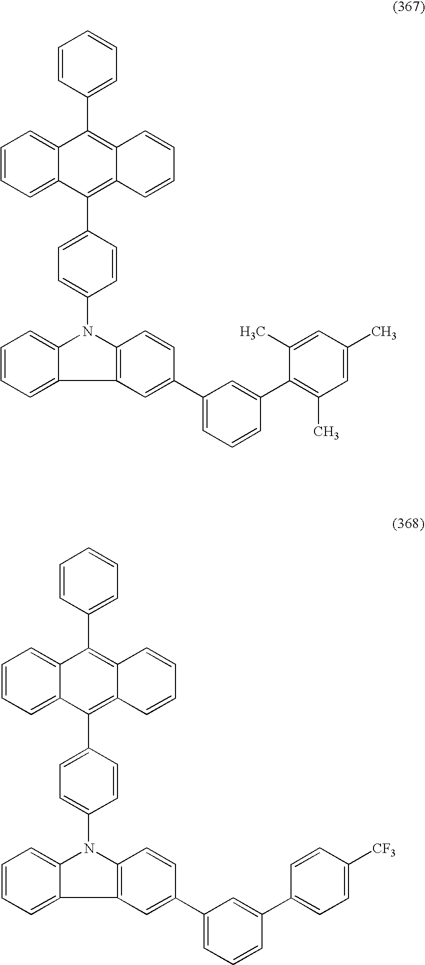

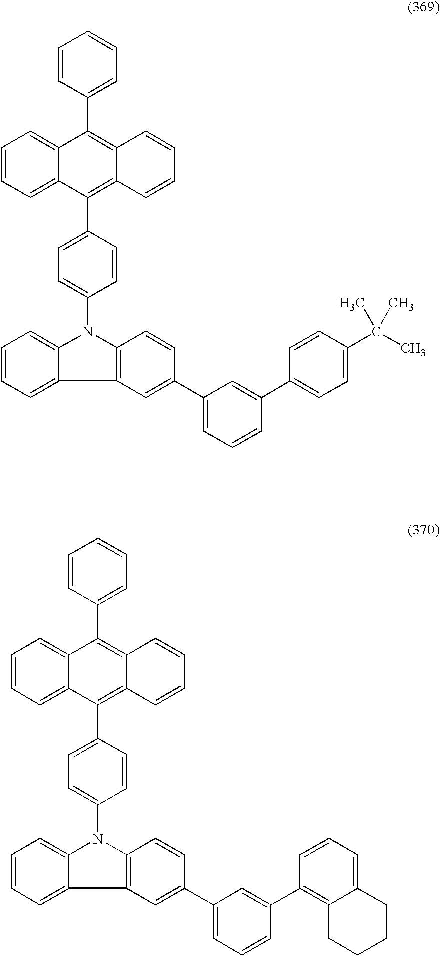

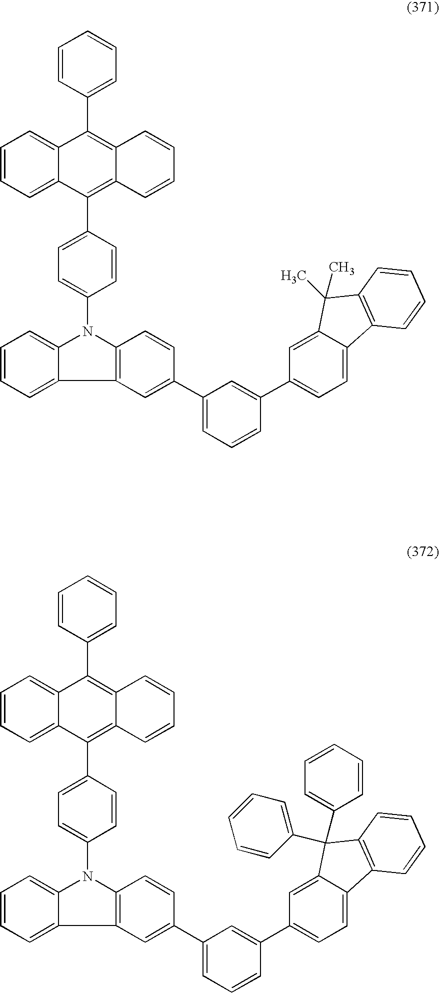

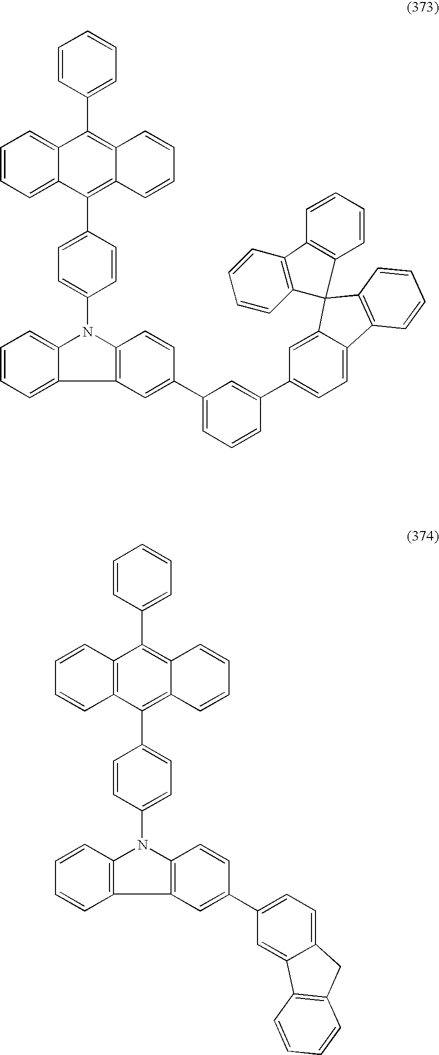

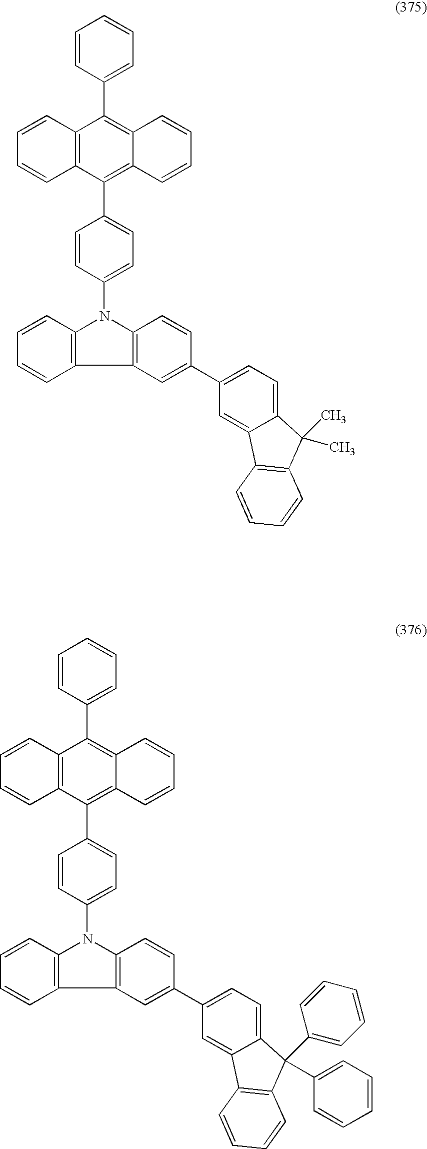

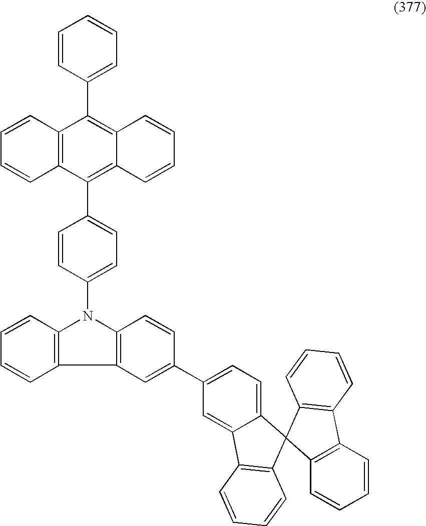

The carbazole derivative represented by the general formula (M1) can be synthesized by the synthesis methods represented by the synthetic schemes (K-1) to (K-3) and (L-1) and (M-1).

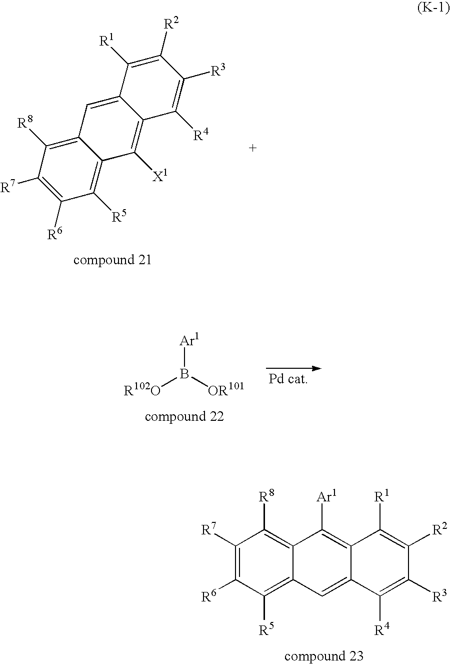

A 9-arylanthracene derivative (compound 23) can be obtained by Suzuki-Miyaura coupling of an anthracene derivative (compound 21) and an arylorganoboron compound such as an arylboronic acid (compound 22) in the presence of a palladium catalyst (the synthetic scheme (K-1)).

In the synthetic scheme (K-1), X¹ represents a halogen or a triflate group and the halogen is preferably iodine, bromine, or chlorine; R¹ to R⁸ independently represent hydrogen or an alkyl group having 1 to 4 carbon atoms; Ar¹ represents an aryl group having 6 to 13 carbon atoms which may have a substituent or substituents which may be bonded to each other to form a ring structure which may be a spiro ring structure; and R¹⁰¹ and R¹⁰² independently represent hydrogen or an alkyl group having 1 to 6 carbon atoms and R¹⁰¹ and R¹⁰² may be bonded to each other to form a ring structure.

Examples of a palladium catalyst which can be used in the synthetic scheme (K-1) include palladium(II) acetate, tetrakis(triphenylphosphine)palladium(0). Examples of a ligand of the palladium catalyst which can be used in the synthetic scheme (K-1) include tri(ortho-tolyl)phosphine, triphenylphosphine, and tricyclohexylphosphine.

Examples of a base which can be used in the synthetic scheme (K-1) include an organic base such as sodium tert-butoxide and an inorganic base such as potassium carbonate.

Examples of a solvent which can be used in the synthetic scheme (K-1) include a mixed solvent of toluene and water; a mixed solvent of toluene, alcohol such as ethanol, and water; a mixed solvent of xylene and water; a mixed solvent of xylene, alcohol such as ethanol, and water; a mixed solvent of benzene and water; a mixed solvent of benzene, alcohol such as ethanol, and water; and a mixed solvent of ether such as ethylene glycol dimethyl ether and water. Note that a mixed solvent of toluene and water or a mixed solvent of toluene, ethanol, and water is more preferable.

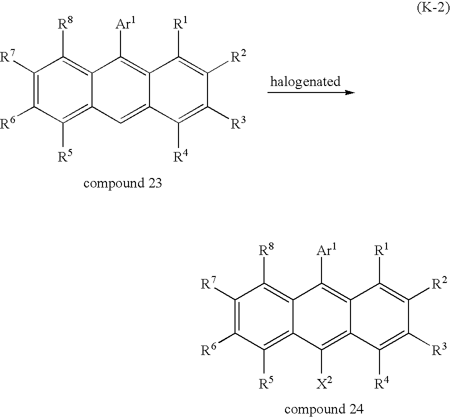

Then, a halogenated arylanthracene derivative (compound 24) can be obtained by halogenating the 9-arylanthracene derivative (compound 23) which is obtained through the synthetic scheme (K-1) (the synthetic scheme (K-2)).

In the synthetic scheme (K-2), X² represents a halogen and the halogen is preferably iodine or bromine; R¹ to R⁸ independently represent hydrogen or an alkyl group having 1 to 4 carbon atoms; Ar¹ represents an aryl group having 6 to 13 carbon atoms which may have a substituent or substituents which may be bonded to each other to form a ring structure which may be a spiro ring structure.

In the case of brominating the 9-arylanthracene derivative (compound 23) in the synthetic scheme (K-2), examples of a brominating agent which can be used include bromine and N-bromosuccinimide. Examples of a solvent which can be used in the case of brominating the 9-arylanthracene derivative (compound 23) using bromine include a halogen-based solvent such as chloroform or carbon tetrachloride. Examples of a solvent which can be used in the case of brominating the 9-arylanthracene derivative (compound 23) using N-bromosuccinimide include ethyl acetate, tetrahydrofuran, dimethylformamide, acetic acid, and water.

In the case of iodinating the 9-arylanthracene derivative (compound 23) in the synthetic scheme (K-2), examples of an iodinating agent which can be used include N-iodosuccinimide, 1,3-diiodo-5,5-dimethylimidazolidine-2,4-dione (DIH), 2,4,6,8-tetraiodo-2,4,6,8-tetraazabicyclo[3,3,0]octane-3,7-dion, and 2-iodo-2,4,6,8-tetraazabicyclo[3,3,0]octane-3,7-dion. Further, examples of a solvent which can be used in the case of iodinating the 9-arylanthracene derivative (compound 23) using any of those iodinating agents include ethyl acetate; acetic acid (glacial acetic acid); water; aromatic hydrocarbons such as benzene, toluene, and xylene; ethers such as 1,2-dimethoxyethane, diethyl ether, methyl-t-butyl ether, tetrahydrofuran, and dioxane; saturated hydrocarbons such as pentane, hexane, heptane, octane, and cyclohexane; halogens such as dichloromethane, chloroform, carbon tetrachloride, 1,2-dichloroethane, and 1,1,1-trichloroethane; nitriles such as acetonitrile and benzonitrile; and esters such as ethyl acetate, methyl acetate, and butyl acetate. Those solvents can be used alone or in combination. When water is used, it is preferably mixed with an organic solvent. In addition, in this reaction, an acid such as sulfuric acid or acetic acid is preferably used as well.

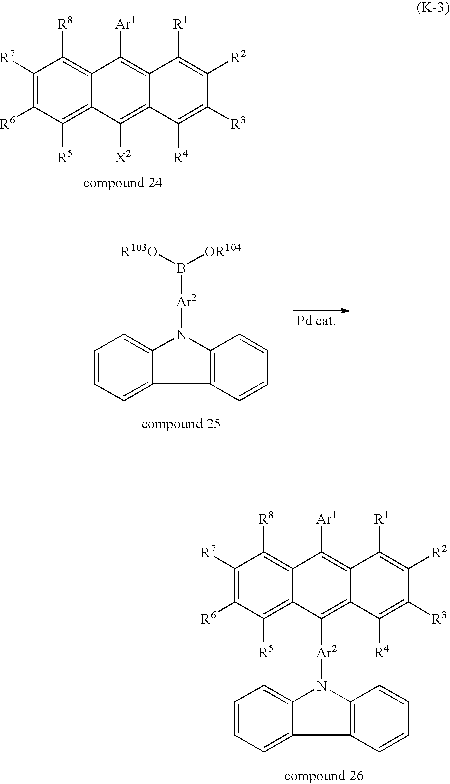

Then, a carbazole derivative (compound 26) can be obtained by Suzuki-Miyaura coupling of the arylanthracene derivative (compound 24) which is obtained through the synthetic scheme (K-2) and an organoboron compound such as an arylboronic acid including a carbazole derivative (compound 25) in the presence of a palladium catalyst (the synthetic scheme (K-3)).

In the synthetic scheme (K-3), X² represents a halogen and the halogen is preferably iodine or bromine; R¹ to R⁸ independently represent hydrogen or an alkyl group having 1 to 4 carbon atoms; Ar¹ represents an aryl group having 6 to 13 carbon atoms which may have a substituent or substituents which may be bonded to each other to form a ring structure which may be a spiro ring structure; Ar² represents an arylene group having 6 to 13 carbon atoms which may have a substituent or substituents which may be bonded to each other to form a ring structure which may be a spiro ring structure; and R¹⁰³ and R¹⁰⁴ independently represent hydrogen or an alkyl group having 1 to 6 carbon atoms and R¹⁰³ and R¹⁰⁴ may be bonded to each other to form a ring structure.

Examples of a palladium catalyst which can be used in the synthetic scheme (K-3) include palladium(II) acetate, tetrakis(triphenylphosphine)palladium(0). Examples of a ligand of the palladium catalyst which can be used in the synthetic scheme (K-3) include tri(ortho-tolyl)phosphine, triphenylphosphine, and tricyclohexylphosphine.

Examples of a base which can be used in the synthetic scheme (K-3) include an organic base such as sodium tert-butoxide and an inorganic base such as potassium carbonate.

Examples of a solvent which can be used in the synthetic scheme (K-3) include a mixed solvent of toluene and water; a mixed solvent of toluene, alcohol such as ethanol, and water; a mixed solvent of xylene and water; a mixed solvent of xylene, alcohol such as ethanol, and water; a mixed solvent of benzene and water; a mixed solvent of benzene, alcohol such as ethanol, and water; and a mixed solvent of ether such as ethylene glycol dimethyl ether and water. Note that a mixed solvent of toluene and water or a mixed solvent of toluene, ethanol, and water is more preferable.

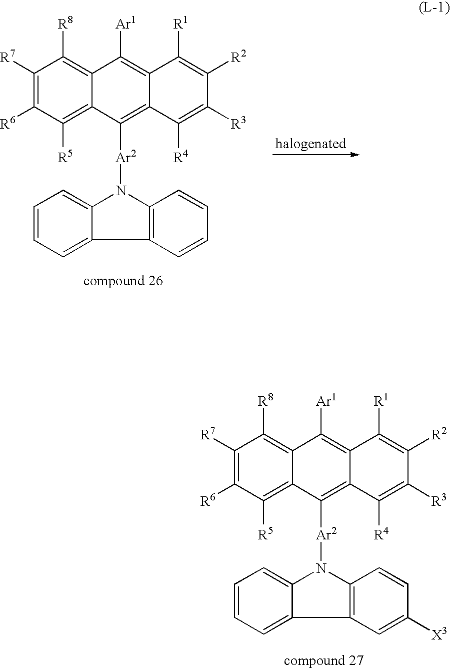

Then, a halogenated carbazole derivative (compound 27) can be obtained by halogenating the carbazole derivative (compound 26) which is obtained through the synthetic scheme (K-3) (the synthetic scheme (L-1)).

In the synthetic scheme (L-1), X³ represents a halogen and the halogen is preferably iodine or bromine; R¹ to R⁸ independently represent hydrogen or an alkyl group having 1 to 4 carbon atoms; Ar¹ represents an aryl group having 6 to 13 carbon atoms which may have a substituent or substituents which may be bonded to each other to form a ring structure which may be a spiro ring structure.

In the case of brominating the carbazole derivative (compound 26) in the synthetic scheme (L-1), examples of a brominating agent which can be used include bromine and N-bromosuccinimide. An example of a solvent which can be used in the case of brominating the carbazole derivative (compound 26) using bromine is a halogen-based solvent such as chloroform or carbon tetrachloride. Examples of a solvent which can be used in the case of brominating the carbazole derivative (compound 26) using N-bromosuccinimide include ethyl acetate, tetrahydrofuran, dimethylformamide, acetic acid, and water.

In the case of iodinating the carbazole derivative (compound 26) in the synthetic scheme (L-1), examples of an iodinating agent which can be used include N-iodosuccinimide, 1,3-diiodo-5,5-dimethylimidazolidine-2,4-dione (DIH), 2,4,6,8-tetraiodo-2,4,6,8-tetraazabicyclo[3,3,0]octane-3,7-dion, and 2-iodo-2,4,6,8-tetraazabicyclo[3,3,0]octane-3,7-dion. Further, examples of a solvent which can be used in the case of iodinating the carbazole derivative (compound 26) using any of those iodinating agents include ethyl acetate; acetic acid (glacial acetic acid); water; aromatic hydrocarbons such as benzene, toluene, and xylene; ethers such as 1,2-dimethoxyethane, diethyl ether, methyl-t-butyl ether, tetrahydrofuran, and dioxane; saturated hydrocarbons such as pentane, hexane, heptane, octane, and cyclohexane; halogens such as dichloromethane, chloroform, carbon tetrachloride, 1,2-dichloroethane, and 1,1,1-trichloroethane; nitriles such as acetonitrile and benzonitrile; and esters such as ethyl acetate, methyl acetate, and butyl acetate. Those solvents can be used alone or in combination. When water is used, it is preferably mixed with an organic solvent. In addition, in this reaction, an acid such as sulfuric acid or acetic acid is preferably used as well.

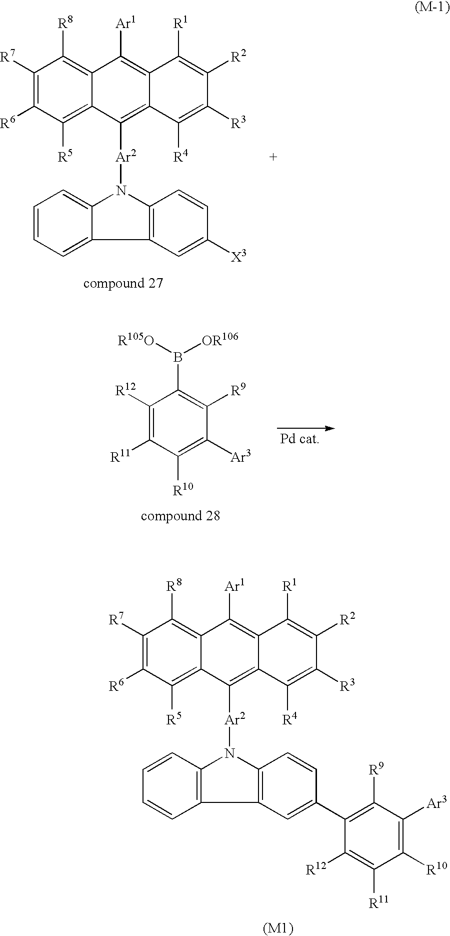

Then, a carbazole derivative (represented by the general formula (M1)) which is the object can be obtained by Suzuki-Miyaura coupling of the carbazole derivative (compound 27) which is obtained through the synthetic scheme (L-1) and an arylorganoboron compound such as an arylboronic acid (compound 28) in the presence of a palladium catalyst (the synthetic scheme (M-1)).

In the synthetic scheme (M-1), X³ represents a halogen and the halogen is preferably iodine or bromine; R¹ to R⁸ independently represent hydrogen or an alkyl group having 1 to 4 carbon atoms; Ar¹ represents an aryl group having 6 to 13 carbon atoms which may have a substituent or substituents which may be bonded to each other to form a ring structure which may be a spiro ring structure; Ar² represents an arylene group having 6 to 13 carbon atoms which may have a substituent or substituents which may be bonded to each other to form a ring structure which may be a spiro ring structure; and R¹⁰⁵ and R¹⁰⁶ independently represent hydrogen or an alkyl group having 1 to 6 carbon atoms and R¹⁰⁵ and R¹⁰⁶ may be bonded to each other to form a ring structure.

Examples of a palladium catalyst which can be used in the synthetic scheme (M-1) include palladium(II) acetate, tetrakis(triphenylphosphine)palladium(0).

Examples of a ligand of the palladium catalyst which can be used in the synthetic scheme (M-1) include tri(ortho-tolyl)phosphine, triphenylphosphine, and tricyclohexylphosphine.

Examples of a base which can be used in the synthetic scheme (M-1) include an organic base such as sodium tert-butoxide and an inorganic base such as potassium carbonate.

Examples of a solvent which can be used in the synthetic scheme (M-1) include a mixed solvent of toluene and water; a mixed solvent of toluene, alcohol such as ethanol, and water; a mixed solvent of xylene and water; a mixed solvent of xylene, alcohol such as ethanol, and water; a mixed solvent of benzene and water; a mixed solvent of benzene, alcohol such as ethanol, and water; and a mixed solvent of ether such as ethylene glycol dimethyl ether and water. Note that a mixed solvent of toluene and water or a mixed solvent of toluene, ethanol, and water is more preferable.

As described thus far, the carbazole derivative according to this embodiment can be synthesized.

The carbazole derivative of this embodiment has a large band gap, and therefore light with a short wavelength can be exhibited. Accordingly, blue-light emission with good color purity can be exhibited. In addition, the carbazole derivative of this embodiment is a bipolar material having electron- and hole-injecting transporting properties. In addition, the carbazole derivative of this embodiment has high electrochemical stability and thermal stability.

The carbazole derivative in this embodiment can be used alone as a light-emitting substance in a light-emitting layer. Further, the carbazole derivative in this embodiment can also be used as a host in a light-emitting layer. Light emission from a dopant that functions as a light-emitting substance can be obtained with a structure in which the dopant is dispersed in the carbazole derivative of this embodiment. When the carbazole derivative of this embodiment is used as a host in a light-emitting layer, blue-light emission with good color purity can be obtained.

Further, a light-emitting element can be manufactured in which the carbazole derivative of this embodiment is added to a layer formed from a material (hereinafter, referred to as a host) which has a larger band gap than the carbazole derivative of this embodiment. In that case, light emission from the carbazole derivative of this embodiment can be obtained. That is, the carbazole derivative of this embodiment can also function as a dopant. At this time, since the carbazole derivative of this embodiment has a large band gap and light with a short wavelength can be exhibited, blue-light emission with good color purity can be exhibited. Accordingly, a highly reliable light-emitting element can be manufactured.

The carbazole derivative of this embodiment can be used as a carrier-transporting material contained in a functional layer of a light-emitting element. For example, the carbazole derivative of this embodiment can be used in a carrier-transporting layer such as a hole-transporting layer, a hole-injecting layer, an electron-transporting layer, and an electron-injecting layer.

### Embodiment 4

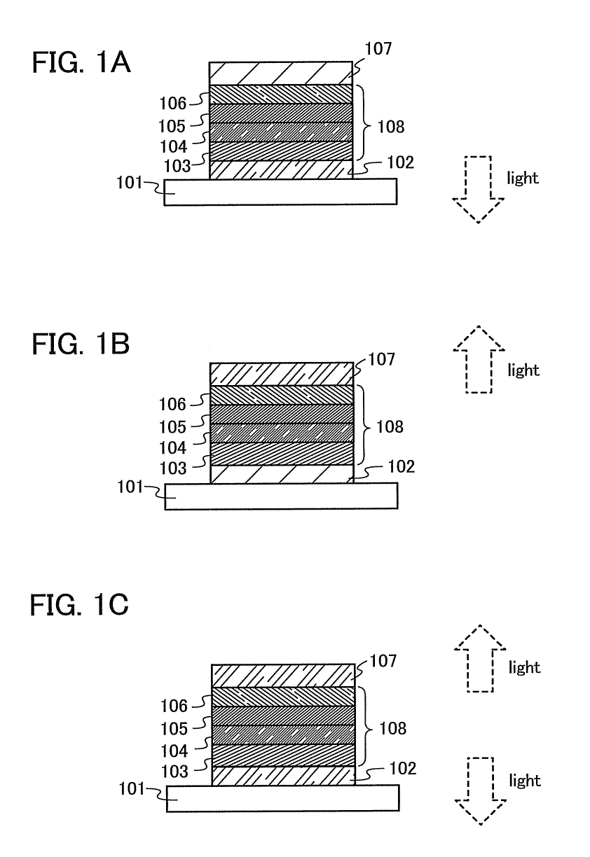

One mode of a light-emitting element including the carbazole derivative of the present invention will be described below with reference to FIGS. 1A to 1C.

In the light-emitting element of the present invention, an EL layer which includes a layer containing a light-emitting substance (the layer is also referred to as a light-emitting layer) is interposed between a pair of electrodes. The EL layer may also include a plurality of layers in addition to the layer containing a light-emitting substance. The plurality of layers is a combination of layers formed from a material having a high carrier-injecting property and a material having a high carrier-transporting property. Those layers are stacked so that a light-emitting region is formed in a region away from the electrodes, that is, carriers are recombined in a region away from the electrodes. In this specification, the layer formed from a substance having a high carrier-injecting property or a substance having a high carrier-transporting property is also referred to as a functional layer which functions, for example, to inject or transport carriers. For the functional layer, it is possible to use a layer containing a substance having a high hole-injecting property (also referred to as a hole-injecting layer), a layer containing a substance having a high hole-transporting property (also referred to as a hole-transporting layer), a layer containing a substance having a high electron-injecting property (also referred to as an electron-injecting layer), a layer containing a substance having a high electron-transporting property (also referred to as an electron-transporting layer), and the like.

In the light-emitting element of this embodiment illustrated in FIGS. 1A to 1C, an EL layer 108 is provided between a pair of electrodes: a first electrode 102 and a second electrode 107. The EL layer 108 has a first layer 103, a second layer 104, a third layer 105, and a fourth layer 106. The light-emitting elements in FIGS. 1A to 1C include a first electrode 102 over a substrate 101; the first layer 103, the second layer 104, the third layer 105, and the fourth layer 106 stacked in that order over the first electrode 102; and a second electrode 107 provided thereover. Note that in this embodiment, the following description will be made on the assumption that the first electrode 102 functions as an anode and that the second electrode 107 functions as a cathode.

The substrate 101 is used as a support of the light-emitting element. For example, glass, quartz, plastic, or the like can be used for the substrate 101. Alternatively, a flexible substrate may be used. A flexible substrate is a substrate that can be bent, for example, a plastic substrate made of polycarbonate, polyarylate, and polyether sulfone can be given. Alternatively, a film (made of polypropylene, polyester, vinyl, polyvinyl fluoride, vinyl chloride, or the like), an inorganic evaporated film, or the like can be used. Note that other substrates may also be used as long as they function as a support in a manufacturing process of the light-emitting element.

It is preferable that the first electrode 102 be formed using a metal, an alloy, or a conductive compound with a high work function (specifically, equal to or higher than 4.0 eV), a mixture thereof, or the like. Specifically, for example, indium tin oxide (ITO), indium tin oxide containing silicon or silicon oxide, indium zinc oxide (IZO), indium oxide containing tungsten oxide and zinc oxide (IWZO), and the like are given. Films of those conductive metal oxides are generally formed by sputtering, but they may be formed by a sol-gel method or the like. For example, a film of indium zinc oxide (IZO) can be formed by a sputtering method using a target in which zinc oxide is added to indium oxide at 1 wt % to 20 wt %. A film of indium oxide containing tungsten oxide and zinc oxide (IWZO) can be formed by a sputtering method using a target in which tungsten oxide and zinc oxide are added to indium oxide at 0.5 wt % to 5 wt % and 0.1 wt % to 1 wt %, respectively. In addition, gold (Au), platinum (Pt), nickel (Ni), tungsten (W), chromium (Cr), molybdenum (Mo), iron (Fe), cobalt (Co), copper (Cu), palladium (Pd), nitride of a metal material (such as titanium nitride), and the like can be given.

The first layer 103 contains a substance having a high hole-injecting property. Molybdenum oxide, vanadium oxide, ruthenium oxide, tungsten oxide, manganese oxide, or the like can be used. Alternatively, the first layer 103 can be formed using any of the following materials: phthalocyanine-based compounds such as phthalocyanine (H₂ Pc) and copper phthalocyanine (CuPc), aromatic amine compounds such as 4,4′-bis[N-(4-diphenylaminophenyl)-N-phenylamino]biphenyl (DPAB) and 4,4′-bis(N-{4-[N-(3-methylphenyl)-N-phenylamino]phenyl}-N-phenylamino)biphenyl (DNTPD), high molecular compounds such as poly(3,4-ethylenedioxythiophene)/poly(styrenesulfonic acid) (PEDOT/PSS), and the like.

Further, the first layer 103 can be formed using a tris(p-enamine-substituted-aminophenyl)amine compound, a 2,7-diamino-9-fluorenylidene compound, a tri(p-N-enamine-substituted-aminophenyl)benzene compound, a pyrene compound having one or two ethenyl groups having at least one aryl group, N,N′-di(biphenyl-4-yl)-N,N′-diphenylbiphenyl-4,4′-diamine, N,N,N′,N′-tetra(biphenyl-4-yl)biphenyl-4,4′-diamine, N,N,N′,N′-tetra(biphenyl-4-yl)-3,3′-diethylbiphenyl-4,4′-diamine, 2,2′-(methylenedi-4,1-phenylene)bis[4,5-bis(4-methoxyphenyl)-2H-1,2,3-triazole], 2,2′-(biphenyl-4,4′-diyl)bis(4,5-diphenyl-2H-1,2,3-triazole), 2,2′-(3,3′-dimethylbipheny-4,4′-diyl)bis(4,5-diphenyl-2H-1,2,3-triazole), bis[4-(4,5-diphenyl-2H-1,2,3-triazol-2-yl)phenyl](methyl)amine, or the like.

Further, the first layer 103 can be formed from a composite material formed by a composition of an organic compound and an inorganic compound. In particular, a composite material which contains an organic compound and an inorganic compound showing an electron-accepting property to the organic compound is excellent in a hole-injecting property and a hole-transporting property since electrons are transferred between the organic compound and the inorganic compound and carrier density is increased.

In the case of using the composite material formed by composition of an organic compound and an inorganic compound for the first layer 103, the first layer 103 can achieve an ohmic contact with the first electrode 102; therefore, a material of the first electrode can be selected regardless of the work function.

As the inorganic compound which is used for the composite material, an oxide of a transition metal is preferable. In addition, an oxide of metals that belong to Group 4 to Group 8 of the periodic table can be given. Specifically, vanadium oxide, niobium oxide, tantalum oxide, chromium oxide, molybdenum oxide, tungsten oxide, manganese oxide, and rhenium oxide are preferable because of their high electron-accepting properties. Among them, molybdenum oxide is preferable because it can be easily handled due to its stableness in the atmosphere and low hygroscopic property.

As the organic compound which is used for the composite material, any of various compounds such as an aromatic amine compound, a carbazole derivative, aromatic hydrocarbon, or a high molecular compound (an oligomer, a dendrimer, a polymer, or the like) can be used. Note that the organic compound which is used for the composite material is preferably an organic compound having a high hole-transporting property. Specifically, a substance having a hole mobility of 10⁻⁶ cm²/Vs or higher is preferable. However, any other substance whose hole-transporting property is higher than the electron-transporting property may be used. The organic compounds that can be used for the composite material is specifically given below.

Examples of an aromatic amine compound which can be used for the composite material specifically include N,N′-di(p-tolyl)-N,N′-diphenyl-p-phenylenediamine (DTDPPA), 4,4′-bis[N-(4-diphenylaminophenyl)-N-phenylamino]biphenyl (DPAB), 4,4′-bis(N-{4-[N-(3-methylphenyl)-N-phenylamino]phenyl}-N-phenylamino)biphenyl (DNTPD), and 1,3,5-tris[N-(4-diphenylaminophenyl)-N-phenylamino]benzene (DPA3B).

Examples of a carbazole derivative which can be used for the composite material specifically include 3-[N-(9-phenylcarbazol-3-yl)-N-phenylamino]-9-phenylcarbazole (PCzPCA1), 3,6-bis[N-(9-phenylcarbazol-3-yl)-N-phenylamino]-9-phenylcarbazole (PCzPCA2), and 3-[N-(1-naphthyl)-N-(9-phenylcarbazol-3-yl)amino]-9-phenylcarbazole (PCzPCN1).

In addition, the following can also be used: 4,4′-di(N-carbazolyl)biphenyl (CBP), 1,3,5-tris[4-(N-carbazolyl)phenyl]benzene (TCPB), 9-[4-(N-carbazolyl)]phenyl-10-phenylanthracene (CzPA), and 1,4-bis[4-(N-carbazolyl)phenyl]-2,3,5,6-tetraphenylbenzene.

Further, examples of an aromatic hydrocarbon which can be used for the composite material include 2-tert-butyl-9,10-di(2-naphthyl)anthracene (t-BuDNA), 2-tert-butyl-9,10-di(1-naphthyl)anthracene, 9,10-bis(3,5-diphenylphenyl)anthracene (DPPA), 2-tert-butyl-9,10-bis(4-phenylphenyl)anthracene (t-BuDBA), 9,10-di(2-naphthyl)anthracene (DNA), 9,10-diphenylanthracene (DPAnth), 2-tert-butylanthracene (t-BuAnth), 9,10-bis(4-methyl-1-naphthyl)anthracene (DMNA), 2-tert-butyl-9,10-bis[2-(1-naphthyl)phenyl]anthracene, 9,10-bis[2-(1-naphthyl)phenyl]anthracene, 2,3,6,7-tetramethyl-9,10-di(1-naphthyl)anthracene, 2,3,6,7-tetramethyl-9,10-di(2-naphthyl)anthracene, 9,9′-bianthryl, 10,10′-diphenyl-9,9′-bianthryl, 10,10′-bis(2-phenylphenyl)-9,9′-bianthryl, 10,10′-bis[(2,3,4,5,6-pentaphenyl)phenyl]-9,9′-bianthryl, anthracene, tetracene, rubrene, perylene, and 2,5,8,11-tetra(tert-butyl)perylene. Besides, pentacene, coronene, or the like can be used. Thus, an aromatic hydrocarbon having a hole mobility of 1×10⁻⁶ cm²/Vs or higher and having 14 to 42 carbon atoms is preferable.

Note that an aromatic hydrocarbon which can be used for the composite material may have a vinyl skeleton. Examples of an aromatic hydrocarbon having a vinyl group include 4,4′-bis(2,2-diphenylvinyl)biphenyl (DPVBi) and 9,10-bis[4-(2,2-diphenylvinyl)phenyl]anthracene (DPVPA).

Further, a high molecular compound such as poly(N-vinylcarbazole) (PVK) or poly(4-vinyltriphenylamine) (PVTPA) can also be used.

As a substance for forming the second layer 104, a substance having a high hole-transporting property, specifically, an aromatic amine compound (that is, a compound having a benzene ring-nitrogen bond) is preferable. Examples of materials which are widely used include 4,4′-bis[N-(3-methylphenyl)-N-phenylamino]biphenyl, a derivative thereof such as 4,4′-bis[N-(1-napthyl)-N-phenylamino]biphenyl (hereinafter referred to as NPB); and a starburst aromatic amine compound such as 4,4′,4″-tris(N,N-diphenyl-amino)triphenylamine and 4,4′,4″-tris[N-(3-methylphenyl)-N-phenylamino]triphenylamine. Most of the substances mentioned here have a hole mobility of 10⁻⁶ cm²/Vs or higher. However, any other material whose hole-transporting property is higher than the electron-transporting property may be used. Note that the second layer 104 is not limited to a single layer, and may be a mixed layer of any of the above substances, or a stacked layer which comprises two or more layers each formed from any of the above substances.

Alternatively, a hole-transporting property material may be added to a high molecular compound that is electrically inactive, such as PMMA.

Further, a high molecular compound such as poly(N-vinylcarbazole) (PVK), poly(4-vinyltriphenylamine) (PVTPA), poly[N-(4-{N′-[4-(4-diphenylamino)phenyl]phenyl-N′-phenylamino}phenyl)methacrylamide] (PTPDMA), or poly[N,N′-bis(4-butylphenyl)-N,N′-bis(phenyl)benzidine (poly-TPD) may be used, and further, a hole-transporting material may be added to the above high molecular compounds as appropriate.

Further, the second layer 104 can also be formed using a tris(p-enamine-substituted-aminophenyl)amine compound, a 2,7-diamino-9-fluorenylidene compound, a tri(p-N-enamine-substituted-aminophenyl)benzene compound, a pyrene compound having one or two ethenyl groups having at least one aryl group, N,N′-di(biphenyl-4-yl)-N,N′-diphenylbiphenyl-4,4′-diamine, N,N,N′,N′-tetra(biphenyl-4-yl)biphenyl-4,4′-diamine, N,N,N′,N′-tetra(biphenyl-4-yl)-3,3′-diethylbiphenyl-4,4′-diamine, 2,2′-(methylenedi-4,1-phenylene)bis[4,5-bis(4-methoxyphenyl)-2H-1,2,3-triazole], 2,2′-(biphenyl-4,4′-diyl)bis(4,5-diphenyl-2H-1,2,3-triazole), 2,2′-(3,3′-dimethylbipheny-4,4′-diyl)bis(4,5-diphenyl-2H-1,2,3-triazole), bis[4-(4,5-diphenyl-2H-1,2,3-triazol-2-yl)phenyl](methyl)amine, or the like.

The third layer 105 is a layer containing a light-emitting substance (the layer is also referred to as a light-emitting layer). In this embodiment, the third layer 105 is formed using any of the carbazole derivatives which are described in Embodiment 1. The carbazole derivatives which are described in Embodiments 1 to 3 exhibit blue-light emission, and thus can be preferably used as a light-emitting substance for a light-emitting element.

Further, in the third layer 105, any of the carbazole derivatives which are described in Embodiments 1 to 3 can also be used as a host. Light emission from a dopant that functions as a light-emitting substance can be obtained with a structure in which the dopant is dispersed in the carbazole derivative which is described in Embodiments 1 to 3.

When any of the carbazole derivatives which are described in Embodiments 1 to 3 is used as a material in which another light-emitting substance is dispersed, emission color originating from the light-emitting substance can be obtained. Further, it is possible to obtain a mixed color of an emission color originating from the carbazole derivative which is described in Embodiments 1 to 3 and an emission color originating from the light-emitting substance which is dispersed in the carbazole derivative.

Further, a light-emitting element in which any of the carbazole derivatives which are described in Embodiments 1 to 3 is added to a layer formed from a material (a host) which has a larger band gap than the carbazole derivative which is described in Embodiments 1 to 3 can be manufactured. In that case, light emission from the carbazole derivative which is described in Embodiments 1 to 3 can be obtained. That is, the carbazole derivative which is described in Embodiments 1 to 3 can also function as a dopant. At this time, since the carbazole derivative which is described in Embodiments 1 to 3 has an extremely large band gap and light with a short wavelength can be exhibited, a light-emitting element that can exhibit blue-light emission with good color purity can be manufactured.

Note that by doping the light-emitting layer with an alkali metal salt of a carboxyl acid having a pyridine ring, a pyridine derivative including an alkali metal, or an alkali metal salt of a phenol-based compound, low driving voltage of a light-emitting element which can be realized in addition to the above effects.

Here, any of a variety of materials can be used as the light-emitting substance which is dispersed in the carbazole derivative which is described in Embodiments 1 to 3. Specifically, fluorescent substances that emit fluorescence can be given: 9,10-diphenyl-2-[N-phenyl-N-(9-phenyl-9H-carbazol-3-yl)amino]anthracene (2PCAPA), 4-(dicyanomethylene)-2-methyl-6-(p-dimethylaminostyryl)-4H-pyran (DCM1), 4-(dicyanomethylene)-2-methyl-6-(julolidin-4-yl-vinyl)-4H-pyran (DCM2), N,N-dimethylquinacridone (DMQd), rubrene, N,N′-bis[4-(9H-carbazol-9-yl)phenyl]-N,N′-diphenylstilbene-4,4′-diamine (YGA2S), 4-(9H-carbazol-9-yl)-4′-(10-phenyl-9-anthryl)triphenylamine (YGAPA), bis[3-(1H-benzimidazol-2-yl)fluoren-2-olato]zinc(II), bis[3-(1H-benzimidazol-2-yl)fluoren-2-olato]beryllium(II), bis[2-(1H-benzimidazol-2-yl)dibenzo[b,d]furan-3-olato](phenolato)aluminium(III), bis[2-(benzoxazol-2-yl)-7,8-methylenedioxydibenzo[b,d]furan-3-olato](2-naphtholato)aluminium(III), and the like. Further, a compound including terphenyl with six or more aryl groups can be used. Further, phosphorescent substances that emit phosphorescence can be used: (acetylacetonato)bis[2,3-bis(4-fluorophenyl)quinoxalinato]iridium(III) (Ir(Fdpq)₂ (acac)), 2,3,7,8,12,13,17,18-octaethyl-21H,23H-porphineplatinum(II) (PtOEP), and the like.

The fourth layer 106 can be formed from a substance having a high electron-transporting property. For example, the fourth layer 106 is formed from a metal complex having a quinoline skeleton or a benzoquinoline skeleton, such as tris(8-quinolinolato)aluminum (Alq), tris(4-methyl-8-quinolinolato)aluminum (Almq₃), bis(10-hydroxybenzo[h]-quinolinato)beryllium (BeBq₂), or bis(2-methyl-8-quinolinolato)(4-phenylphenolato)aluminum (BAlq). Other examples which can be used are metal complexes having an oxazole-based ligand or a thiazole-based ligand, such as bis[2-(2-hydroxyphenyl)benzoxazolato]zinc (Zn(BOX)₂) and bis[2-(2-hydroxyphenyl)benzothiazolato]zinc (Zn(BTZ)₂). Furthermore, as an alternative to metal complexes, the following can also be used: 2-(4-biphenylyl)-5-(4-tert-butylphenyl)-1,3,4-oxadiazole (PBD), 1,3-bis[5-(p-tert-butylphenyl)-1,3,4-oxadiazol-2-yl]benzene (OXD-7), 3-(4-biphenylyl)-4-phenyl-5-(4-tert-butylphenyl)-1,2,4-triazole (TAZ), bathophenanthroline (BPhen), bathocuproine (BCP), bis[3-(1H-benzimidazol-2-yl)fluoren-2-olato]zinc(II), bis[3-(1H-benzimidazol-2-yl)fluoren-2-olato]beryllium(II), bis[2-(1H-benzimidazol-2-yl)dibenzo[b,d]furan-3-olato](phenolato)aluminium(III), bis[2-(benzoxazol-2-yl)-7,8-methylenedioxydibenzo[b,d]furan-3-olato](2-naphtholato)aluminium(III), and the like. Most of the substances mentioned here have an electron mobility of 10⁻⁶ cm²/Vs or higher. Note that any other material whose electron-transporting property is higher than the hole-transporting property may be used for an electron-transporting layer. Further the third layer 105 is not limited to a single layer, and may be a stacked layer which comprises two or more layers each formed from any of the above substances.

Further, a layer having a function of promoting electron injection (an electron-injecting layer) may be provided between the fourth layer 106 and the second electrode 107. For a layer having a function of promoting electron injection, an alkali metal, an alkaline earth metal, or a compound thereof, such as lithium fluoride (LiF), cesium fluoride (CsF), or calcium fluoride (CaF₂), can be used. For example, a layer in which an alkali metal, an alkaline earth metal, or a compound thereof is contained in a substance having an electron-transporting property, for example, a layer formed from Alq in which magnesium (Mg) is contained can be used. Note that by using a layer in which an alkali metal or an alkaline earth metal is contained in a substance having an electron-transporting property, electrons can be injected efficiently from the second electrode 107, which is preferable. In addition, by using, as the electron-injecting layer, a layer formed from a substance having an electron-transporting property in which an alkali metal salt of a carboxyl acid having a pyridine ring, a pyridine derivative including an alkali metal, or an alkali metal salt of a phenol compound is contained, a light-emitting element which can be driven at low voltage can be realized.

As a substance for forming the second electrode 107, a metal, an alloy, an electroconductive compound, a mixture thereof, or the like having a low work function (specifically, 3.8 eV or lower) can be used. Specific examples of such a cathode material are given below: elements belonging to Group 1 and Group 2 of the periodic table, that is, alkali metals such as lithium (Li) and cesium (Cs) and alkaline earth metals such as magnesium (Mg), calcium (Ca), and strontium (Sr); alloys thereof (e.g., MgAg and AlLi); rare earth metals such as europium (Eu) and ytterbium (Yb); and alloys thereof. However, by providing a layer having a function of promoting electron injection between the second electrode 107 and the fourth layer 106 so that it is stacked with the second electrode, any of a variety of conductive materials such as Al, Ag, ITO, and ITO containing silicon or silicon oxide can be used for the second electrode 107, regardless of the work function.

Further, any of the carbazole derivatives which are described in Embodiments 1 to 3 can also be used for the functional layer of the light-emitting element.

For the formation of the first layer 103, the second layer 104, the third layer 105, and the fourth layer 106, any of a variety of methods such as an evaporation method, a sputtering method, a droplet discharge method (an inkjet method), a spin coating method, or a printing method can be employed. Further, a different film formation method may be used to form each electrode or each layer.

A case where a thin film is formed by a wet process using a liquid composition in which any of the carbazole derivatives which are described in Embodiments 1 to 3 is dissolved is described. A material for forming the thin film which includes the carbazole derivative which is described in Embodiments 1 to 3 is dissolved in a solvent. The liquid composition is attached to a region where the thin film is to be formed. Then, the solvent is removed and the resulting material is solidified, whereby the thin film is formed.

For a wet process, any of the following methods can be employed: a spin coating method, a roll coating method, a spray method, a casting method, a dipping method, a droplet discharge (ejection) method (an inkjet method), a dispenser method, any of a variety of printing methods (a method by which a thin film can be formed into a desired pattern, such as screen (stencil) printing, offset (planographic) printing, letterpress printing, gravure (intaglio) printing, or the like). Note that the method in which a composition including the carbazole derivative which is described in Embodiments 1 to 3 can be used is not limited to the above method. Any method in which a liquid composition is used can be employed.

Further, any of a variety of solvents can be used in the above composition. For example, the above carbazole derivative can be dissolved in a solvent that has an aromatic ring (e.g., a benzene ring), such as toluene, xylene, methoxybenzene (anisole), dodecylbenzene, or a mixed solvent of dodecylbenzene and tetralin. Further, the above carbazole derivative can also be dissolved in an organic solvent which does not include an aromatic ring, such as dimethylsulfoxide (DMSO), dimethylformamide (DMF), or chloroform.

Further, there are other solvents such as ketone-based solvents such as acetone, methyl ethyl ketone, diethyl ketone, n-propyl methyl ketone, and cyclohexanone; ester-based solvents such as ethyl acetate, n-propyl acetate, n-butyl acetate, ethyl propionate, γ-butyrolactone, and diethyl carbonate; ether solvents such as diethylether, tetrahydrofuran and dioxane; and alcohol solvents such as ethanol, isopropanol, 2-methoxyethanol, and 2-ethoxyethanol.

Further, a composition which is described in this embodiment may also contain another organic material. As the organic material, an aromatic compound or a heteroaromatic compound which is solid at room temperature can be given. For the organic material, a low molecular compound or a high molecular compound can be used. When a low molecular compound is used, a low molecular compound (which may be referred to as a medium molecular compound) including a substituent which can increase the solubility in a solvent is preferably used.

The composition may further include a binder in order to improve the quality of a film which is formed. A high molecular compound that is electrically inactive is preferably used as the binder. Specifically, polymethylmethacrylate (PMMA), polyimide, or the like can be used.

In the light-emitting element of this embodiment which has the structure as described above, the potential difference between the first electrode 102 and the second electrode 107 makes current flow, whereby holes and electrons recombine in the third layer 105 containing a substance with a high light-emitting property and thus light is emitted. That is, a light-emitting region is formed in the third layer 105.

Emitted light is extracted out through one or both of the first electrode 102 and the second electrode 107. Accordingly, either or both the first electrode 102 and the second electrode 107 are formed from a light-transmitting substance. When only the first electrode 102 is formed from a light-transmitting substance, light emission is extracted from the substrate side through the first electrode 102 as illustrated in FIG. 1A. When only the second electrode 107 is formed from a light-transmitting substance, light emission is extracted from the side opposite to the substrate through the second electrode 107 as illustrated in FIG. 1B. When both the first electrode 102 and the second electrode 107 are formed from a light-transmitting substance, light emission is extracted from both the substrate side and the opposite side to the substrate, through the first electrode 102 and the second electrode 107, respectively, as illustrated in FIG. 1C.

Note that while FIGS. 1A to 1C illustrate a structure in which the first electrode 102 which functions as an anode is located on the substrate side, the second electrode 107 which functions as a cathode may be located on the substrate side. Note that in that case, a TFT which is connected to the second electrode 107 is preferably an n-channel TFT.

Note that the structure of the layers provided between the first electrode 102 and the second electrode 107 is not limited to the above example. A structure other than the above may alternatively be employed as long as a light-emitting region in which holes and electrons are recombined is provided in a portion away from the first electrode 102 and the second electrode 107 in order to prevent quenching due to proximity of the light-emitting region to a metal.

In other words, there is no particular limitation on the stacked structure of the layers. The light-emitting layer containing the carbazole derivative which is described in Embodiments 1 to 3 may be freely combined with layers containing a substance with a high electron-transporting property, a substance having a high hole-transporting property, a substance with a high electron-injecting property, a substance having a high hole-injecting property, a bipolar substance (a substance with a high electron-transporting and hole-transporting property), a hole-blocking material, and the like.

For example, a structure may be employed in which a hole-transporting layer is not provided and an electron-injection suppression layer is provided for suppressing injection of electrons from the hole-injecting layer containing an acceptor and a light-emitting layer. In that case, it is preferable that the electron affinity of a material for forming the electron-injection suppression layer be smaller than that of a material for forming the light-emitting layer and the acceptor. Further, a structure may be employed in which not an electron-transporting layer but a hole-injection suppression layer is provided for suppressing injection of holes from the electron-injecting layer and from the light-emitting layer. In that case, it is preferable that the ionization potential of a material for forming the hole-injection suppression layer be larger than that of a material for forming the light-emitting layer and the donor.

Further, a light-emitting element which is described in this embodiment may have a structure in which two or more layers containing a substance having a high hole-injecting property and two or more layers containing a substance having a high hole-transporting property which are described above are alternately stacked. Further, the electrode which functions as a cathode may have a three-layer structure in which a second metal electrode which prevents oxidation is interposed between an oxide transparent conductive film and a metal electrode.

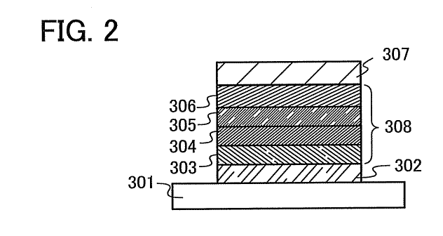

In a light-emitting element illustrated in FIG. 2, over a substrate 301, an EL layer 308 is provided between a pair of electrodes: a first electrode 302 and a second electrode 307. The EL layer 308 includes a first layer 303 formed from a substance having a high electron-transporting property, a second layer 304 containing a light-emitting substance, a third layer 305 formed from a substance having a high hole-transporting property, and a fourth layer 306 formed from a substance having a high hole-injecting property. The first electrode 302 which functions as a cathode, the first layer 303 formed from a substance having a high electron-transporting property, the second layer 304 containing a light-emitting substance, the third layer 305 formed from a substance having a high hole-transporting property, the fourth layer 306 formed from a substance having a high hole-injecting property, and the second electrode 307 which functions as an anode are stacked in that order.

A specific method for forming a light-emitting element is described below.

In the light-emitting element of this embodiment, an EL layer is interposed between a pair of electrodes. The EL layer includes at least a layer containing a light-emitting substance formed using any of the carbazole derivatives which are described in Embodiments 1 to 3 (the layer is also referred to as a light-emitting layer). In addition to the layer containing a light-emitting substance, the EL layer may include a functional layer (e.g., a hole-injecting layer, a hole-transporting layer, an electron-transporting layer, or an electron-injecting layer). The electrodes (the first electrode and the second electrode), the layer containing a light-emitting substance, and the functional layers may be formed by a wet processes such as a droplet discharge method (an inkjet method), a spin coating method, or a printing method, or by a dry process such as a vacuum evaporation method, a CVD method, or a sputtering method. The use of a wet process enables the formation at atmospheric pressure using a simple apparatus and process, and thus effects of simplifying the process and improving the productivity can be obtained. In contrast, in a dry process, dissolution of a material is not needed, and thus, a material that has low solubility in a solution can be used, which leads to expansion of material choices.

All the thin films included in the light-emitting element may be formed by a wet process. In this case, the light-emitting element can be manufactured with only facilities needed for a wet process. Alternatively, formation of the stacked layers up to formation of the layer containing a light-emitting substance may be performed by a wet process whereas the functional layer, the second electrode, and the like which are stacked over the layer containing a light-emitting substance may be formed by a dry process. Further alternatively, the first electrode and the functional layers may be formed by a dry process before the formation of the layer containing a light-emitting substance and the layer containing a light-emitting substance, and the functional layer stacked thereover and the second electrode may be formed by a wet process. It is needles to say that the present invention is not limited thereto. The light-emitting element can be formed by appropriate selection from a wet process and a dry process depending on a material that is to be used, a required film thickness, and an interface state.

In this embodiment, the light-emitting element is manufactured over a substrate made of glass, plastic, or the like. When a plurality of such light-emitting elements are manufactured over one substrate, a passive matrix light-emitting device can be manufactured. Alternatively, for example, thin film transistors (TFTs) are formed over a substrate formed using glass, plastic, or the like, and then, light-emitting elements may be manufactured over an electrode that is electrically connected to the TFTs. Thus, an active matrix light-emitting device in which drive of the light-emitting elements is controlled by the TFTs can be manufactured. Note that there is no particular limitation on the structure of the TFT. Either a staggered TFT or an inverted staggered TFT may be employed. Further, there is no particular limitation on the crystallinity of a semiconductor used for forming the TFTs, and an amorphous semiconductor or a crystalline semiconductor may be used. In addition, a driver circuit formed over a TFT substrate may be formed using n-channel and p-channel TFTs, or using either n-channel or p-channel TFTs.

Any of the carbazole derivatives which are described in Embodiments 1 to 3 has an extremely large band gap. Therefore, even when a dopant material which emits light with a relatively short wavelength, especially, which emits blue-light is used, light emission not from the carbazole derivative which is described in Embodiments 1 to 3 but from the dopant material can efficiently be obtained.

Any of the carbazole derivatives which are described in Embodiments 1 to 3 has a large band gap and is a bipolar material which lets both holes and electrons flow. Therefore, by using the carbazole derivative which is described in Embodiments 1 to 3 for a light-emitting element, a highly reliable light-emitting element with good carrier balance can be obtained.

Further, by using any of the carbazole derivatives which are described in Embodiments 1 to 3, a highly reliable light-emitting device and electronic device can be obtained.

### Embodiment 5

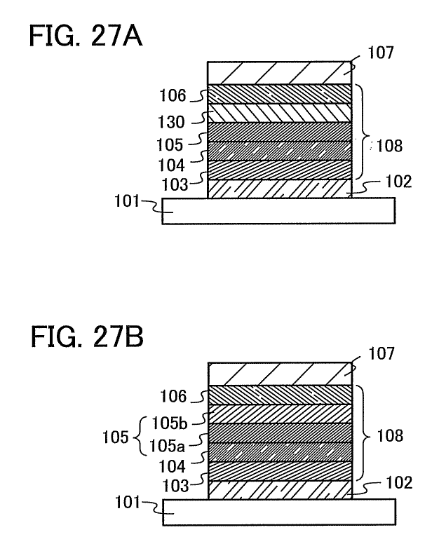

In this embodiment, a light-emitting element having a different structure from the structure described in Embodiment 4 will be described with reference to FIGS. 27A and 27B.

A layer which controls movement of electron carriers may be provided between an electron-transporting layer and a light-emitting layer. FIG. 27A illustrate a structure in which a layer 130 which controls movement of electron carriers is provided between a fourth layer 106 which functions as an electron-transporting layer and a third layer 105 which functions as an light-emitting layer (the third layer 105 is also referred to as a light-emitting layer 105). The layer 130 which controls movement of electron carriers is a layer which is formed by adding a small amount of substance having a high electron-trapping property to the above material having a high electron-transporting property, or a layer formed by adding a material having a hole-transporting property with a low lowest unoccupied molecular orbital (LUMO) energy level to a material having a high electron-trapping property. With such a layer, movement of electron carriers is controlled, whereby carrier balance can be adjusted. Such a structure is very effective in suppressing a problem (such as shortening of element lifetime) caused when electrons pass through the third layer 105.

Further, another structure may be employed in which the light-emitting layer 105 includes two or more layers. FIG. 27B illustrates an example in which the light-emitting layer 105 includes two layers: a first light-emitting layer 105a and a second light-emitting layer 105b.

If the first light-emitting layer 105a and the second light-emitting layer 105b are stacked in that order over the second layer 104 which functions as hole-transporting layer to form the light-emitting layer 105, for example, a substance having a hole-transporting property can be used as a host material of the first light-emitting layer 105a and a substance having an electron-transporting property can be used for the second light-emitting layer 105b.

Any of the carbazole derivatives which are described in Embodiments 1 to 3 can be used alone for a light-emitting layer. Further, the carbazole derivative which is described in Embodiments 1 to 3 can also be used as a host material and a dopant material.

If any of the carbazole derivatives which are described in Embodiments 1 to 3 is used as a host material, light emission from a dopant material that functions as a light-emitting substance can be obtained with a structure in which the dopant material that functions as a light-emitting substance is dispersed in the carbazole derivative which is described in Embodiments 1 to 3.

On the other hand, when any of the carbazole derivatives which are described in Embodiments 1 to 3 is used as a dopant material, light emission from the carbazole derivative which is described in Embodiments 1 to 3 can be obtained with a structure in which the carbazole derivative which is described in Embodiments 1 to 3 is added to a layer formed from a material (a host) which has a larger band gap than the carbazole derivative which is described in Embodiments 1 to 3.

Further, any of the carbazole derivatives which are described in Embodiments 1 to 3 has both a hole-transporting property and an electron-transporting property, that is, a bipolar property. When the carbazole derivative has a hole-transporting property, it can be used for the first light-emitting layer 105a. When the carbazole derivative has an electron-transporting property, it can be used for the second light-emitting layer 105b. The carbazole derivative which is described in Embodiments 1 to 3 can be used alone for the first light-emitting layer 105a or the second light-emitting layer 105b or can be used as a host material or a dopant material of the first light-emitting layer 105a or the second light-emitting layer 105b. When the carbazole derivative is used alone for a light-emitting layer or is used as a host material, whether the carbazole derivative is used for the first light-emitting layer 105a having a hole-transporting property or the second light-emitting layer 105b having an electron-transporting property may be determined depending on the carrier-transporting property.

Note that this embodiment can be combined as appropriate with another embodiment.

### Embodiment 6

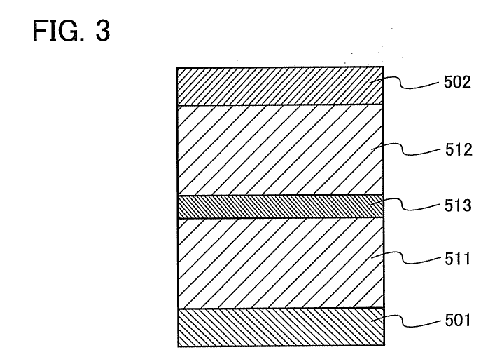

In this embodiment, one mode of a light-emitting element having a structure in which a plurality of light-emitting units according to the present invention are stacked (hereinafter this type of light-emitting element is referred to as a stacked element) will be described with reference to FIG. 3. This light-emitting element has a plurality of light-emitting units between a first electrode and a second electrode.

In FIG. 3, a first light-emitting unit 511 and a second light-emitting unit 512 are stacked between a first electrode 501 and a second electrode 502. As for the first electrode 501 and the second electrode 502, electrodes similar to those described in Embodiment 4 or 5 can be used. The structures of the first light emitting unit 511 and the second light emitting unit 512 may be the same or different. Their structures can be similar to that described in Embodiment 4 or 5.

The charge generation layer 513 contains a composite material of an organic compound and a metal oxide. This composite material of an organic compound and a metal oxide is a composite material described in Embodiment 4 or 5 and includes an organic compound and a metal oxide such as V₂O₅, MoO₃ or WO₃. As the organic compound, any of variety of compounds such as an aromatic amine compound, a carbazole derivative, aromatic hydrocarbon, and a high molecular compound (an oligomer, a dendrimer, a polymer, or the like) can be given. An organic compound having a hole mobility of 10⁻⁶ cm²/Vs or higher is preferably used as a hole-transporting organic compound. Note that any organic compound other than the above substance may also be used as long as its hole-transporting property is higher than its electron-transporting property. The composite material of an organic compound and a metal oxide is excellent in a carrier-injecting property and a carrier-transporting property; therefore, low-voltage driving and low-current driving can be achieved.

Note that the charge generation layer 513 may be formed by a combination of a composite material of an organic compound and a metal oxide and another material. For example, a layer containing the composite material of an organic compound and a metal oxide may be used in combination with a layer containing a compound selected from an electron-donating substance and a compound having a high electron-transporting property. Further, a layer containing the composite material of an organic compound and a metal oxide may be used in combination with a transparent conductive film.

In any case, any layer can be employed as the charge generation layer 513 interposed between the first light-emitting unit 511 and the second light-emitting unit 512 as long as the layer injects electrons into one of these light-emitting units and holes into the other when voltage is applied to the first electrode 501 and the second electrode 502.

Although the light-emitting element having two light-emitting units is described in this embodiment, a light-emitting element in which three or more light-emitting units are stacked can be employed in a similar way. When the charge generation layer is provided between the pair of electrodes so as to partition the plural light-emitting units like in the light-emitting element of this embodiment, the element can have a long lifetime in a high luminance region while the current density is kept low. Further, in the case where the light-emitting element is applied to lighting, voltage drop due to resistance of an electrode material can be reduced. Accordingly, light can be uniformly emitted from a large area. Moreover, a light-emitting device of low power consumption which can be driven at low voltage can be achieved.

Note that this embodiment can be combined as appropriate with another embodiment.

### Embodiment 7

In this embodiment, one mode of a light-emitting device manufactured using any of the carbazole derivatives which are described in Embodiments 1 to 3 will be described.

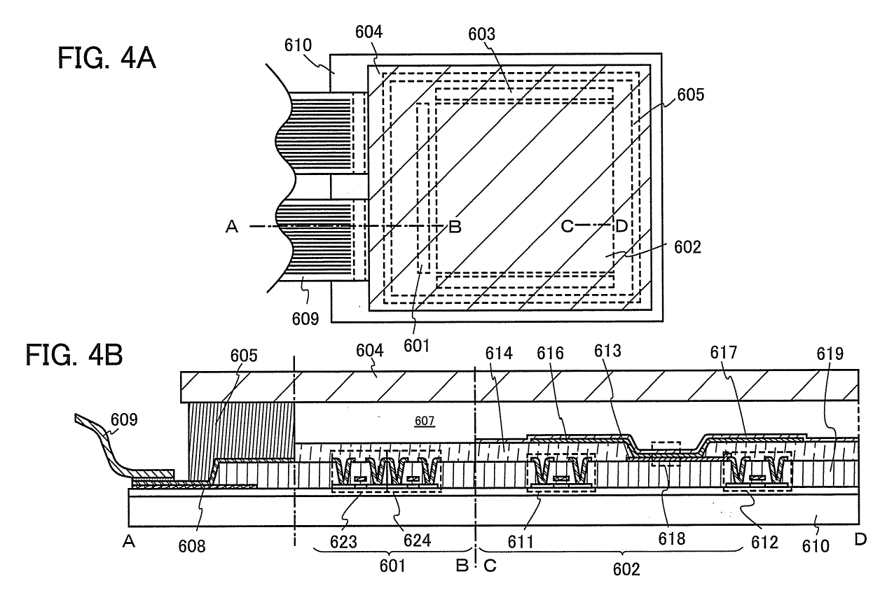

In this embodiment, one mode of a light-emitting device manufactured using any of the carbazole derivatives which are described in Embodiments 1 to 3 is described with reference to FIGS. 4A and 4B. Note that FIG. 4A is a top view of the light-emitting device, and FIG. 4B is a cross-sectional view taken along lines A-B and C-D of FIG. 4A. Reference numerals 601, 602, and 603 denote a driver circuit portion (a source side driver circuit), a pixel portion, and a driver circuit portion (a gate side driver circuit), respectively, which are indicated by dotted lines. Further, reference numeral 604 denotes a sealing substrate and reference numeral 605 denotes a sealant. A portion surrounded by the sealant 605 is a space 607.

Note that a lead wiring 608 is a wiring for transmitting signals to be input into the source side driver circuit 601 and the gate side driver circuit 603 and for receiving signals such as a video signal, a clock signal, a start signal, and a reset signal from an FPC (flexible printed circuit) 609 serving as an external input terminal. Although only the FPC is illustrated here, this FPC may be provided with a printed wiring board (PWB). The light-emitting device in this specification refers to not just a light-emitting device itself but a light-emitting device provided with an FPC or a PWB.

Next, a cross-sectional structure is described with reference to FIG. 4B. Among the driver circuit portions and the pixel portion formed over an element substrate 610, the source side driver circuit 601, which is a driver circuit portion, and one pixel in the pixel portion 602 are illustrated here.

Note that a CMOS circuit in which an n-channel TFT 623 and a p-channel TFT 624 are formed in combination is formed in the source side driver circuit 601. The driver circuit may be formed by a variety of CMOS circuits, PMOS circuits, or NMOS circuits. Although the driver integrated device which has the driver circuit formed over the substrate is described in this embodiment, the driver circuit does not always have to be formed over the substrate. It is also possible to form the driver circuit not over the substrate but outside the substrate.

Moreover, the pixel portion 602 includes a plurality of pixels including a switching TFT 611, a current control TFT 612, and a first electrode 613 electrically connected to a drain of the current control TFT 612. Note that an insulator 614 is formed covering an end of the first electrode 613. Here, a positive photosensitive acrylic resin film is used for the insulator 614.

In order to improve the coverage, the insulator 614 is formed to have a curved surface with a curvature at its upper or lower end portion. For example, in the case of using positive photosensitive acrylic as a material for the insulator 614, only the upper end portion of the insulator 614 preferably has a curved surface with a radius of curvature (0.2 μm to 3 μm). Further, the insulator 614 can be formed using either a negative type that becomes insoluble in an etchant by light irradiation or a positive type that becomes soluble in an etchant by light irradiation.

A layer 616 containing a light-emitting substance and a second electrode 617 are formed over the first electrode 613. Here, the first electrode 613 serving as an anode is preferably formed of a material with a high work function. For example, a single-layer film of an ITO film, an indium tin oxide film containing silicon, an indium oxide film containing zinc oxide at 2 wt % to 20 wt %, a titanium nitride film, a chromium film, a tungsten film, a Zn film, a Pt film, or the like can be used. Alternatively, a stack of a titanium nitride film and a film containing aluminum as its main component, a stack of three layers of a titanium nitride film, a film containing aluminum as its main component, and a titanium nitride film, or the like can be used. Note that when the first electrode 613 has a stacked-layer structure, the resistance can be reduced as a wiring and a good ohmic contact can be obtained.

The layer 616 containing a light-emitting substance is formed by any of a variety of methods such as an evaporation method using an evaporation mask, a droplet discharge method such as an inkjet method, a printing method, and a spin coating method. The layer 616 containing a light-emitting substance contains any of the carbazole derivatives which are described in Embodiments 1 to 3. As another material contained in the layer 616 containing a light-emitting substance, a low molecular material, a medium molecular material (including an oligomer and a dendrimer), or a high molecular material may be used.

Further, as a material used for the second electrode 617, which is formed over the layer 616 containing a light-emitting substance and functions as a cathode, a material having a low work function (Al, Mg, Li, Ca, or an alloy or a compound thereof such as MgAg, MgIn, AlLi, LiF, or CaF₂) is preferably used. In the case where light generated in the layer 616 containing a light-emitting substance passes through the second electrode 617, the second electrode 617 is preferably formed using a stack of a thin metal film having a reduced thickness and a transparent conductive film (such as ITO, indium oxide containing zinc oxide at 2 wt % to 20 wt %, indium tin oxide containing silicon or silicon oxide, or zinc oxide (ZnO)).

By attaching the sealing substrate 604 to the element substrate 610 using the sealant 605, the light-emitting element 618 is provided in the space 607 which is surrounded by the element substrate 610, the sealing substrate 604, and the sealant 605. Note that the space 607 is filled with filler. The space is sometimes filled with an inert gas (such as nitrogen or argon) or the sealant 605.

Note that an epoxy-based resin is preferably used for the sealant 605. In addition, it is desirable to use a material that allows permeation of moisture or oxygen as little as possible. As the sealing substrate 604, a plastic substrate formed from fiberglass-reinforced plastics (FRP), polyvinyl fluoride (PVF), polyester, acrylic, or the like can be used besides a glass substrate or a quartz substrate.

In this manner, the light-emitting device manufactured using any of the carbazole derivatives which are described in Embodiments 1 to 3 can be obtained.

Any of the carbazole derivatives which are described in Embodiments 1 to 3 has a large band gap and is a bipolar material which lets both holes and electrons flow. Therefore, by using the carbazole derivative which is described in Embodiments 1 to 3 for a light-emitting element, a highly reliable light-emitting element with good carrier balance can be obtained.

Further, by using any of the carbazole derivatives which are described in Embodiments 1 to 3, a highly reliable light-emitting device and electronic device can be obtained.

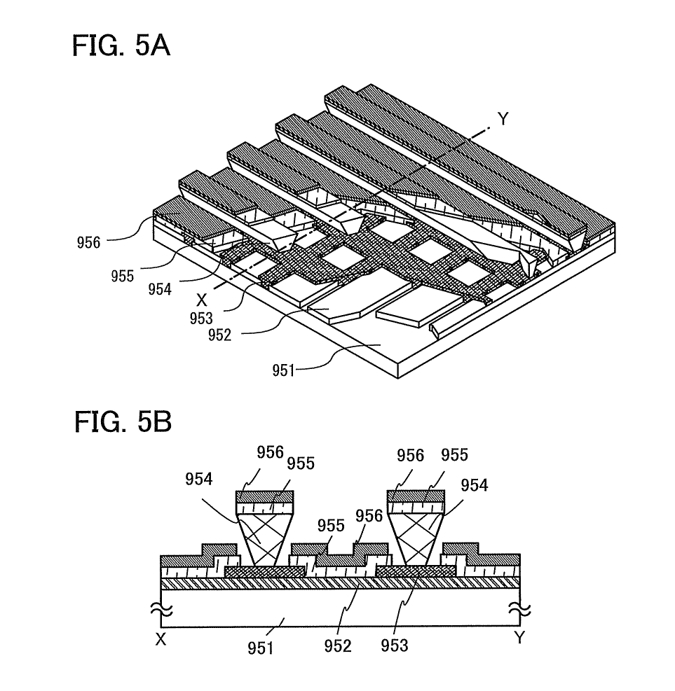

Although an active matrix light-emitting device in which operation of a light-emitting element is controlled with a transistor is described in this embodiment, a passive matrix light-emitting device may alternatively be used. FIGS. 5A and 5B illustrate a passive matrix light-emitting device as one mode of the present invention which is manufactured by applying a light-emitting element. In FIGS. 5A and 5B, a layer 955 containing a light-emitting substance is provided over a substrate 951 and between an electrode 952 and an electrode 956. An edge portion of the electrode 952 is covered with an insulating layer 953. A partition layer 954 is provided over the insulating layer 953. The sidewalls of the partition layer 954 are aslope so that the distance between the sidewalls is gradually reduced toward the surface of the substrate. That is, a cross section in a short-side direction of the partition layer 954 is a trapezoidal shape, and the bottom side (the side which faces a direction similar to a plane direction of the insulating layer 953 and is in contact with the insulating layer 953) is shorter than the top side (the side which faces a direction similar to the plane direction of the insulating layer 953 and is not in contact with the insulating layer 953). By the provision of the partition layer 954 in this manner, defects of the light-emitting element due to static charge or the like can be prevented. Also in the case of a passive matrix light-emitting device, a highly reliable light-emitting device can be obtained by provision of the light-emitting element disclosed by one mode of the present invention.

### Embodiment 8

In this embodiment, modes of electronic devices of the present invention each of which includes a light-emitting device which is described in Embodiment 7 will be described. Electronic devices according to the present invention include any of the carbazole derivatives which are described in Embodiments 1 to 3 and have a highly reliable display portion.

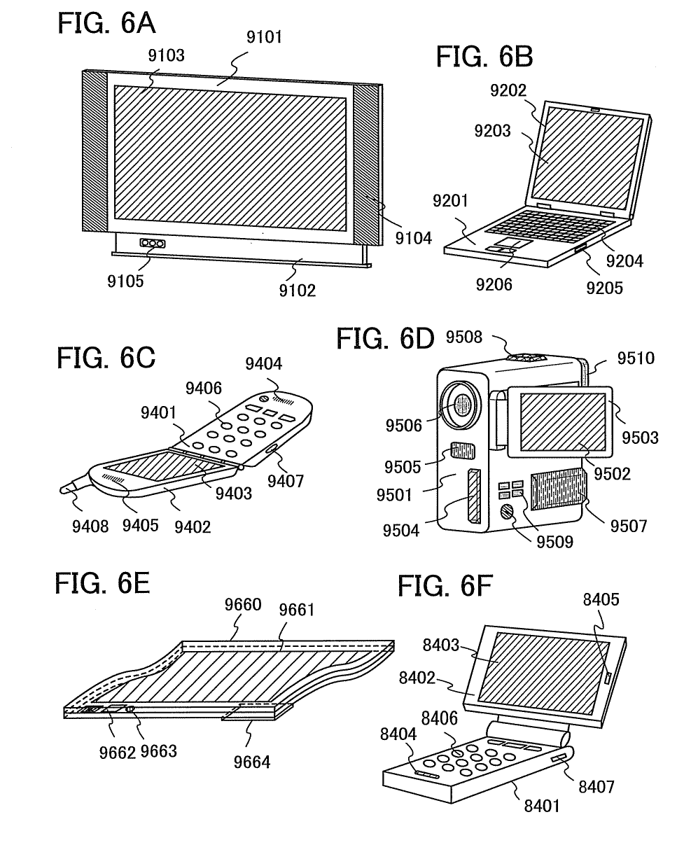

Examples of electronic devices each manufactured using any of the carbazole derivatives which are described in Embodiments 1 to 3 include cameras such as video cameras or digital cameras, goggle type displays, navigation systems, audio playback devices (e.g., car audio systems and other audio systems), computers, game machines, portable information terminals (e.g., mobile computers, cellular phones, portable game machines, and electronic books), image playback devices provided with recording media (devices that are capable of playing back recording media such as digital versatile discs (DVDs) and equipped with display devices that can display the image), and the like. Some specific examples thereof are illustrated in FIGS. 6A to 6F.

FIG. 6A illustrates a television device which is one example of a display device according the present invention. The television device includes a housing 9101, a supporting base 9102, a display portion 9103, a speaker portion 9104, a video input terminal 9105, and the like.

Note that the category of the display device according to the present invention covers all types of information display devices, for example, display devices for a personal computer, for TV broadcast reception, for advertisement display, and the like.

In the display portion 9103 of this television device, light-emitting elements similar to those described in Embodiment 4 or 5 are arranged in a matrix. The light-emitting elements have a feature of high reliability. Accordingly, the display portion 9103 which includes the light-emitting elements has similar features. Therefore, this television device is highly reliable and the image quality is hardly deteriorated. With such features, deterioration compensation function and a power supply circuit can be significantly reduced or downsized in the television device; therefore, reduction in size and weight of the housing 9101 and the supporting base 9102 can be achieved. In the television device according to the present invention, high image quality and reduction in size and weight are achieved; therefore, a product which is suitable for living environment can be provided.

FIG. 6B illustrates a computer according to the present invention. The computer includes a main body 9201, a housing 9202, a display portion 9203, a keyboard 9204, an external connection port 9205, a pointing device 9206, and the like. In the display portion 9203 of this computer, light-emitting elements similar to those described in Embodiment 4 or 5 are arranged in a matrix. The light-emitting elements have a feature of high reliability. Accordingly, the display portion 9203 which includes the light-emitting elements has similar features. Therefore, this computer is highly reliable and the image quality is hardly deteriorated. With such features, deterioration compensation function and a power supply circuit can be significantly reduced or downsized in the computer; therefore, reduction in size and weight of the main body 9201, and the housing 9202 can be achieved. In the computer according to the present invention, high image quality and reduction in size and weight are achieved; therefore, a product which is suitable for environment can be provided.

FIGS. 6C and 6F each illustrate a cellular phone according the present invention. The cellular phone illustrated in FIG. 6C includes a main body 9401, a housing 9402, a display portion 9403, an audio input portion 9404, an audio output portion 9405, operation keys 9406, an external connection port 9407, an antenna 9408, and the like. The cellular phone illustrated in FIG. 6F includes a main body 8401, a housing 8402, a display portion 8403, an audio input portion 8404, an audio output portion 8405, operation keys 8406, an external connection port 8407, and the like.

In the display portion 9403 and the display portion 8403 of those cellular phones, light-emitting elements similar to those described in Embodiment 4 or 5 are arranged in a matrix. The light-emitting elements have a feature of high reliability. Accordingly, the display portion 9403 and the display portion 8403 which include the light-emitting elements have similar features. Therefore, those cellular phones are highly reliable and the image quality is hardly deteriorated. With such features, deterioration compensation function and a power supply circuit can be significantly reduced or downsized in those cellular phones; therefore, reduction in size and weight of the main bodies 9401 and 8401 and the housings 9402 and 8402 can be achieved. High image quality and reduction in size and weight or those cellular phones according to the present invention are achieved; therefore, products which are suitable for being carried around can be provided.

FIG. 6D illustrates a camera according to the present invention which includes a main body 9501, a display portion 9502, a housing 9503, an external connection port 9504, a remote control receiving portion 9505, an image receiving portion 9506, a battery 9507, an audio input portion 9508, operation keys 9509, an eye piece portion 9510, and the like. In the display portion 9502 of the camera, light-emitting elements similar to those described in Embodiment 4 or 5 are arranged in a matrix. The light-emitting elements have a feature of high reliability. Accordingly, the display portion 9502 which includes the light-emitting elements has similar features. Therefore, this camera is highly reliable and the image quality is hardly deteriorated. With such features, deterioration compensation function and a power supply circuit can be significantly reduced or downsized in the camera; therefore, reduction in size and weight of the main body 9501 can be achieved. High image quality and reduction in size and weight of the camera according to the present invention are achieved; therefore, a product which is suitable for being carried around can be provided.

FIG. 6E illustrates an electronic paper according to the present invention which may have a flexible property. The electronic paper includes a main body 9660, a display portion 9661 which displays images, a driver IC 9662, a receiver 9663, a film battery 9664, and the like. The driver IC, the receiver, or the like may be mounted using a semiconductor component. In the electronic paper of the present invention, the main body 9660 is formed using a flexible material such as plastic or a film. In the display portion 9661 of the electronic paper, light-emitting elements similar to those described in Embodiment 4 or 5 are arranged in a matrix. The light-emitting elements have a feature of long lifetime and low power consumption. Accordingly, the display portion 9661 which includes the light-emitting elements has similar features. Therefore, this electronic paper is highly reliable and low power consumption.

Furthermore, such an electronic paper is extremely light and flexible and can be rolled into a cylindrical shape as well; thus, the electronic paper is a display device that has a great advantage in terms of portability. The electronic device of the present invention allows a display medium having a large screen to be freely carried.

The electronic paper illustrated in FIG. 6E can be used as a display means of a navigation system, an audio reproducing device (such as a car audio or an audio component), a personal computer, a game machine, and a portable information terminal (such as a mobile computer, a cellular phone, a portable game machine, or an electronic book reader). In addition, the display device can be used as a means for mainly displaying still images for electrical home appliances such as a refrigerator, a washing machine, a rice cooker, a fixed telephone, a vacuum cleaner, or a clinical thermometer; hanging advertisements in trains; and large-sized information displays such as arrival and departure boards in railroad stations and airports.

As described above, the applicable range of the light-emitting device of the present invention is so wide that the light-emitting device can be applied to electronic devices in various fields. By using any of the carbazole derivatives which are described in Embodiments 1 to 3, the electronic device having a highly reliable display portion can be obtained.

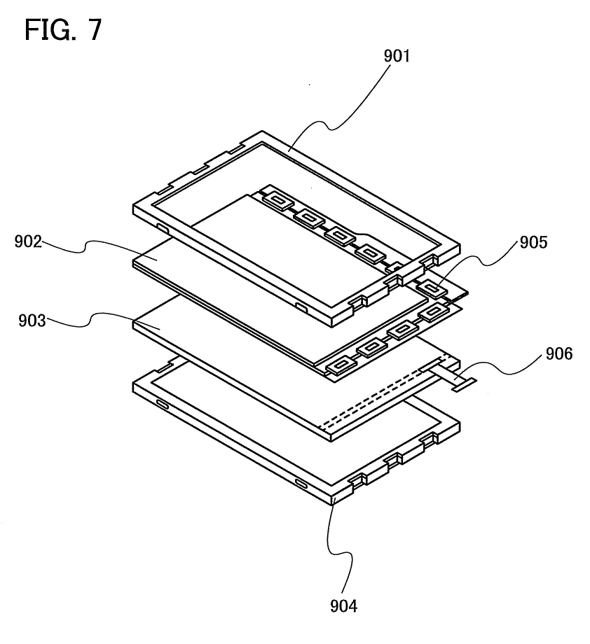

The light-emitting device of the present invention can also be used as a lighting device. An example in which the light-emitting device of the present invention is used as a lighting device is described with reference to FIG. 7.

FIG. 7 illustrates an example of a liquid crystal display device using a light-emitting device of the present invention as a backlight. The liquid crystal display device illustrated in FIG. 7 includes a housing 901, a liquid crystal layer 902, a backlight 903, and a housing 904. The liquid crystal layer 902 is connected to a driver IC 905. The light-emitting device of the present invention is used as the backlight 903 to which current is supplied through a terminal 906.

By using the light-emitting device of the present invention for a backlight of a liquid crystal display device, a highly reliable backlight can be obtained. Further, the light-emitting device of the present invention can be applied to a lighting device of plane light emission and can have a large area. Therefore, the backlight can have a large area, and a liquid crystal display device having a large area can be obtained. Furthermore, since the light-emitting device of the present invention is thin, the thickness of a display device can also be reduced.

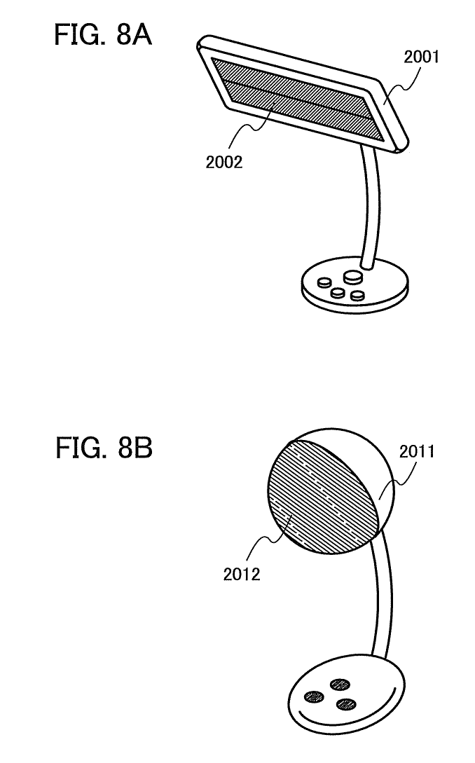

FIGS. 8A and 8B illustrate examples in which a light-emitting device to which the present invention is applied is used as a table lamp, which is a kind of lighting device. The table lamps illustrated in FIGS. 8A and 8B each include a housing 2001 and a light source 2002. The light-emitting device of the present invention is used as the light source 2002. Since the light-emitting device of the present invention is highly reliable, the table lamps are also highly reliable.

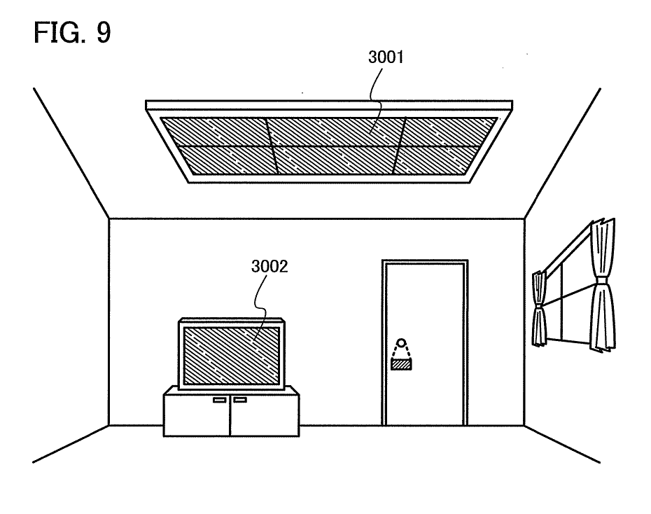

FIG. 9 illustrates an example in which a light-emitting device to which the present invention is applied is used as an indoor lighting device 3001. Since the light-emitting device of the present invention can have a large area, the light-emitting device can be used as a large-area lighting device. Further, since the light-emitting device of the present invention is thin, the light-emitting device of the present invention can be used as a lighting device having a reduced thickness. In a room where the light-emitting device of the present invention is used as the indoor lighting device 3001 in this manner, a television device 3002 according to the present invention, which is similar to the one illustrated in FIG. 6A, can be placed so that public broadcasting and movies can be watched.

### Example 1

In this example, a synthesis method of 3-(1-naphthyl)-9-[4-(10-phenyl-9-anthryl)phenyl]-9H-carbazole (CzPAαN) represented by the structural formula (101), which is one mode of the carbazole derivative of the present invention, will be specifically described.

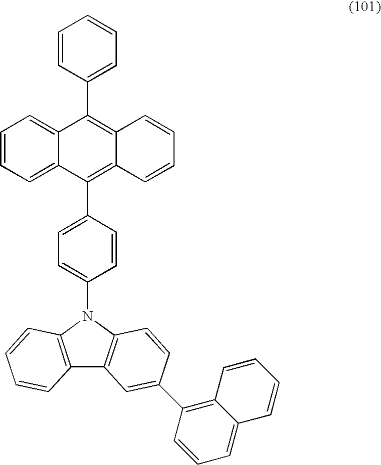

### Synthesis Example 1

First, Synthesis Example 1 will be described.

### [Step 1] Synthesis of 3-(1-naphthyl)-9H-carbazole

A synthetic scheme of 3-(1-naphthyl)-9H-carbazole is shown in (A-1).

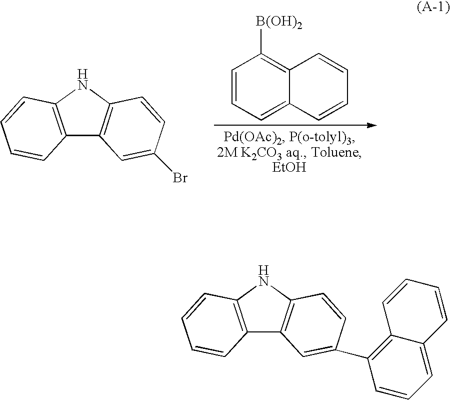

Into a 100 mL three-neck flask were put 0.50 g (2.0 mmol) of 3-bromo-9H-carbazole, 0.35 g (2.0 mmol) of 1-naphthylboronic acid, and 0.15 g (0.50 mmol) of tri(ortho-tolyl)phosphine, and the air in the flask was replaced with nitrogen. To this mixture were added 30 mL of toluene, 10 mL of ethanol, and 2.0 mL of a potassium carbonate aqueous solution (2.0 mol/L). This mixture was stirred to be degassed while the pressure was reduced. To the mixture was added 23 mg (0.10 mmol) of palladium(II) acetate, and the mixture was stirred at 80° C. under a nitrogen stream for 2 hours. After the stir, the aqueous layer of the mixture was extracted with toluene and the extracted solution and the organic layer were washed together with saturated saline. The organic layer was dried with magnesium sulfate, and this mixture was subjected to gravity filtration. The obtained filtrate was concentrated to give a solid. The obtained solid was dissolved in about 10 mL of toluene. The solution was subjected to suction filtration through Celite (Catalog No. 531-16855, manufactured by Wako Pure Chemical Industries, Ltd.), alumina, and Florisil (Catalog No. 540-00135, manufactured by Wako Pure Chemical Industries, Ltd.). The obtained filtrate was concentrated to give a white solid. This solid was washed with hexane to give 0.32 g of white powder, which was the object, at a yield of 53%.

### [Step 2] Synthesis Method of CzPAαN

A synthetic scheme of CzPAαN is shown in (A-2).

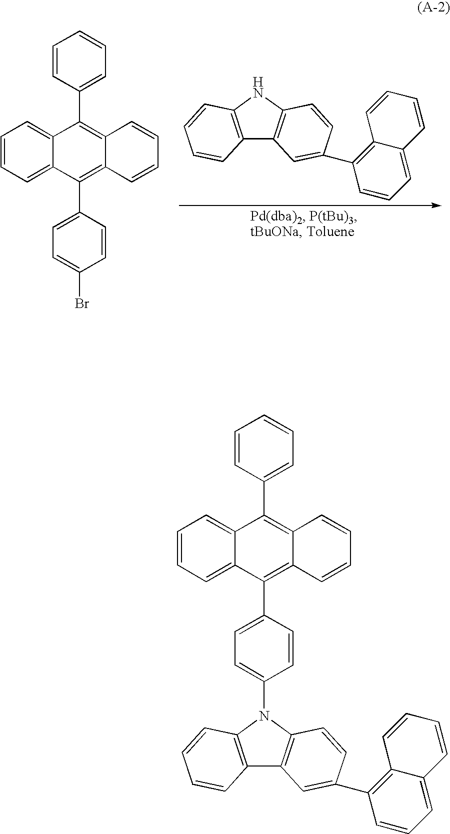

Into a 100 mL three-neck flask were put 0.45 g (1.1 mmol) of 9-(4-bromophenyl)-10-phenylanthracene, 0.32 g (1.1 mmol) of 3-(1-naphthyl)-9H-carbazole which was synthesized in Step 1 of Example 1, and 0.21 g (2.2 mmol) of sodium-tert-butoxide. The air in the flask was replaced with nitrogen. Then, to the mixture were added 20 mL of toluene and 0.20 mL of tri(tert-butyl)phosphine (a 10 wt % hexane solution). This mixture was stirred to be degassed while the pressure was reduced. After the degassing, 32 mg (0.055 mmol) of bis(dibenzylideneacetone)palladium(0) was added to the mixture. This mixture was stirred under a nitrogen stream at 110° C. for 2 hours. After the stir, the mixture was subjected to suction filtration through Celite (Catalog No. 531-16855, manufactured by Wako Pure Chemical Industries, Ltd.), alumina, and Florisil (Catalog No. 540-00135, manufactured by Wako Pure Chemical Industries, Ltd.). The obtained filtrate was concentrated to give a solid. The obtained solid was purified by silica gel column chromatography (a developing solvent was a mixed solvent of hexane and toluene (hexane:toluene=5:1)) to give a light yellow solid. The obtained light yellow solid was recrystallized with toluene/hexane to give 0.31 g of light yellow powder, which was the object, at a yield of 46%.

0.31 g of the obtained light yellow powder was sublimated and purified by train sublimation. The sublimation purification was performed under such conditions that the light yellow powder was heated at 310° C. with an argon gas applied at a flow rate of 4.0 mL/min under reduced pressure. After the sublimation purification, 0.25 g of a light yellow solid of CzPAαN was recovered, at a yield of 80%.

### Synthesis Example 2

In Synthesis Example 2, a synthesis method of CzPAαN, which is different from the synthesis method in Synthesis Example 1, will be specifically described.

### [Step 1] Synthesis of 3-bromo-9-[4-(10-phenyl-9-anthryl)phenyl]-9H-carbazole

A synthetic scheme of 3-bromo-9-[4-(10-phenyl-9-anthryl)phenyl]-9H-carbazole is shown in (B-1).

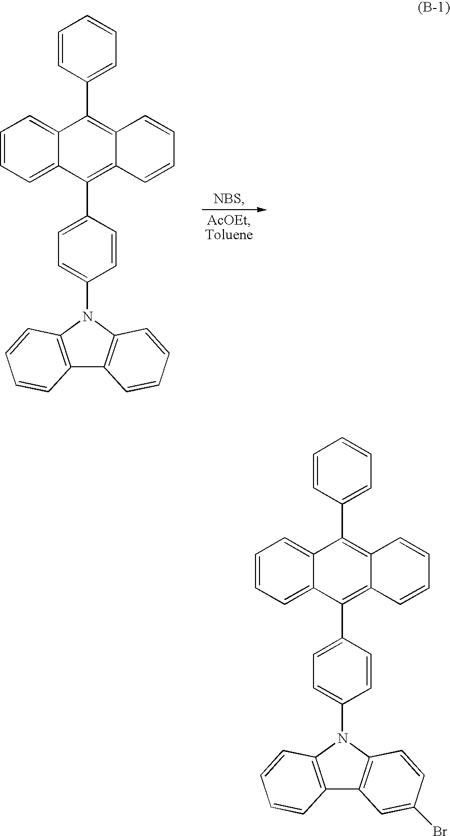

Into a 1 L Erlenmeyer flask were put 5.0 g (10 mmol) of 9-[4-(10-phenyl-9-anthryl)phenyl]-9H-carbazole (CzPA), 600 mL of ethyl acetate, and 150 mL of toluene. This mixture was stirred while being heated at about 50° C. or higher, and dissolution of CzPA was confirmed. To this solution was added 1.8 g (10 mmol) of N-bromo succinimide (NBS). This solution was stirred at a room temperature under the atmosphere for 5 days. After the stir, about 150 mL of a sodium thiosulfate aqueous solution was added to this solution and the solution was stirred for 1 hour. After the organic layer of this mixture was washed with water, the aqueous layer was extracted with toluene and the extracted solution and the organic layer were washed together with saturated saline. The organic layer was dried with magnesium sulfate, and this mixture was subjected to gravity filtration. The obtained filtrate was concentrated to give a light yellow solid. The obtained solid was recrystallized with a mixed solvent of toluene and hexane to give 5.2 g of light yellow powder, which was the object, at a yield of 90%.

### [Step 2] Synthesis of CzPAαN

A synthetic scheme of CzPAαN is shown in (B-2).

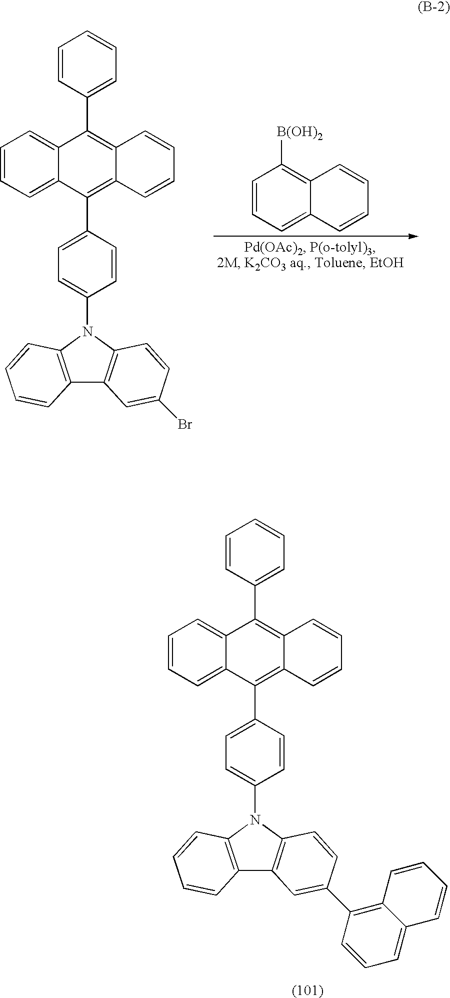

Into a 200 mL three-neck flask were put 2.8 g (4.9 mmol) of 3-bromo-9-[4-(10-phenyl-9-anthryl)phenyl]-9H-carbazole, 0.84 g (4.9 mmol) of 1-naphthyl boronic acid, and 0.36 g (1.2 mmol) of tri(ortho-tolyl)phosphine, and the air in the flask was replaced with nitrogen. To this mixture were added 5.0 mL of a potassium carbonate aqueous solution (2.0 mol/L), 60 mL of toluene, and 20 mL of ethanol. This mixture was stirred to be degassed while the pressure was reduced. To the mixture was added 55 mg (0.24 mmol) of palladium(II) acetate, and the mixture was stirred at 80° C. under a nitrogen stream for 4 hours. After the stir, the aqueous layer of the mixture was extracted with toluene and the extracted solution and the organic layer were washed together with saturated saline. The organic layer was dried with magnesium sulfate, and this mixture was subjected to gravity filtration. The obtained filtrate was concentrated to give an oily substance. The oily substance was dissolved in about 10 mL of toluene. The solution was subjected to suction filtration through Celite (Catalog No. 531-16855, manufactured by Wako Pure Chemical Industries, Ltd.), alumina, and Florisil (Catalog No. 540-00135, manufactured by Wako Pure Chemical Industries, Ltd.). The obtained filtrate was concentrated to give an oily substance. The obtained oily substance was purified by silica gel column chromatography (a developing solvent was a mixed solvent of hexane and toluene (hexane:toluene=5:1)) to give a light yellow oily substance. The obtained oily substance was recrystallized with toluene/hexane to give 1.8 g of light yellow powder, which was the object, at a yield of 60%.

1.8 g of the obtained light yellow powder of CzPAαN was sublimated and purified by train sublimation. The sublimation purification was performed under such conditions that the light yellow powder was heated at 320° C. with an argon gas applied at a flow rate of 4.0 mL/min under reduced pressure. After the sublimation purification, 1.7 g of a light yellow solid of CzPAαN was recovered, at a yield of 94%.

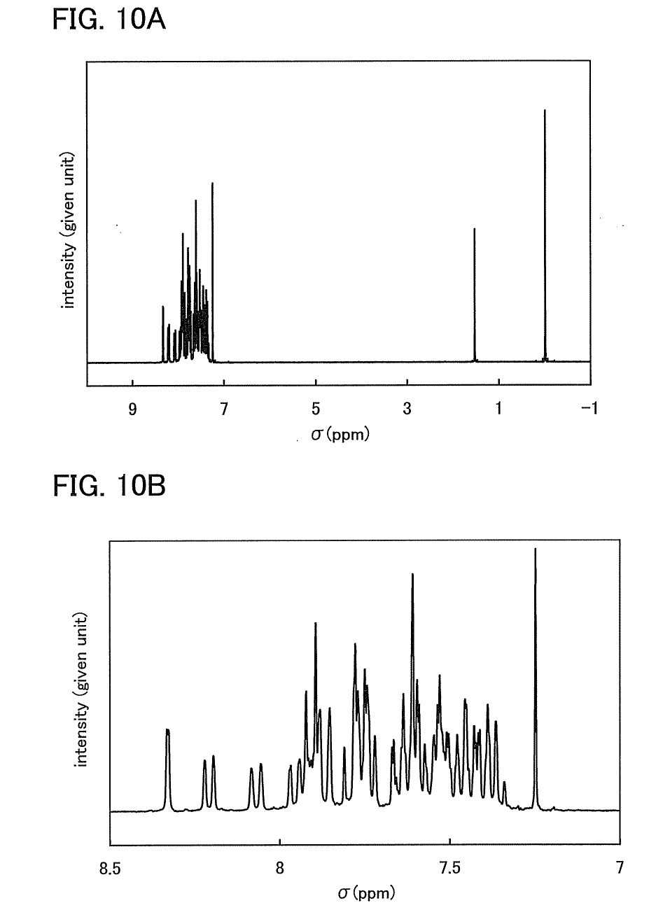

Next, the compound obtained by the above synthesis method was identified as CzPAαN by nuclear magnetic resonance (NMR). ¹H NMR data of CzPAαN is shown below. ¹H NMR (CDCl₃, 300 MHz): δ=7.34-7.67 (m, 16H), 7.72-7.81 (m, 6H), 7.85-7.96 (m, 6H), 8.07 (d, J=8.4 Hz, 1H), 8.20 (d, J=7.8 Hz, 1H), 8.32 (d, J=1.5 Hz, 1H). The ¹H NMR chart is illustrated in FIGS. 10A and 10B. Note that FIG. 10B is a chart showing an enlarged portion of FIG. 10A in the range of from 7.0 ppm to 8.5 ppm.

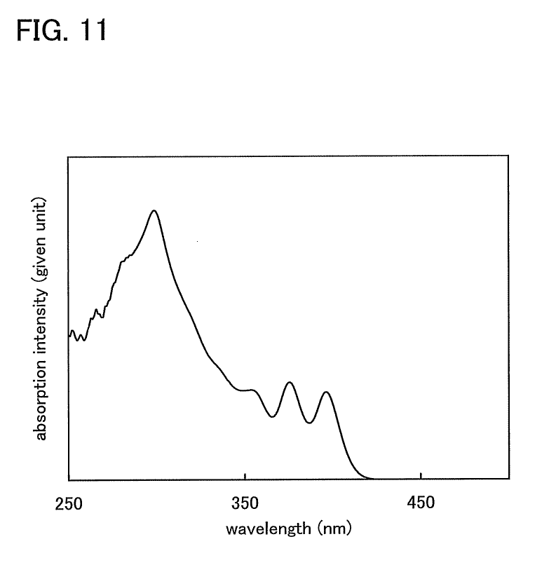

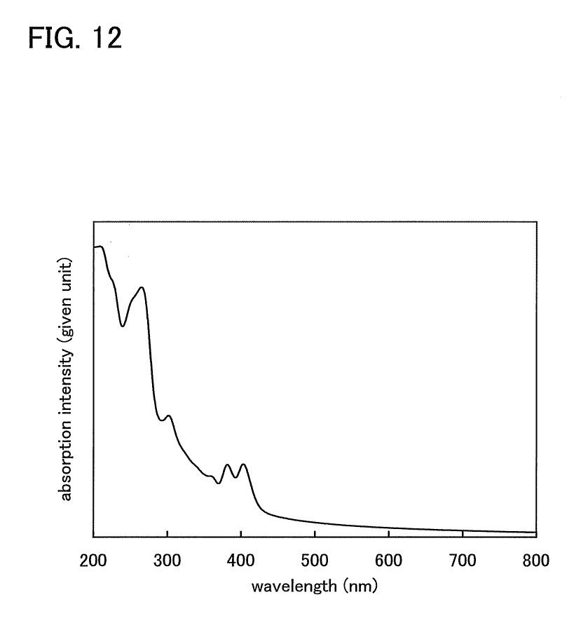

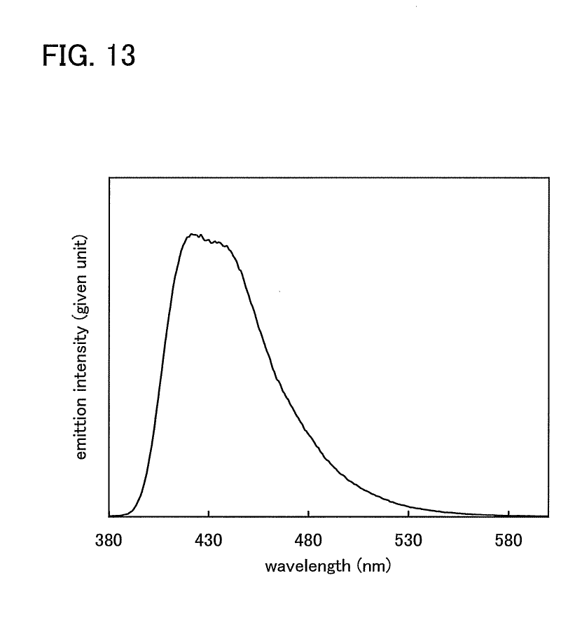

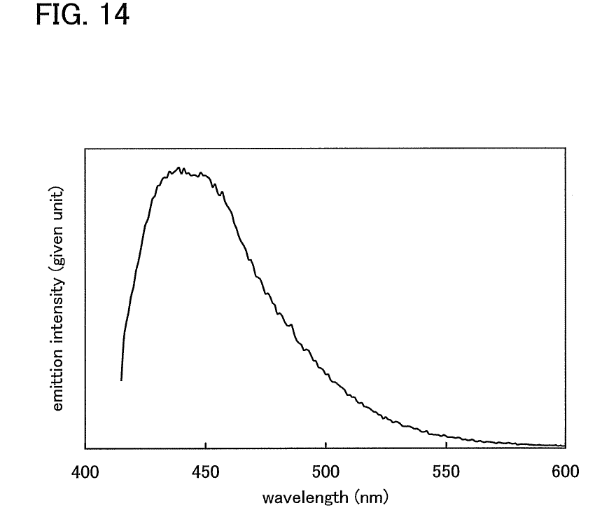

FIG. 11 illustrates an absorption spectrum of CzPAαN included in a toluene solution. FIG. 12 illustrates an absorption spectrum of a thin film of CzPAαN. An ultraviolet-visible spectrophotometer (V-550, manufactured by JASCO Corporation) was used for the measurement. The solution was put in a quartz cell and the thin film was formed by evaporation onto a quartz substrate to manufacture a sample. As for the spectrum of the solution, the absorption spectrum obtained by subtraction of the absorption spectrum of the quartz cell including only toluene is illustrated in FIG. 11. As for the spectrum of the thin film, the absorption spectrum obtained by subtraction of the absorption spectrum of the quartz substrate is illustrated in FIG. 12. In FIG. 11 and FIG. 12, the horizontal axis represents wavelength (nm) and the vertical axis represents absorption intensity (given unit). In the case of the toluene solution, absorption was observed at around 299 nm, 354 nm, 376 nm, and 396 nm. In the case of the thin film, absorption was observed at around 209 nm, 265 nm, 302 nm, 361 nm, 382 nm, and 403 nm. The emission spectrum of the toluene solution of CzPAαN (excitation wavelength: 376 nm) is illustrated in FIG. 13. The emission spectrum of the thin film of CzPAαN (excitation wavelength: 401 nm) is illustrated in FIG. 14. In FIG. 13 and FIG. 14, the horizontal axis represents wavelength (nm), and the vertical axis represents emission intensity (given unit). In the case of the toluene solution, the maximum emission wavelength was 423 nm (excitation wavelength: 376 nm). In the case of the thin film, the maximum emission wavelength was 439 nm (excitation wavelength: 401 nm).

The results of measuring the thin film of CzPAαN by photoelectron spectrometer (AC-2, manufactured by Riken Keiki Co., Ltd.) in the atmosphere indicated that the HOMO level of CzPAαN was −5.77 eV. Moreover, the absorption edge was obtained from Tauc plot, with an assumption of direct transition, using data on the absorption spectrum of the thin film of CzPAαN in FIG. 12. When the absorption edge was estimated as an optical energy gap, the energy gap was 2.93 eV. The LUMO level, which was estimated from the HOMO level and the energy gap, was −2.84 eV.

Further, oxidation-reduction reaction properties of CzPAαN were measured. The oxidation-reduction reaction properties were measured by cyclic voltammetry (CV) measurement. An electrochemical analyzer (ALS model 600A, manufactured by BAS Inc.) was used for the measurement.

A solution used in the CV measurement was prepared in such a manner that dehydrated dimethylformamide (DMF) (99.8%, catalog number; 22705-6, manufactured by Sigma-Aldrich Co.) was used as a solvent, tetra-n-butylammonium perchlorate (n-Bu₄ NClO₄) (catalog number; T0836, manufactured by Tokyo Kasei Kogyo Co., Ltd.), which was a supporting electrolyte, was dissolved in the solvent so as to have a concentration of 100 mmol/L, and an object to be measured was dissolved so as to have a concentration of 1 mmol/L. Further, a platinum electrode (a PTE platinum electrode, manufactured by BAS Inc.) was used as a working electrode. A platinum electrode (a VC-3 Pt counter electrode (5 cm), manufactured by BAS Inc.) was used as an auxiliary electrode. An Ag/Ag⁺ electrode (an RE5 nonaqueous solvent reference electrode, manufactured by BAS Inc.) was used as a reference electrode. The measurement was performed at a room temperature.

The oxidation reaction characteristics of CzPAαN were measured as follows. A scan in which the potential of the working electrode with respect to the reference electrode was changed to 1.20 V from −0.01 V and then the potential was changed to −0.01 V from 1.20 V was set as one cycle, and 100 cycle measurements were performed. Note that the scan speed of the CV measurement was set at 0.1 V/s.

The reduction reaction characteristics of CzPAαN were measured as follows. A scan in which the potential of the working electrode with respect to the reference electrode was changed to −2.40 V from −1.49 V and then the potential was changed to −1.49 V from −2.40 V was set as one cycle, and 100 cycle measurements were performed. Note that the scan speed of the CV measurement was set at 0.1 V/s.

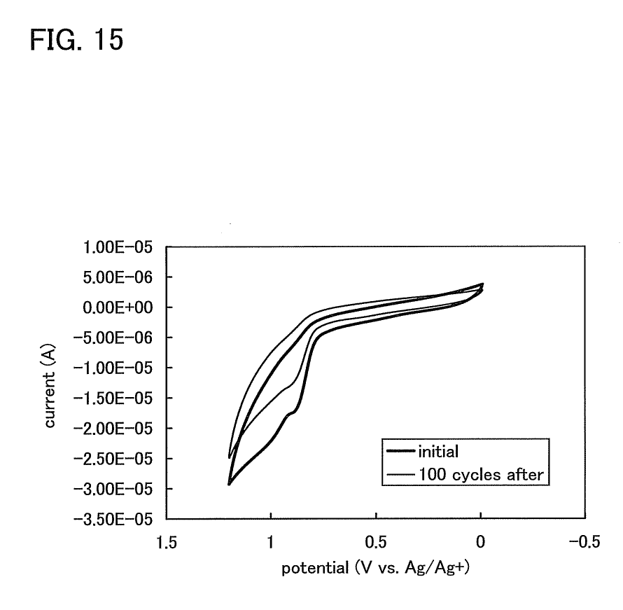

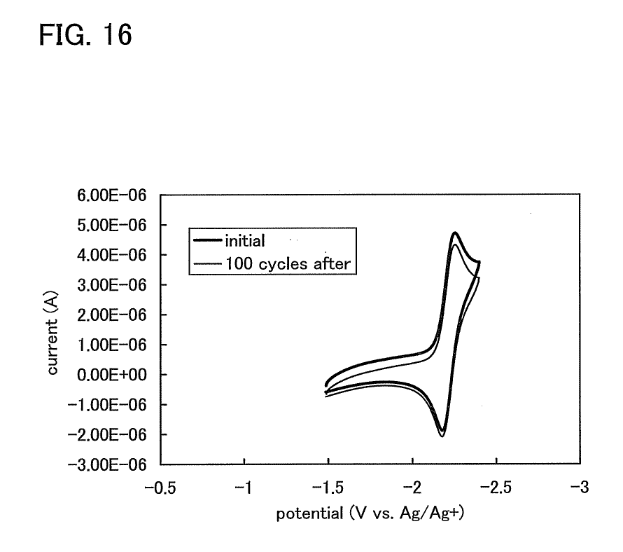

FIG. 15 illustrates CV measurement results on the oxidation reaction characteristic of CzPAαN and FIG. 16 illustrates CV measurement results on the reduction reaction characteristic of CzPAαN. In each of FIG. 15 and FIG. 16, the horizontal axis represents potential (V) of the working electrode with respect to the reference electrode, and the vertical axis represents current value (A) that flowed between the working electrode and the counter electrode. According to FIG. 15, a current indicating oxidation was observed at around +0.84 V (vs. Ag/Ag⁺ electrode). According to FIG. 16, a current indicating reduction was observed at around −2.22 V (vs. Ag/Ag⁺ electrode).

In spite of the fact that as many as 100 cycles of scan were performed, a peak position and a peak intensity of the CV curve scarcely changed in the oxidation reaction and the reduction reaction, which shows that the carbazole derivative of the present invention is extremely stable against repetition of oxidation-reduction reactions.

### Example 2

In this example, a synthesis method of 3-(2-naphthyl)-9-[4-(10-phenyl-9-anthryl)phenyl]-9H-carbazole (CzPAβN) represented by the structural formula (201), which is the carbazole derivative of the present invention, will be specifically described.

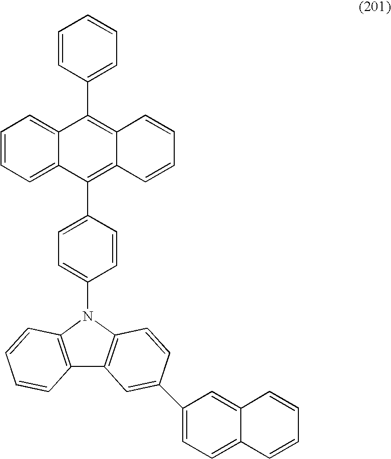

A synthetic scheme of CzPAβN is shown in (C-1).

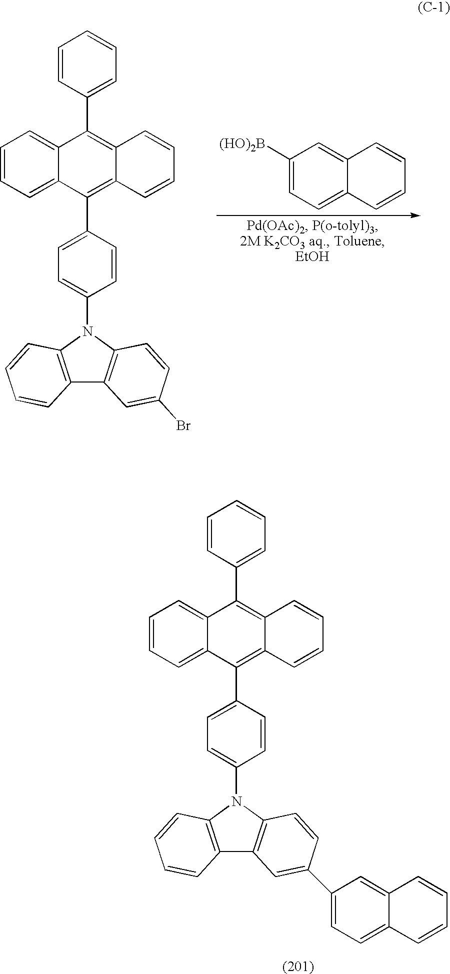

Into a 100 mL three-neck flask were put 1.0 g (1.7 mmol) of 3-bromo-9-[4-(10-phenyl-9-anthryl)phenyl]-9H-carbazole, 0.30 g (1.7 mmol) of 2-naphthylboronic acid, and 0.13 g (0.42 mmol) of tri(ortho-tolyl)phosphine, and the air in the flask was replaced with nitrogen. To this mixture were added 30 mL of toluene, 10 mL of ethanol, and 2.0 mL of a potassium carbonate aqueous solution (2.0 mol/L). This mixture was stirred to be degassed while the pressure was reduced. To this mixture was added 19 mg (0.085 mmol) of palladium(II) acetate, and the mixture was stirred at 80° C. under a nitrogen stream for 3 hours. After the stir, the aqueous layer of the mixture was extracted with toluene and the extracted solution and the organic layer were washed together with saturated saline. The organic layer was dried with magnesium sulfate, and this mixture was subjected to gravity filtration. The obtained filtrate was concentrated to give an oily substance. The obtained oily substance was dissolved in about 10 mL of toluene. The solution was subjected to suction filtration through Celite (Catalog No. 531-16855, manufactured by Wako Pure Chemical Industries, Ltd.), alumina, and Florisil (Catalog No. 540-00135, manufactured by Wako Pure Chemical Industries, Ltd.). The obtained filtrate was concentrated to give an oily substance. The obtained oily substance was purified by silica gel column chromatography (a developing solvent was a mixed solvent of hexane and toluene (hexane:toluene=5:1)) to give a light yellow solid. The obtained light yellow solid was recrystallized with toluene/hexane to give 0.73 g of light yellow powder, which was the object, at a yield of 69%.

0.71 g of the obtained light yellow powder (of CzPAβN) was sublimated and purified by train sublimation. The sublimation purification was performed under such conditions that the light yellow powder was heated at 310° C. with an argon gas applied at a flow rate of 4.0 mL/min under reduced pressure. After the sublimation purification, 0.64 g of a light yellow solid of CzPAβN was recovered, at a yield of 90%.

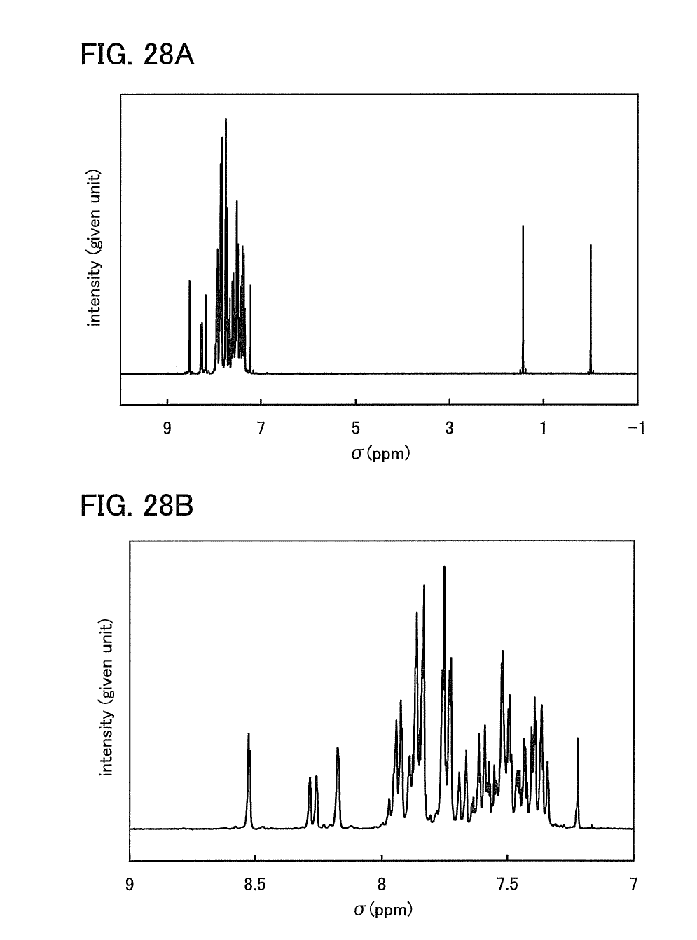

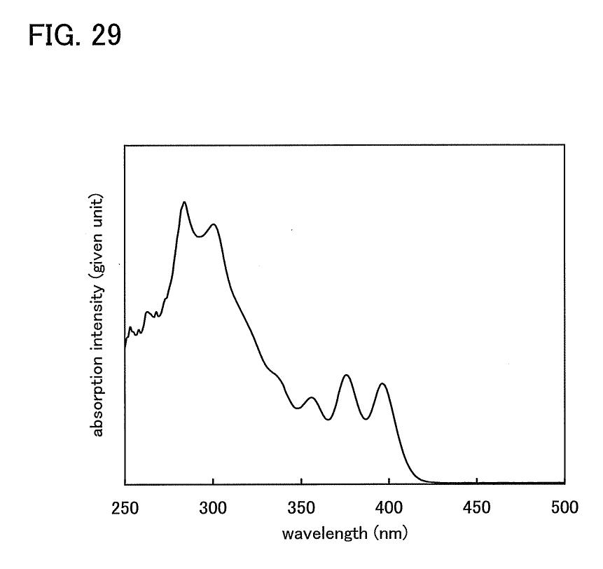

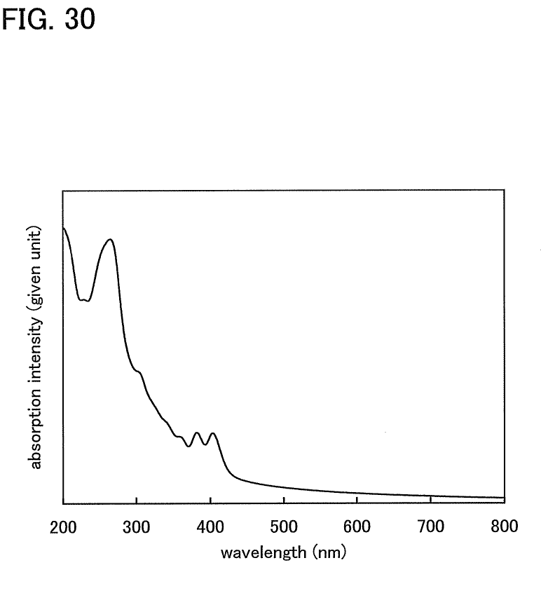

This compound was identified as CzPAβN by nuclear magnetic resonance (NMR). ¹H NMR data of CzPAβN is shown below. ¹H NMR (CDCl₃, 300 MHz): δ=7.37-7.66 (m, 13H), 7.70-7.80 (m, 6H), 7.85-8.00 (m, 9H), 8.20 (s, 1H), 8.30 (d, J=4.8 Hz, 1H), 8.54 (s, 1H). In addition, the ¹H NMR chart is illustrated in FIGS. 28A and 28B. Note that FIG. 28B is a chart showing an enlarged portion of FIG. 28A in the range of from 7.0 ppm to 9.0 ppm.

The thermogravimetry-differential thermal analysis (TG-DTA) was performed on the obtained CzPAβN. The measurement was performed with use of a high vacuum differential type differential thermal balance (TG/DTA 2410SA, manufactured by Bruker AXS K.K.). The measurement was performed under normal pressure in a nitrogen stream (at a flow rate of 200 mL/min) at a rate of temperature increase of 10° C./min. The temperature under atmospheric pressure at which the weight was reduced to 95% of the weight at the beginning of the measurement (hereinafter, the temperature is referred to as “5% weight loss temperature”) was 465° C.

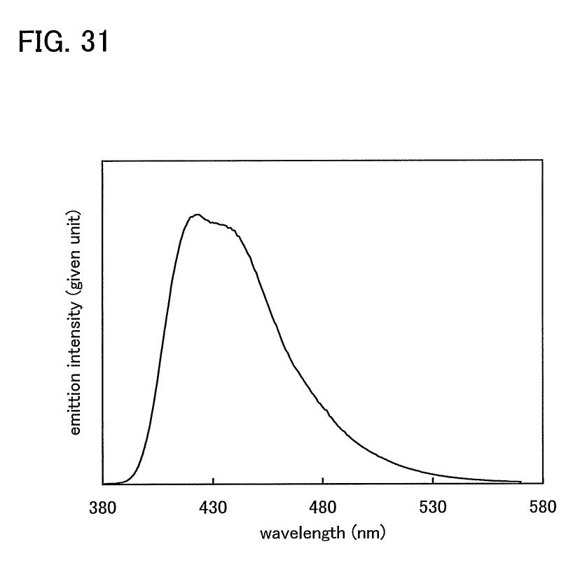

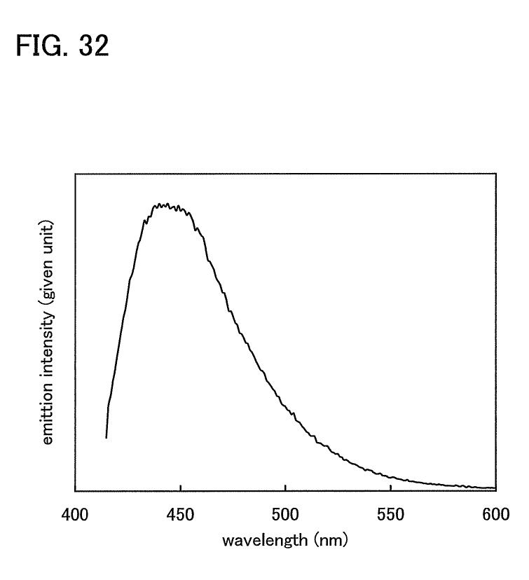

FIG. 29 illustrates an absorption spectrum of CzPAβN included in a toluene solution. FIG. 30 illustrates an absorption spectrum of a thin film of CzPAβN. An ultraviolet-visible spectrophotometer (V-550, manufactured by JASCO Corporation) was used for the measurement. The solution was put in a quartz cell and the thin film was formed by evaporation onto a quartz substrate to manufacture a sample. As for the spectrum of the solution, the absorption spectrum obtained by subtraction of the absorption spectrum of the quartz cell including only toluene is illustrated in FIG. 29. As for the spectrum of the thin film, the absorption spectrum obtained by subtraction of the absorption spectrum of the quartz substrate is illustrated in FIG. 30. In FIG. 29 and FIG. 30, the horizontal axis represents wavelength (nm) and the vertical axis represents absorption intensity (given unit). In the case of the toluene solution, absorption was observed at around 300 nm, 356 nm, 376 nm, and 396 nm. In the case of the thin film, absorption was observed at around 304 nm, 360 nm, 382 nm, and 403 nm. The emission spectrum of the toluene solution of CzPAβN (excitation wavelength: 376 nm) is illustrated in FIG. 31. The emission spectrum of the thin film of CzPAβN (excitation wavelength: 401 nm) is illustrated in FIG. 32. In FIG. 31 and FIG. 32, the horizontal axis represents wavelength (nm), and the vertical axis represents emission intensity (given unit). In the case of the toluene solution, the maximum emission wavelength was 423 nm (excitation wavelength: 376 nm), and in the case of the thin film, the maximum emission wavelength was 443 nm (excitation wavelength: 401 nm).

The results of measuring the thin film of CzPAβN by photoelectron spectrometer (AC-2, manufactured by Riken Keiki Co., Ltd.) in the atmosphere indicated that the HOMO level of CzPAβN was −5.68 eV. Moreover, the absorption edge was obtained from Tauc plot, with an assumption of direct transition, using data on the absorption spectrum of the thin film of CzPAβN in FIG. 30. When the absorption edge was estimated as an optical energy gap, the energy gap was 2.92 eV. The LUMO level, which was estimated from the HOMO level and the energy gap, was −2.76 eV.

Further, oxidation-reduction reaction properties of CzPAβN were measured. The oxidation-reduction reaction properties were measured by cyclic voltammetry (CV) measurement. An electrochemical analyzer (ALS model 600A, manufactured by BAS Inc.) was used for the measurement.

A solution used in the CV measurement was prepared in such a manner that dehydrated dimethylformamide (DMF) (99.8%, catalog number; 22705-6, manufactured by Sigma-Aldrich Co.) was used as a solvent, tetra-n-butylammonium perchlorate (n-Bu₄ NClO₄) (catalog number; T0836, manufactured by Tokyo Kasei Kogyo Co., Ltd.), which was a supporting electrolyte, was dissolved in the solvent so as to have a concentration of 100 mmol/L, and an object to be measured was dissolved so as to have a concentration of 1 mmol/L. Further, a platinum electrode (a PTE platinum electrode, manufactured by BAS Inc.) was used as a working electrode. A platinum electrode (a VC-3 Pt counter electrode (5 cm), manufactured by BAS Inc.) was used as an auxiliary electrode. An Ag/Ag⁺ electrode (an RE5 nonaqueous solvent reference electrode, manufactured by BAS Inc.) was used as a reference electrode. The measurement was performed at a room temperature.

The oxidation reaction characteristics of CzPAβN were measured as follows. A scan in which the potential of the working electrode with respect to the reference electrode was changed to 0.97 V from −0.05 V and then the potential was changed to −0.05 V from 0.97 V was set as one cycle, and 100 cycle measurements were performed. Note that the scan speed of the CV measurement was set at 0.1 V/s.

The reduction reaction characteristics of CzPAβN were measured as follows. A scan in which the potential of the working electrode with respect to the reference electrode was changed to −2.39 V from −1.13 V and then the potential was changed to −1.13 V from −2.39 V was set as one cycle, and 100 cycle measurements were performed. Note that the scan speed of the CV measurement was set at 0.1 V/s.

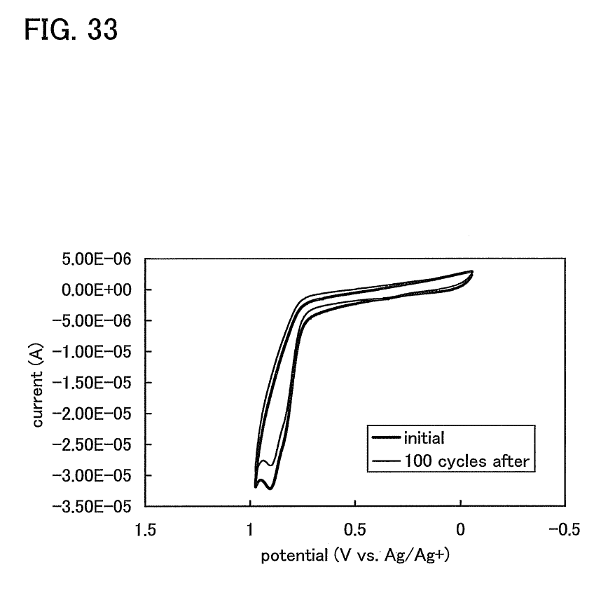

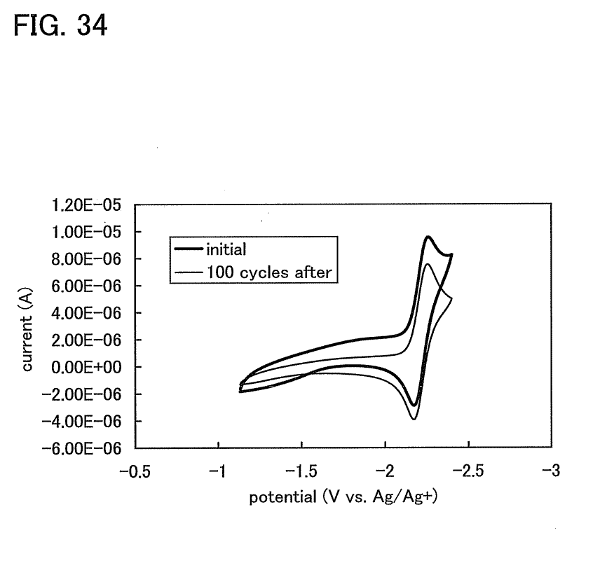

FIG. 33 illustrates CV measurement results on the oxidation reaction characteristic of CzPAβN and FIG. 34 illustrates CV measurement results on the reduction reaction characteristic of CzPAβN. In each of FIG. 33 and FIG. 34, the horizontal axis represents potential (V) of the working electrode with respect to the reference electrode, and the vertical axis represents current value (A) that flowed between the working electrode and the counter electrode. According to FIG. 33, a current indicating oxidation was observed at around +0.79 V (vs. Ag/Ag⁺ electrode). According to FIG. 34, a current indicating reduction was observed at around −2.22 V (vs. Ag/Ag⁺ electrode).

In spite of the fact that as many as 100 cycles of scan were performed, a peak position and a peak intensity of the CV curve scarcely changed in the oxidation reaction and the reduction reaction, which shows that the carbazole derivative of the present invention is extremely stable against repetition of oxidation-reduction reactions.

### Example 3

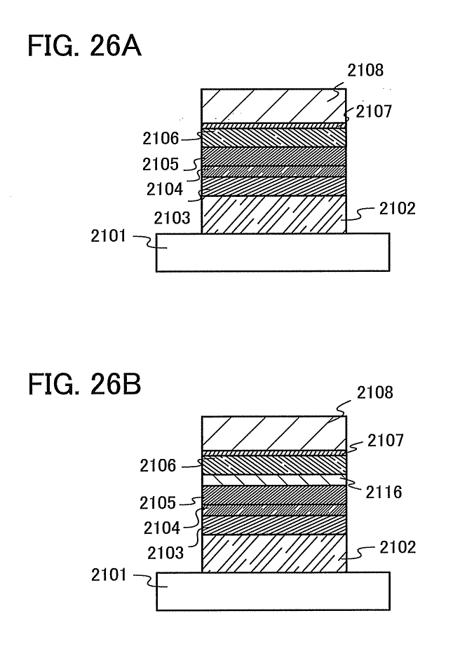

In this example, a light-emitting element of the present invention will be described with reference to FIGS. 26A and 26B.

The element structures of light-emitting elements 1-1 to 1-3 manufactured in this example are shown in Table 1. In Table 1, the mixture ratios are all represented in weight ratios.

TABLE 1

| | first | first | second | | | | fifth | second |
|---|---|---|---|---|---|---|---|---|
| | electrode | layer | layer | third layer | fourth layer | | layer | electrode |
| | 2102 | 2103 | 2104 | 2105 | 2106 | | 2107 | 2108 |
| light- | ITSO | NPB:MoOx | NPB | CzPAαN:PCBAPA | Alq | Bphen | LiF | Al |
| emitting | 110 nm | (=4:1) | 10 nm | (=1:0.1) | 10 nm | 20 nm | 1 nm | 200 nm |
| element | | 50 nm | | 30 nm | | | | |
| 1-1 | | | | | | | | |
| light- | ITSO | NPB:MoOx | NPB | CzPAαN:2PCAPA | Alq | Bphen | LiF | Al |
| emitting | 110 nm | (=4:1) | 10 nm | (=1:0.05) | 10 nm | 20 nm | 1 nm | 200 nm |
| element | | 50 nm | | 30 nm | | | | |
| 1-2 | | | | | | | | |
| light- | ITSO | NPB:MoOx | NPB | CzPAβN:PCBAPA | Alq | Bphen | LiF | Al |
| emitting | 110 nm | (=4:1) | 10 nm | (=1:0.1) | 10 nm | 20 nm | 1 nm | 200 nm |
| element | | 50 nm | | 30 nm | | | | |
| 1-3 | | | | | | | | |

Hereinafter, manufacturing methods of the light-emitting elements 1-1 to 1-3 of this example will be described.

For the light-emitting elements 1-1 to 1-3, indium tin oxide containing silicon oxide (ITSO) was deposited over a glass substrate 2101 by a sputtering method, whereby a first electrode 2102 was formed. The thickness of the first electrode 2102 was 110 nm, and the area thereof was 2 mm×2 mm.

Next, the substrate over which the first electrode was formed was fixed to a substrate holder provided in a vacuum evaporation apparatus so that a surface of the substrate on which the first electrode was formed faced downward. The pressure was reduced to about 10⁻⁴ Pa, and then 4,4′-bis[N-(1-naphthyl)-N-phenylamino]biphenyl (NPB) and molybdenum(VI) oxide were co-evaporated on the first electrode 2102, whereby a layer 2103 containing a composite material of an organic compound and an inorganic compound was formed as a first layer 2103. The thickness of the first layer 2103 was 50 nm and the weight ratio between NPB and molybdenum(VI) oxide was adjusted to be 4:1 (=NPB:molybdenum oxide). Note that co-evaporation is an evaporation method in which evaporation is performed at the same time from a plurality of evaporation sources in one treatment chamber.

Next, NPB was evaporated to a thickness of 10 nm, whereby a second layer 2104 was formed as a hole-transporting layer.

For the light-emitting element 1-1, CzPAαN synthesized in Example 1 and 4-(10-phenyl-9-anthryl)-4′-(9-phenyl-9H-carbazol-3-yl)triphenylamine (PCBAPA) were co-evaporated onto the second layer 2104 so that the weight ratio between CzPAαN and PCBAPA was 1:0.1 (=CzPAαN:PCBAPA), whereby a third layer 2105 was formed as a light-emitting layer. The thickness of the third layer 2105 was 30 nm.

In the light-emitting element 1-2, CzPAαN synthesized in Example 1 and 9,10-diphenyl-2-[N-phenyl-N-(9-phenyl-9H-carbazol-3-yl)amino]anthracene (2PCAPA) were co-evaporated onto the second layer 2104 so that the weight ratio between CzPAαN and 2PCAPA was 1:0.05 (=CzPAαN:2PCAPA), whereby a third layer 2105 was formed as a light-emitting layer. The thickness of the third layer 2105 was 30 nm.

For the light-emitting element 1-3, CzPAβN synthesized in Example 2 and 4-(10-phenyl-9-anthryl)-4′-(9-phenyl-9H-carbazol-3-yl)triphenylamine(PCBAPA) were co-evaporated onto the second layer 2104 so that the weight ratio between CzPAβN and PCBAPA was 1:0.1 (=CzPAβN:PCBAPA), whereby a third layer 2105 was formed as a light-emitting layer. The thickness of the third layer 2105 was 30 nm.

Next, for the light-emitting elements 1-1 to 1-3, Alq was evaporated onto the third layer 2105 to a thickness of 10 nm, and then Bphen was evaporated to a thickness of 20 nm to form a stacked layer, whereby a fourth layer 2106 was formed as an electron-transporting layer. Further, lithium fluoride (LiF) was evaporated onto the fourth layer 2106 to a thickness of 1 nm, whereby a fifth layer 2107 was formed as an electron-injecting layer. Lastly, aluminum was evaporated to a thickness of 200 nm as a second electrode 2108 which functions as a cathode. Accordingly, the light-emitting elements 1-1 to 1-3 of this example were obtained. Note that in all of the above evaporation steps, a resistance heating method was used. In addition, structural formulas of NPB, PCBAPA, 2PCAPA, Alq, and Bphen are shown below.

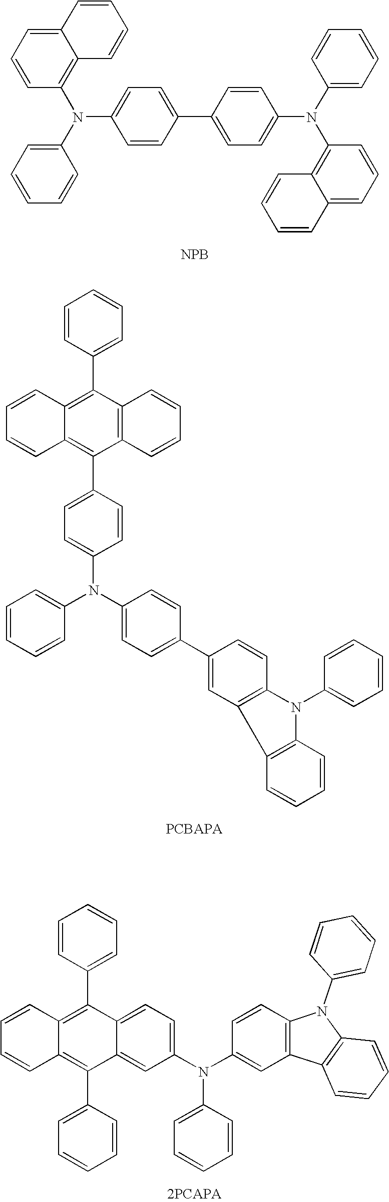

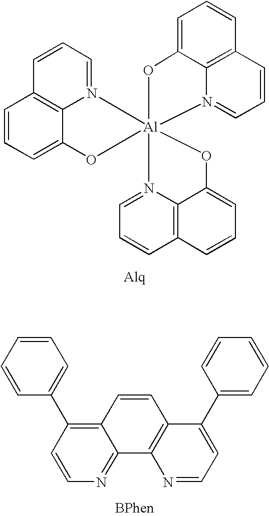

The light-emitting elements 1-1 to 1-3 obtained in the above manner were sealed in a glove box under a nitrogen atmosphere without being exposed to the atmosphere. After that, the operating characteristics of the light-emitting elements 1-1 to 1-3 were measured. The measurement was performed at a room temperature (in the atmosphere in which the temperature was kept at 25° C.).

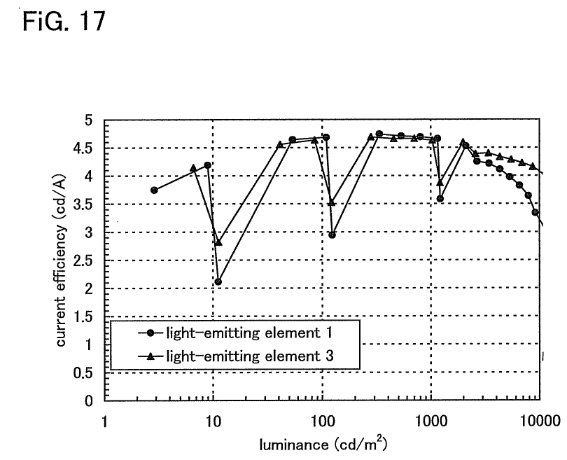

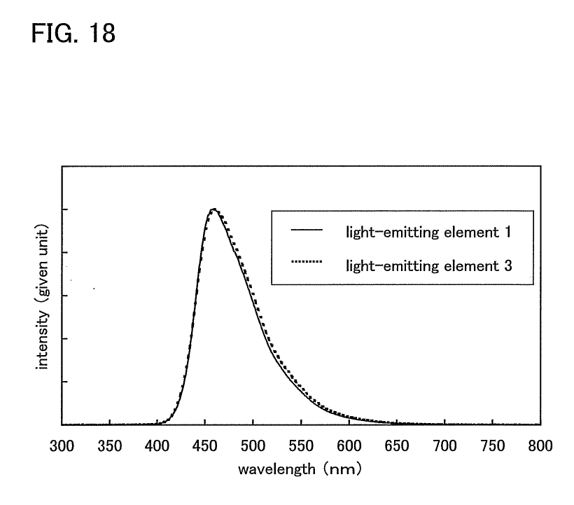

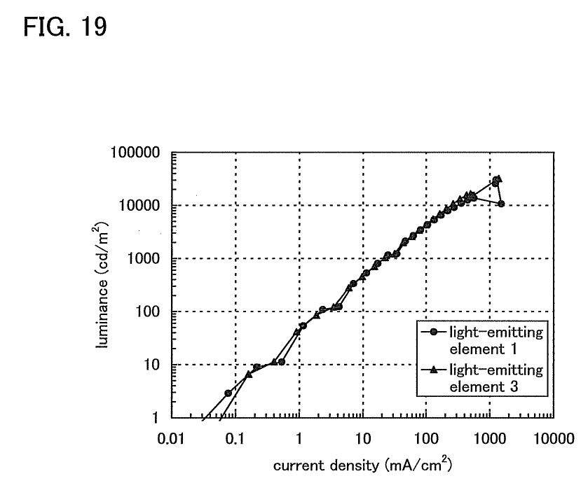

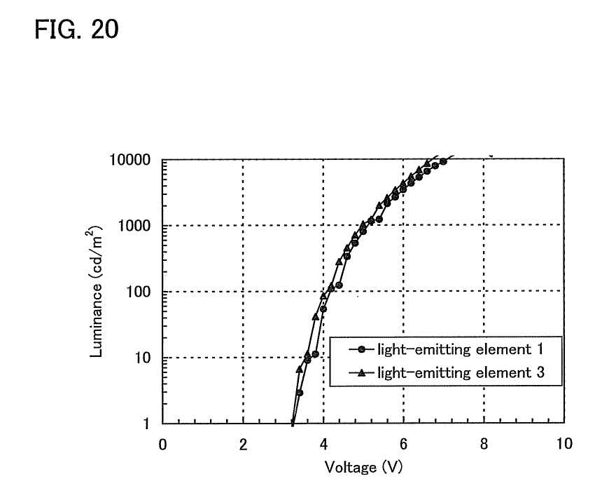

FIG. 17 illustrates the luminance-current efficiency characteristics of the light-emitting element 1-1 and the light-emitting element 1-3, FIG. 19 illustrates the current density-luminance characteristics thereof, and FIG. 20 illustrates the voltage-luminance characteristics thereof. In addition, FIG. 18 illustrates the emission spectrum at a current of 1 mA.

According to FIG. 18, as for the light-emitting element 1-1, favorable blue-light emission having a peak at 465 nm was obtained from PCBAPA. The light-emitting element 1-1 exhibited favorable blue-light emission where the CIE chromaticity coordinates were x=0.16 and y=0.17 when the luminance was 1160 cd/m². In addition, when the luminance was 1160 cd/m², the current efficiency was 4.7 cd/A, the external quantum efficiency was 3.5%, the voltage was 5.2 V, the current density was 24.8 mA/cm², and the power efficiency was 2.8 lm/W.

According to FIG. 18, as for the light-emitting element 1-3, favorable blue-light emission having a peak at 465 nm was obtained from PCBAPA. The light-emitting element 1-1 exhibited favorable blue-light emission where the CIE chromaticity coordinates were x=0.16 and y=0.18 when the luminance was 1030 cd/m². In addition, when the luminance was 1030 cd/m², the current efficiency was 4.6 cd/A, the external quantum efficiency was 3.3%, the voltage was 5.0 V, the current density was 22.3 mA/cm², and the power efficiency was 2.9 lm/W.

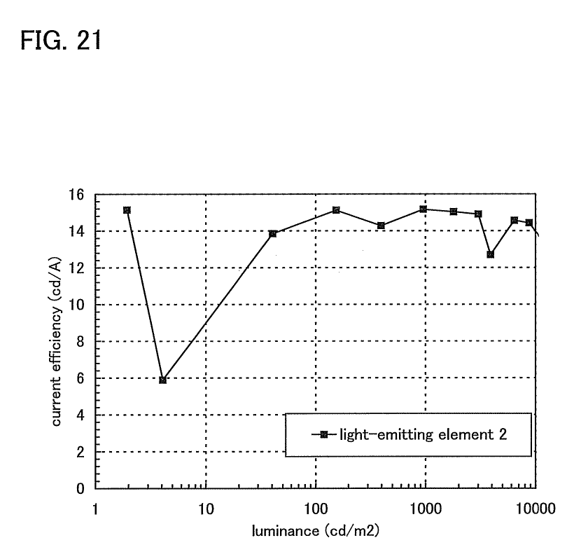

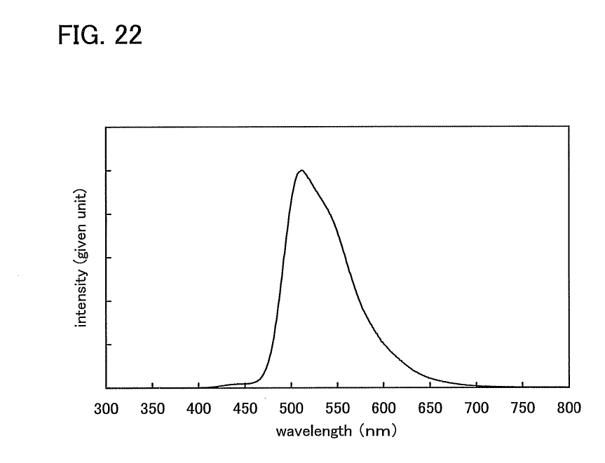

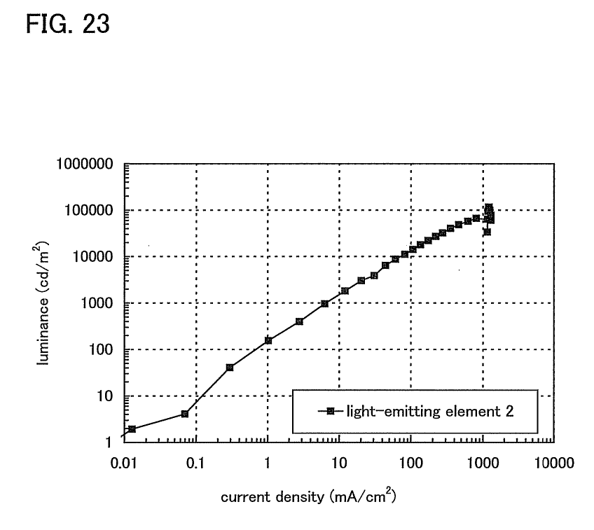

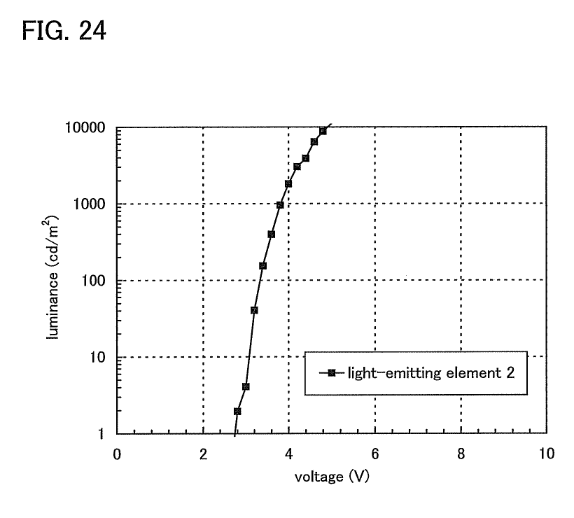

FIG. 21 illustrates the luminance-current efficiency characteristics of the light-emitting element 1-2, FIG. 23 illustrates the current density-luminance characteristics thereof, and FIG. 24 illustrates the voltage-luminance characteristics thereof. In addition, FIG. 22 illustrates the emission spectrum which was obtained at a current of 1 mA. According to FIG. 22, as for the light-emitting element 1-2, favorable green-light emission having a peak at 515 nm was obtained from 2PCAPA. The light-emitting element 1-2 exhibited favorable green-light emission where the CIE chromaticity coordinates were x=0.28 and y=0.60 when the luminance was 960 cd/m². In addition, when the luminance was 960 cd/m², the current efficiency was 15.2 cd/A, the external quantum efficiency was 4.6%, the voltage was 3.8 V, the current density was 6.31 mA/cm², and the power efficiency was 12.5 lm/W.

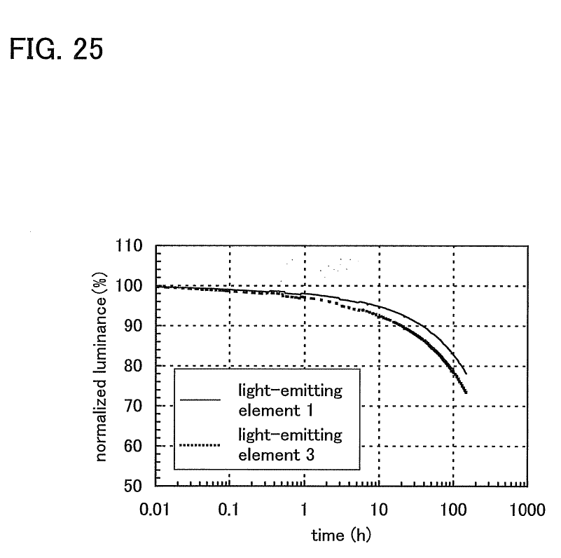

Further, reliability tests of the manufactured light-emitting element 1-1 and light-emitting element 1-3 were performed. The reliability tests were performed as follows. The current with which the light-emitting element 1 in an initial state emitted light at a luminance of 1000 cd/m² was kept constantly applied and luminance was measured at certain time intervals. Results obtained by the reliability tests of the light-emitting element 1-1 and the light-emitting element 1-3 are illustrated in FIG. 25. FIG. 25 illustrates a change in luminance over time. Note that in FIG. 25, the horizontal axis represents current flow time (hour) and the vertical axis represents the proportion of luminance with respect to the initial luminance at each time, that is, normalized luminance (%).

As described above, the highly reliable light-emitting elements 1-1 to 1-3 were obtained in this example.

According to this example, it was confirmed that the light-emitting element of the present invention has characteristics as a light-emitting element and sufficiently functions. Further, from the results of the reliability tests, a highly reliable light-emitting element was obtained in which a short circuit due to defects of the film or the like is not caused even if the light-emitting element is continuously made to emit light.

### Example 4

### Synthesis Example 1

In this synthesis example, a synthesis method of 3-(biphenyl-4-yl)-9-[4-(10-phenyl-9-anthryl)phenyl]-9H-carbazole (CzPApB) represented by the following structural formula (31) will be described.

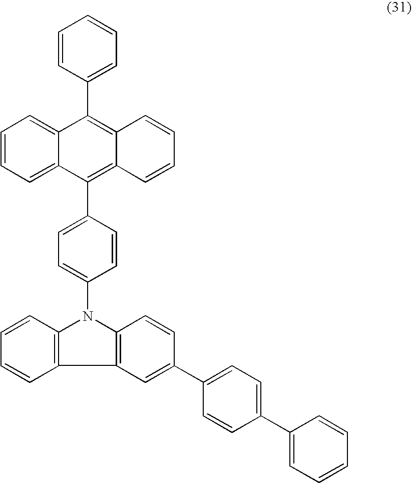

### Step 1

### Synthesis of 3-(biphenyl-4-yl)-9H-carbazole

A synthetic scheme of 3-(biphenyl-4-yl)-9H-carbazole is shown in (D-1).

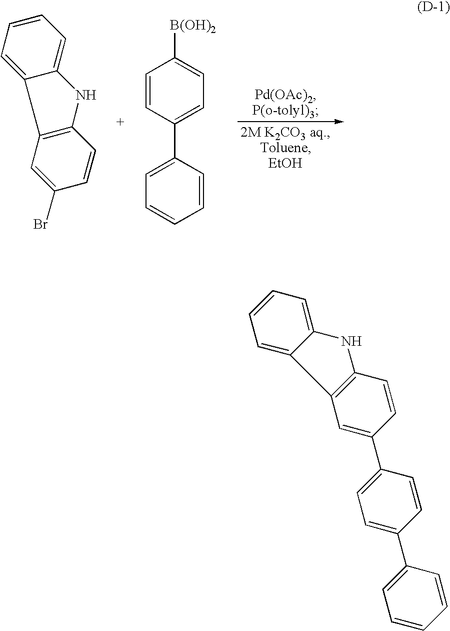

Into a 100 mL three-neck flask were put 0.50 g (2.0 mmol) of 3-bromo-9H-carbazole, 0.40 g (2.0 mmol) of 4-biphenylboronic acid, and 0.15 g (0.50 mmol) of tri(ortho-tolyl)phosphine, and the air in the flask was replaced with nitrogen. To this mixture were added 30 mL of toluene, 10 mL of ethanol, and 2.0 mL of a potassium carbonate aqueous solution (2.0 mol/L). This mixture was stirred to be degassed while the pressure was reduced. To the mixture was added 23 mg (0.10 mmol) of palladium(II) acetate, and the mixture was stirred at 80° C. under a nitrogen stream for 2 hours. After the stir, the aqueous layer of the mixture was extracted with toluene and the extracted solution and the organic layer were washed together with saturated saline. The organic layer was dried with magnesium sulfate, and this mixture was subjected to gravity filtration. The obtained filtrate was concentrated to give a solid. The obtained solid was dissolved in about 10 mL of toluene. The solution was subjected to suction filtration through Celite (Catalog No. 531-16855, manufactured by Wako Pure Chemical Industries, Ltd.), alumina, and Florisil (Catalog No. 540-00135, manufactured by Wako Pure Chemical Industries, Ltd.). The obtained filtrate was concentrated to give a white solid. This solid was washed with hexane to give 0.20 g of white powder of 3-(biphenyl-4-yl)-9H-carbazole, which was the object, at a yield of 31%.

### Step 2

### Synthesis of CzPApB

A synthetic scheme of CzPApB is shown in (D-2).

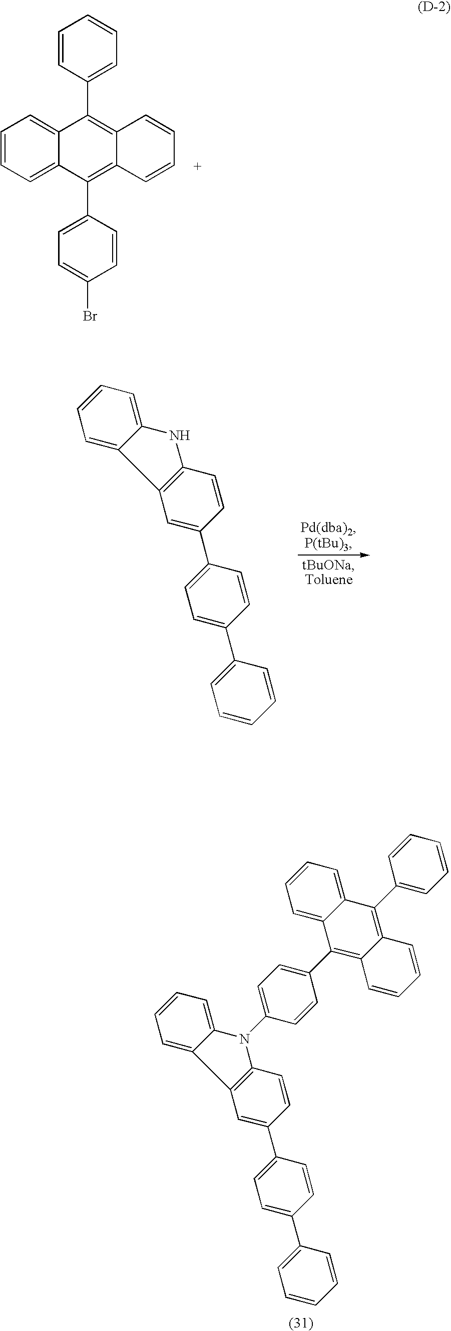

Into a 100 mL three-neck flask were put 0.24 g (0.59 mmol) of 9-(4-bromophenyl)-10-phenylanthracene, 0.19 g (0.59 mmol) of 3-(biphenyl-4-yl)-9H-carbazole, and 0.11 g (1.2 mmol) of sodium tert-butoxide. The air in the flask was replaced with nitrogen. Then, to the mixture were added 20 mL of toluene and 0.20 mL of tri(tert-butyl)phosphine (a 10 wt % hexane solution). The mixture was stirred to be degassed while the pressure was reduced. After the degassing, 32 mg (0.055 mmol) of bis(dibenzylideneacetone)palladium(0) was added to the mixture. This mixture was stirred under a nitrogen stream at 110° C. for 2 hours. After the stir, the mixture was subjected to suction filtration through Celite (Catalog No. 531-16855, manufactured by Wako Pure Chemical Industries, Ltd.), alumina, and Florisil (Catalog No. 540-00135, manufactured by Wako Pure Chemical Industries, Ltd.) to obtain a filtrate. The obtained filtrate was concentrated to give a solid. The obtained solid was purified by silica gel column chromatography (a developing solvent was a mixed solvent of hexane and toluene (hexane:toluene=5:1)) to give a light yellow solid. The obtained light yellow solid was recrystallized with a mixed solvent of toluene and hexane to give 0.29 g of light yellow powder, which was the object, at a yield of 76%.

Sublimation purification by train sublimation was performed on 0.29 g of the obtained light yellow powder. The sublimation purification was performed under such conditions that the light yellow powder was heated at 320° C. with an argon gas applied at a flow rate of 4.0 mL/min under reduced pressure. After the sublimation purification, 0.27 g of a light yellow solid, which was the object, was recovered, at a yield of 93%.

The compound was identified as 3-(biphenyl-4-yl)-9-[4-(10-phenyl-9-anthryl)phenyl]-9H-carbazole (CzPApB) by nuclear magnetic resonance (NMR). ¹H NMR data of the obtained compound is shown below. ¹H NMR (CDCl₃, 300 MHz): δ=7.35-7.80 (m, 25H), 7.82-7.88 (m, 6H), 8.27 (d, J=7.8 Hz, 1H), 8.47 (d, J=1.5 Hz, 1H).

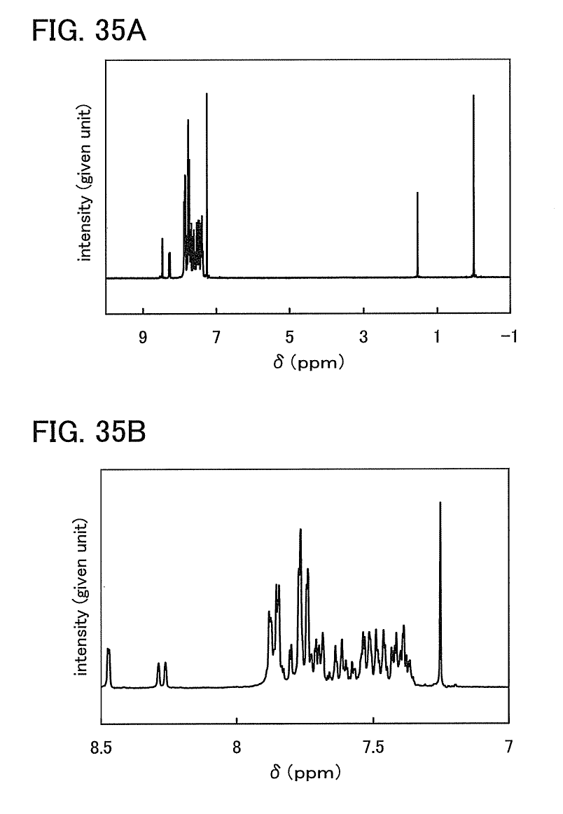

Further, the ¹H NMR chart is illustrated in FIGS. 35A and 35B. Note that FIG. 35B is a chart showing an enlarged portion of FIG. 35A in the range of from 7.0 ppm to 8.5 ppm.

The thermogravimetry-differential thermal analysis (TG-DTA) was performed on the obtained CzPApB. According to the measurement with a thermo-gravimetric/differential thermal analyzer (TG/DTA 320, manufactured by Seiko Instrument Inc.), 5% weight loss temperature was 460° C. Accordingly, CzPApB was found to be a material having favorable heat resistance.

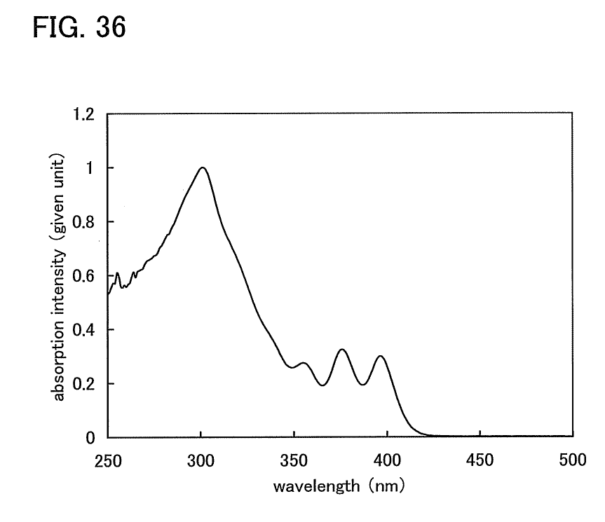

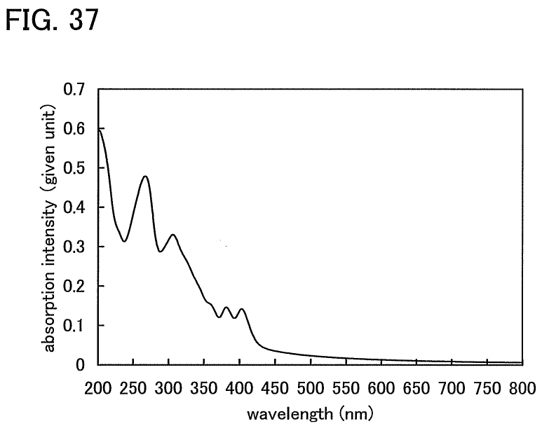

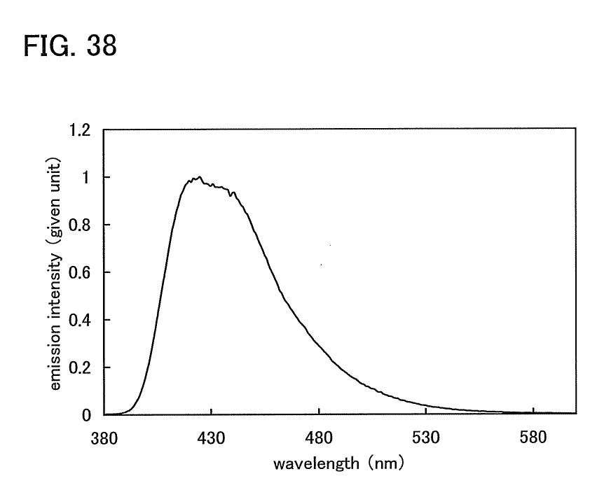

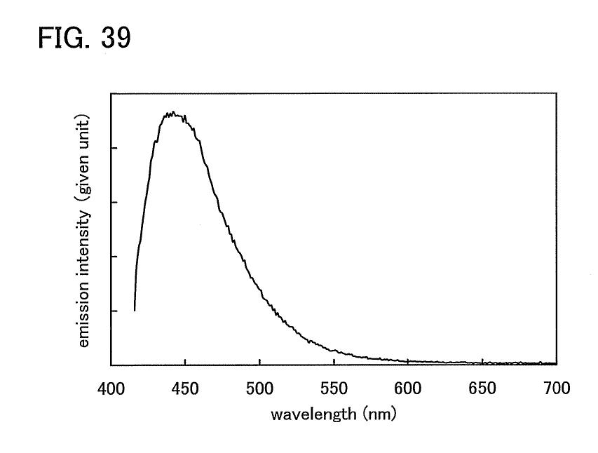

FIG. 36 illustrates an absorption spectrum of CzPApB included in a toluene solution. FIG. 37 illustrates an absorption spectrum of a thin film of CzPApB. An ultraviolet-visible spectrophotometer (V-550, manufactured by JASCO Corporation) was used for the measurement. The solution was put in a quartz cell and the thin film was formed by evaporation onto a quartz substrate to manufacture a sample. As for the spectrum of the solution, the absorption spectrum obtained by subtraction of the absorption spectrum of the quartz cell including only toluene is illustrated in FIG. 36. As for the spectrum of the thin film, the absorption spectrum obtained by subtraction of the absorption spectrum of the quartz substrate is illustrated in FIG. 37. In FIG. 36 and FIG. 37, the horizontal axis represents wavelength (nm) and the vertical axis represents absorption intensity (given unit). In the case of the toluene solution, absorption was observed at around 301 nm, 355 nm, 376 nm, and 396 nm. In the case of the thin film, absorption was observed at around 267 nm, 306 nm, 361 nm, 382 nm, and 403 nm. The emission spectrum of the toluene solution of CzPApB (excitation wavelength: 376 nm) is illustrated in FIG. 38. The emission spectrum of the thin film of CzPApB (excitation wavelength: 401 nm) is illustrated in FIG. 39. In FIG. 38 and FIG. 39, the horizontal axis represents wavelength (nm) and the vertical axis represents emission intensity (given unit). It was found that in the case of the toluene solution, the maximum emission wavelength was 421 nm (excitation wavelength: 376 nm), and in the case of the thin film, the maximum emission wavelength was 442 nm (excitation wavelength: 401 nm), and blue-light emission was obtained.

Further, the HOMO level and LUMO level of CzPApB in the thin film state were measured. The HOMO level was obtained by conversion of a value of ionization potential measured with a photoelectron spectrometer (AC-2, manufactured by Riken Keiki Co., Ltd.) in the atmosphere into a negative value. The LUMO level was obtained in such a manner that the absorption edge was obtained from Tauc plot, with an assumption of direct transition, using data on the absorption spectrum of the thin film of CzPApB in FIG. 37, and the obtained absorption edge was added to the HOMO level as an optical energy gap. As a result, the HOMO level and LUMO level of CzPApB were found to be −5.78 eV and −2.84 eV, respectively, and the band gap was found to be 2.94 eV.

### Synthesis Example 2

In this synthesis example, another synthesis method of 3-(biphenyl-4-yl)-9-[4-(10-phenyl-9-anthryl)phenyl]-9H-carbazole (CzPApB) represented by the following structural formula (31) will be described.

### Step 1

### Synthesis of 3-bromo-9-[4-(10-phenyl-9-anthryl)phenyl]-9H-carbazole

A synthetic scheme is shown in the following (F-1).

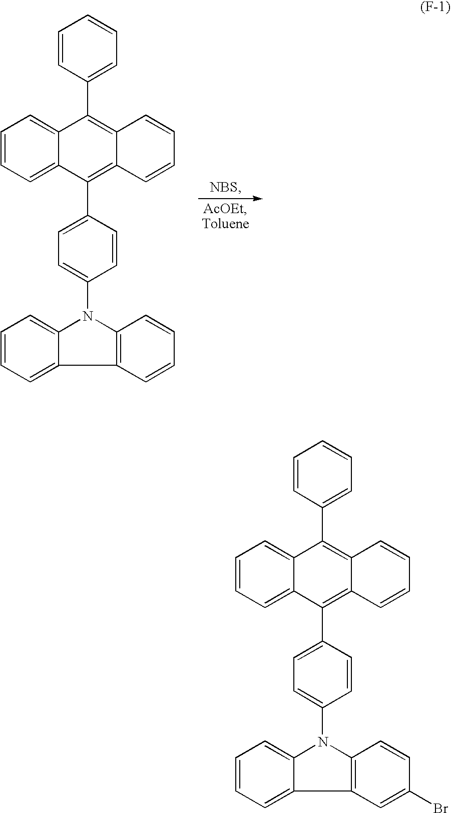

Into a 1 L Erlenmeyer flask were put 5.0 g (10 mmol) of 9-[4-(10-phenyl-9-anthryl)phenyl]-9H-carbazole(CzPA), 600 mL of ethyl acetate, and 150 mL of toluene. This mixture was stirred while being heated at about 50° C. or higher, and dissolution of CzPA was confirmed. To this solution was added 1.8 g (10 mmol) of N-bromo succinimide (NBS). This solution was stirred at a room temperature under the atmosphere for 5 days. After the stir, about 150 mL of a sodium thiosulfate aqueous solution was added to this solution and the solution was stirred for 1 hour. After the organic layer of this mixture was washed with water, the aqueous layer was extracted with toluene and the extracted solution and the organic layer were washed together with saturated saline. The organic layer was dried with magnesium sulfate, and this mixture was subjected to gravity filtration. The obtained filtrate was concentrated to give a light yellow solid. The obtained solid was recrystallized with a mixed solvent of toluene and hexane to give 5.2 g of light yellow powder, which was the object, at a yield of 90%.

### Step 2

### Synthesis of 3-(biphenyl-4-yl)-9-[4-(10-phenyl-9-anthryl)phenyl]-9H-carbazole (CzPApB)

A synthetic scheme is shown in the following (F-2).

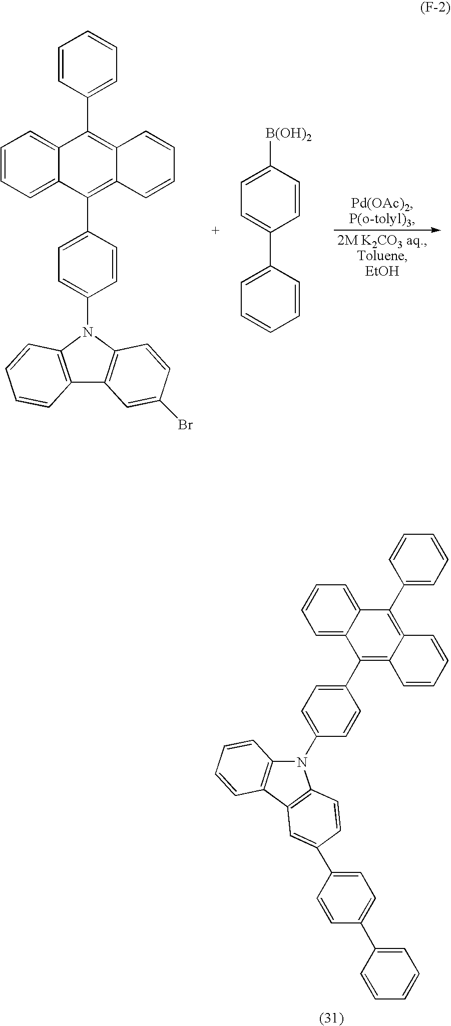

Into a 300 mL three-neck flask were put 3.0 g (5.2 mmol) of 3-bromo-9-[4-(10-phenyl-9-anthryl)phenyl]-9H-carbazole, 1.0 g (5.2 mmol) of 4-biphenylboronic acid, and 0.40 g (1.3 mmol) of tri(ortho-tolyl)phosphine, and the air in the flask was replaced with nitrogen. To this mixture were added 60 mL of toluene, 20 mL of ethanol, and 5.0 mL of a potassium carbonate aqueous solution (2.0 mol/L). This mixture was stirred to be degassed while the pressure was reduced. To the mixture was added 58 mg (0.26 mmol) of palladium(II) acetate, and the mixture was stirred at 80° C. under a nitrogen stream for 3 hours, whereby a light black solid was precipitated. This mixture was cooled down to a room temperature, and then the precipitated solid was subjected to suction filtration to be collected. The collected solid was dissolved in about 100 mL of toluene. The solution was subjected to suction filtration through Celite (Catalog No. 531-16855, manufactured by Wako Pure Chemical Industries, Ltd.), alumina, and Florisil (Catalog No. 540-00135, manufactured by Wako Pure Chemical Industries, Ltd.). The obtained filtrate was concentrated to give a light yellow powdered solid. The obtained solid was recrystallized with toluene to give 2.0 g of a light yellow powdered solid at a yield of 59%. Sublimation purification by train sublimation was performed on 1.8 g of the obtained light yellow powdered solid. The sublimation purification was performed under such conditions that the light yellow powder was heated at 320° C. with an argon gas applied at a flow rate of 4.0 mL/min. After the sublimation purification, 1.5 g of a light yellow solid, which was the object, was obtained at a yield of 84%.

As in Synthesis Example 1, this compound was identified as 3-(biphenyl-4-yl)-9-[4-(10-phenyl-9-anthryl)phenyl]-9H-carbazole (CzPApB) which was the object by nuclear magnetic resonance (NMR).

### Example 5

In this example, a synthesis method of 3-[4-(1-naphthyl)phenyl]-9-[4-(10-phenyl-9-anthryl)phenyl]-9H-carbazole (CzPAαNP) represented by the following structural formula (63) will be described.

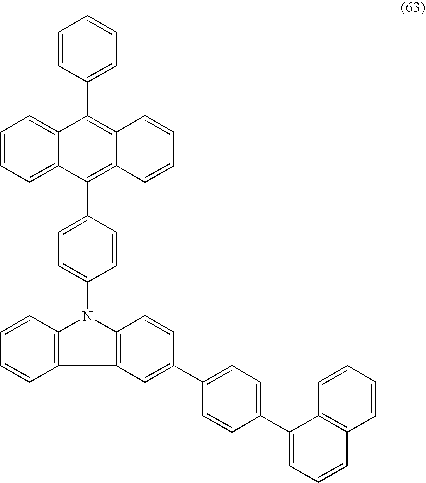

A synthetic scheme is shown in the following (G-1).

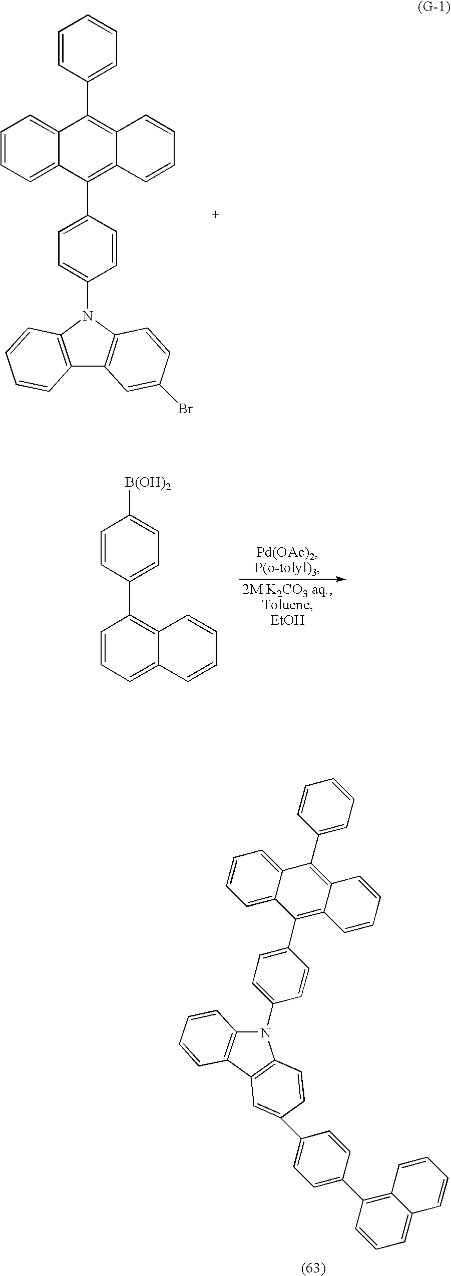

Into a 200 mL three-neck flask were put 2.5 g (4.4 mmol) of 3-bromo-9-[4-(10-phenyl-9-anthryl)phenyl]-9H-carbazole, 1.1 g (4.4 mmol) of 4-(1-naphthyl)phenylboronic acid, and 0.33 g (1.1 mmol) of tri(ortho-tolyl)phosphine, and the air in the flask was replaced with nitrogen. To this mixture were added 5.0 mL (2.0 mol/L) of a potassium carbonate aqueous solution, 60 mL of toluene, and 20 mL of ethanol. This mixture was stirred to be degassed while the pressure was reduced. To the mixture was added 49 mg (0.22 mmol) of palladium(II) acetate, and the mixture was stirred at 80° C. under a nitrogen stream for 5 hours. After the stir, the aqueous layer of the mixture was extracted with toluene and the extracted solution and the organic layer were washed together with saturated saline. After that, the organic layer was dried with magnesium sulfate, and this mixture was subjected to gravity filtration. The obtained filtrate was concentrated to give an oily substance. The obtained oily substance was dissolved in about 10 mL of toluene. This solution was subjected to suction filtration through Celite (Catalog No. 531-16855, manufactured by Wako Pure Chemical Industries, Ltd.), alumina, and Florisil (Catalog No. 540-00135, manufactured by Wako Pure Chemical Industries, Ltd.). The obtained filtrate was concentrated to give an oily substance. The obtained oily substance was purified by silica gel column chromatography (a developing solvent was a mixed solvent of hexane and toluene (hexane:toluene=5:1)) to give a light yellow oily substance. The oily substance was recrystallized with a mixed solvent of toluene and hexane to give 2.4 g of light yellow powder, which was the object, at a yield of 79%

Sublimation purification by train sublimation was performed on 2.3 g of the obtained light yellow powder. The sublimation purification was performed under such conditions that the light yellow powder was heated at 340° C. with an argon gas applied at a flow rate of 4.0 mL/min under reduced pressure. After the sublimation purification, 2.2 g of a light yellow solid, which was the objective compound, was obtained at a yield of 95%.

This compound was identified as 3-[4-(1-naphthyl)phenyl]-9-[4-(10-phenyl-9-anthryl)phenyl]-9H-carbazole (CzPAαNP) which was the object by nuclear magnetic resonance (NMR). ¹H NMR data of the obtained compound is shown below. ¹H NMR (CDCl₃, 300 MHz): δ=7.37-7.67 (m, 17H), 7.70-7.80 (m, 6H), 7.85-7.96 (m, 9H), 8.06 (d, J=8.1 Hz, 1H), 8.29 (d, J=7.8 Hz, 1H), 8.52 (d, J=0.90 Hz, 1H)

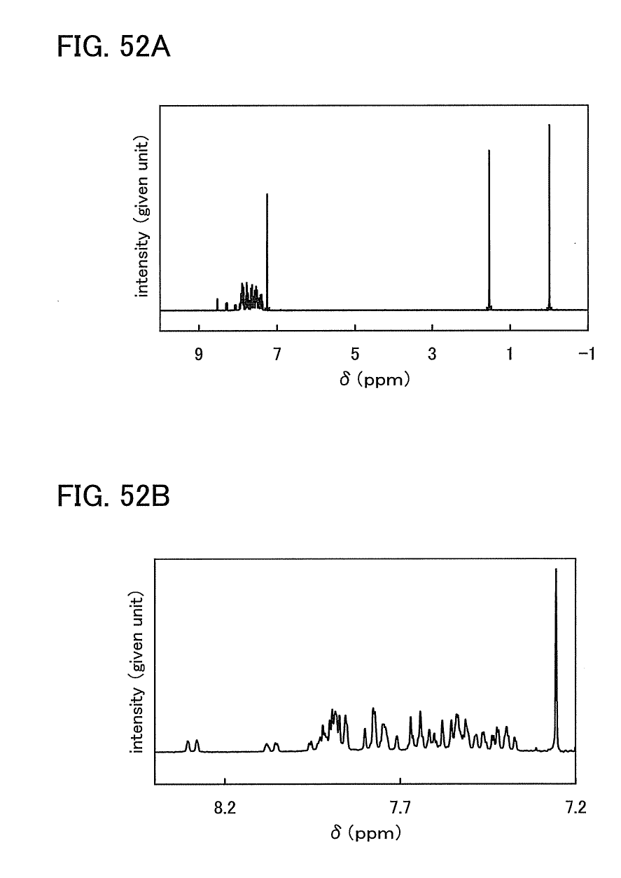

Further, the ¹H NMR chart is illustrated in FIGS. 52A and 52B. Note that FIG. 52B is a chart showing an enlarged portion of FIG. 52A in the range of from 7.2 ppm to 8.4 ppm.

The thermogravimetry-differential thermal analysis (TG-DTA) was performed on the obtained CzPAαNP. The measurement was performed with use of a high vacuum differential type differential thermal balance (TG-DTA2410SA, manufactured by Bruker AXS K.K.). According to the measurement, 5% weight loss temperature was 496° C. Accordingly, CzPAαNP was found to be a material having very favorable heat resistance.

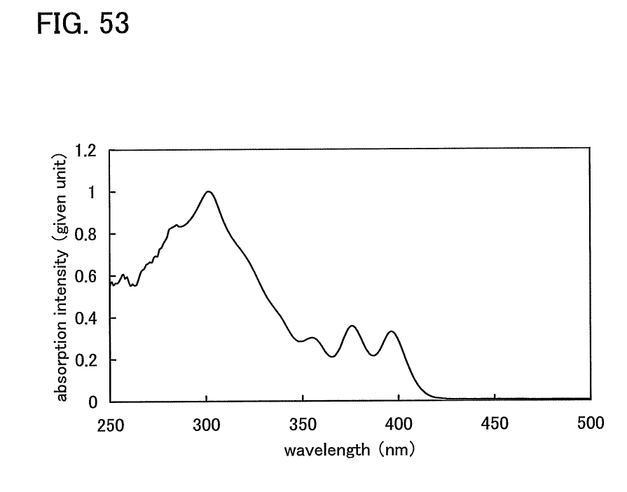

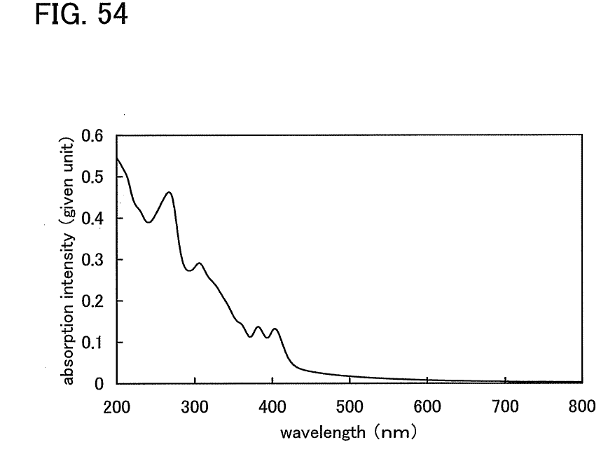

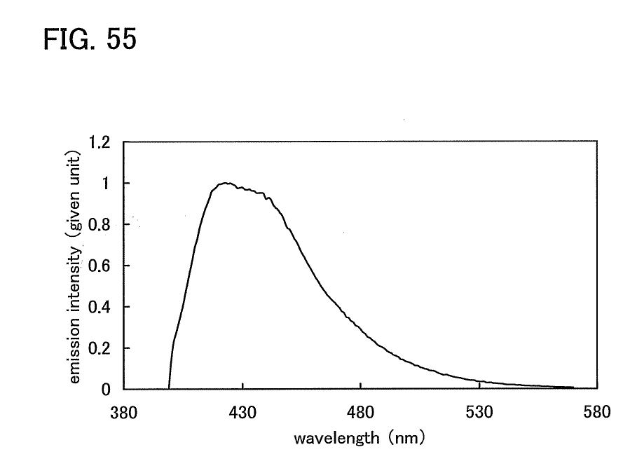

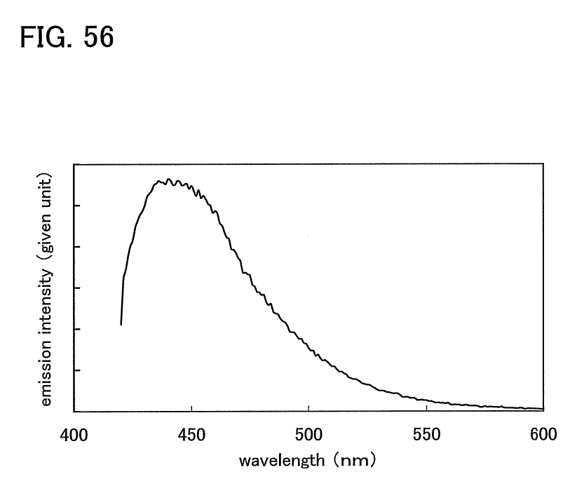

FIG. 53 illustrates an absorption spectrum of CzPAαNP included in a toluene solution. FIG. 54 illustrates an absorption spectrum of a thin film of CzPAαNP. An ultraviolet-visible spectrophotometer (V-550, manufactured by JASCO Corporation) was used for the measurement. The solution was put in a quartz cell and the thin film was formed by evaporation onto a quartz substrate to manufacture a sample. As for the spectrum of the solution, the absorption spectrum obtained by subtraction of the absorption spectrum of the quartz cell including only toluene is illustrated in FIG. 53. As for the spectrum of the thin film, the absorption spectrum obtained by subtraction of the absorption spectrum of the quartz substrate is illustrated in FIG. 54. In FIG. 53 and FIG. 54, the horizontal axis represents wavelength (nm) and the vertical axis represents absorption intensity (given unit). In the case of the toluene solution, absorption was observed at around 302 nm, 355 nm, 376 nm, and 396 nm. In the case of the thin film, absorption was observed at around 267 nm, 306 nm, 358 nm, 382 nm, and 403 nm. The emission spectrum of the toluene solution of CzPAαNP (excitation wavelength: 376 nm) is illustrated in FIG. 55. The emission spectrum of the thin film of CzPAαNP (excitation wavelength: 401 nm) is illustrated in FIG. 56. In FIG. 55 and FIG. 56, the horizontal axis represents wavelength (nm), and the vertical axis represents emission intensity (given unit). In the case of the toluene solution, the maximum emission wavelength was 424 nm (excitation wavelength: 376 nm), and in the case of the thin film, the maximum emission wavelength was 440 nm (excitation wavelength: 401 nm).

The results of measuring the thin film of CzPAαNP by photoelectron spectrometry (AC-2, manufactured by Riken Keiki Co., Ltd.) in the atmosphere indicated that the HOMO level of CzPAαNP was −5.73 eV. Moreover, the absorption edge was obtained from Tauc plot, with an assumption of direct transition, using data on the absorption spectrum of the thin film of CzPAαNP in FIG. 54. When the absorption edge was estimated as an optical energy gap, the energy gap was 2.94 eV. The LUMO level, which was estimated from the HOMO level of the CzPAαNP and the energy gap, was −2.79 eV.

Further, oxidation-reduction reaction characteristics of CzPAαNP were measured. The oxidation-reduction reaction characteristics were measured by cyclic voltammetry (CV) measurement. An electrochemical analyzer (ALS model 600A, manufactured by BAS Inc.) was used for the measurement.

A solution used in the CV measurement was prepared in such a manner that dehydrated dimethylformamide (DMF) (99.8%, catalog number; 22705-6, manufactured by Sigma-Aldrich Co.) was used as a solvent, tetra-n-butylammonium perchlorate (n-Bu₄ NClO₄) (catalog number; T0836, manufactured by Tokyo Kasei Kogyo Co., Ltd.), which was a supporting electrolyte, was dissolved in the solvent so as to have a concentration of 100 mmol/L, and an object to be measured was dissolved so as to have a concentration of 1 mmol/L. Further, a platinum electrode (a PTE platinum electrode, manufactured by BAS Inc.) was used as a working electrode. A platinum electrode (a VC-3 Pt counter electrode (5 cm), manufactured by BAS Inc.) was used as an auxiliary electrode. An Ag/Ag⁺ electrode (an RE5 nonaqueous solvent reference electrode, manufactured by BAS Inc.) was used as a reference electrode. The measurement was performed at a room temperature.

The oxidation reaction characteristics of CzPAαNP were measured as follows. A scan in which the potential of the working electrode with respect to the reference electrode was changed to 1.02 V from 0.30 V and then the potential was changed to 0.30 V from 1.02 V was set as one cycle, and 100 cycle measurements were performed. Note that the scan speed of the CV measurement was set at 0.1 V/s.

The reduction reaction characteristics of CzPAαNP were measured as follows. A scan in which the potential of the working electrode with respect to the reference electrode was changed to −2.44 V from −1.33 V and then the potential was changed to −1.33 V from −2.44 V was set as one cycle, and 100 cycle measurements were performed. Note that the scan speed of the CV measurement was set at 0.1 V/s.

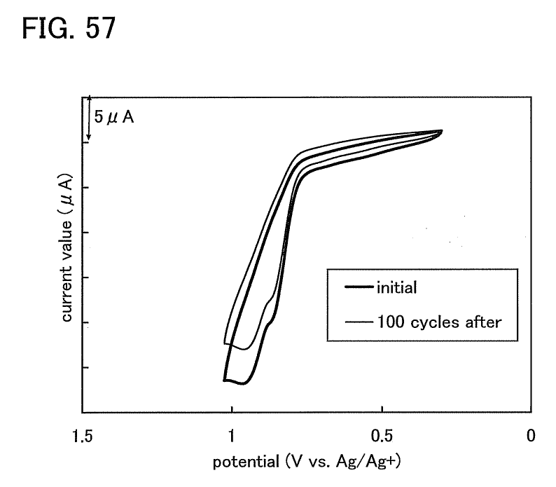

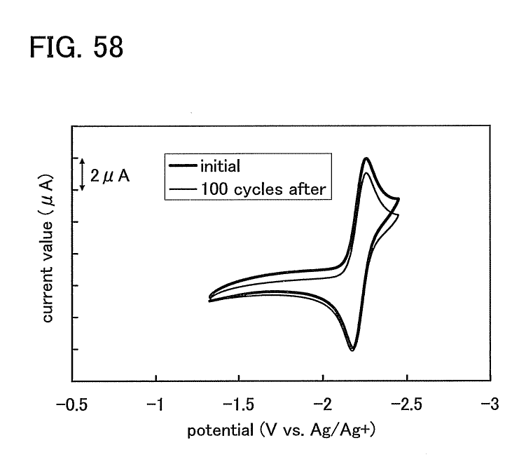

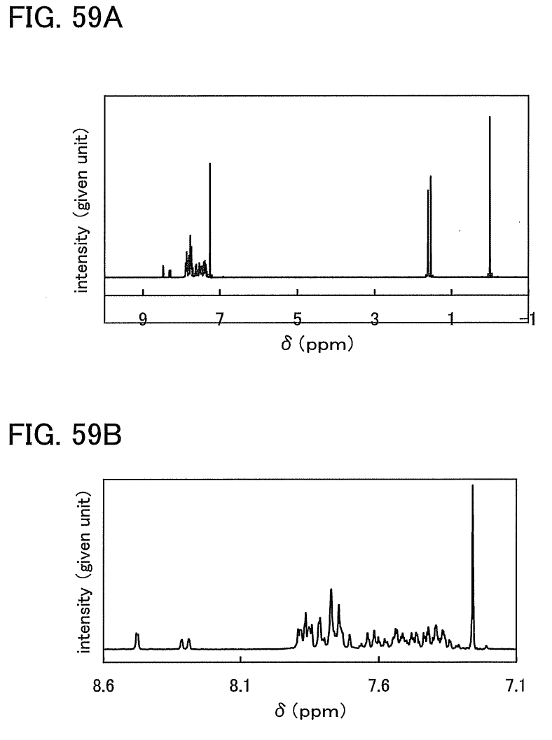

FIG. 57 illustrates CV measurement results on the oxidation reaction characteristic of CzPAαNP and FIG. 58 illustrates CV measurement results on the reduction reaction characteristic of CzPAαNP. In each of FIG. 57 and FIG. 58, the horizontal axis represents potential (V) of the working electrode with respect to the reference electrode and the vertical axis represents current value (μA) that flowed between the working electrode and the auxiliary electrode. According to FIG. 57, a current indicating oxidation was observed at around +0.82 V (vs. Ag/Ag⁺ electrode). According to FIG. 58, a current indicating reduction was observed at around −2.22 V (vs. Ag/Ag⁺ electrode).

In spite of the fact that as many as 100 cycles of scan were performed, a peak position and a peak intensity of the CV curve did not significantly change in the oxidation reaction and the reduction reaction. The peak intensity remained 87% of the initial state of the oxidation reaction and 89% of the initial state of the reduction reaction. Accordingly, the carbazole derivative of the present invention was found stable against repetition of oxidation-reduction reactions.

### Example 6

In this example, a synthesis method of 3-(9,9-dimethylfluoren-2-yl)-9-[4-(10-phenyl-9-anthryl)phenyl]-9H-carbazole (CzPAFL) represented by the following structural formula (76) will be described.

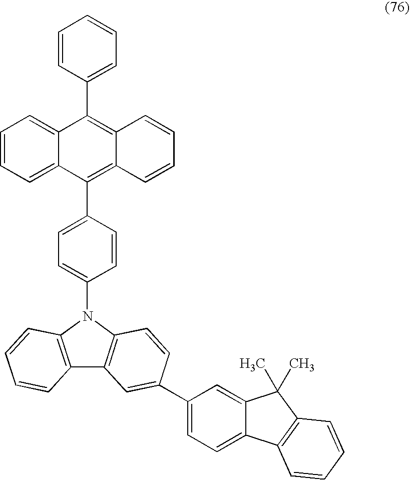

A synthetic scheme is shown in the following (H-1).

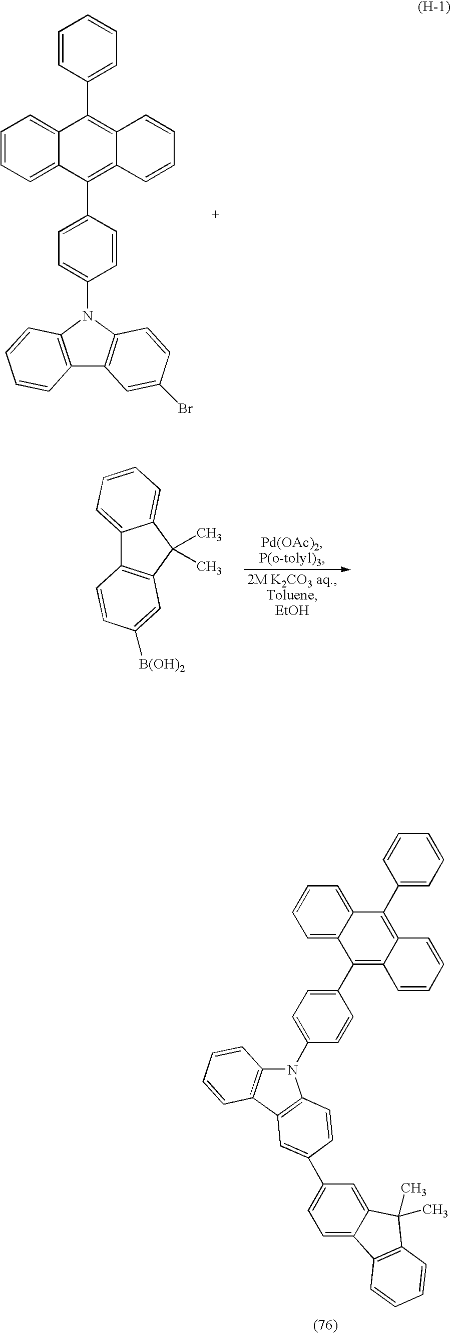

Into a 100 mL three-neck flask were put 0.80 g (1.4 mmol) of 3-bromo-9-[4-(10-phenyl-9-anthryl)phenyl]-9H-carbazole, 0.33 g (1.4 mmol) of 9,9-dimethylfluorene-2-boronic acid, and 0.11 g (0.35 mmol) of tri(ortho-tolyl)phosphine, and the air in the flask was replaced with nitrogen. To this mixture were added 2.0 mL (2.0 mol/L) of a potassium carbonate aqueous solution, 30 mL of toluene, and 10 mL of ethanol. The mixture was stirred to be degassed while the pressure was reduced. To the mixture was added 16 mg (0.070 mmol) of palladium(II) acetate, and the mixture was stirred at 80° C. under a nitrogen stream for 4 hours, whereby a light black solid was precipitated. This mixture was cooled down to a room temperature, and then the precipitated solid was subjected to suction filtration to be collected. The collected solid was dissolved in about 50 mL of toluene and added to the filtrate obtained after the suction filtration. The aqueous layer of the mixture was extracted with toluene and the extracted solution and the organic layer were washed together with saturated saline. The organic layer was dried with magnesium sulfate, and the mixture was subjected to gravity filtration. The obtained filtrate was concentrated to give a solid. The obtained solid was dissolved in about 50 mL of toluene. This solution was subjected to suction filtration through Celite (Catalog No. 531-16855, manufactured by Wako Pure Chemical Industries, Ltd.), alumina, and Florisil (Catalog No. 540-00135, manufactured by Wako Pure Chemical Industries, Ltd.). The obtained filtrate was concentrated to give a solid. The obtained solid was purified by silica gel column chromatography (a developing solvent was a mixed solvent of hexane and toluene (hexane:toluene=5:1)) to give a light yellow solid. The solid was recrystallized with a mixed solvent of toluene and hexane to give 0.57 g of light yellow powder, which was the object, at a yield of 54%.

Sublimation purification by train sublimation was performed on 0.54 g of the obtained light yellow powder. The sublimation purification was performed under such conditions that the yellow powder was heated at 330° C. with an argon gas applied at a flow rate of 4.0 mL/min under reduced pressure. After the sublimation purification, 0.50 g of a light yellow solid, which was the objective compound, was recovered in 93% yield.

This compound was identified as 3-(9,9-dimethylfluoren-2-yl)-9-[4-(10-phenyl-9-anthryl)phenyl]-9H-carbazole (CzPAFL) which was the object by nuclear magnetic resonance (NMR). ¹H NMR data of the obtained compound is shown below. ¹H NMR (CDCl₃, 300 MHz): δ=1.61 (s, 6H), 7.34-7.54 (m, 11H), 7.57-7.66 (m, 3H), 7.70-7.81 (m, 10H), 7.84-7.89 (m, 5H), 8.30 (d, J=7.5 Hz, 1H), 8.47 (s, 1H)

Further, the ¹H NMR chart is illustrated in FIGS. 59A and 59B. Note that FIG. 59B is a chart showing an enlarged portion of FIG. 59A in the range of from 7.1 ppm to 8.6 ppm.

The thermogravimetry-differential thermal analysis (TG-DTA) was performed on the obtained CzPAFL. The measurement was performed with use of a high vacuum differential type differential thermal balance (TG-DTA2410SA, manufactured by Bruker AXS K.K.). According to the measurement, 5% weight loss temperature was 471° C. Accordingly, CzPAFL was found to be a material having very favorable heat resistance.

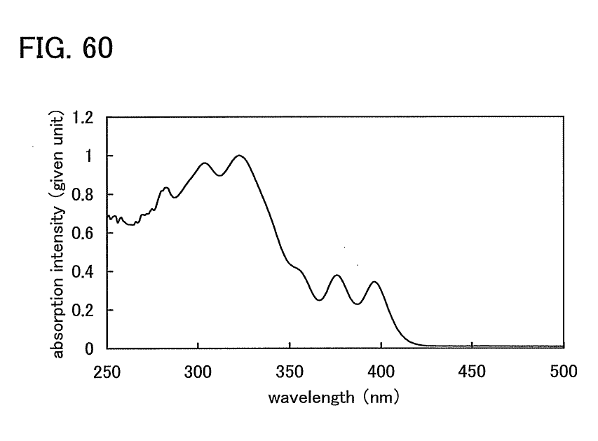

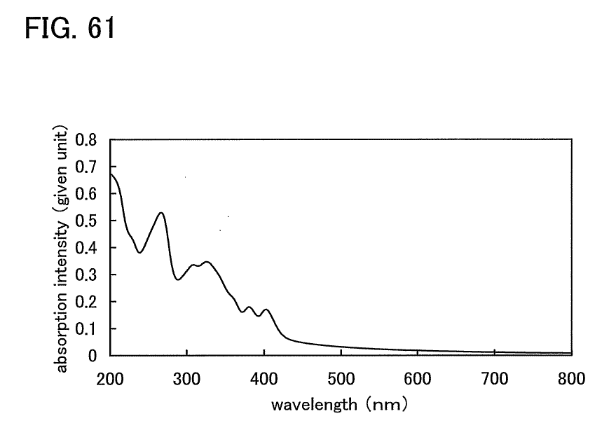

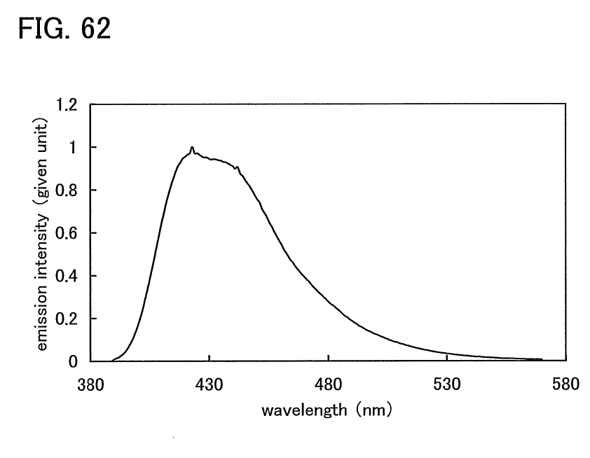

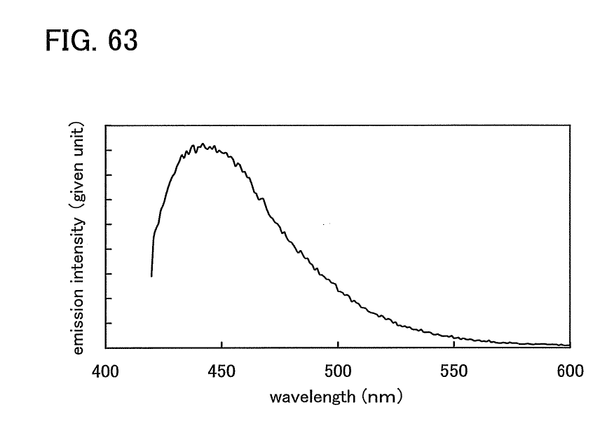

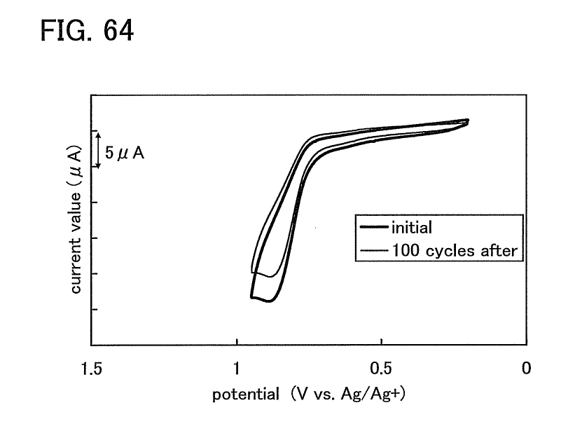

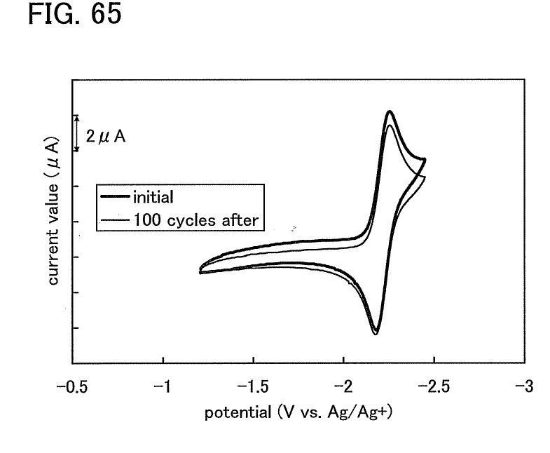

FIG. 60 illustrates an absorption spectrum of CzPAFL included in a toluene solution. FIG. 61 illustrates an absorption spectrum of a thin film of CzPAFL. An ultraviolet-visible spectrophotometer (V-550, manufactured by JASCO Corporation) was used for the measurement. The solution was put in a quartz cell and the thin film was formed by evaporation onto a quartz substrate to manufacture a sample. As for the spectrum of the solution, the absorption spectrum obtained by subtraction of the absorption spectrum of the quartz cell including only toluene is illustrated in FIG. 60. As for the spectrum of the thin film, the absorption spectrum obtained by subtraction of the absorption spectrum of the quartz substrate is illustrated in FIG. 61. In FIG. 60 and FIG. 61, the horizontal axis represents wavelength (nm) and the vertical axis represents absorption intensity (given unit). In the case of the toluene solution, absorption was observed at around 304 nm, 323 nm, 376 nm, and 396 nm. In the case of the thin film, absorption was observed at around 309 nm, 326 nm, 357 nm, 381 nm, and 402 nm. The emission spectrum of the toluene solution of CzPAFL (excitation wavelength: 376 nm) is illustrated in FIG. 62. The emission spectrum of the thin film of CzPAFL (excitation wavelength: 400 nm) is illustrated in FIG. 63. In FIG. 62 and FIG. 63, the horizontal axis represents wavelength (nm) and the vertical axis represents emission intensity (given unit). In the case of the toluene solution, the maximum emission wavelength was 423 nm (excitation wavelength: 376 nm). In the case of the thin film, the maximum emission wavelength was 443 nm (excitation wavelength: 400 nm).

The results of measuring the thin film of CzPAFL by photoelectron spectrometry (AC-2, manufactured by Riken Keiki Co., Ltd.) in the atmosphere indicated that the HOMO level of CzPAFL was −5.62 eV. Moreover, the absorption edge was obtained from Tauc plot, with an assumption of direct transition, using data on the absorption spectrum of the thin film of CzPAFL in FIG. 61. When the absorption edge was estimated as an optical energy gap, the energy gap was 2.93 eV. The LUMO level, which was estimated from the HOMO level (of CzPAFL) and the energy gap, was −2.69 eV.

Further, oxidation-reduction reaction characteristics of CzPAFL were measured. The oxidation-reduction reaction properties were measured by cyclic voltammetry (CV) measurement. An electrochemical analyzer (ALS model 600A, manufactured by BAS Inc.) was used for the measurement.

A solution used in the CV measurement was prepared in such a manner that dehydrated dimethylformamide (DMF) (99.8%, catalog number; 22705-6, manufactured by Sigma-Aldrich Co.) was used as a solvent, tetra-n-butylammonium perchlorate (n-Bu₄ NClO₄) (catalog number; T0836, manufactured by Tokyo Kasei Kogyo Co., Ltd.), which was a supporting electrolyte, was dissolved in the solvent so as to have a concentration of 100 mmol/L, and an object to be measured was dissolved so as to have a concentration of 1 mmol/L. Further, a platinum electrode (a PTE platinum electrode, manufactured by BAS Inc.) was used as a working electrode. A platinum electrode (a VC-3 Pt counter electrode (5 cm), manufactured by BAS Inc.) was used as an auxiliary electrode. An Ag/Ag⁺ electrode (an RE5 nonaqueous solvent reference electrode, manufactured by BAS Inc.) was used as a reference electrode. The measurement was performed at a room temperature.

The oxidation reaction characteristics of CzPAFL were measured as follows. A scan in which the potential of the working electrode with respect to the reference electrode was changed to 0.95 V from 0.20 V and then the potential was changed to 0.20 V from 0.95 V was set as one cycle, and 100 cycle measurements were performed. Note that the scan speed of the CV measurement was set at 0.1 V/s.

The reduction reaction characteristics of CzPAFL were measured as follows. A scan in which the potential of the working electrode with respect to the reference electrode was changed to −2.44 V from −1.21 V and then the potential was changed to −1.21 V from −2.44 V was set as one cycle, and 100 cycle measurements were performed. Note that the scan speed of the CV measurement was set at 0.1 V/s.

FIG. 64 illustrates CV measurement results on the oxidation reaction characteristic of CzPAFL and FIG. 65 illustrates CV measurement results on the reduction reaction characteristic of CzPAFL. In each of FIG. 64 and FIG. 65, the horizontal axis represents potential (V) of the working electrode with respect to the reference electrode and the vertical axis represents current value (μA) that flowed between the working electrode and the auxiliary electrode. According to FIG. 64, a current indicating oxidation was observed at around +0.82 V (vs. Ag/Ag⁺ electrode). According to FIG. 65, a current indicating reduction was observed at around −2.22 V (vs. Ag/Ag⁺ electrode).

In spite of the fact that as many as 100 cycles of scan were performed, the peak position and the peak intensity of the CV curve did not significantly change in the oxidation reaction and the reduction reaction. The peak intensity remained 86% of the initial state of the oxidation reaction and 91% of the initial state of the reduction reaction. Accordingly, the carbazole derivative of the present invention was found stable against repetition of oxidation-reduction reactions.

### Example 7

In this example, a light-emitting element of the present invention will be described with reference to FIG. 26A.

Table 2 shows element structures of a light-emitting element 2-1 and a comparative light-emitting element 2-1 which were manufactured in this example. In Table 2, the mixture ratios are all represented by weight ratios.

TABLE 2

| | first | first | second | third | | | fifth | second |
|---|---|---|---|---|---|---|---|---|
| | electrode | layer | layer | layer | fourth layer | | layer | electrode |
| | 2102 | 2103 | 2104 | 2105 | 2106 | | 2107 | 2108 |
| light | ITSO | NPB:MoOx | NPB | CzPApB:PCBAPA | Alq | Bphen | LiF | Al |
| emitting | 110 nm | (=4:1) | 10 nm | (=1:0.1) | 10 nm | 20 nm | 1 nm | 200 nm |
| element | | 50 nm | | 30 nm | | | | |
| 2-1 | | | | | | | | |
| comparative | ITSO | NPB:MoOx | NPB | CzPAoB:PCBAPA | Alq | Bphen | LiF | Al |
| light | 110 nm | (=4:1) | 10 nm | (=1:0.1) | 10 nm | 20 nm | 1 nm | 200 nm |
| emitting | | 50 nm | | 30 nm | | | | |
| element 2-1 | | | | | | | | |

Hereinafter, manufacturing methods of the light-emitting element 2-1 and the comparative light-emitting element 2-1 of this example will be described.

First, the light-emitting element 2-1 will be described. For the light-emitting element 2-1, indium tin oxide containing silicon oxide (ITSO) was deposited over a glass substrate 2101 by a sputtering method, whereby a first electrode 2102 was formed. The thickness of the first electrode 2102 was 110 nm and the area thereof was 2 mm×2 mm.

Next, the substrate over which the first electrode was formed was fixed to a substrate holder provided in a vacuum evaporation apparatus so that a surface of the substrate on which the first electrode was formed faced downward. The pressure was reduced to be about 10⁻⁴ Pa. Then, 4,4′-bis[N-(1-naphthyl)-N-phenylamino]biphenyl (NPB) and molybdenum(VI) oxide were co-evaporated on the first electrode 2102, whereby a layer 2103 containing a composite material of an organic compound and an inorganic compound was formed as a first layer 2103. The thickness of the first layer 2103 was 50 nm and the weight ratio between NPB and molybdenum(VI) oxide was adjusted to be 4:1 (=NPB:molybdenum oxide). Note that co-evaporation is an evaporation method in which evaporation is performed at the same time from a plurality of evaporation sources in one treatment chamber.

Next, NPB was evaporated to a thickness of 10 nm, whereby a second layer 2104 was formed as a hole-transporting layer.

Next, CzPApB synthesized in Example 4 and 4-(10-phenyl-9-anthryl)-4′-(9-phenyl-9H-carbazol-3-yl)triphenylamine (PCBAPA) were co-evaporated onto the second layer 2104 so that the weight ratio between CzPApB and PCBAPA was 1:0.1 (=CzPApB:PCBAPA), whereby a third layer 2105 was formed as a light-emitting layer. The thickness of the third layer 2105 was 30 nm.

Next, Alq was evaporated onto the third layer 2105 to a thickness of 10 nm, and then Bphen was evaporated to a thickness of 20 nm to form a stacked layer, whereby a fourth layer 2106 was formed as an electron-transporting layer. Further, lithium fluoride (LiF) was evaporated onto the fourth layer 2106 to a thickness of 1 nm, whereby a fifth layer 2107 was formed as an electron-injecting layer. Lastly, aluminum was evaporated to a thickness of 200 nm for a second electrode 2108 which functions as a cathode. Accordingly, the light-emitting element 2-1 of this example was obtained.

Next, the comparative light-emitting element 2-1 will be described. The comparative light-emitting element 2-1 was formed in a manner similar to that of the light-emitting element 2-1 except a third layer 2105. For the comparative light-emitting element 2-1, 3-(biphenyl-2-yl)-9-[4-(10-phenyl-9-anthryl)phenyl]-9H-carbazole (CzPAoB) represented by the following structural formula (400) and PCBAPA were co-evaporated onto the second layer 2104 so that the weight ratio between CzPAoB and PCBAPA was 1:0.1 (=CzPAoB:PCBAPA), whereby the third layer 2105 was formed as a light-emitting layer. The thickness of the third layer 2105 was 30 nm. Accordingly, the comparative light-emitting element 2-1 of this example was obtained.

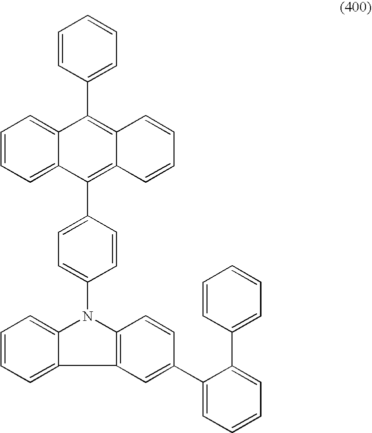

Note that in all of the above evaporation steps, a resistance heating method was used.

The thus obtained light-emitting element 2-1 and comparative light-emitting element 2-1 were sealed in a glove box under a nitrogen atmosphere without being exposed to the atmosphere. After that, operating characteristics of the light-emitting element 2-1 and the comparative light-emitting element 2-1 were measured. The measurement was performed at a room temperature (in the atmosphere in which the temperature was kept at 25° C.).

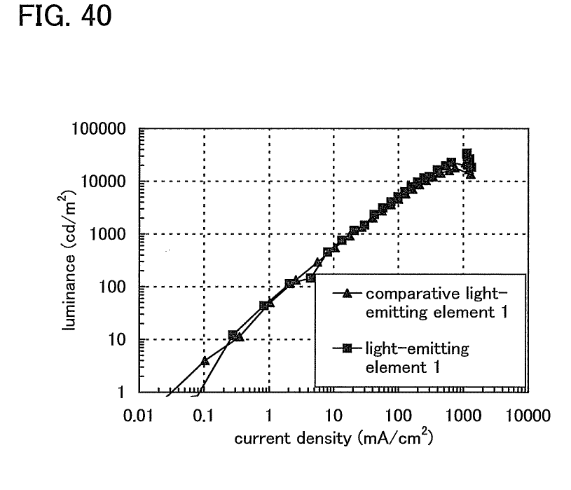

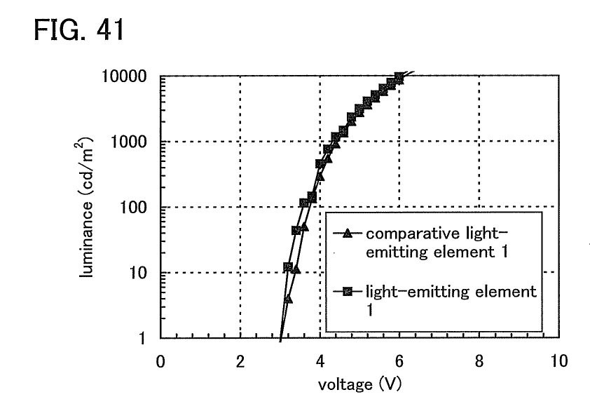

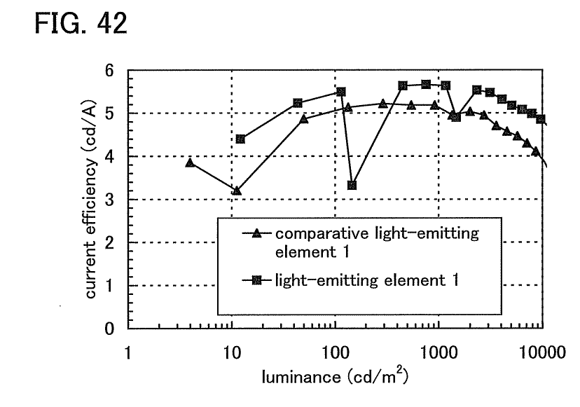

FIG. 40 illustrates the current density-luminance characteristics of the light-emitting element 2-1 and the comparative light-emitting element 2-1. In FIG. 40, the horizontal axis represents current density (mA/cm²) and the vertical axis represents luminance (cd/m²). In addition, FIG. 41 illustrates the voltage-luminance characteristics. In FIG. 41, the horizontal axis represents applied voltage (V) and the vertical axis represents luminance (cd/m²). In addition, FIG. 42 illustrates the luminance-current efficiency characteristics. In FIG. 42, the horizontal axis represents luminance (cd/m²) and the vertical axis represents current efficiency (cd/A). According to FIG. 42, the light-emitting element 2-1 in which the carbazole derivative of the present invention is used has higher current efficiency than the light-emitting element 2-1 in which CzPAoB is used.

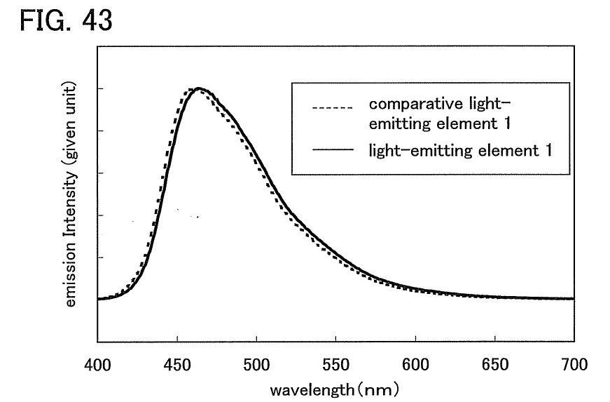

FIG. 43 illustrates emission spectra at a current of 1 mA. According to FIG. 43, light emission derived from a blue light-emitting material PCBAPA was observed both from the manufactured light-emitting element 2-1 and comparative light-emitting element 2-1. The light-emitting element 2-1 exhibited favorable blue-light emission where the CIE chromaticity coordinates were x=0.16 and y=0.21 when the luminance was 1170 cd/m². Further, when the luminance was 1170 cd/cm², the current efficiency was 5.6 cd/A, the external quantum efficiency was 3.6%, the voltage was 4.4 V, the current density was 20.8 mA/cm², and the power efficiency was 4.0 lm/W. The comparative light-emitting element 2-1 exhibited favorable blue-light emission where the CIE chromaticity coordinates were x=0.16 and y=0.20 when the luminance was 920 cd/m². Further, when the luminance was 920 cd/cm², the current efficiency was 5.2 cd/A, the external quantum efficiency was 3.5%, the voltage was 4.4 V, the current density was 17.7 mA/cm², and the power efficiency was 3.7 lm/W.

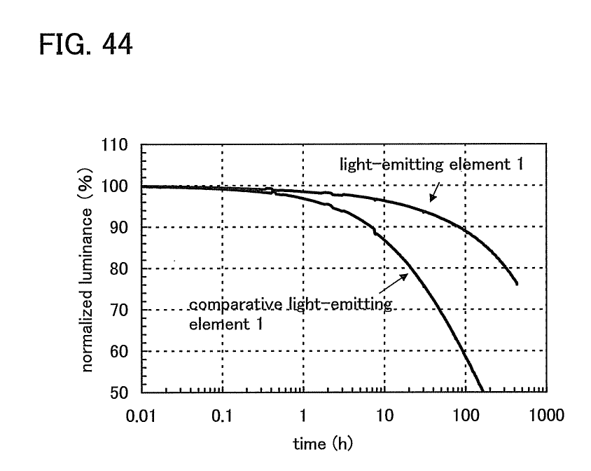

Further, reliability tests of the manufactured light-emitting element 2-1 and comparative light-emitting element 2-1 were performed. The reliability tests were performed as follows. The current with which the light-emitting element 2-1 and comparative light-emitting element 2-1 in an initial state emitted light at a luminance of 1000 cd/m² was kept constantly applied and luminance was measured at certain time intervals. Results obtained by the reliability tests of the light-emitting element 2-1 and the comparative light-emitting element 2-1 are illustrated in FIG. 44. FIG. 44 illustrates a change in luminance over time. Note that in FIG. 44, the horizontal axis represents current flow time (hour) and the vertical axis represents the proportion of luminance with respect to the initial luminance at each time, that is, normalized luminance (%).

According to FIG. 44, decline in luminance over time of the light-emitting element 2-1 is less likely to occur than that of the comparative light-emitting element 2-1 and the light-emitting element 2-1 has long life. Even 430 hours later, the light-emitting element 2-1 kept 76% of the initial luminance and decline in the luminance over time of the light-emitting element 2-1 hardly occurred. Therefore, the light-emitting element 2-1 is a light-emitting element having long life.

This example confirmed that the light-emitting element of the present invention has characteristics as a light-emitting element and fully functions. In addition, it was found that when the carbazole derivative of the present invention was used as a host of a light-emitting layer which emits blue light, a light-emitting element which exhibits favorable blue-light emission was obtained. Further, according to the results of the reliability tests, a highly reliable light-emitting element in which a short circuit due to defects of the film or the like is not caused even if the element is continuously made to emit light.

### Example 8

In this example, a light-emitting element of the present invention will be described with reference to FIG. 26A. In the structure of this example described below, the same reference numerals are commonly given to the same components as in the light-emitting element described in Example 7, which is one mode of the present invention, or components having similar functions to the components of the light-emitting element described in Example 7 and the description of them will not be repeated.

Element structures of a light-emitting element 2-2 and a light-emitting element 2-3 manufactured in this example are shown in Table 3. In Table 3, the mixture ratios are all represented by weight ratios.

TABLE 3

| | first | first | second | | | | fifth | second |
|---|---|---|---|---|---|---|---|---|
| | electrode | layer | layer | third layer | fourth layer | | layer | electrode |
| | 2102 | 2103 | 2104 | 2105 | 2106 | | 2107 | 2108 |
| light | ITSO | NPB:MoOx | NPB | CzPAαNP:PCBAPA | Alq | Bphen | LiF | Al |
| emitting | 110 nm | (=4:1) | 10 nm | (=1:0.1) | 10 nm | 20 nm | 1 nm | 200 nm |
| element | | 50 nm | | 30 nm | | | | |
| 2-2 | | | | | | | | |
| light | ITSO | NPB:MoOx | NPB | CzPAFL:PCBAPA | Alq | Bphen | LiF | Al |
| emitting | 110 nm | (=4:1) | 10 nm | (=1:0.1) | 10 nm | 20 nm | 1 nm | 200 nm |
| element | | 50 nm | | 30 nm | | | | |
| 2-3 | | | | | | | | |

Manufacturing methods of the light-emitting element 2-2 and the light-emitting element 2-3 of this example will be described below. Note that the light-emitting element 2-2 and the light-emitting element 2-3 were manufactured in manners similar to those of the light-emitting element 2-1 and the comparative light-emitting element 2-1 described with reference to FIG. 26A in Example 7, except a third layer 2105.

For the light-emitting element 2-2, CzPAαNP synthesized in Example 5 and 4-(10-phenyl-9-anthryl)-4′-(9-phenyl-9H-carbazol-3-yl)triphenylamine (PCBAPA) were co-evaporated onto the second layer 2104 so that the weight ratio between CzPAαNP and PCBAPA was 1:0.1 (=CzPAαNP:PCBAPA), whereby a third layer 2105 was formed as a light-emitting layer. The thickness of the third layer 2105 was 30 nm. Accordingly, the light-emitting element 2-2 of this example was obtained.

For the light-emitting element 2-3, CzPAFL synthesized in Example 6 and PCBAPA were co-evaporated onto the second layer 2104 so that the weight ratio between CzPAFL and PCBAPA was 1:0.1 (=CzPAFL:PCBAPA), whereby a third layer 2105 was formed as a light-emitting layer. The thickness of the third layer 2105 was 30 nm. Accordingly, the light-emitting element 2-3 of this example was obtained.

Note that in all of the above evaporation steps, a resistance heating method was used.

The thus obtained light-emitting element 2-2 and light-emitting element 2-3 were sealed in a glove box under a nitrogen atmosphere without being exposed to the atmosphere. After that, operating characteristics of the light-emitting element 2-2 and the light-emitting element 2-3 were measured. The measurement was performed at a room temperature (in the atmosphere in which the temperature was kept at 25° C.).

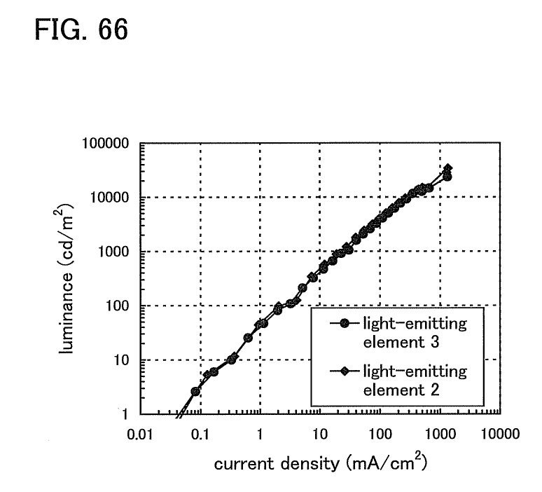

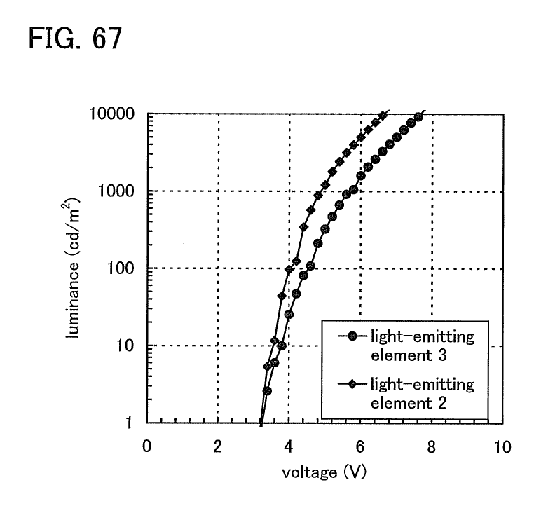

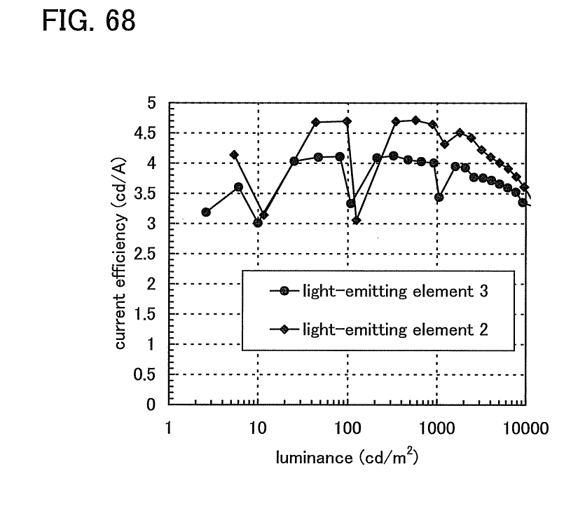

FIG. 66 illustrates the current density-luminance characteristics of the light-emitting element 2-2 and the light-emitting element 2-3. In FIG. 66, the horizontal axis represents current density (mA/cm²) and the vertical axis represents luminance (cd/m²). In addition, FIG. 67 illustrates the voltage-luminance characteristics. In FIG. 67, the horizontal axis represents applied voltage (V) and the vertical axis represents luminance (cd/m²). In addition, FIG. 68 illustrates the luminance-current efficiency characteristics. In FIG. 68, the horizontal axis represents luminance (cd/m²) and the vertical axis represents current efficiency (cd/A).

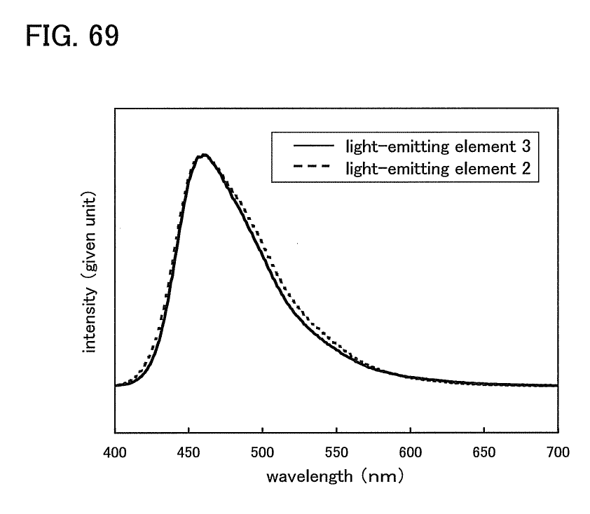

FIG. 69 illustrates emission spectra at a current of 1 mA. According to FIG. 69, light emission derived from a blue light-emitting material PCBAPA was observed both from the manufactured light-emitting element 2-2 and light-emitting element 2-3. The light-emitting element 2-2 exhibited favorable blue-light emission where the CIE chromaticity coordinates were x=0.16 and y=0.19 when the luminance was 880 cd/m². Further, when the luminance was 880 cd/cm², the current efficiency was 4.6 cd/A, the external quantum efficiency was 3.3%, the voltage was 4.8 V, the current density was 19.0 mA/cm², and the power efficiency was 3.0 lm/W. The light-emitting element 2-2 exhibited favorable blue-light emission where the CIE chromaticity coordinates were x=0.16 and y=0.18 when the luminance was 920 cd/m². Further, when the luminance was 920 cd/cm², the current efficiency was 4.0 cd/A, the external quantum efficiency was 2.9%, the voltage was 5.6 V, the current density was 22.8 mA/cm², and the power efficiency was 2.2 lm/W.

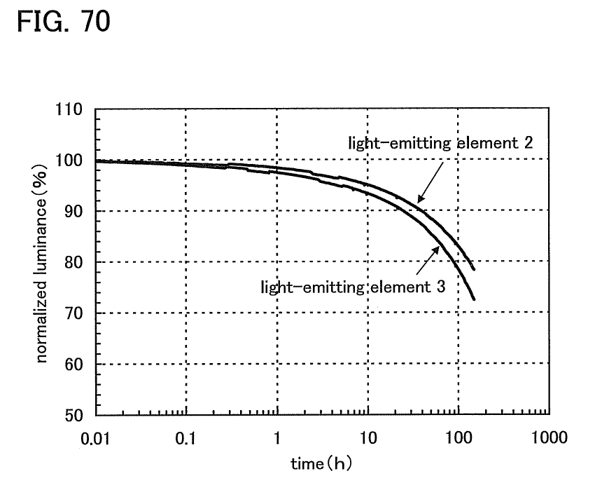

Further, reliability tests of the manufactured light-emitting element 2-2 and light-emitting element 2-3 were performed. The reliability tests were performed as follows. The current with which the light-emitting element 2-2 and light-emitting element 2-3 in an initial state emitted light at a luminance of 1000 cd/m² was kept constantly applied and luminance was measured at certain time intervals. Results obtained by the reliability tests of the light-emitting element 2-2 and the light-emitting element 2-3 are illustrated in FIG. 70. FIG. 70 illustrates a change in luminance over time. Note that in FIG. 70, the horizontal axis represents current flow time (hour) and the vertical axis represents the proportion of luminance with respect to the initial luminance at each time, that is, normalized luminance (%).

As illustrated in FIG. 70, even 150 hours later, the light-emitting element 2-2 kept 78% of the initial luminance, decline in luminance over time of the light-emitting element 2-2 hardly occurred. Therefore, the light-emitting element 2-2 is a light-emitting element having long life. Further, as illustrated in FIG. 70, even 150 hours later, the light-emitting element 2-3 kept 72% of the initial luminance and decline in luminance over time of the light-emitting element 2-3 hardly occurred. Therefore, the light-emitting element 2-3 is a light-emitting element having long life.

This example confirmed that the light-emitting element of the present invention has characteristics as a light-emitting element and fully functions. In addition, it was found that when the carbazole derivative of the present invention was used as a host of a light-emitting layer which emits blue light, a light-emitting element which exhibits favorable blue-light emission was obtained. Further, according to the results of the reliability tests, a highly reliable light-emitting element in which a short circuit due to defects of the film or the like is not caused even if the element is continuously made to emit light.

### Example 9

In this example, a synthesis method of 3-(biphenyl-3-yl)-9-[4-(10-phenyl-9-anthryl)phenyl]-9H-carbazole (CzPAmB) represented by the structural formula (331) will be described.

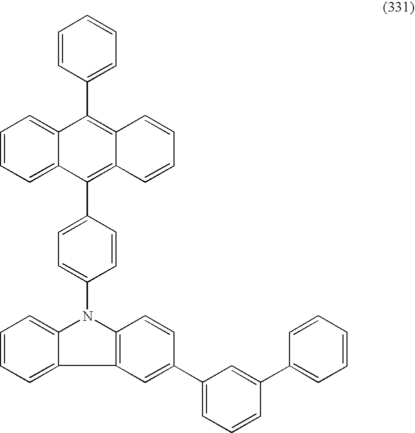

### Step 1

### Synthesis of 3-bromo-9-[4-(10-phenyl-9-anthryl)phenyl]-9H-carbazole

A synthetic scheme is shown in the following (N-1).

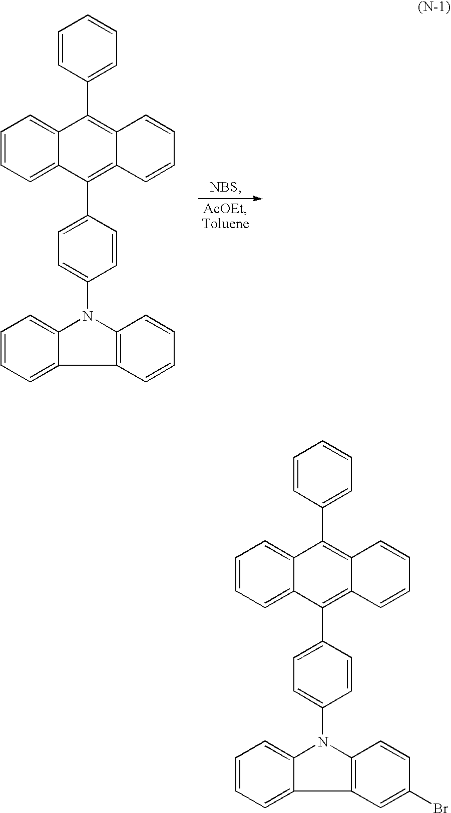

Into a 1 L Erlenmeyer flask were put 5.0 g (10 mmol) of 9-[4-(10-phenyl-9-anthryl)phenyl]-9H-carbazole (CzPA), 600 mL of ethyl acetate, and 150 mL of toluene. This mixture was stirred while being heated at about 50° C., and dissolution of CzPA was dissolved. To this solution was added 1.8 g (10 mmol) of N-bromo succinimide (NBS). This solution was stirred at a room temperature under the atmosphere for 5 days. After the stir, about 150 mL of a sodium thiosulfate aqueous solution was added to this solution and the solution was stirred for 1 hour. After the organic layer of this mixture was washed with water, the aqueous layer was extracted with toluene and the extracted solution and the organic layer were washed together with saturated saline. After the organic layer was dried with magnesium sulfate, this mixture was subjected to gravity filtration. The obtained filtrate was concentrated to give a light yellow solid. The obtained solid was recrystallized with a mixed solvent of toluene and hexane to give 5.2 g of light yellow powder, which was the object, at a yield of 90%.

### Step 2

### Synthesis of 3-biphenylboronic acid

A synthetic scheme is shown in the following (N-2).

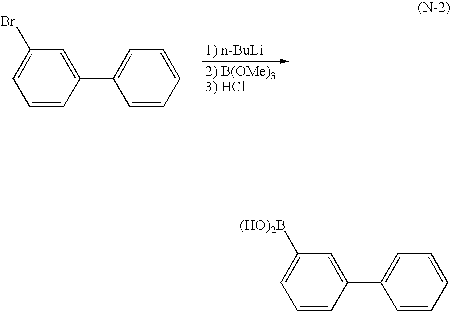

Into a 300 mL three-neck flask was put 3.8 g (16 mmol) of 3-bromobiphenyl was put, and the air in the flask was replaced with nitrogen. To the flask was added 100 mL of tetrahydrofuran (THF) was added, and this solution was cooled down to −80° C. To this solution was added 11 mL (18 mmol) of n-butyllithium (a 1.6 mol/L hexane solution) by being dropped with a syringe. After the dropping was completed, this solution was stirred at the same temperature for 1 hour. After the stir, 2.2 mL (20 mmol) of trimethyl borate was added thereto, and the mixture was stirred for 4 hours while the temperature of the mixture was brought back to a room temperature. After the stir, about 50 mL of dilute hydrochloric acid (1.0 mol/L) was added to the solution, and then the solution was stirred for 2 hours. After stir, the aqueous layer of the mixture was extracted with ethyl acetate and the extracted solution and the organic layer were washed together with a saturated sodium bicarbonate solution and saturated saline. The organic layer was dried with magnesium sulfate, and this mixture was subjected to gravity filtration to obtain a filtrate. The obtained filtrate was concentrated to give an oily substance. Hexane was added to the oily substance, whereby a white solid was precipitated. The obtained solid was collected to give 1.7 g of white powder, which was the object, at a yield of 55%.

### Step 3

### Synthesis of 3-(biphenyl-3-yl)-9-[4-(10-phenyl-9-anthryl)phenyl]-9H-carbazole (CzPAmB)

A synthetic scheme is shown in the following (N-3).

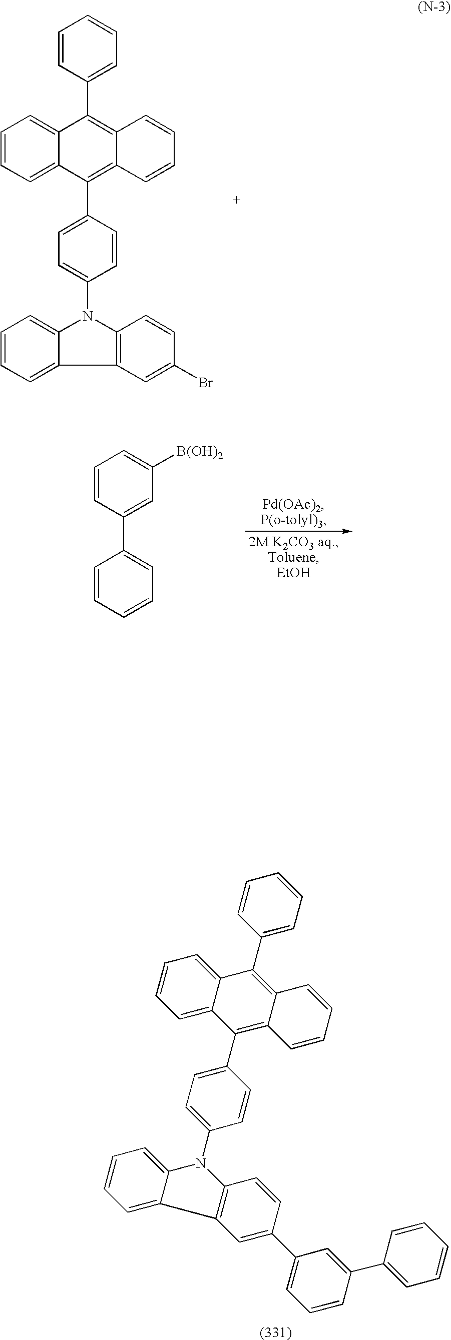

Into a 300 mL three-neck flask were put 2.5 g (4.3 mmol) of 3-bromo-9-[4-(10-phenyl-9-anthryl)phenyl]-9H-carbazole, 0.86 g (4.3 mmol) of 3-biphenylboronic acid, and 0.32 g (1.0 mmol) of tri(ortho-tolyl)phosphine, and the air in the flask was replaced with nitrogen. To the mixture were added 60 mL of toluene, mL of ethanol, and 5.0 mL of a potassium carbonate aqueous solution (2.0 mol/L). This mixture was stirred to be degassed while the pressure was reduced. To the mixture was added 48 mg (0.21 mmol) of palladium(II) acetate, and the mixture was stirred at 80° C. under a nitrogen stream for 3 hours. After the stir, the aqueous layer of the mixture was extracted with toluene and the extracted solution and the organic layer were washed together with saturated saline. The organic layer was dried with magnesium sulfate, and this mixture was subjected to gravity filtration. The obtained filtrate was concentrated to give an oily substance. The oily substance was dissolved in about 10 mL of toluene. The solution was subjected to suction filtration through Celite (Catalog No. 531-16855, manufactured by Wako Pure Chemical Industries, Ltd.), alumina, and Florisil (Catalog No. 540-00135 manufactured by Wako Pure Chemical Industries, Ltd.).

The obtained filtrate was concentrated to give an oily substance. The obtained oily substance was purified by silica gel column chromatography (a developing solvent was a mixed solvent of hexane and toluene (hexane:toluene=5:1)) to give a light yellow solid. The light yellow solid obtained after the purification was recrystallized with a mixed solvent of toluene and hexane to give 2.2 g of light yellow powder, which was the object, at a yield of 80%.

Sublimation purification by train sublimation was performed on 2.2 g of the obtained light yellow powder. The sublimation purification was performed under such conditions that the yellow powder was heated at 330° C. with an argon gas applied at a flow rate of 4.0 mL/min under reduced pressure. After the sublimation purification, 2.1 g of a light yellow solid, which was the object, was recovered, at a yield of 97%.

This compound was identified as 3-(biphenyl-3-yl)-9-[4-(10-phenyl-9-anthryl)phenyl]-9H-carbazole (CzPAmB) which was the objective compound by nuclear magnetic resonance (NMR). ¹H NMR data of the obtained compound is shown below. ¹H NMR (300 MHz, CDCl₃): δ(ppm)=7.36-7.67 (m, 16H), 7.70-7.88 (m, 14H), 7.98 (s, 1H), 7.27 (d, J=7.2 Hz, 1H), 8.47 (d, J=1.5 Hz, 1H)

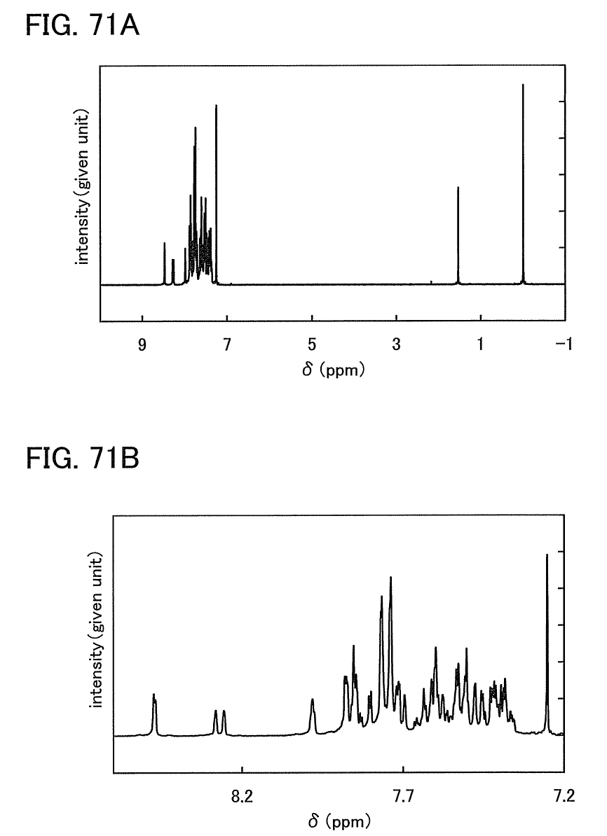

Further, the ¹H NMR chart is illustrated in FIGS. 71A and 71B. Note that FIG. 71B is a chart showing an enlarged portion of FIG. 71A in the range of from 7.2 ppm to 8.6 ppm.

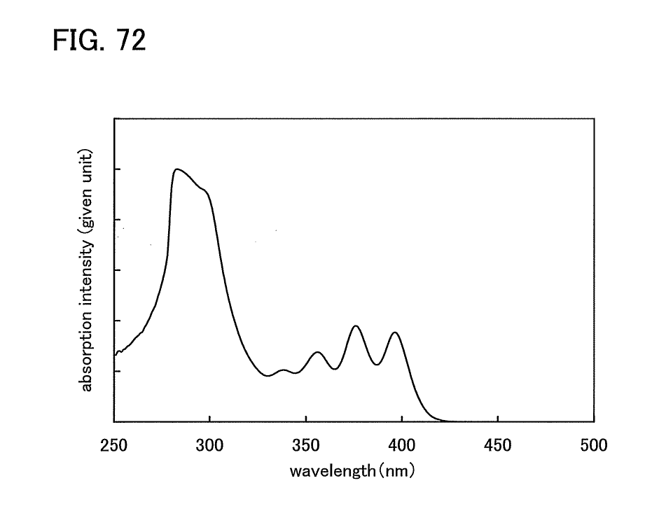

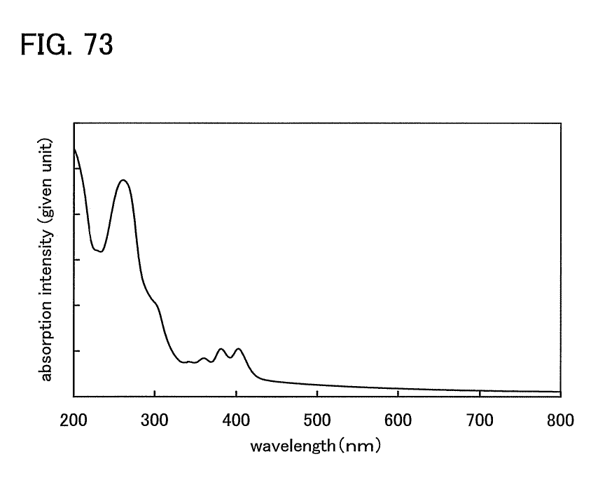

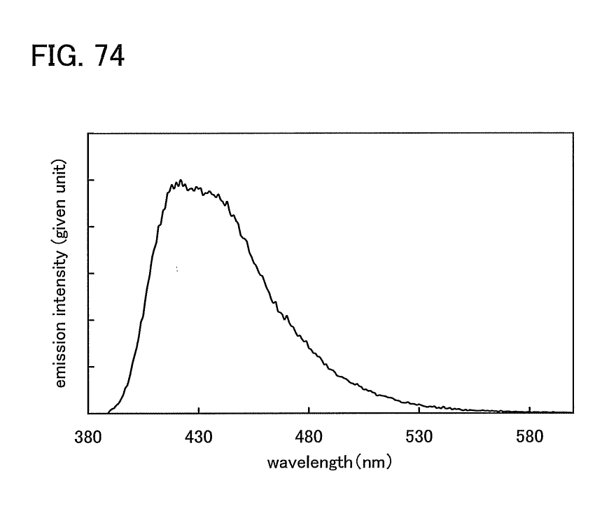

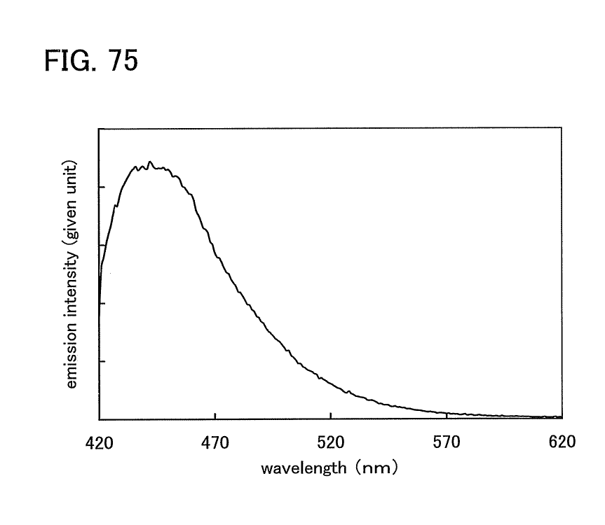

FIG. 72 illustrates an absorption spectrum of CzPAmB included in a toluene solution. FIG. 73 illustrates an absorption spectrum of a thin film of CzPAmB. An ultraviolet-visible spectrophotometer (V-550, manufactured by JASCO Corporation) was used for the measurement. The solution was put in a quartz cell and the thin film was formed by evaporation onto a quartz substrate to manufacture a sample. As for the spectrum of the solution, the absorption spectrum obtained by subtraction of the absorption spectrum of the quartz cell including only toluene is illustrated in FIG. 72. As for the spectrum of the thin film, the absorption spectrum obtained by subtraction of the absorption spectrum of the quartz substrate is illustrated in FIG. 73. In FIG. 72 and FIG. 73, the horizontal axis represents wavelength (nm) and the vertical axis represents absorption intensity (given unit). In the case of the toluene solution, absorption was observed at around 339 nm, 356 nm, 376 nm, and 396 nm. In the case of the thin film, absorption was observed at around 341 nm, 360 nm, 381 nm, and 403 nm. The emission spectrum of the toluene solution of CzPAmB (excitation wavelength: 376 nm) is illustrated in FIG. 74. The emission spectrum of the thin film of CzPAmB (excitation wavelength: 400 nm) is illustrated in FIG. 75. In FIG. 74 and FIG. 75, the horizontal axis represents wavelength (nm), and the vertical axis represents emission intensity (given unit). In the case of the toluene solution, the maximum emission wavelength was 423 nm (excitation wavelength: 376 nm), and in the case of the thin film, the maximum emission wavelength was 443 nm (excitation wavelength: 400 nm), and thus blue light emission can be obtained.

Further, the HOMO level and LUMO level of CzPAmB in the thin film state of were measured. The HOMO level was obtained by conversion of a value of ionization potential measured with a photoelectron spectrometer (AC-2, manufactured by Riken Keiki Co., Ltd.) in the atmosphere into a negative value. The LUMO level was obtained in such a manner that the absorption edge was obtained from Tauc plot, with an assumption of direct transition, using data on the absorption spectrum of the thin film of CzPAmB in FIG. 73, and the obtained absorption edge was added to the HOMO level as an optical energy gap. As a result, the HOMO level and LUMO level of CzPAmB were found to be −5.77 eV and −2.83 eV, respectively, and the band gap was found to be 2.94 eV.

Further, oxidation-reduction reaction properties of CzPAmB were measured. The oxidation-reduction reaction properties were measured by cyclic voltammetry (CV) measurement. An electrochemical analyzer (ALS model 600A, manufactured by BAS Inc.) was used for the measurement.

A solution used in the CV measurement was prepared in such a manner that dehydrated dimethylformamide (DMF) (99.8%, catalog number; 22705-6, manufactured by Sigma-Aldrich Co.) was used as a solvent, tetraperchlorate-n-butylammonium (n-Bu₄ NClO₄) (catalog number; T0836, manufactured by Tokyo Kasei Kogyo Co., Ltd.), which was a supporting electrolyte, was dissolved in the solvent so as to have a concentration of 100 mmol/L, and an object to be measured was dissolved so as to have a concentration of 1 mmol/L. Further, a platinum electrode (a PTE platinum electrode, manufactured by BAS Inc.) was used as a working electrode. A platinum electrode (a VC-3 Pt counter electrode (5 cm), manufactured by BAS Inc.) was used as an auxiliary electrode. An Ag/Ag⁺ electrode (an RE5 nonaqueous solvent reference electrode, manufactured by BAS Inc.) was used as a reference electrode. The measurement was performed at a room temperature.

The oxidation reaction characteristics of CzPAmB were measured as follows. A scan in which the potential of the working electrode with respect to the reference electrode was changed to 1.10 V from −0.06 V and then the potential was changed to −0.06 V from 1.10 V was set as one cycle, and 100 cycle measurements were performed. Note that the scan speed of the CV measurement was set at 0.1 V/s.

The reduction reaction characteristics of CzPAmB were measured as follows. A scan in which the potential of the working electrode with respect to the reference electrode was changed to −2.33 V from −1.29 V and then the potential was changed to −1.29 V from −2.33 V was set as one cycle, and 100 cycle measurements were performed. Note that the scan speed of the CV measurement was set at 0.1 V/s.

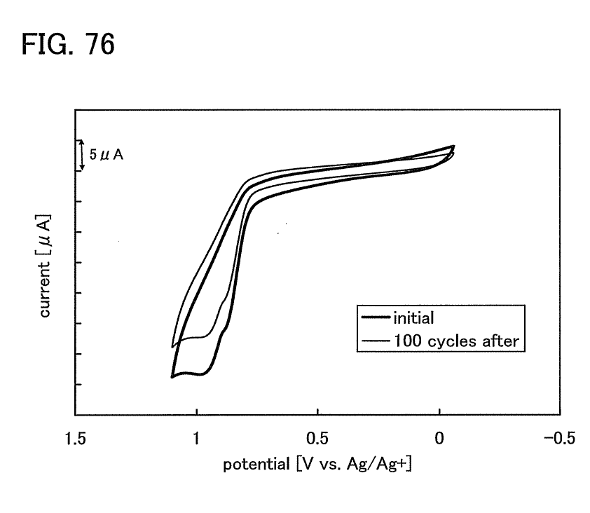

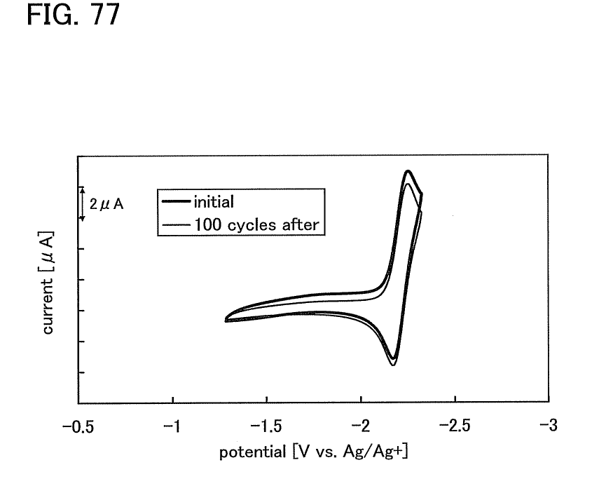

FIG. 76 illustrates CV measurement results on the oxidation reaction characteristic of CzPAmB and FIG. 77 illustrates CV measurement results on the reduction reaction characteristic of CzPAmB. In each of FIG. 76 and FIG. 77, the horizontal axis represents potential (V) of the working electrode with respect to the reference electrode and the vertical axis represents current value (μA) that flowed between the working electrode and the counter electrode. According to FIG. 76, a current indicating oxidation was observed at around +0.84 V (vs. Ag/Ag⁺ electrode). According to FIG. 77, a current indicating reduction was observed at around −2.21 V (vs. Ag/Ag⁺ electrode).

In spite of the fact that as many as 100 cycles of scan were performed, the peak position and the peak intensity of the CV curve did not significantly change in the oxidation reaction and the reduction reaction. The peak intensity remained 82% of the initial state of the oxidation reaction and 91% of the initial state of the reduction reaction. Accordingly, the carbazole derivative which is one mode of the present invention was found stable against repetition of oxidation-reduction reactions.

### Example 10

In this example, a light-emitting element, which is one mode of the present invention, will be described with reference to FIG. 26A.

Table 4 shows element structures of a light-emitting element 3-1 and a comparative light-emitting element 3-1 which were manufactured in this example. In Table 4, the mixture ratios are all represented by weight ratios.

TABLE 4

| | first | first | second | | | | fifth | second |
|---|---|---|---|---|---|---|---|---|
| | electrode | layer | layer | third layer | fourth layer | | layer | electrode |
| | 2102 | 2103 | 2104 | 2105 | 2106 | | 2107 | 2108 |
| light | ITSO | NPB:MoOx | NPB | CzPAmB:PCBAPA | Alq | Bphen | LiF | Al |
| emitting | 110 nm | (=4:1) | 10 nm | (=1:0.1) | 10 nm | 20 nm | 1 nm | 200 nm |
| element | | 50 nm | | 30 nm | | | | |
| 3-1 | | | | | | | | |
| comparative | ITSO | NPB:MoOx | NPB | CzPAoB:PCBAPA | Alq | Bphen | LiF | Al |
| light | 110 nm | (=4:1) | 10 nm | (=1:0.1) | 10 nm | 20 nm | 1 nm | 200 nm |
| emitting | | 50 nm | | 30 nm | | | | |
| element 3-1 | | | | | | | | |

Hereinafter, manufacturing methods of the light-emitting element 3-1 and the comparative light-emitting element 3-1 of this example will be described.

### (Light-Emitting Element 3-1)

First, the light-emitting element 3-1 will be described. For the light-emitting element 3-1, indium tin oxide containing silicon oxide (ITSO) was deposited over a glass substrate 2101 by a sputtering method, whereby a first electrode 2102 was formed. The thickness of the first electrode 2102 was 110 nm, and the area thereof was 2 mm×2 mm.

Next, the substrate over which the first electrode was formed was fixed to a substrate holder provided in a vacuum evaporation apparatus so that a surface of the substrate on which the first electrode was formed faced downward. The pressure was reduced to be about 10⁻⁴ Pa. Then, 4,4′-bis[N-(1-naphthyl)-N-phenylamino]biphenyl (NPB) and molybdenum(VI) oxide were co-evaporated on the first electrode 2102, whereby a layer containing a composite material of an organic compound and an inorganic compound was formed as a first layer 2103. The thickness of the first layer 2103 was 50 nm and the weight ratio between NPB and molybdenum(VI) oxide was adjusted to be 4:1 (=NPB:molybdenum oxide). Note that co-evaporation is an evaporation method in which evaporation is performed at the same time from a plurality of evaporation sources in one treatment chamber.

Next, NPB was evaporated to a thickness of 10 nm, whereby a second layer 2104 was formed as a hole-transporting layer.

Next, CzPAmB synthesized in Example 9 and 4-(10-phenyl-9-anthryl)-4′-(9-phenyl-9H-carbazol-3-yl)triphenylamine (PCBAPA) were co-evaporated onto the second layer 2104 so that the weight ratio between CzPApB and PCBAPA was 1:0.1 (=CzPApB:PCBAPA), whereby a third layer 2105 was formed as a light-emitting layer. The thickness of the third layer 2105 was 30 nm.

Next, Alq was evaporated onto the third layer 2105 to a thickness of 10 nm, and then Bphen was evaporated to a thickness of 20 nm to form a stacked layer, whereby a fourth layer 2106 was formed as an electron-transporting layer. Further, lithium fluoride (LiF) was evaporated onto the fourth layer 2106 to a thickness of 1 nm, whereby a fifth layer 2107 was formed as an electron-injecting layer. Lastly, aluminum was evaporated to a thickness of 200 nm for a second electrode 2108 which functions as a cathode. Accordingly, the light-emitting element 3-1 of this example was obtained.

### (Comparative Light-Emitting Element 3-1)

Next, the comparative light-emitting element 3-1 will be described. The comparative light-emitting element 3-1 was formed in a manner similar to that of the light-emitting element 3-1 except a third layer 2105. For the comparative light-emitting element 3-1, 3-(biphenyl-2-yl)-9-[4-(10-phenyl-9-anthryl)phenyl]-9H-carbazole (CzPAoB) represented by the following structural formula (400) and PCBAPA were co-evaporated onto the second layer 2104 so that the weight ratio between CzPAoB and PCBAPA was 1:0.1 (=CzPAoB:PCBAPA), whereby the third layer 2105 was formed as a light-emitting layer. The thickness of the third layer 2105 was 30 nm. Accordingly, the comparative light-emitting element 3-1 of this example was obtained.

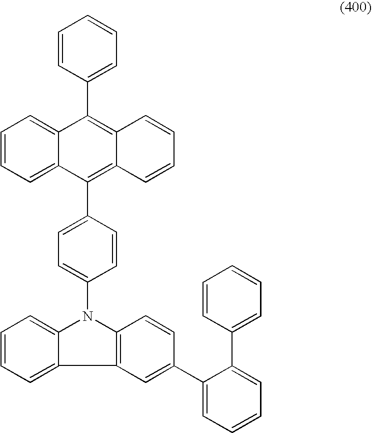

Note that in all of the above evaporation steps, a resistance heating method was used.

The thus obtained light-emitting element 3-1 and comparative light-emitting element 3-1 were sealed in a glove box under a nitrogen atmosphere without being exposed to the atmosphere. After that, operating characteristics of the light-emitting element 3-1 and the comparative light-emitting element 3-1 were measured. The measurement was performed at a room temperature (in the atmosphere in which the temperature was kept at 25° C.).

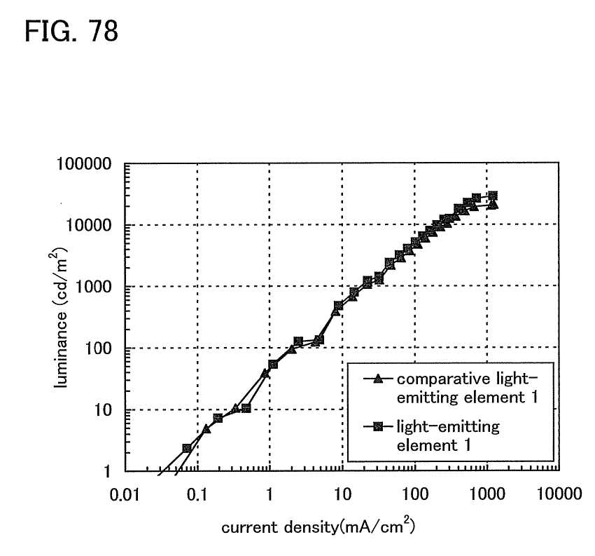

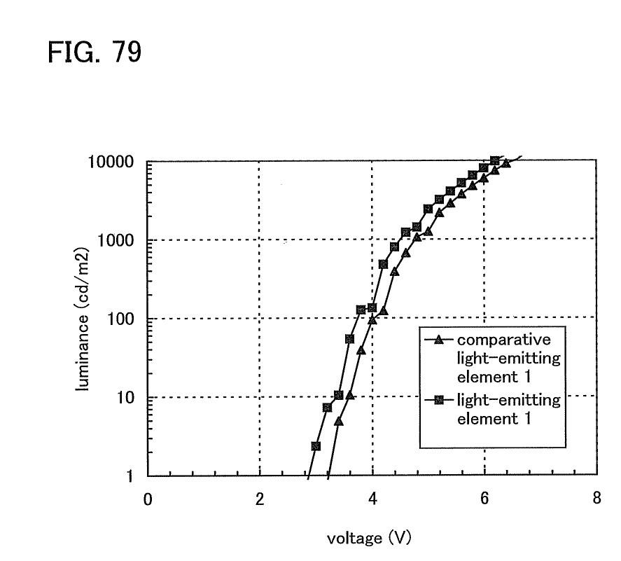

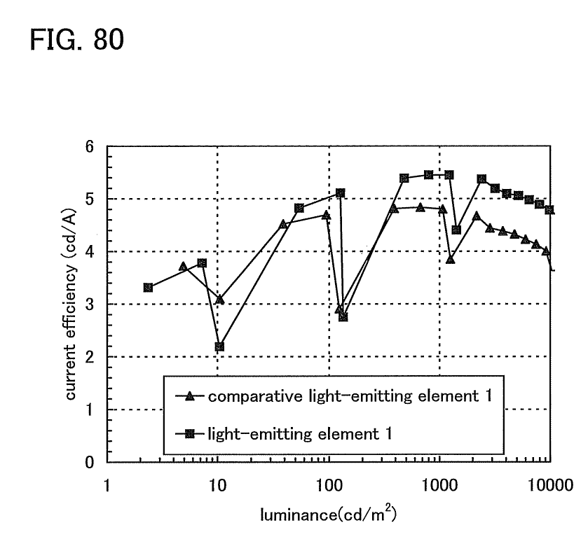

FIG. 78 illustrates the current density-luminance characteristics of the light-emitting element 3-1 and the comparative light-emitting element 3-1. In FIG. 78, the horizontal axis represents current density (mA/cm²) and the vertical axis represents luminance (cd/m²). In addition, FIG. 79 illustrates the voltage-luminance characteristics. In FIG. 79, the horizontal axis represents applied voltage (V) and the vertical axis represents luminance (cd/m²). In addition, FIG. 80 illustrates the luminance-current efficiency characteristics. In FIG. 80, the horizontal axis represents luminance (cd/m²) and the vertical axis represents current efficiency (cd/A).

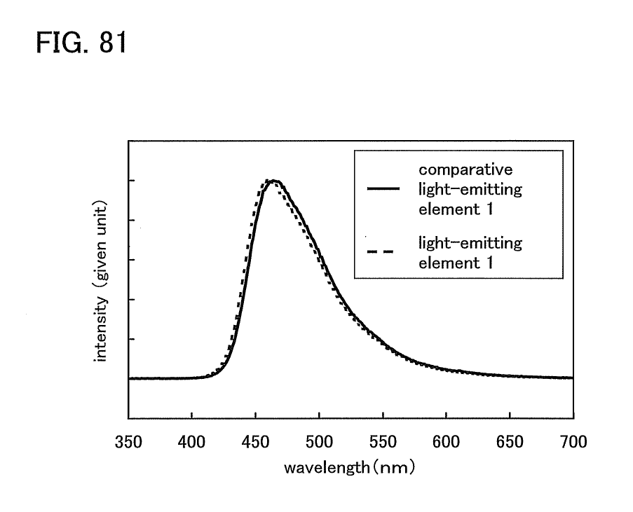

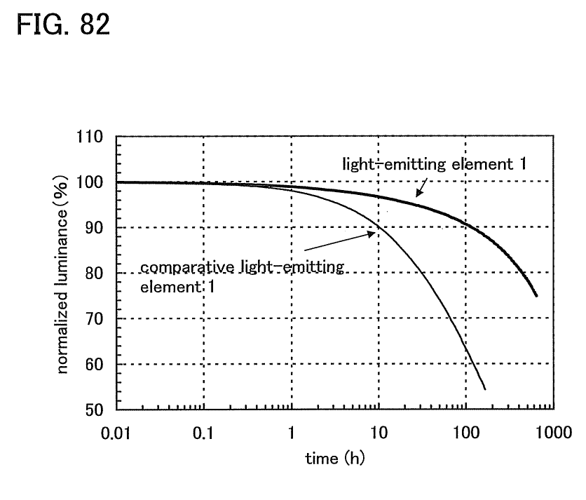

FIG. 81 illustrates emission spectra at a current of 1 mA. According to FIG. 81, light emission derived from a blue light-emitting material PCBAPA was observed both from the manufactured light-emitting element 3-1 and comparative light-emitting element 3-1. The light-emitting element 3-1 exhibited favorable blue-light emission where the CIE chromaticity coordinates were x=0.16 and y=0.21 when the luminance was 800 cd/m². Further, when the luminance was 800 cd/cm², the current efficiency was 5.4 cd/A, the external quantum efficiency was 3.7%, the voltage was 4.4 V, the current density was 14.7 mA/cm², and the power efficiency was 3.9 lm/W. The comparative light-emitting element 3-1 exhibited favorable blue-light emission where the CIE chromaticity coordinates were x=0.16 and y=0.19 when the luminance was 1070 cd/m². Further, when the luminance was 1070 cd/cm², the current efficiency was 4.8 cd/A, the external quantum efficiency was 3.4%, the voltage was 4.8 V, the current density was 22.2 mA/cm², and the power efficiency was 3.1 lm/W.

Further, reliability tests of the manufactured light-emitting element 3-1 and comparative light-emitting element 3-1 were performed. The reliability tests were performed as follows. The current with which the light-emitting element 3-1 and comparative light-emitting element 3-1 in an initial state emitted light at a luminance of 1000 cd/m² was kept constantly applied and luminance was measured at certain time intervals. Results obtained by the reliability tests of the light-emitting element 3-1 and the comparative light-emitting element 3-1 are illustrated in FIG. 82. FIG. 82 illustrates a change in luminance over time. Note that in FIG. 82, the horizontal axis represents current flow time (hour) and the vertical axis represents the proportion of luminance with respect to the initial luminance at each time, that is, normalized luminance (%).

According to FIG. 82, decline in the luminance over time of the light-emitting element 3-1 is less likely to occur than that of the comparative light-emitting element 3-1 and the light-emitting element 3-1 has long life. Even 640 hours later, the light-emitting element 3-1 kept 75% of the initial luminance and decline in the luminance over time of the light-emitting element 3-1 hardly occurred. Therefore, the light-emitting element 3-1 is a light-emitting element having long life.

This example confirmed that the light-emitting element which is one mode of the present invention has characteristics as a light-emitting element and fully functions. In addition, it was found that when the carbazole derivative of the present invention was used as a host of a light-emitting layer which emits blue light, a light-emitting element which exhibits favorable blue-light emission was obtained. Further, according to the results of the reliability tests, a highly reliable light-emitting element in which a short circuit due to defects of the film or the like is not caused even if the element is continuously made to emit light.

### Example 11

In this example, a light-emitting element having a structure different from that in above Example 10, which is one mode of the present invention, will be described with reference to FIG. 26A. In the structure of this example described below, the same reference numerals are commonly given to the same components as in the light-emitting element described in Example 10, which is one mode of the present invention, or components having similar functions to the components of the light-emitting element described in Example 10 and the description of them will not be repeated.

Element structures of a light-emitting element 3-2 and a comparative light-emitting element 3-2 manufactured in this example are shown in Table 5. In Table 5, the mixture ratios are all represented by weight ratios.

TABLE 5

| | first | first | second | | | | fifth | second |
|---|---|---|---|---|---|---|---|---|
| | electrode | layer | layer | third layer | fourth layer | | layer | electrode |
| | 2102 | 2103 | 2104 | 2105 | 2106 | | 2107 | 2108 |
| light | ITSO | NPB:MoOx | NPB | CzPAmB:2PCAPA | Alq | Bphen | LiF | Al |
| emitting | 110 nm | (=4:1) | 10 nm | (=1:0.05) | 10 nm | 20 nm | 1 nm | 200 nm |
| element | | 50 nm | | 30 nm | | | | |
| 3-2 | | | | | | | | |
| comparative | ITSO | NPB:MoOx | NPB | CzPAoB:2PCAPA | Alq | Bphen | LiF | Al |
| light | 110 nm | (=4:1) | 10 nm | (=1:0.05) | 10 nm | 20 nm | 1 nm | 200 nm |
| emitting | | 50 nm | | 30 nm | | | | |
| element 3-2 | | | | | | | | |

Manufacturing methods of the light-emitting element 3-2 and the comparative light-emitting element 3-2 of this example will be described below.

### (Light-Emitting Element 3-2)

First, the light-emitting element 3-2 will be described. The light-emitting element 3-2 was formed in a manner similar to that of the light-emitting element 3-1 described in Example 10, except a third layer 2105. For the light-emitting element 3-2, CzPAmB synthesized in Example 9 and 9,10-diphenyl-2-[N-phenyl-N-(9-phenyl-9H-carbazol-3-yl)amino]anthracene (2PCAPA) were co-evaporated onto the second layer 2104 so that the weight ratio between CzPAmB and 2PCAPA was 1:0.05 (=CzPAmB:2PCAPA), whereby the third layer 2105 was formed as a light-emitting layer. The thickness of the third layer 2105 was 30 nm. Accordingly, the light-emitting element 3-2 of this example was obtained.

### (Comparative Light-Emitting Element 3-2)

Next, the comparative light-emitting element 3-2 will be described. The comparative light-emitting element 3-2 was formed in a manner similar to that of the light-emitting element 3-1 described in Example 10, except a third layer 2105. For the comparative light-emitting element 3-2, 3-(biphenyl-2-yl)-9-[4-(10-phenyl-9-anthryl)phenyl]-9H-carbazole (CzPAoB) and 2PCAPA were co-evaporated onto the second layer 2104 so that the weight ratio between CzPAoB and 2PCAPA was 1:0.05 (=CzPAoB:2PCAPA), whereby the third layer 2105 was formed as a light-emitting layer. The thickness of the third layer 2105 was 30 nm. Accordingly, the comparative light-emitting element 3-2 of this example was obtained.

Note that in all of the above evaporation steps, a resistance heating method was used.

The thus obtained light-emitting element 3-2 and comparative light-emitting element 3-2 were sealed in a glove box under a nitrogen atmosphere without being exposed to the atmosphere. After that, operating characteristics of the light-emitting element 3-2 and the comparative light-emitting element 3-2 were measured. The measurement was performed at a room temperature (in the atmosphere in which the temperature was kept at 25° C.).

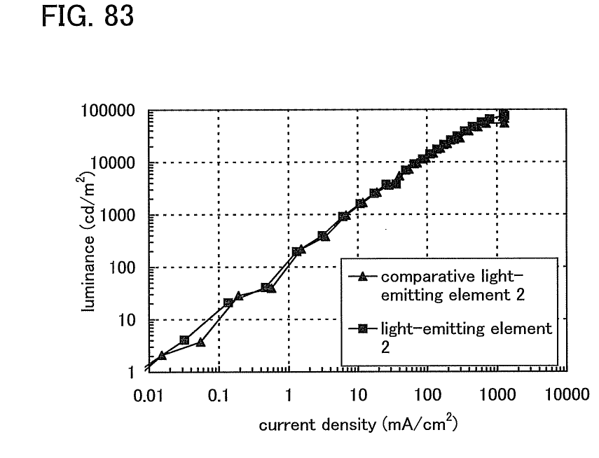

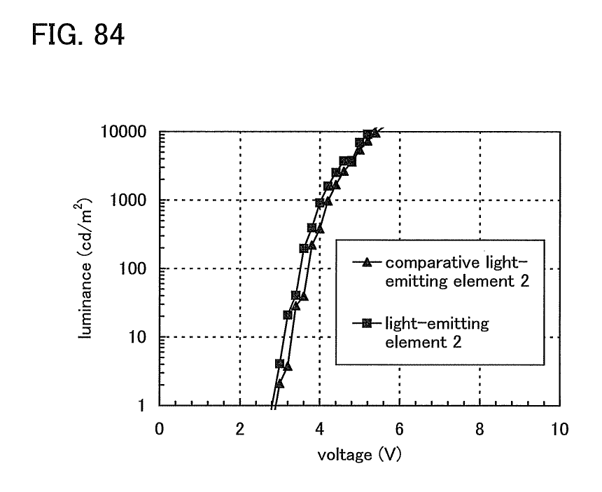

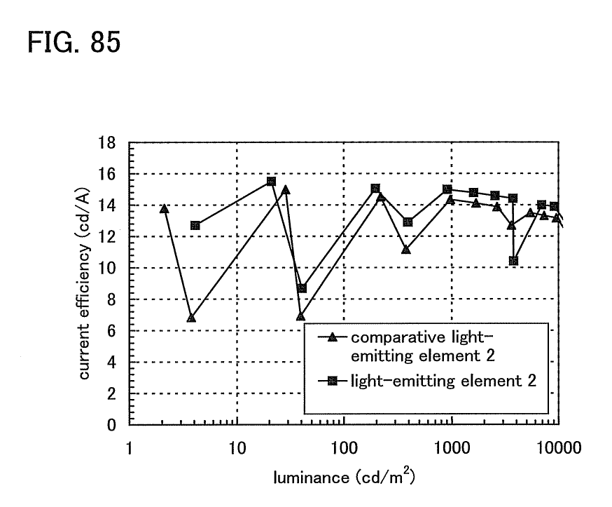

FIG. 83 illustrates the current density-luminance characteristics of the light-emitting element 3-2 and the comparative light-emitting element 3-2. In FIG. 83, the horizontal axis represents current density (mA/cm²) and the vertical axis represents luminance (cd/m²). In addition, FIG. 84 illustrates the voltage-luminance characteristics. In FIG. 84, the horizontal axis represents applied voltage (V) and the vertical axis represents luminance (cd/m²). In addition, FIG. 85 illustrates the luminance-current efficiency characteristics. In FIG. 85, the horizontal axis represents luminance (cd/m²) and the vertical axis represents current efficiency (cd/A).

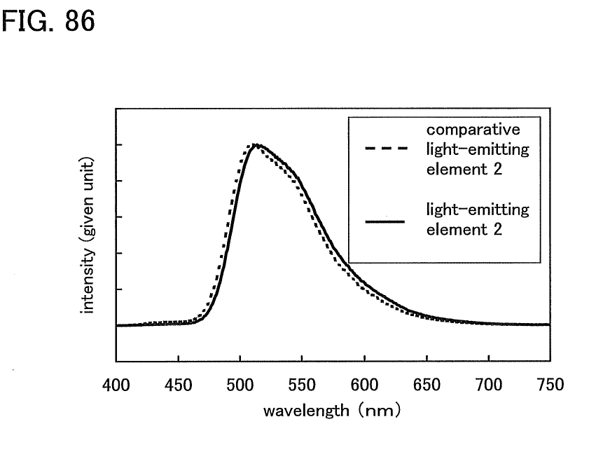

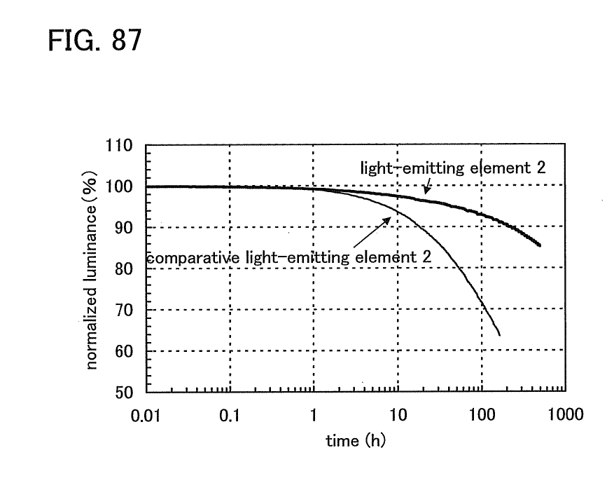

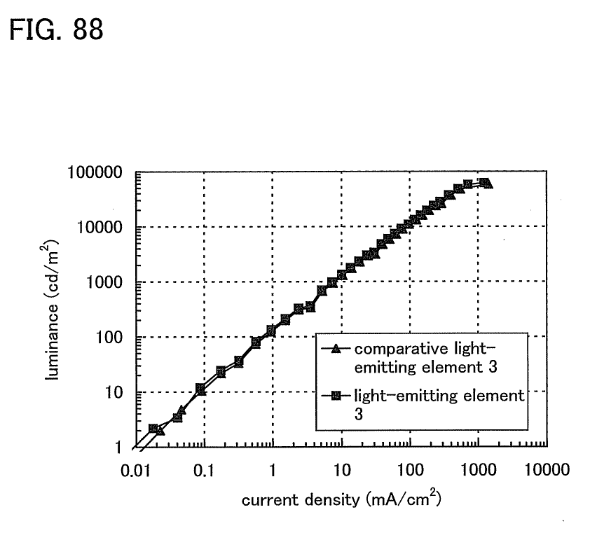

FIG. 86 illustrates emission spectra at a current of 1 mA. According to FIG. 86, light emission derived from a green light-emitting material 2PCAPA was observed both from the manufactured light-emitting element 3-2 and comparative light-emitting element 3-2. The light-emitting element 3-2 exhibited favorable green-light emission where the CIE chromaticity coordinates were x=0.30 and y=0.61 when the luminance was 2530 cd/m². Further, when the luminance was 2530 cd/cm², the current efficiency was 14.6 cd/A, the voltage was 4.4 V, the current density was 17.4 mA/cm², and the power efficiency was 10.4 lm/W. The comparative light-emitting element 3-2 exhibited favorable green-light emission where the CIE chromaticity coordinates were x=0.28 and y=0.60 when the luminance was 2650 cd/m². Further, when the luminance was 2650 cd/cm², the current efficiency was 13.9 cd/A, the voltage was 4.6 V, the current density was 19.1 mA/cm², and the power efficiency was 9.5 lm/W.

Further, reliability tests of the manufactured light-emitting element 3-2 and comparative light-emitting element 3-2 were performed. The reliability tests were performed as follows. The current with which the light-emitting element 3-2 and comparative light-emitting element 3-2 in an initial state emitted light at a luminance of 3000 cd/m² was kept constantly applied and luminance was measured at certain time intervals. Results obtained by the reliability tests of the light-emitting element 3-2 and the comparative light-emitting element 3-2 are illustrated in FIG. 87. FIG. 87 illustrates a change in luminance over time. Note that in FIG. 87, the horizontal axis represents current flow time (hour) and the vertical axis represents the proportion of luminance with respect to the initial luminance at each time, that is, normalized luminance (%).

According to FIG. 87, decline in luminance over time of the light-emitting element 3-2 is less likely to occur than that of the comparative light-emitting element 3-2 and the light-emitting element 3-2 has long life. Even 500 hours later, the light-emitting element 3-2 kept 85% of the initial luminance and decline in the luminance over time of the light-emitting element 3-2 hardly occurred. Therefore, the light-emitting element 3-2 is a light-emitting element having long life.

This example confirmed that the light-emitting element which is one mode of the present invention has characteristics as a light-emitting element and fully functions. In addition, it was found that the carbazole derivative of the present invention was used as a host of a light-emitting layer which emits green light, a light-emitting element which exhibits favorable green-light emission was obtained. Further, according to the results of the reliability tests, a highly reliable light-emitting element in which a short circuit due to defects of the film or the like is not caused even if the element is continuously made to emit light.

### Example 12

In this example, a light-emitting element having a structure different from that in above Example 10 and Example 11, which is one mode of the present invention, will be described with reference to FIG. 26B. In the structure of this example described below, the same reference numerals are commonly given to the same components as in the light-emitting element described in Example 10 and Example 11, which is one mode of the present invention, or components having similar functions to the components of light-emitting element described in Example 10 and Example 11 and the description of them will not be repeated.

A light-emitting element 3-3 of this example has a structure in which a layer 2116 which controls movement of electron carriers is provided between the third layer 2105 (the light-emitting layer) and the fourth layer 2106 (the electron-transporting layer) of the light-emitting element 3-2 described in Example 11. In addition, a comparative light-emitting element 3-3 of this example has a structure in which a layer 2116 which controls movement of electron carriers is provided between the third layer 2105 (the light-emitting layer) and the fourth layer 2106 (the electron-transporting layer) of the comparative light-emitting element 3-2 described in Example 11. Element structures of the light-emitting element 3-3 and the comparative light-emitting element 3-3 which were manufactured in this example are shown in Table 6. In Table 6, mixture ratios are all represented by weight ratios.

TABLE 6

| | | | | | layer | | | |
|---|---|---|---|---|---|---|---|---|
| | | | | | which | | | |
| | | | | | controls | | | |
| | | | | | movement | | | |
| | | | | | of | | | |
| | first | first | second | | electron | fourth | fifth | second |
| | electrode | layer | layer | third layer | carriers | layer | layer | electrode |
| | 2102 | 2103 | 2104 | 2105 | 2116 | 2106 | 2107 | 2108 |
| light | ITSO | NPB:MoOx | NPB | CzPAmB:2PCAPA | Alq:DPQd | Bphen | LiF | Al |
| emitting | 110 nm | (=4:1) | 10 nm | (=1:0.05) | (=1:0.005) | 20 nm | 1 nm | 200 nm |
| element | | 50 nm | | 30 nm | 10 nm | | | |
| 3-3 | | | | | | | | |
| comparative | ITSO | NPB:MoOx | NPB | CzPAoB:2PCAPA | Alq:DPQd | Bphen | LiF | Al |
| light | 110 nm | (=4:1) | 10 nm | (=1:0.05) | (=1:0.005) | 20 nm | 1 nm | 200 nm |
| emitting | | 50 nm | | 30 nm | 10 nm | | | |
| element 3-3 | | | | | | | | |

Manufacturing methods of the light-emitting element 3-3 and the comparative light-emitting element 3-3 of this example will be described below.

### (Light-Emitting Element 3-3)

First, the light-emitting element 3-3 will be described. The light-emitting element 3-3 was formed in a manner similar to that of the light-emitting element 3-2 described in Example 11 up to the formation of the third layer 2105. After the formation of the third layer 2105, Alq and N,N′-diphenylquinacridone (DPQd) were co-evaporated onto the third layer 2105 so that the weight ratio between Alq and DPQd was 1:0.005 (=Alq:DPQd), whereby the layer 2116 having a thickness of 10 nm which controls movement of electron carriers was formed.

Next, Bphen was evaporated to a thickness of 20 nm, whereby a fourth layer 2106 which functions as an electron-transporting layer was formed. Furthermore, lithium fluoride was evaporated onto the fourth layer 2106 to a thickness of 1 mm, whereby a fifth layer 2107 was formed as an electron-injecting layer. Lastly, aluminum was evaporated to a thickness of 200 nm, whereby a second electrode 2108 which functions as a cathode was formed. Accordingly, the light-emitting element 3-3 of this example was obtained.

### (Comparative Light-Emitting Element 3-3)

Next, the comparative light-emitting element 3-3 will be described. The comparative light-emitting element 3-3 was formed in a manner similar to that of the comparative light-emitting element 3-2 described in Example 11 up to the formation of the third layer 2105. After the formation of the third layer 2105, Alq and DPQd were co-evaporated to a thickness of 10 nm onto the third layer 2105 so that the weight ratio between Alq and DPQd was 1:0.005 (=Alq:DPQd), whereby the layer 2116 which controls movement of electron carriers was formed.

Next, Bphen was evaporated to a thickness of 20 nm, whereby a fourth layer 2106 which functions as an electron-transporting layer was formed. Furthermore, lithium fluoride was evaporated onto the fourth layer 2106 to a thickness of 1 nm, whereby a fifth layer 2107 was formed as an electron-injecting layer. Lastly, aluminum was evaporated to a thickness of 200 nm, whereby a second electrode 2108 which functions as a cathode was formed. Accordingly, the comparative light-emitting element 3-3 of this example was obtained.

Note that in all of the above evaporation steps, a resistance heating method was used. In addition, the structural formula of DPQd is shown below.

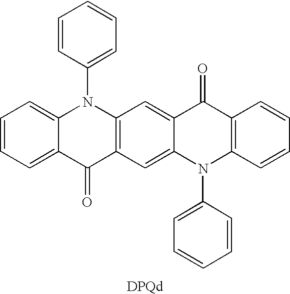

The thus obtained light-emitting element 3-3 and comparative light-emitting element 3-3 were sealed in a glove box under a nitrogen atmosphere without being exposed to the atmosphere. After that, operating characteristics of the light-emitting element 3-3 and the comparative light-emitting element 3-3 were measured. The measurement was performed at a room temperature (in the atmosphere in which the temperature was kept at 25° C.).

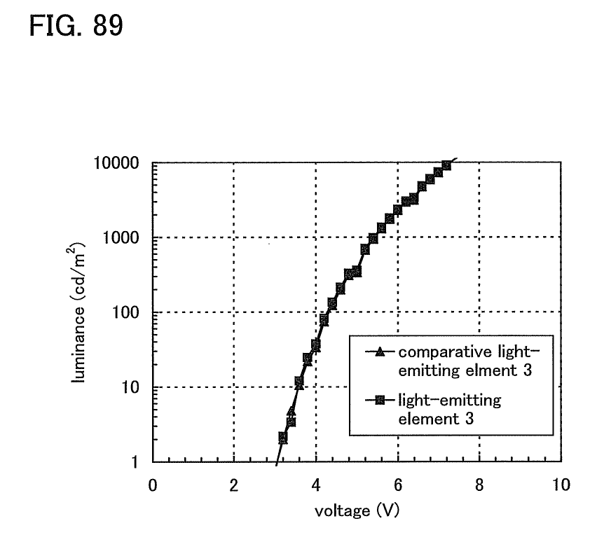

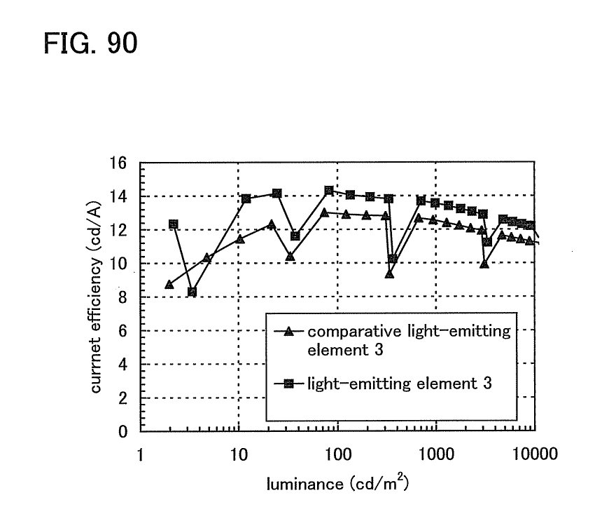

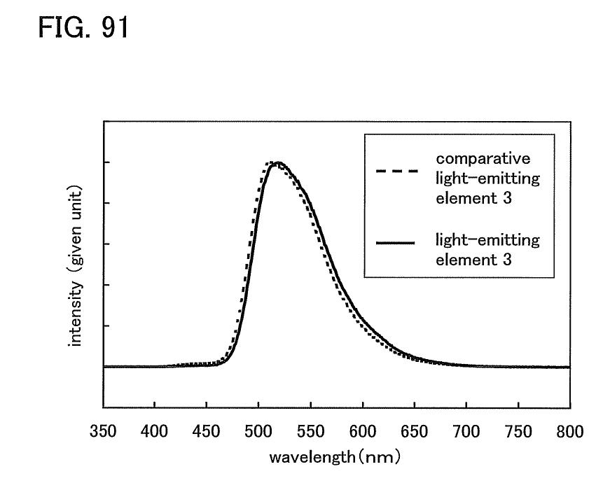

FIG. 88 illustrates the current density-luminance characteristics of the light-emitting element 3-3 and the comparative light-emitting element 3-3. In FIG. 88, the horizontal axis represents current density (mA/cm²) and the vertical axis represents luminance (cd/m²). In addition, FIG. 89 illustrates the voltage-luminance characteristics. In FIG. 89, the horizontal axis represents applied voltage (V) and the vertical axis represents luminance (cd/m²). In addition, FIG. 90 illustrates the luminance-current efficiency characteristics. In FIG. 90, the horizontal axis represents luminance (cd/m²) and the vertical axis represents current efficiency (cd/A).

FIG. 91 illustrates emission spectra at a current of 1 mA. According to FIG. 91, light emission derived from a green light-emitting material 2PCAPA was observed both from the manufactured light-emitting element 3-3 and comparative light-emitting element 3-3. The light-emitting element 3-3 exhibited favorable green-light emission where the CIE chromaticity coordinates were x=0.30 and y=0.61 when the luminance was 3040 cd/m². Further, when the luminance was 3040 cd/cm², the current efficiency was 12.9 cd/A, the voltage was 6.2 V, the current density was 23.6 mA/cm², and the power efficiency was 6.5 lm/W. The comparative light-emitting element 3-3 exhibited favorable green-light emission where the CIE chromaticity coordinates were x=0.28 and y=0.60 when the luminance was 2950 cd/m². Further, when the luminance was 2950 cd/cm², the current efficiency was 11.9 cd/A, the voltage was 6.2 V, the current density was 24.7 mA/cm², and the power efficiency was 6.0 lm/W.

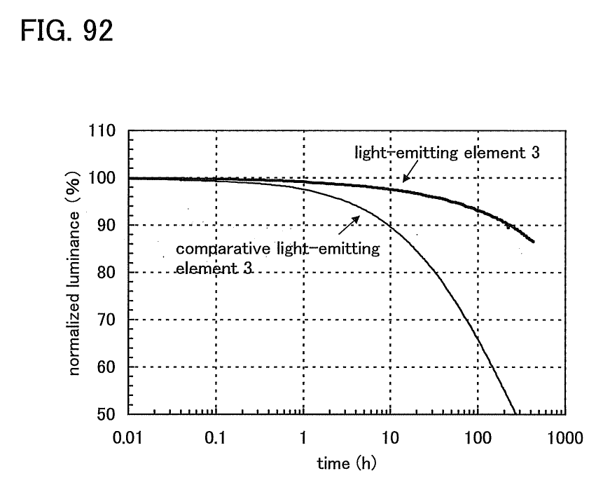

Further, reliability tests of the manufactured light-emitting element 3-3 and comparative light-emitting element 3-3 were performed. The reliability tests were performed as follows. The current with which the light-emitting element 3-3 and comparative light-emitting element 3-3 in an initial state emitted light at a luminance of 5000 cd/m² was kept constantly applied and luminance was measured at certain time intervals. Results obtained by the reliability tests of the light-emitting element 3-3 and the comparative light-emitting element 3-3 are illustrated in FIG. 92. FIG. 92 illustrates a change in luminance over time. Note that in FIG. 92, the horizontal axis represents current flow time (hour) and the vertical axis represents the proportion of luminance with respect to the initial luminance at each time, that is, normalized luminance (%).

According to FIG. 92, decline in luminance over time of the light-emitting element 3-3 is less likely to occur than that of the comparative light-emitting element 3-3 and the light-emitting element 3-3 has long life. Even 430 hours later, the light-emitting element 3-3 kept 86% of the initial luminance, decline in the luminance over time of the light-emitting element 3-3 hardly occurred. Therefore, the light-emitting element 3-3 is a light-emitting element having long life.

This example confirmed that the light-emitting element which is one mode of the present invention has characteristics as a light-emitting element and fully functions. In addition, it was found that when the carbazole derivative of the present invention was used as a host of a light-emitting layer which emits green light, a light-emitting element which exhibits favorable green-light emission was obtained. Further, according to the results of the reliability tests, a highly reliable light-emitting element in which a short circuit due to defects of the film or the like is not caused even if the element is continuously made to emit light.

Further, the life of the light-emitting element 3-3 of this example, even in the case where the reliability test was performed at a higher luminance than in the reliability test of the light-emitting element 3-2 of Example 10, was as long as that of the light-emitting element 3-2, which showed that when the light-emitting element 3-3 is used together with a functional layer which controls movement of electron carriers, a light-emitting element having longer life can be obtained.

### Example 13

In this example, the material used in other examples will be described.

### Synthesis Example of PCBAPA

Hereinafter, a synthesis method of 4-(10-phenyl-9-anthryl)-4′-(9-phenyl-9H-carbazol-3-yl)triphenylamine (PCBAPA) used in the above examples will be described.

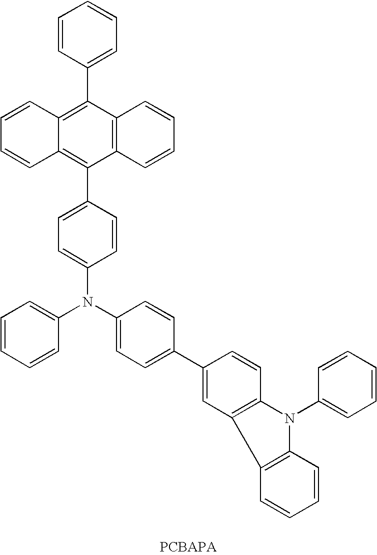

### Step 1: Synthesis of 9-phenyl-9H-carbazole-3-boronic acid

Into a 500 mL three-neck flask was put 10 g (31 mmol) of 3-bromo-9-phenyl-9H-carbazole, and the air in the flask was replaced with nitrogen. Into the flask was put 150 mL of tetrahydrofuran (THF) so that 3-bromo-9-phenyl-9H-carbazole was dissolved therein. This solution was cooled to −80° C. Into this solution was dropped 20 mL (32 mmol) of n-butyllithium (a 1.58 mol/L hexane solution) with a syringe. After the dropping, the solution was stirred at the same temperature for 1 hour. After the stir, 3.8 mL (34 mmol) of trimethyl borate was added to the solution, and the solution was stirred for about 15 hours while the temperature of the solution was brought back to a room temperature. After the stir, about 150 mL (1.0 mol/L) of dilute hydrochloric acid was added to the solution, and then the solution was stirred for 1 hour. After the stir, the aqueous layer of the mixture was extracted with ethyl acetate and the extracted solution and the organic layer were washed together with a saturated sodium bicarbonate solution. The organic layer was dried with magnesium sulfate, and this mixture was subjected to gravity filtration. The obtained filtrate was concentrated to give an oily light brown substance. The obtained oily substance was dried under reduced pressure to give 7.5 g of a light brown solid, which was the object, at a yield of 86%. The synthetic scheme of Step 1 is shown in the following (X-1).

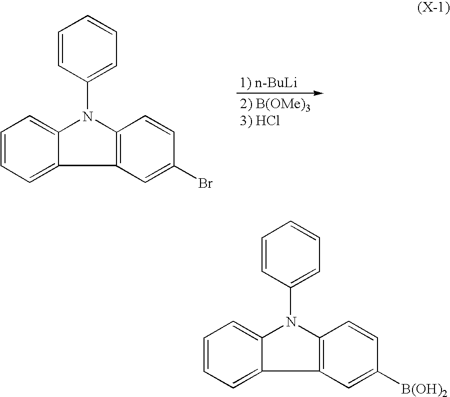

### Step 2: Synthesis of 4-(9-phenyl-9H-carbazol-3-yl)diphenylamine (PCBA)

Into a 500 mL three-neck flask were put 6.5 g (26 mmol) of 4-bromodiphenylamine, 7.5 g (26 mmol) of 9-phenyl-9H-carbazole-3-boronic acid, and 400 mg (1.3 mmol) of tri(ortho-tolyl)phosphine, and the air in the flask was replaced with nitrogen. To this mixture were added 100 mL of toluene, 50 mL of ethanol, and 14 mL of a potassium carbonate solution (2.0 mol/L). The mixture was stirred to be degassed while the pressure was reduced. After the degassing, 67 mg (30 mmol) of palladium(II) acetate was added. This mixture was refluxed at 100° C. for 10 hours. After the reflux, the aqueous layer of the mixture was extracted with toluene and the extracted solution and the organic layer were washed together with saturated saline. The organic layer was dried with magnesium sulfate, and this mixture was subjected to gravity filtration. The obtained filtrate was concentrated to give an oily light brown substance. The obtained oily substance was purified by silica gel column chromatography (a developing solvent was a mixed solvent of hexane and toluene (hexane:toluene=4:6)) to give a white solid. The white solid was recrystallized with a mixed solvent of dichloromethane and hexane to give 4.9 g of a white solid, which was the object, at a yield of 45%. The synthetic scheme of Step 2 is shown in the following (X-2).

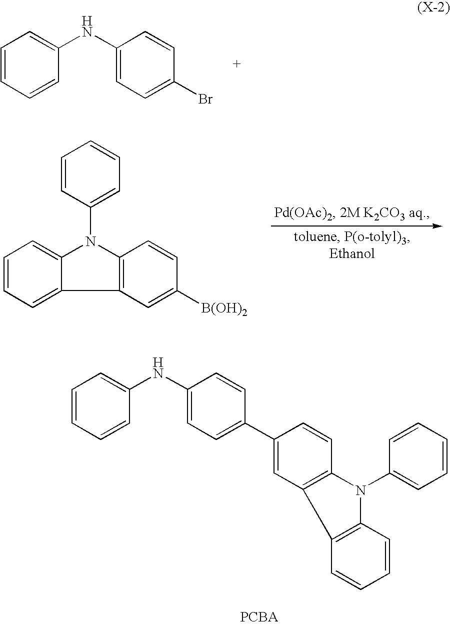

Note that the solid obtained in above Step 2 was analyzed by nuclear magnetic resonance (NMR). The measurement data of ¹H NMR is shown below. The measurement result shows that PCBA, which serves as a source material of synthesis of PCBAPA, was obtained.

¹H NMR (DMSO-d₆, 300 MHz): δ=6.81-6.86 (m, 1H), 7.12 (dd, J₁=0.9 Hz, J₂=8.7 Hz, 2H), 7.19 (d, J=8.7 Hz, 2H), 7.23-7.32 (m, 3H), 7.37-7.47 (m, 3H), 7.51-7.57 (m, 1H), 7.61-7.73 (m, 7H) 8.28 (s, 1H), 8.33 (d, J=7.2 Hz, 1H), 8.50 (d, J=1.5 Hz, 1H)

### Step 3: Synthesis of PCBAPA

Into a 300 mL three-neck flask were put 7.8 g (12 mmol) of 9-(4-bromophenyl)-10-phenylanthracene, 4.8 g (12 mmol) of PCBA, and 5.2 g (52 mmol) of sodium tert-butoxide, and the air in the flask was replaced with nitrogen. To this mixture were added 60 mL of toluene and 0.30 mL of tri(tert-butyl)phosphine (a 10 wt % hexane solution). This mixture was stirred to be degassed while the pressure was reduced. After the degassing, 136 mg (0.24 mmol) of bis(dibenzylideneacetone)palladium(0) acetate was added. This mixture was stirred at 100° C. for 3 hours. After the stir, about 50 mL of toluene was added to this mixture, and the mixture was subjected to suction filtration through Celite (Catalog No. 531-16855, manufactured by Wako Pure Chemical Industries, Ltd.), alumina, and Florisil (Catalog No. 540-00135 manufactured by Wako Pure Chemical Industries, Ltd.). The obtained filtrate was concentrated to give a yellow solid. This solid was recrystallized with a mixed solvent of toluene and hexane to give 6.6 g of light yellow powder of PCBAPA, which was the object, at a yield of 75%. The synthetic scheme of Step 3 is shown in the following (X-3).

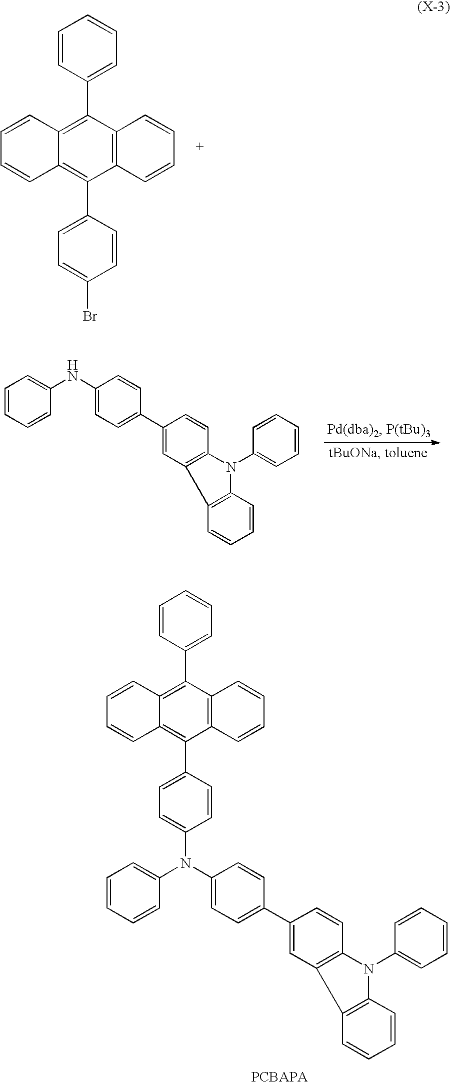

Note that the solid obtained in above Step 3 was analyzed by nuclear magnetic resonance (NMR). The measurement data of ¹H NMR is shown below. The measurement result shows that PCBAPA was obtained.

¹H NMR (CDCl₃, 300 MHz): δ=7.09-7.14 (m, 1H), 7.28-7.72 (m, 33H), 7.88 (d, J=8.4 Hz, 2H), 8.19 (d, J=7.2 Hz, 1H), 8.37 (d, J=1.5 Hz, 1H)

### Synthesis Example 1 of CzPAoB

Hereinafter, a synthesis method of 3-(biphenyl-2-yl)-9-[4-(10-phenyl-9-anthryl)phenyl]-9H-carbazole (CzPAoB) represented by the following structural formula (400) will be described.

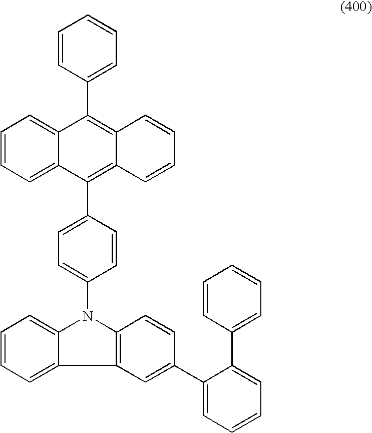

### Step 1

A synthesis method of 3-(biphenyl-2-yl)-9H-carbazole will be described.

Into a 100 mL three-neck flask were put 0.50 g (2.0 mmol) of 3-bromo-9H-carbazole, 0.40 g (2.0 mmol) of 2-biphenylboronic acid, and 0.15 g (0.50 mmol) of tri(ortho-tolyl)phosphine, and the air in the flask was replaced with nitrogen. To the mixture were added 30 mL of toluene, 10 mL of ethanol, and 2.0 mL of a potassium carbonate solution (0.2 mol/L). This mixture was stirred to be degassed while the pressure was reduced. To the mixture was added 23 mg (0.10 mmol) of palladium(II) acetate, and the mixture was stirred at 80° C. under a nitrogen stream for 2 hours. After the stir, the aqueous layer was extracted with toluene and the extracted solution and the organic layer were washed together with saturated saline. The organic layer was dried with magnesium sulfate, and this mixture was subjected to gravity filtration. The obtained filtrate was concentrated to give a solid. The solid was dissolved in about 10 mL of toluene. The solution was subjected to suction filtration through Celite (Catalog No. 531-16855 manufactured by Wako Pure Chemical Industries, Ltd.), alumina, and Florisil (Catalog No. 540-00135 manufactured by Wako Pure Chemical Industries, Ltd.). The obtained filtrate was concentrated to give a white solid. The obtained white solid was recrystallized with a mixed solvent of toluene and hexane to give 0.40 g of white power, which was the object, at a yield of 61%.

A synthetic scheme (E-1) of 3-(biphenyl-2-yl)-9H-carbazole is shown below.

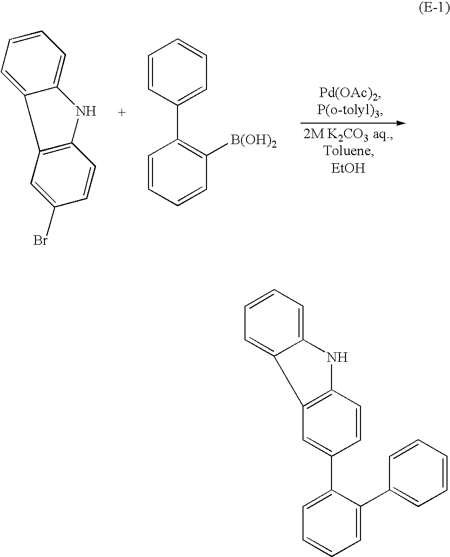

### Step 2

A synthesis example of CzPAoB will be described.

Into a 100 mL three-neck flask were put 0.51 g (1.2 mmol) of 9-(4-bromophenyl)-10-phenylanthracene, 0.40 g (1.2 mmol) of 3-(biphenyl-2-yl)-9H-carbazole, and 0.24 g (2.5 mmol) of sodium tert-butoxide. The air in the flask was replaced with nitrogen. Then, to the mixture was added 20 mL of toluene and 0.20 mL of tri(tert-butyl)phosphine (a 10 wt % hexane solution). This mixture was stirred to be degassed while the pressure was reduced. After the degassing, 36 mg (0.062 mmol) of bis(dibenzylideneacetone)palladium(0) was added to the mixture. The mixture was stirred at 110° C. under a nitrogen stream for 2 hours. After the stir, the mixture was subjected to suction filtration through Celite (Catalog No. 531-16855, manufactured by Wako Pure Chemical Industries, Ltd.), alumina, and Florisil (Catalog No. 540-00135, manufactured by Wako Pure Chemical Industries, Ltd.). The obtained filtrate was concentrated to give a solid. The solid was purified by silica gel column chromatography (a developing solvent was a mixed solvent of hexane and toluene (hexane:toluene=5:1)) to give a light yellow solid. The obtained light yellow solid was recrystallized with a mixed solvent of toluene and hexane to give 0.48 g of yellow powder, which was the object, at a yield of 60%.

Sublimation purification by train sublimation was performed on 0.47 g of the obtained light yellow powder. The sublimation purification was performed under such conditions that the light yellow powder was heated at 280° C. with an argon gas applied at a flow rate of 4.0 mL/min under reduced pressure. After the sublimation purification, 0.42 g of a light yellow solid, which was the object, was obtained at a yield of 89%. This compound was found to be 3-(biphenyl-2-yl)-9-[4-(10-phenyl-9-anthryl)phenyl]-9H-carbazole (CzPAoB), which was the object, by nuclear magnetic resonance (NMR).

A synthetic scheme (E-2) of CzPAoB is shown below.

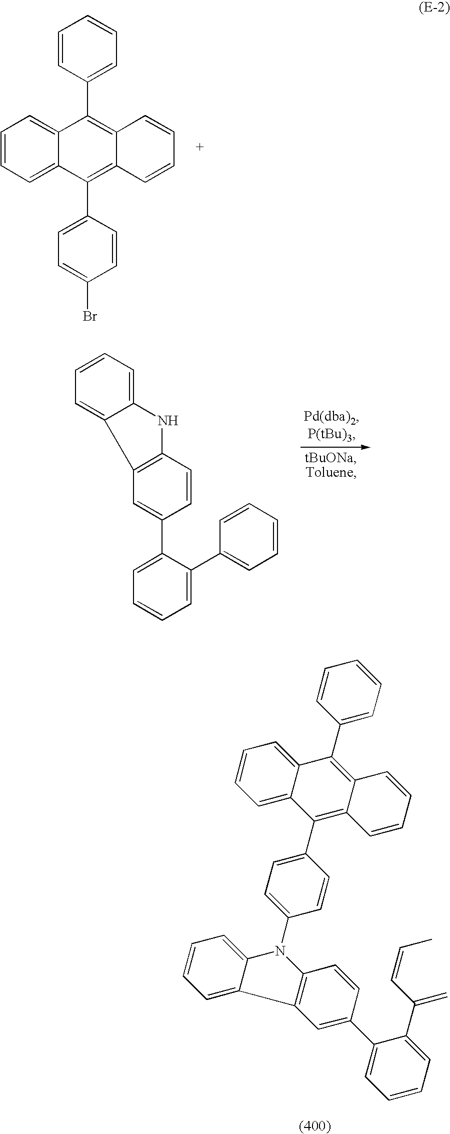

¹H NMR data of the obtained solid is shown below. ¹H NMR (DMSO-d₆, 300 MHz): δ=7.14-7.27 (m, 6H), 7.33 (t, J=7.5 Hz, 1H), 7.45-7.81 (m, 22H), 7.87 (d, J=8.1 Hz, 2H), 8.21 (d, J=9.0 Hz, 2H)

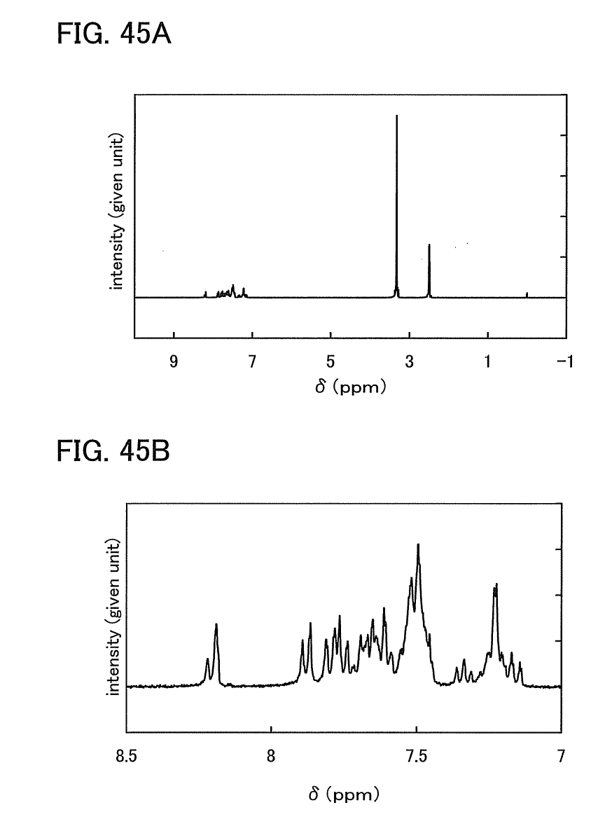

Further, the ¹H NMR chart is illustrated in FIGS. 45A and 45B. Note that FIG. 45B is a chart showing an enlarged portion of FIG. 45A in the range of from 7.0 ppm to 8.5 ppm.

Further, according to measurement of the thermophysical properties of the obtained CzPAoB under atmospheric pressure with a high vacuum differential type differential thermal balance (TG/DTA 2410SA, manufactured by Bruker AXS K.K.), the 5% weight loss temperature was 439° C. and CzPAoB was a material having favorable heat resistance.

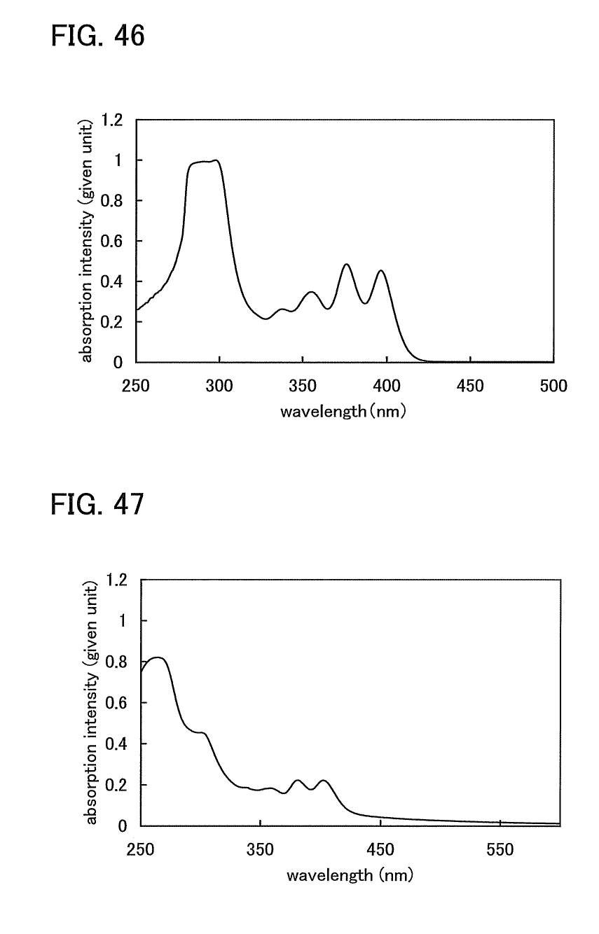

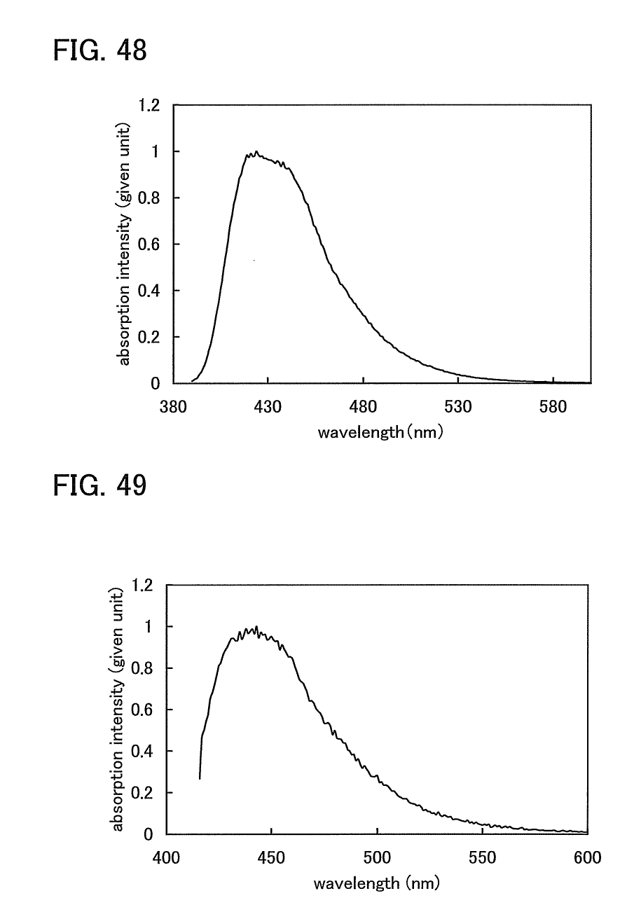

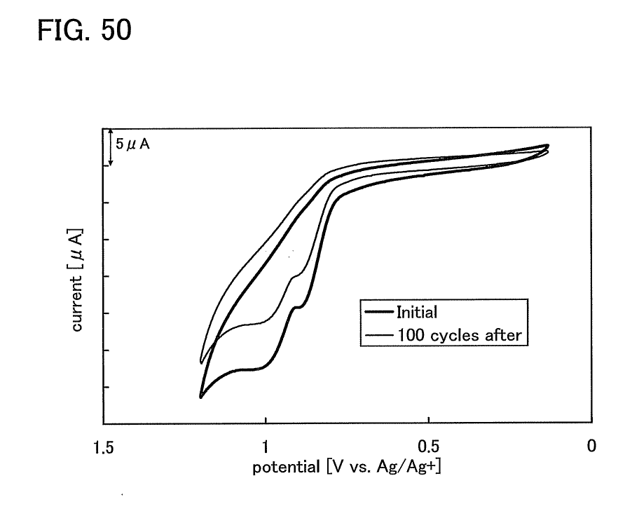

FIG. 46 illustrates an absorption spectrum of CzPAoB included in a toluene solution. FIG. 47 illustrates an absorption spectrum of a thin film of CzPAoB. An ultraviolet-visible spectrophotometer (V-550, manufactured by JASCO Corporation) was used for the measurement. The solution was put in a quartz cell and the thin film was formed by evaporation onto a quartz substrate to manufacture a sample. As for the spectrum of the solution, the absorption spectrum obtained by subtraction of the absorption spectrum of the quartz cell including only toluene is illustrated in FIG. 46. As for the spectrum of the thin film, the absorption spectrum obtained by subtraction of the absorption spectrum of the quartz substrate is illustrated in FIG. 47. In FIG. 46 and FIG. 47, the horizontal axis represents wavelength (nm) and the vertical axis represents absorption intensity (given unit). In the case of the toluene solution, absorption was observed at around 335 nm, 355 nm, 376 nm, and 397 nm. In the case of the thin film, absorption was observed at around 264 nm, 300 nm, 337 nm, 358 nm, 381 nm, and 403 nm. The emission spectrum of the toluene solution of CzPAoB (excitation wavelength: 371 nm) is illustrated in FIG. 48. The emission spectrum of the thin film of CzPAoB (excitation wavelength: 401 nm) is illustrated in FIG. 49. In FIG. 48 and FIG. 49, the horizontal axis represents wavelength (nm) and the vertical axis represents emission intensity (given unit). In the case of the toluene solution, the maximum emission wavelength was 422 nm (excitation wavelength: 371 nm). In the case of the thin film, the maximum emission wavelength was 442 nm (excitation wavelength: 401 nm).

The results of measuring the thin film of CzPAoB by photoelectron spectrometry (AC-2, manufactured by Riken Keiki Co., Ltd.) in the atmosphere indicated that the HOMO level of CzPAoB was −5.84 eV. Moreover, the absorption edge was obtained from Tauc plot, with an assumption of direct transition, using data on the absorption spectrum of the thin film of CzPAoB in FIG. 47. When the absorption edge was estimated as an optical energy gap, the energy gap was 2.96 eV. The LUMO level, which was estimated from the HOMO level of CzPAoB and the energy gap, was −2.88 eV.

Further, oxidation-reduction reaction properties of CzPAoB were measured. The oxidation-reduction reaction properties were measured by cyclic voltammetry (CV) measurement. An electrochemical analyzer (ALS model 600A, manufactured by BAS Inc.) was used for the measurement.

A solution used in the CV measurement was prepared in such a manner that dehydrated dimethylformamide (DMF) (99.8%, catalog number; 22705-6, manufactured by Sigma-Aldrich Co.) was used as a solvent, tetraperchlorate-n-butylammonium (n-Bu₄ NClO₄) (catalog number; T0836, manufactured by Tokyo Kasei Kogyo Co., Ltd.), which was a supporting electrolyte, was dissolved in the solvent so as to have a concentration of 100 mmol/L, and an object to be measured was dissolved so as to have a concentration of 1 mmol/L. Further, a platinum electrode (a PTE platinum electrode, manufactured by BAS Inc.) was used as a working electrode. A platinum electrode (a VC-3 Pt counter electrode (5 cm), manufactured by BAS Inc.) was used as an auxiliary electrode. An Ag/Ag⁺ electrode (an RE5 nonaqueous solvent reference electrode, manufactured by BAS Inc.) was used as a reference electrode. The measurement was performed at a room temperature.

The oxidation reaction characteristics of CzPAoB were measured as follows. A scan in which the potential of the working electrode with respect to the reference electrode was changed to 1.20 V from 0.13 V and then the potential was changed to 0.13 V from 1.20 V was set as one cycle, and 100 cycle measurements were performed. Note that the scan speed of the CV measurement was set at 0.1 V/s.

The reduction reaction characteristics of CzPAoB were measured as follows. A scan in which the potential of the working electrode with respect to the reference electrode was changed to −2.39 V from −1.11 V and then the potential was changed to −1.11 V from −2.39 V was set as one cycle, and 100 cycle measurements were performed. Note that the scan speed of the CV measurement was set at 0.1 V/s.

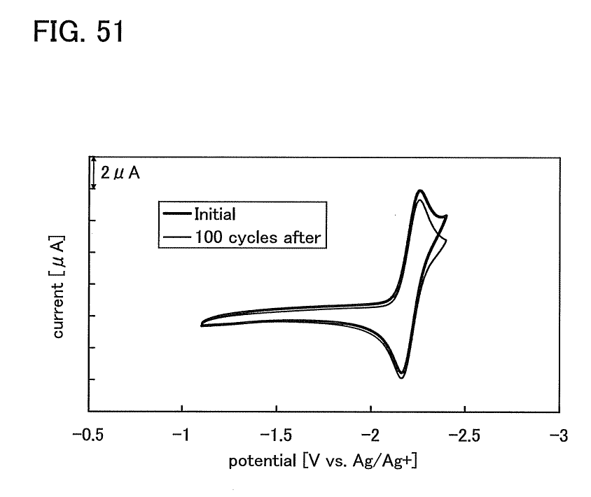

FIG. 50 illustrates CV measurement results on the oxidation reaction characteristic of CzPAoB and FIG. 51 illustrates CV measurement results on the reduction reaction characteristic of CzPAoB. In each of FIG. 50 and FIG. 51 the horizontal axis represents potential (V) of the working electrode with respect to the reference electrode, and the vertical axis represents current value (μA) that flowed between the working electrode and the counter electrode. According to FIG. 50, a current indicating oxidation was observed at around +0.85 V (vs. Ag/Ag⁺ electrode). According to FIG. 51, a current indicating reduction was observed at around −2.21 V (vs. Ag/Ag⁺ electrode).

### Synthesis Example 2 of CzPAoB

Hereinafter, another synthesis method of 3-(biphenyl-2-yl)-9-[4-(10-phenyl-9-anthryl)phenyl]-9H-carbazole (CzPAoB) represented by the structural formula (400) will be described.

A synthetic scheme is shown in the following (R-1).

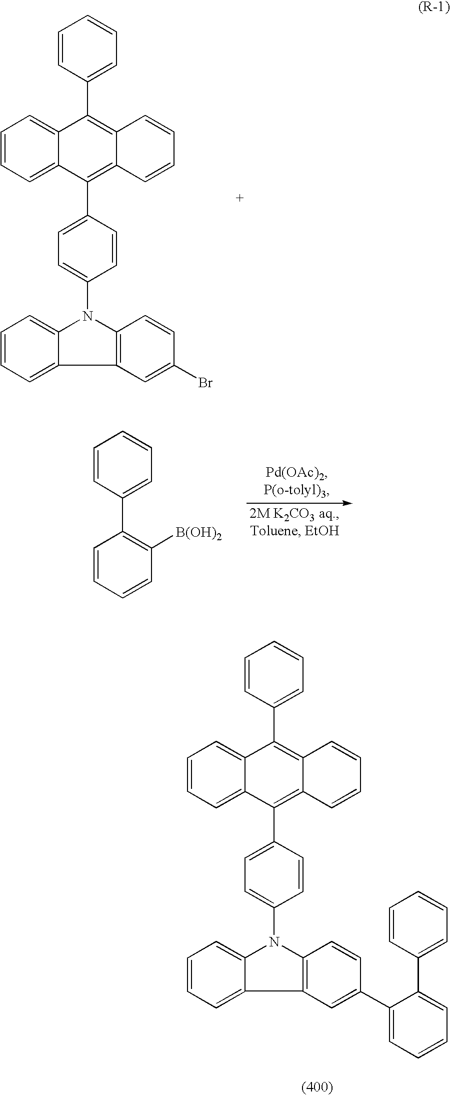

Into a 300 mL three-neck flask were put 3.0 g (5.2 mmol) of 3-bromo-9-[4-(10-phenyl-9-anthryl)phenyl]-9H-carbazole, 1.0 g (5.2 mmol) of 2-biphenylboronic acid, and 0.40 g (1.3 mmol) of tri(ortho-tolyl)phosphine, and the air in the flask was replaced with nitrogen. To the mixture were added 60 mL of toluene, mL of ethanol, and 5.0 mL of a potassium carbonate solution (0.2 mol/L). This mixture was stirred to be degassed while the pressure was reduced. After the degassing, the air in the flask was replaced with nitrogen. Then, to the mixture was added 58 mg (0.26 mmol) of palladium(II) acetate. The mixture was stirred at 80° C. under a nitrogen stream for 3 hours. After the stir, the aqueous layer of the mixture was extracted with toluene and the extracted solution and the organic layer were washed together with saturated saline. The organic layer was dried with magnesium sulfate, and this mixture was subjected to gravity filtration. The obtained filtrate was concentrated to give an oily substance. The oily substance was dissolved in about 10 mL of toluene. The solution was subjected to suction filtration through Celite (Catalog No. 531-16855, manufactured by Wako Pure Chemical Industries, Ltd.), alumina, and Florisil (Catalog No. 540-00135, manufactured by Wako Pure Chemical Industries, Ltd.). The obtained filtrate was concentrated to give an oily substance. The obtained oily substance was purified by silica gel column chromatography (a developing solvent was a mixed solvent of hexane and toluene (hexane:toluene=5:1)) to give a light yellow oily substance. The obtained light yellow oily substance was recrystallized with a mixed solvent of toluene and hexane to give 2.0 g of light yellow powder, which was the object, at a yield of 67%.

Sublimation purification by train sublimation was performed on 2.0 g of the obtained light yellow powder. The sublimation purification was performed under such conditions that the light yellow powder was heated at 280° C. with an argon gas applied at a flow rate of 4.0 mL/min under reduced pressure. After the sublimation purification, 1.9 g of a light yellow solid, which was the objective compound, was recovered in 93% yield.

As in Synthesis Example 1 of CzPAoB, this compound was found to be 3-(biphenyl-2-yl)-9-[4-(10-phenyl-9-anthryl)phenyl]-9H-carbazole (CzPAoB), which was the object, by nuclear magnetic resonance (NMR).

This application is based on Japanese Patent Application serial no. 2008-177836 filed with Japan Patent Office on Jul. 8, 2008, Patent Application serial no. 2008-181752 filed with Japan Patent Office on Jul. 11, 2008, Patent Application serial no. 2008-240355 filed with Japan Patent Office on Sep. 19, 2008, Patent Application serial no. 2008-240529 filed with Japan Patent Office on Sep. 19, 2008, and Patent Application serial no. 2008-331007 filed with Japan Patent Office on Dec. 25, 2008, the entire contents of which are hereby incorporated by reference.

### EXPLANATION OF REFERENCE

101: substrate, 102: first electrode, 103: first layer, 104: second layer, 105: third layer, 106: fourth layer, 107: second electrode, 108: EL layer, 130: layer which controls movement of electron carriers, 301: substrate, 302: first electrode, 303: first layer, 304: second layer, 305: third layer, 306: fourth layer, 307: electrode, 308: EL layer, 501: first electrode, 502: second electrode, 511: light-emitting unit, 512: light-emitting unit, 513: charge generation layer, 601: source side driver circuit, 602: pixel portion, 603: gate side driver circuit, 604: sealing substrate, 605: sealant, 607: space, 608: wiring, 610: element substrate, 611: switching TFT, 612: current control TFT, 613: first electrode, 614: insulator, 616: layer containing light-emitting substance, 617: second electrode, 618: light-emitting element, 623: n-channel TFT, 624: p-channel TFT, 901: housing, 902: liquid crystal layer, 903: backlight, 904: housing, 905: driver IC, 906: terminal, 951: substrate, 952: electrode, 953: insulating layer, 954: partition layer, 955: layer containing light-emitting substance, 956: electrode, 105a: light-emitting layer, 105b: light-emitting layer, 2001: housing, 2002: light source, 2101: glass substrate, 2102: first electrode, 2103: first layer, 2104: second layer, 2105: third layer, 2106: fourth layer, 2107: fifth layer, 2108: second electrode, 2116: layer which controls movement of electron carriers, 3001: lighting device, 3002: television device, 8401: main body, 8402: housing, 8403: display portion, 8404: audio input portion, 8405: audio output portion, 8406: operation key, 8407: external connection port, 9101: housing, 9102: supporting base, 9103: display portion, 9104: speaker portion, 9105: video input terminal, 9201: main body, 9202: housing, 9203: display portion, 9204: keyboard, 9205: external connection port, 9206: pointing device, 9401: main body, 9402: housing, 9403: display portion, 9404: audio input portion, 9405: audio output portion, 9406: operation key, 9407: external connection port, 9408: antenna, 9501: main body, 9502: display portion, 9503: housing, 9504: external connection port, 9505: remote control receiving portion, 9506: image receiving portion, 9507: battery, 9508: audio input portion, 9509: operation key, 9510: eye piece portion, 9660: main body, 9661: display portion, 9662: driver IC, 9663: receiver, 9664: film battery.

1. A carbazole derivative represented by a formula (1), 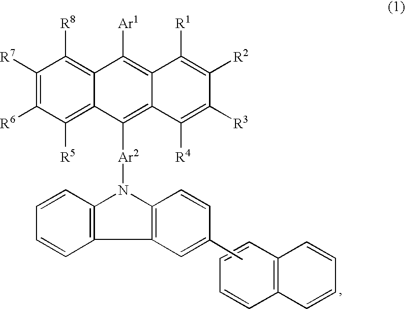
where:
Ar¹ represents an aryl group having 6 to 13 carbon atoms;
Ar² represents an arylene group having 6 to 13 carbon atoms; and
R¹ to R⁸ independently represent hydrogen or an alkyl group having 1 to 4 carbon atoms.

2. The carbazole derivative according to claim 1, wherein the carbazole derivative has the following formula (2),

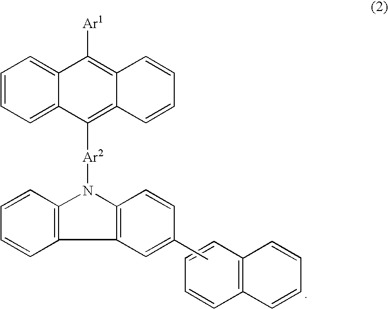

3. The carbazole derivative according to claim 1, wherein the carbazole derivative has the following formula (3), 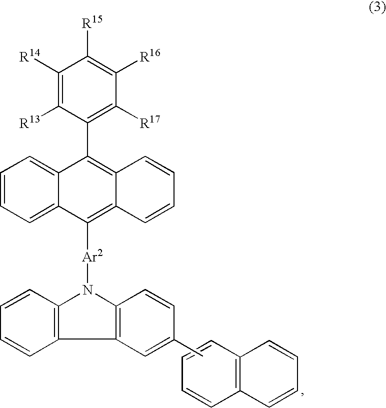
where R¹³ to R¹⁷ independently represent hydrogen, an alkyl group having 1 to 4 carbon atoms, or an aryl group having 6 to 10 carbon atoms.

4. The carbazole derivative according to claim 1, wherein the carbazole derivative has the following formula (4), 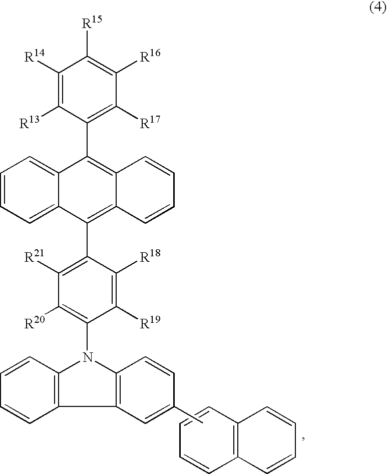
where R¹³ to R²¹ independently represent hydrogen, an alkyl group having 1 to 4 carbon atoms, or an aryl group having 6 to 10 carbon atoms.

5. The carbazole derivative according to claim 1, wherein the carbazole derivative has the following formula (101),

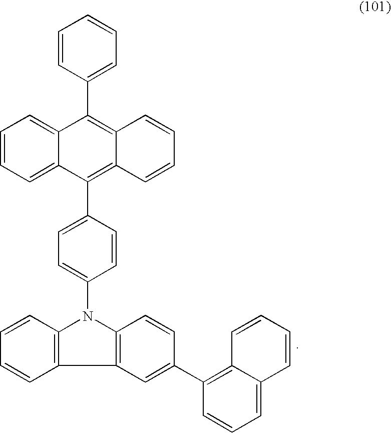

6. The carbazole derivative according to claim 1, wherein the carbazole derivative has the following formula (201),

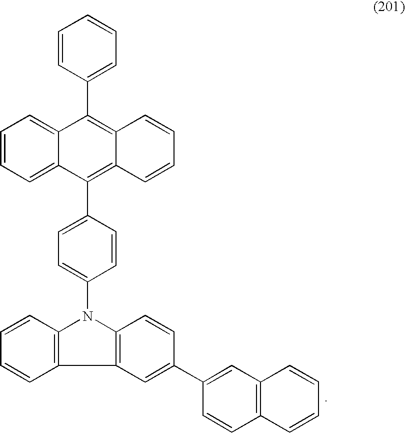

7. The carbazole derivative according to claim 1, wherein at least one of Ar¹ and Ar² has a substituent.

8. The carbazole derivative according to claim 1, wherein at least one of Ar¹ and Ar² has two or more substituents, and
wherein the two of the substituents are bonded to each other to form a ring structure.

9. A carbazole derivative represented by a formula (P1), 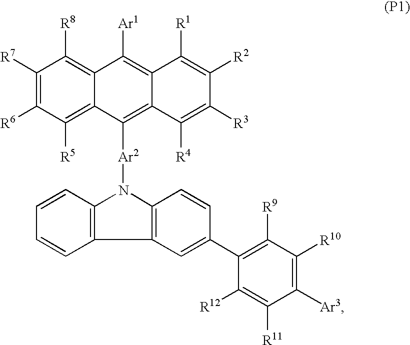
where:
R¹ to R¹² independently represent hydrogen or an alkyl group having 1 to 4 carbon atoms;
Ar¹ and Ar³ independently represent an aryl group having 6 to 13 carbon atoms; and
Ar² represents an arylene group having 6 to 13 carbon atoms.

10. The carbazole derivative according to claim 9, wherein the carbazole derivative has the following formula (P2),

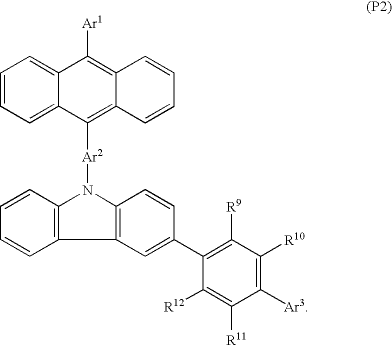

11. The carbazole derivative according to claim 9, wherein the carbazole derivative has the following formula (P3), 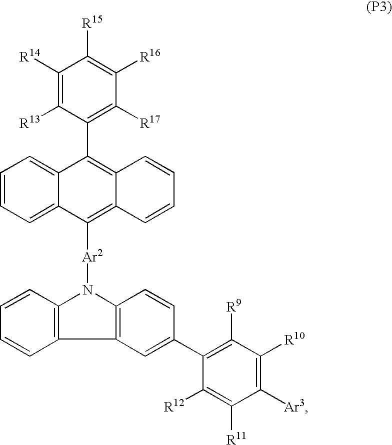
where R¹³ to R¹⁷ independently represent hydrogen, an alkyl group having 1 to 4 carbon atoms, or an aryl group having 6 to 10 carbon atoms.

12. The carbazole derivative according to claim 9, wherein the carbazole derivative has the following formula (P4), 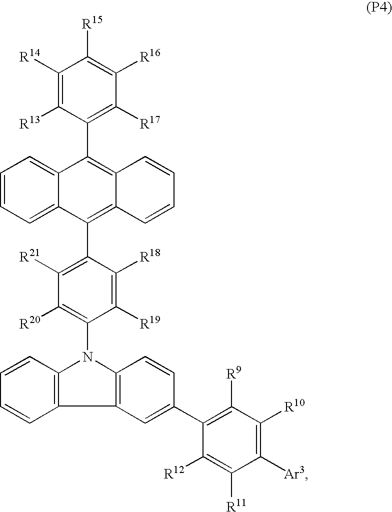
where R¹³ to R²¹ independently represent hydrogen or an alkyl group having 1 to 4 carbon atoms.

13. The carbazole derivative according to claim 9, wherein the carbazole derivative has the following formula (31),

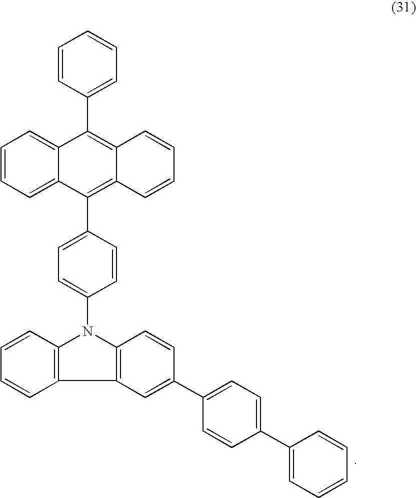

14. The carbazole derivative according to claim 9, wherein the carbazole derivative has the following formula (63),

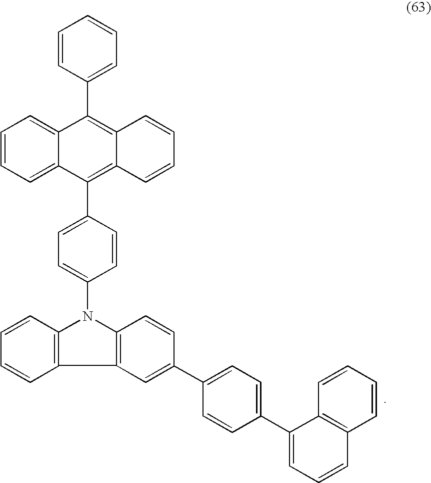

15. The carbazole derivative according to claim 9, wherein the carbazole derivative has the following formula (76),

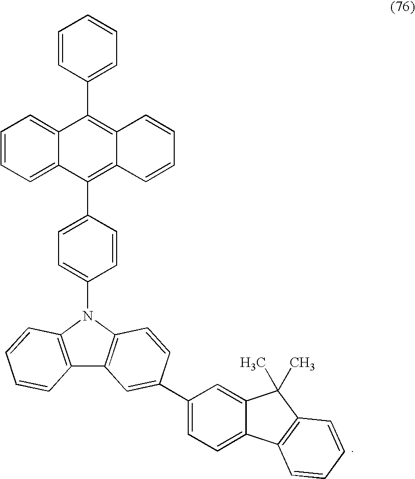

16. The carbazole derivative according to claim 9, wherein at least one of Ar¹, Ar² and Ar³ has a substituent.

17. The carbazole derivative according to claim 9, wherein at least one of Ar¹, Ar² and Ar³ has two or more substituents, and
wherein the two of the substituents are bonded to each other to form a ring structure.

18. The carbazole derivative according to claim 9, wherein Ar³ has a substituent, and
wherein the substituent of Ar³ is bonded to R¹⁰ or R¹¹ to form a ring structure.

19. A carbazole derivative represented by a formula (M1), 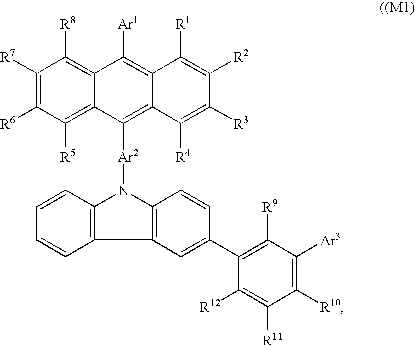
where:
R¹ to R¹² independently represent hydrogen or an alkyl group having 1 to 4 carbon atoms;
Ar¹ and Ar³ independently represent an aryl group having 6 to 13 carbon atoms; and
Ar² represents an arylene group having 6 to 13 carbon atoms.

20. The carbazole derivative according to claim 19, wherein the carbazole derivative has the following formula (M2),

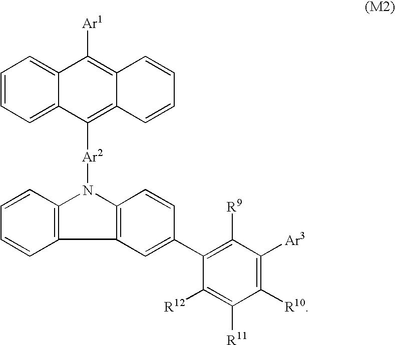

21. The carbazole derivative according to claim 19, wherein the carbazole derivative has the following formula (M3), 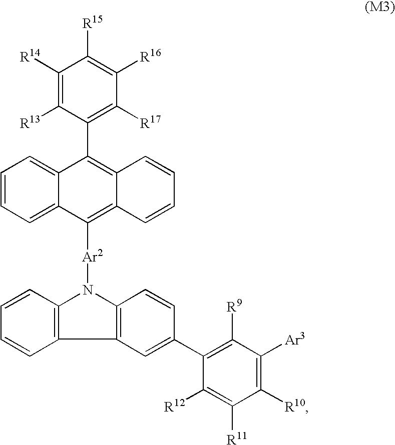
where R¹³ to R¹⁷ independently represent hydrogen, an alkyl group having 1 to 4 carbon atoms, or an aryl group having 6 to 10 carbon atoms.

22. The carbazole derivative according to claim 19, wherein the carbazole derivative has the following formula (M4), 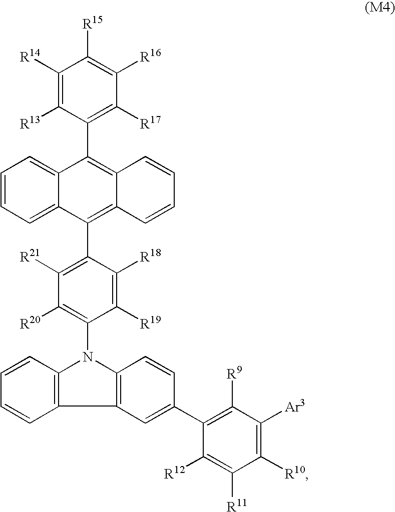
where R¹³ to R²¹ independently represent hydrogen or an alkyl group having 1 to 4 carbon atoms.

23. The carbazole derivative according to claim 19, wherein the carbazole derivative has the following formula (331),

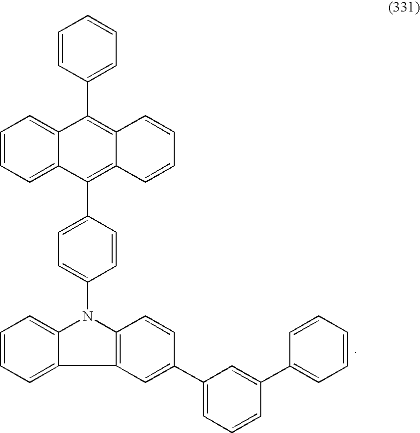

24. The carbazole derivative according to claim 19, wherein at least one of Ar¹, Ar² and Ar³ has a substituent.

25. The carbazole derivative according to claim 19, wherein at least one of Ar¹, Ar² and Ar³ has two or more substituents, and
wherein the two of the substituents are bonded to each other to form a ring structure.

26. The carbazole derivative according to claim 19, wherein Ar³ has a substituent, and
wherein the substituent of Ar³ is bonded to R⁹ or R¹⁰ to form a ring structure.

27. A light-emitting element comprising the carbazole derivative according to claim 1.

28. A light-emitting element comprising the carbazole derivative according to claim 9.

29. A light-emitting element comprising the carbazole derivative according to claim 19.
